(19) United States
(12) Patent Application Publication
(10) Pub. No.: US 2011/0077399 A1
(43) Pub. Date: Mar. 31, 2011

(54) Substituted Azole Derivatives as Therapeutic Agents

(75) Inventors: Adnan M. M. Mjalli, Oak Ridge, NC (US); Robert C. Andrews, Jamestown, NC (US); Ravindra R. Yarragunta, Greensboro, NC (US); Rongyuan Xie, Greensboro, NC (US); Govindan Subramanian, Belle Mead, NJ (US); James C. Quada, JR., High Point, NC (US); Murty N. Arimilli, Oak Ridge, NC (US); Dharma R. Polisetti, High Point, NC (US)

(73) Assignee: TRANSTECH PHARMA, INC., High Point, NC (US)

(21) Appl. No.: 12/958,237

(22) Filed: Dec. 1, 2010

Related U.S. Application Data
(63) Continuation of application No. 10/777,488, filed on Feb. 12, 2004, now abandoned.
(60) Provisional application No. 60/446,977, filed on Feb. 12, 2003.

(51) Int. Cl. C07D 413/04; C07D 233/64; C07D 405/10; C07D 401/04

(52) U.S. Cl. 544/139; 548/343.5; 548/235; 548/311.7; 548/311.4; 546/143; 546/274.1

(57) ABSTRACT

This invention provides azoles which may be useful as inhibitors of protein tyrosine phosphatases (PTPases). The present invention provides compounds of Formula (I), methods of their preparation, pharmaceutical compositions comprising the compounds and their use in treating human or animal disorders. The compounds of the invention may be useful as inhibitors of protein tyrosine phosphatases and thus can be useful for the management, treatment, control and adjunct treatment of diseases mediated by PTPase activity. Such diseases include Type I diabetes, Type II diabetes.

### STATEMENT OF RELATED APPLICATION

The present application is a continuation application of U.S. patent application Ser. No. 10/777,488, filed Feb. 12, 2004, which in turn claims the benefit of priority from U.S. Provisional Patent Application No. 60/446,977, filed Feb. 12, 2003. The disclosures of each of the aforementioned US patent applications is incorporated by reference into the present application as though each were fully set forth herein.

### FIELD OF THE INVENTION

This invention relates to compounds which may be inhibitors of protein tyrosine phosphatases (PTPases), which can be useful for the management, treatment, control, or adjunct treatment of diseases caused by over-activity of PTPases.

### BACKGROUND OF THE INVENTION

The process of protein phosphorylation is now recognized as central to the fundamental processes of cellular signal transduction. Alterations in protein phosphorylation, may therefore constitute either a physiological or pathological change in an in vivo system. Protein de-phosphorylation, mediated by phosphatases, is also central to certain signal transduction processes.

The two major classes of phosphatases are (a) protein serine/threonine phosphatases (PSTPases), which catalyze the dephosphorylation of serine and/or threonine residues on proteins or peptides; and (b) the protein tyrosine phosphatases (PTPases), which catalyze the dephosphorylation of tyrosine residues on proteins and/or peptides. A third class of phosphatases is the dual specificity phosphatases, or DSP's, which possess the ability to act both as PTPases and as PSTPases.

Among the PTPases there exist two important families, the intracellular PTPases, and the transmembrane PTPases. The intracellular PTPases include PTP1B, STEP, PTPD1, PTPD2, PTPMEG1, T-cell PTPase, PTPH1, FAP-1/BAS, PTP1D, and PTP1C. The transmembrane PTPases include LAR, CD45, PTPα, PTPβ, PTPδ, PTPε, PTPξ, PTPκ, PTPμ, PTPσ, HePTP, SAP-1, and PTP-U2. The dual-specificity phosphatases include KAP, cdc25, MAPK phosphatase, PAC-1, and rVH6.

The PTPases, especially PTP1B, are implicated in insulin insensitivity characteristic of type II diabetes (Kennedy, B. P.; Ramachandran, C. Biochem. Pharm. 2000, 60, 877-883). The PTPases, notably CD45 and HePTP, are also implicated in immune system function, and in particular T-cell function. Certain PTPases, notably TC-PTP, DEP-1, SAP-1, and CDC25, are also implicated in certain cancers. Certain PTPases, notably the bone PTPase OST-PTP, are implicated in osteoporosis. PTPases are implicated in mediating the actions of somatostatin on target cells, in particular the secretion of hormone and/or growth factor secretion.

Thus, there is a need for agents which inhibit the action of protein tyrosine phosphatases. Such agents would be useful for the treatment of Type I diabetes, Type II diabetes, immune dysfunction, AIDS, autoimmunity, glucose intolerance, obesity, cancer, psoriasis, allergic diseases, infectious diseases, inflammatory diseases, diseases involving the modulated synthesis of growth hormone or the modulated synthesis of growth factors or cytokines which affect the production of growth hormone, or Alzheimer's disease.

### SUMMARY OF THE INVENTION

This invention provides azoles which are useful as inhibitors of PTPases. In an embodiment, the present invention provides compounds of Formula (I) as depicted below, methods of their preparation, pharmaceutical compositions comprising the compounds and their use in treating human or animal disorders. The compounds of the invention are useful as inhibitors of protein tyrosine phosphatases and thus are useful for the management, treatment, control and adjunct treatment of diseases mediated by PTPase activity. Such diseases include Type I diabetes, Type II diabetes, immune dysfunction, AIDS, autoimmunity, glucose intolerance, obesity, cancer, psoriasis, allergic diseases, infectious diseases, inflammatory diseases, diseases involving the modulated synthesis of growth hormone or the modulated synthesis of growth factors or cytokines which affect the production of growth hormone, or Alzheimer's disease.

### DETAILED DESCRIPTION OF THE INVENTION

In a first aspect, the present invention provides azole inhibitors of protein tyrosine phosphatases (PTPases) which can be useful for the management and treatment of disease caused by PTPases.

In a second aspect, the present invention provides compounds of Formula (I):

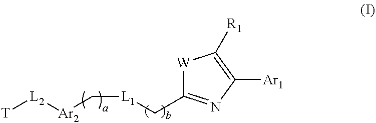

wherein a and b are, independently, equal to 0, 1, or 2, wherein the values of 0, 1, and 2 represent a direct bond, —CH₂—, and —CH₂CH₂—, respectively, and wherein the —CH₂— and —CH₂CH₂— groups are optionally substituted 1 to 2 times with a substituent group, wherein said substituent group(s) comprise: -alkyl, -aryl, -alkylene-aryl, -arylene-alkyl, -alkylene-arylene-alkyl, —O-alkyl, —O-aryl, and -hydroxyl. In an embodiment, a and b are equal to 0.
W comprises —O—, —S—, or —N(R₂)—,

wherein
R₂ comprises
   a) -hydrogen;
   b) -alkyl;
   c) -L₃-D-G
   d) -L₃-D-alkyl:
   e) -L₃-D-aryl;
   f) -L₃-D-heteroaryl;
   g) -L₃-D-cycloalkyl;
   h) -L₃-D-heterocyclyl;
   i) -L₃-D-arylene-alkyl;
   j) -L₃-D-alkylene-arylene-alkyl; and
   k) -L₃-D-alkylene-aryl;
   l) -L₃-D-alkyl-G;
   m) -L₃-D-aryl-G;
   n) -L₃-D-heteroaryl-G;
   o) -L₃-D-cycloalkyl-G;
   p) -L₃-D-heterocyclyl-G;
   q) -L₃-D-arylene-alkyl-G;
   r) -L₃-D-alkylene-arylene-alkyl-G; or
   s) -L₃-D-alkylene-arylene-G;
wherein
L₃ comprises a direct bond, -alkylene, -alkenylene, or alkynylene;
D comprises a direct bond, —CH₂—, —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, or —N(R₅)SO₂N(R₆)—, —N═N—, or —N(R₅)—N(R₆)—;
   wherein
      R₅ and R₆ independently comprise: -hydrogen, -alkyl, -aryl, -arylene-alkyl, -alkylene-aryl, or -alkylene-arylene-alkyl; and
      G comprises hydrogen, —CN, —SO₃H, —P(O)(OH)₂, —P(O)(O-alkyl)(OH), —CO₂H, —CO₂-alkyl, an acid isostere, —NR₇R₈, or

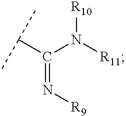

wherein
   R₇ and R₈ independently comprise: hydrogen, -alkyl, -L₄-E-alkyl, -L₄-E-aryl, —C(O)-alkyl, —C(O)-aryl, —SO₂-alkyl, —SO₂-aryl, or

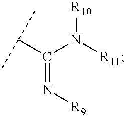

wherein
R₉, R₁₀, and R₁₁ independently comprise: -hydrogen, -alkyl, -aryl, -arylene-alkyl, -alkylene-aryl, and -alkylene-arylene-alkyl;
L₄ comprises a direct bond, -alkylene, -alkenylene, or -alkynylene;
E comprises a direct bond, —CH₂—, —O—, —N(R₁₂)—, —C(O)—, —CON(R₁₂)—, —N(R₁₂)C(O)—, —N(R₁₂)CON(R₁₃)—, —N(R₁₂)C(O)O—, —OC(O)N(R₁₂)—, —N(R₁₂)SO₂—, —SO₂N(R₁₂)—, —C(O)—O—, —O—C(O)—, —S—, —S(O)—, —S(O₂)—, —N(R₁₂)SO₂N(R₁₃)—, —N═N—, or —N(R₁₂)—N(R₁₃)—
wherein
R₁₂ and R₁₃ independently comprise: -hydrogen, -alkyl, -aryl, -arylene-alkyl, -alkylene-aryl, or -alkylene-arylene-alkyl.

In further embodiments, W comprises —O— or —N(R₂)—, wherein R₂ comprises hydrogen, alkyl, or -L₃-D-alkylene-aryl, wherein L₃ comprises alkylene, and D comprises —CO(NR₅)—, wherein R₅ comprises hydrogen. In other embodiments, W comprises —N(R₂)—, wherein R₂ comprises hydrogen.

R₁ comprises
kk) -hydrogen;
ll) -fluoro;
mm) -chloro;
nn) -bromo;
oo) -iodo;
pp) -cyano;
qq) -alkyl;
rr) -aryl;
ss) -alkylene-aryl;
tt) -heteroaryl;
uu) -alkylkene-heteroaryl;
vv) -cycloalkyl;
ww) -alkylene-cycloalkyl
xx) -heterocyclyl; or
yy) -alkylene-heterocyclyl;

In another embodiment, R₁ comprises hydrogen or aryl.

L₁ comprises:

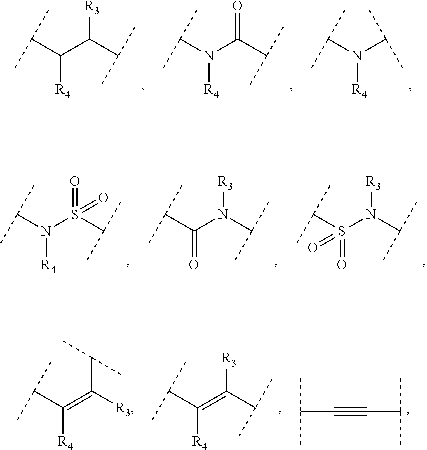

or a direct bond;
wherein R₃ and R₄ independently comprise: hydrogen, chloro, fluoro, bromo, alkyl, aryl, -alkylene-aryl, -cycloalkyl, -alkylene-cycloalkyl, -heterocyclyl, -alkylene-heterocyclyl, or -alkynylene. In another embodiment, L₁ comprises

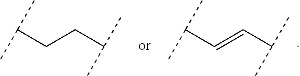

In another embodiment, L₁ comprises

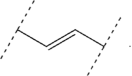

Ar₁ comprises an aryl, heteroaryl, fused cycloalkylaryl, fused cycloalkylheteroaryl, fused heterocyclylaryl, or fused heterocyclylheteroaryl group optionally substituted 1 to 7 times. In an embodiment, Ar₁ comprises a mono- or bicyclic aryl group optionally substituted 1 to 7 times. In another embodiment, Ar₁ comprises a phenyl or naphthyl group optionally having 1 to 5 substituents, wherein the substituents independently comprise:
a) -fluoro;
b) -chloro;
c) -bromo;
d) -iodo;
e) -cyano;
f) -nitro;
g) -perfluoroalkyl;
h) -J-R₁₄;
i) -alkyl;
j) -aryl;
k) -heteroaryl;
l) -heterocyclyl;
m) -cycloalkyl;
n) -L₅-aryl;
o) -L₅-arylene-aryl;
p) -L₅-arylene-alkyl;
q) -arylene-alkyl;
r) -arylene-arylene-alkyl;
s) -J-alkyl;
t) -J-aryl;
u) -J-alkylene-aryl;
v) -J-arylene-alkyl;
w) -J-alkylene-arylene-aryl;
x) -J-arylene-arylene-aryl;
y) -J-alkylene-arylene-alkyl;
z) -L₅-J-alkylene-aryl;
aa) -arylene-J-alkyl;

bb) -L₅-J-aryl;
cc) -L₅-J-heteroaryl;
dd) -L₅-J-cycloalkyl;
ee) -L₅-J-heterocyclyl;
ff) -L₅-J-arylene-alkyl;
gg) -L₅-J-alkylene-arylene-alkyl;
hh) -L₅₋-J-alkyl;
ii) -L₅-J-R₁₄;
jj) -arylene-J-R₁₄; or
s) -hydrogen;
   wherein L₅ comprises a direct bond, -alkylene, -alkenylene, or -alkynylene; and wherein J comprises a direct bond, —CH₂—, —O—, —N(R₁₅)—, —C(O)—, —CON(R₁₅)—, —N(R₁₅)C(O)—, —N(R₁₅)CON(R₁₆)—, —N(R₁₅)C(O)O—, —OC(O)N(R₁₅)—, —N(R₁₅)SO₂—, —SO₂N(R₁₅)—, —C(O)—O—, —O—C(O)—, —S—, —S(O)—, —S(O₂)—, —N(R₁₅)SO₂N(R₁₆)—, —N═N—, or —N(R₁₅)—N(R₁₆)—, and wherein R₁₄, R₁₅, and R₁₆ independently comprise: -hydrogen, -alkyl, -aryl, -arylene-alkyl, -alkylene-aryl, or -alkylene-arylene-alkyl.

In another embodiment, Ar₁ is a phenyl group optionally substituted 1 to 5 times, wherein the substituents independently comprise:
a) -fluoro;
b) -chloro;
c) -bromo;
d) -iodo;
e) -cyano;
f) -nitro; or
g) -aryl.

In another embodiment, Ar₁ comprises a phenyl group substituted 1 to 5 times, wherein the substituents comprise: -chloro or -fluoro.
Ar₂ comprises an arylene, heteroarylene, fused arylcycloalkylene, fused cycloalkylarylene, fused cycloalkylheteroarylene, fused heterocyclylarylene, or fused heterocyclylheteroarylene group optionally substituted 1 to 7 times. Ar₂ may also be taken in combination with R₄ to constitute a fused arylcycloalkylene, fused cycloalkylarylene, fused cycloalkylheteroarylene, fused heterocyclylarylene, or fused heterocyclylheteroarylene group, optionally substituted 1 to 7 times. In an embodiment, Ar₂ comprises an arylene group optionally substituted 1 to 7 times. In another embodiment, Ar₂ comprises a phenylene or naphthylene group optionally having 1 to 5 substituents, wherein the substituents independently comprise:
a) -fluoro;
b) -chloro;
c) -bromo;
d) -iodo;
e) -cyano;
f) -nitro;
g) -perfluoroalkyl;
h) -Q-R₁₇;
i)-alkyl;
j) -aryl;
k) -heteroaryl;
l) -heterocyclyl;
m) -cycloalkyl;
n) -L₆-aryl;
o) -L₆-arylene-aryl;
p) -L₆-arylene-alkyl;
q) -arylene-alkyl;
r) -arylene-arylene-alkyl;
s) -Q-alkyl;
t) -Q-aryl;
u) -Q-alkylene-aryl;
v) -Q-arylene-alkyl;
w) -Q-alkylene-arylene-aryl;
x) -Q-arylene-arylene-aryl;
y) -Q-alkylene-arylene-alkyl;
z) -L₆-Q-alkylene-aryl;
aa) -arylene-Q-alkyl;
bb) -L₆-Q-aryl;
cc) -L₆-Q-heteroaryl;
dd) -L₆-Q-cycloalkyl;
ee) -L₆-Q-heterocyclyl;
ff) -L₆-Q-arylene-alkyl;
gg) -L₆-Q-alkylene-arylene-alkyl;
s) -L₆-Q-alkyl;
t) -L₆-Q-alkylene-aryl-R₁₇;
u) -L₆-Q-alkylene-heteroaryl-R₁₇;
v) -arylene-Q-alkylene-R₁₇;
w) -heteroarylene-Q-alkylene-R₁₇;
x) -L₆-Q-aryl-R₁₇;
y) -L₆-Q-heteroarylene-R₁₇;
z) -L₆-Q-heteroaryl-R₁₇;
aa) -L₆-Q-cycloalkyl-R₁₇;
bb) -L₆-Q-heterocyclyl-R₁₇;
cc) -L₆-Q-arylene-alkyl-R₁₇;
dd) -L₆-Q-heteroarylene-alkyl-R₁₇;
ee) -L₆-Q-alkylene-arylene-alkyl-R₁₇;
ff) -L₆-Q-alkylene-heteroarylene-alkyl-R₁₇;
gg) -L₆-Q-alkylene-cycloalkylene-alkyl-R₁₇;
hh) -L₆-Q-alkylene-heterocyclylene-alkyl-R₁₇;
ii) -L₆-Q-alkyl-R₁₇;
jj) -L₆-Q-R₁₇;
kk) -arylene-Q-R₁₇;
ll)-heteroarylene-Q-R₁₇;
mm) -heterocyclylene-Q-R₁₇;
nn) -Q-alkylene-R₁₇;
oo) -Q-arylene-R₁₇;
pp) -Q-heteroarylene-R₁₇;
qq) -Q-alkylene-arylene-R₁₇;
rr) -Q-alkylene-heteroarylene-R₁₇;
ss)-Q-heteroarylene-alkylene-R₁₇;
tt) -Q-arylene-alkylene-R₁₇;
uu) -Q-cycloalkylene-alkylene-R₁₇;
vv) -Q-heterocyclylene-alkylene-R₁₇
ww) -Q-alkylene-arylene-alkyl-R₁₇;
xx) -Q-alkylene-heteroarylene-alkyl-R₁₇;
lll)

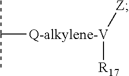

mmm)

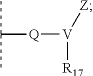

or
nnn) -hydrogen
wherein
   L₆ comprises a direct bond, -alkylene, -alkenylene, or -alkynylene;
   Q comprises a direct bond, —CH₂—, —O—, —N(R₁₈)—, —C(O)—, —CON(R₁₈)—, —N(R₁₈)C(O)—, —N(R₁₈)CON(R₁₉)—, —N(R₁₈)C(O)O—, —OC(O)N(R₁₈)—, —N(R₁₈)SO₂—, —SO₂N(R₁₈)—, —C(O)—O—, —O—C(O)—, —S—, —S(O)—, —S(O₂)—, —N(R₁₈)SO₂N(R₁₉)—, —N═N—, or —N(R₁₈)—N(R₁₉)—;
      wherein
         R₁₈ and R₁₉ independently comprise: -hydrogen, -alkyl, -aryl, -arylene-alkyl, -alkylene-aryl, or -alkylene-arylene-alkyl;
   V comprises

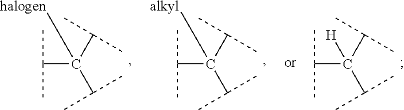

Z comprises hydrogen, -alkylene-aryl, -alkyl, -aryl, -heteroaryl, -heterocyclyl, -cycloalkyl, -alkylene-heteroaryl, or -alkylene-cycloalkyl;
R₁₇ comprises —SO₃H, —P(O)(OH)₂, —P(O)(O-alkyl)(OH), —CO₂H, —CO₂-alkyl, an acid isostere, hydrogen, -alkyl, -aryl, -arylene-alkyl, -alkylene-aryl, acyloxy-alkylene-, or -alkylene-arylene-alkyl.

In another embodiment, Ar₂ comprises a phenyl group or naphthyl group optionally substituted 1 to 5 times, wherein the substituents independently comprise:
a) -fluoro;
b) -chloro;
c) -bromo;
d) -iodo;
h) -Q-R₁₇;
i) -alkyl;
j) -aryl;
q) -arylene-alkyl;
s) -Q-alkyl; or
t) -arylene-Q-alkyl;
wherein
   Q comprises —CH₂—, —O—, —C(O)—, or —C(O)—O—, and
   R₁₇ comprises -hydrogen, -alkyl, -aryl, —CO₂H, or an acid isostere.

In another embodiment, Ar₂ comprises a phenyl group substituted 1 to 5 times, wherein the substituents independently comprise:
a) -fluoro;
b) -chloro;
c) -bromo;
d) -iodo;
e) -Q-R₁₇;
f) -alkyl;
g) -phenyl;
h) -phenylene-alkyl;
i) -Q-alkyl; or
j) -phenylene-Q-alkyl;
wherein
   Q comprises —CH₂—, —O—, —C(O)—, or —C(O)—O—, and
   R₁₇ comprises -hydrogen, -alkyl, -phenyl, or —CO₂H.

L₂ comprises: —CH₂—, —O—, alkylene, alkenylene, alkynylene, -K-alkylene-, -alkylene-K-, -alkylene-K-alkylene-, -alkenylene-K-alkylene-, -alkylene-K-alkenylene-, -arylene-K-alkylene-, alkylene-K-arylene, -heteroarylene-K-alkylene-, alkylene-K-heteroarylene, -arylene-K-, -K-arylene-, or -heteroarylene-K-, -K-heteroarylene,

wherein K comprises a direct bond, —N(R₂₀)—, —C(O)—, —CON(R₂₀)—, —N(R₂₀)C(O)—, —N(R₂₀)CON(R₂₁)—, —N(R₂₀)C(O)O—, —OC(O)N(R₂₀)—, —N(R₂₀)SO₂—, —SO₂N(R₂₀)—, —C(O)—O—, —O—C(O)—, —S—, —S(O)—, —S(O₂)—, —N(R₂₀)SO₂N(R₂₁)—, —N═N—, or —N(R₂₀)—N(R₂₁)—; —N(R₂₀)—, —C(O)—, —CON(R₂₀)—, —N(R₂₀)C(O)—, —N(R₂₀)CON(R₂₁)—, —N(R₂₀)C(O)O—, —OC(O)N(R₂₀)—, —N(R₂₀)SO₂—, —SO₂N(R₂₀)—, —C(O)—O—, —O—C(O)—, —S—, —S(O)—, —S(O₂)—, —N(R₂₀)SO₂N(R₂₁)—, —N═N—, —N(R₂₀)—N(R₂₁)— or a direct bond, wherein R₂₀ and R₂₁ independently comprise: -hydrogen, -alkyl, -aryl, -arylene-alkyl, -alkylene-aryl, or -alkylene-arylene-alkyl.

In an embodiment, L₂ comprises —O—, —O-alkylene-, -alkylene-O, or a direct bond. In another embodiment, L₂ comprises —O-alkylene- or a direct bond.
T comprises selected from the group consisting of: hydrogen, alkyl, cycloalkyl, heterocyclyl, aryl, heteroaryl, fused cycloalkylaryl, fused cycloalkylheteroaryl, fused heterocyclylaryl, or fused heterocyclylheteroaryl group optionally substituted 1 to 7 times. In an embodiment, T comprises an alkyl, -alkylene-aryl, or aryl group optionally substituted 1 to 7 times. In further embodiments, T comprises an aryl group optionally having 1 to 5 substituents, wherein the substituents independently comprise:
s) -fluoro;
t) -chloro;
u) -bromo;
v) -iodo;
w) -cyano;
x) -nitro;
y) -perfluoroalkyl;
z) -U-R₂₂;
aa) -alkyl;
bb) -aryl;
cc) -heteroaryl;
dd) -heterocyclyl;
ee) -cycloalkyl;
ff) -L₇-aryl;
gg) -L₇-arylene-aryl;
hh) -L₇-arylene-alkyl;
ii)-arylene-alkyl;
jj) -arylene-arylene-alkyl;
kk) -U-alkyl;
ll) -U-aryl;
mm) -U-alkylene-aryl;
nn)-U-arylene-alkyl;
oo) -U-alkylene-arylene-aryl;
pp) -U-arylene-arylene-aryl;
qq) -U-alkylene-arylene-alkyl;
rr) -L₇-U-alkylene-aryl;
ss) -arylene-U-alkyl;
tt) -L₇-U-aryl;
uu) -L₇-U-heteroaryl;
vv) -L₇-U-cycloalkyl;
ww) -L₇-U-heterocyclyl;
xx) -L₇-U-arylene-alkyl;
yy) -L₇-U-alkylene-arylene-alkyl;
zz) -L₇-U-alkyl;
aaa) -L₇-U-alkylene-aryl-R₂₂;
bbb) -L₇-U-alkylene-heteroaryl-R₂₂;
ccc) -arylene-U-alkylene-R₂₂;
ddd) -heteroarylene-U-alkylene-R₂₂;
eee) L₇-U-aryl-R₂₂;
fff) -L₇-U-heteroarylene-R₂₂;
ggg) -L₇-U-heteroaryl-R₂₂;
hhh) -L₇-U-cycloalkyl-R₂₂;
iii) -L₇-U-heterocyclyl-R₂₂;
jjj) -L₇-U-arylene-alkyl-R₂₂;
kkk) -L₇-U-heteroarylene-alkyl-R₂₂;
lll) -L₇-U-alkylene-arylene-alkyl-R₂₂;
mmm) -L₇-U-alkylene-heteroarylene-alkyl-R₂₂;
nnn) -L₇-Q-alkylene-cycloalkylene-alkyl-R₂₂;
ooo) -L₇-Q-alkylene-heterocyclylene-alkyl-R₂₂;
ppp) -L₇-U-alkyl-R₂₂;
qqq) -L₇-U-R₂₂;
rrr) -arylene-U-R₂₂;
sss) -heteroarylene-U-R₂₂;
ttt) -heterocyclylene-U-R₂₂;
uuu) -U-alkylene-R₂₂;
vvv) -U-arylene-R₂₂;
www) -U-heteroarylene-R₂₂;
xxx) -U-alkylene-arylene-R₂₂;
yyy)-U-alkylene-heteroarylene-R₂₂;
zzz) -U-heteroarylene-alkylene-R₂₂;
aaaa) -U-arylene-alkylene-R₂₂;
bbbb) -U-cycloalkylene-alkylene-R₂₂;
cccc) -U-heterocyclylene-alkylene-R₂₂;
dddd) -U-alkylene-arylene-alkyl-R₂₂;
eeee) -U-alkylene-heteroarylene-alkyl-R₂₂;
ffff)

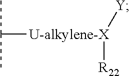

gggg)

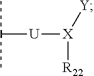

or
hhhh) -hydrogen;
wherein
   L₇ comprises a direct bond, -alkylene, -alkenylene, or -alkynylene;
   U comprises a direct bond, —CH₂—, —O—, —N(R₂₃)—, —C(O)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —C(O)—O—, —O—C(O)—, —S—, —S(O)—, —S(O₂)—, —N(R₂₃)SO₂N(R₂₄)—, —N═N—, or —N(R₂₃)—N(R₂₄)—;
      wherein
         R₂₃ and R₂₄ independently comprise: -hydrogen, -alkyl, -aryl, -arylene-alkyl, -alkylene-aryl, or -alkylene-arylene-alkyl;
   X comprises

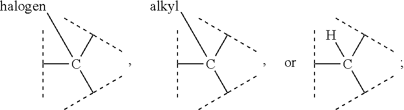

Y comprises hydrogen, -alkylene-aryl, -alkyl, -aryl, -heteroaryl, -heterocyclyl, -cycloalkyl, -alkylene-heteroaryl, or -alkylene-cycloalkyl;
R₂₂ comprises —SO₃H, —P(O)(OH)₂, —P(O)(O-alkyl)(OH), —CO₂H, —CO₂-alkyl, an acid isostere, -hydrogen, -alkyl, -aryl, -arylene-alkyl, -alkylene-aryl, acyloxy-alkylene-, or -alkylene-arylene-alkyl.

In another embodiment, T comprises an aryl group substituted by -U-alkylene-R₂₂, wherein U comprises —O— or a direct bond, and R₂₂ comprises —CO₂H or an acid isostere.

In another embodiment, the present invention provides compounds of Formula (I) wherein

a and b are equal to zero;

L₁ comprises

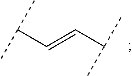

Ar₂ comprises a phenylene group optionally substituted 1 time with a group comprising: -Q-alkyl, wherein Q is —O—;

L₂ comprises a direct bond, O-alkylene, or an alkynylene; and

T comprises an aryl group substituted with at least one substituent comprising:
a) -U-R₂₂;
b) -U-alkylene-arylene-R₂₂;
c) -U-alkylene-R₂₂;
d) -U-arylene-R₂₂;
e) -U-arylene-R₂₂ wherein the arylene is substituted with at least one of a halogen, methanesulfonylamino, or trifluoromethanesulfonylamino group.
f) -U-arylene wherein the arylene is substituted with at least one trifluoromethanesulfonylamino group;
g) —R₂₂; or
h) -halogen
wherein R₂₂ is CO₂H or an acid isotere.

In another embodiment, the present invention provides compounds of Formula (I) wherein
a and b are equal to zero;
R₁ comprises hydrogen
W comprises —N(R₂)—
   wherein R₂ comprises alkyl; and
Ar₁ comprises aryl substituted 2 times wherein the substituent groups comprise -chloro.

In another embodiment of the compound of Formula (I), wherein a and b are equal to 0, and R₁ Ar₁, and W are as defined above, the groups T, L₂, Ar₂, and L₁ together comprise: E)-2-(4-methoxyphenyl)vinyl, (E)-2-(3-methoxyphenyl)vinyl, (E)-2-(2-methoxyphenyl)vinyl, (E)-2-(3,4-dimethoxyphenyl)vinyl, (E)-2-(2,3,4-trimethoxyphenyl)vinyl, (E)-2-(4-ethoxyphenyl)vinyl, (E)-2-phenylvinyl, (E)-2-(4-fluorophenyl)vinyl, (E)-2-(4-chlorophenyl)vinyl, (E)-2-(4-bromophenyl)vinyl, (E)-2-(1,1′-biphenyl-4-yl)vinyl, (E)-2-(1-naphthyl)vinyl, (E)-2-(2-naphthyl)vinyl, 9H-fluoren-9-ylidenemethyl, (E)-2-(4′-methoxy-1,1′-biphenyl-4-yl)vinyl, (E)-2-(3′-methoxy-1,1′-biphenyl-4-yl)vinyl, (E)-2-(4-hydroxyphenyl)vinyl, 2-(4-methoxyphenyl)ethyl, (E)-2-(4′-carboxymethyloxy-1,1′-biphenyl-4-yl)vinyl, (E)-2-(4′-(3-methoxycarbonyl-1-propyloxy)-1,1′-biphenyl-4-yl)vinyl, (E)-2-(4′-(3-carboxy-1-propyloxy)-1,1′-biphenyl-4-yl)vinyl, (E)-2-(4′-phenoxy-1,1′-biphenyl-4-yl)vinyl, or (E)-2-(4′-benzyloxy-1,1′-biphenyl-4-yl)vinyl.

In another embodiment of the compound of Formula (I), Ar₁ comprises 2,4-dichlorophenyl.

In another embodiment of the compound of Formula (I), W comprises —N(R₂)—, wherein R₂ comprises -L₃-D-alkylene-arylene-G, wherein L₃ comprises a direct bond or alkylene, D is a direct bond, or —O—, and G comprises —CN, —SO₃H, —P(O)(OH)₂, —P(O)(O-alkyl)(OH), —CO₂H, —CO₂-alkyl, or an acid isostere.

In another aspect, the present invention provides a pharmaceutically acceptable salt, solvate, or prodrug of compounds of Formula (I).

In the compounds of Formula (I), the various functional groups represented should be understood to have a point of attachment at the functional group having the hyphen. In other words, in the case of -alkylene-aryl, it should be understood that the point of attachment is the alkylene group; an example would be benzyl. In the case of a group such as —C(O)—NH— alkylene-aryl, the point of attachment is the carbonyl carbon.

Also included within the scope of the invention are the individual enantiomers of the compounds represented by Formula (I) above as well as any wholly or partially racemic mixtures thereof. The present invention also covers the individual enantiomers of the compounds represented by formula above as mixtures with diastereoisomers thereof in which one or more stereocenters are inverted.

Compounds of the present invention which are currently preferred for their biological activity are listed by name below in Table 1.

The ability of compounds Formula (I) to potentially treat or inhibit disorders related to insulin resistance or hyperglycemia was established with representative compounds of Formula (I) listed in Table I using a standard primary/secondary assay test procedure that measures the inhibition of PTP-1B activity.

The compounds of this invention can be potentially useful in treating metabolic disorders related to insulin resistance or hyperglycemia, typically associated with obesity or glucose intolerance. The compounds of this invention may therefore be particularly useful in the treatment or inhibition of type II diabetes. The compounds of this invention are also potentially useful in modulating glucose levels in disorders such as type I diabetes.

TABLE 1

| Ex. | Structure | Name |
|---|---|---|
| 1 | 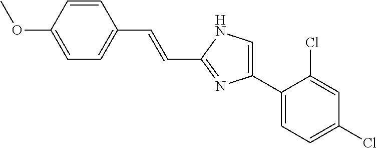 | 4-(2,4-dichloro-phenyl)-2- [2-(4-methoxy-phenyl)-(E)- vinyl]-1H-imidazole |
| 2 | 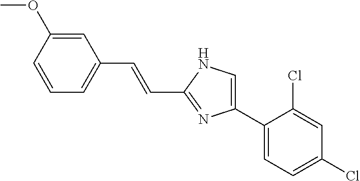 | 4-(2,4-dichloro-phenyl)-2- [2-(3-methoxy-phenyl)-(E)- vinyl]-1H-imidazole |
| 3 | 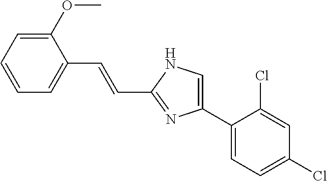 | 4-(2,4-dichloro-phenyl)-2- [2-(2-methoxy-phenyl)-(E)- vinyl]-1H-imidazole |
| 4 | 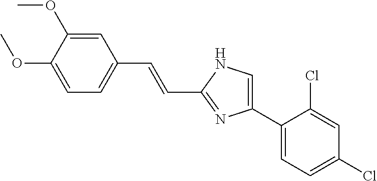 | 4-(2,4-dichloro-phenyl)-2- [2-(3,4-dimethoxy-phenyl)- (E)-vinyl]-1H-imidazole |
| 5 | 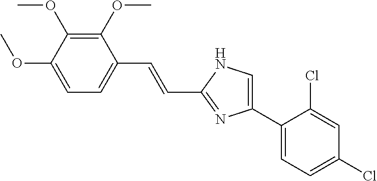 | 4-(2,4-dichloro-phenyl)-2- [2-(2,3,4-trimethoxy- phenyl)-(E)-vinyl]-1H- imidazole |
| 6 | 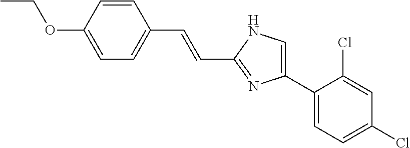 | 4-(2,4-dichloro-phenyl)-2- [2-(4-ethoxy-phenyl)-(E)- vinyl]-1H-imidazole |
| 7 | 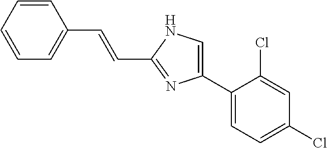 | 4-(2,4-dichloro-phenyl)-2- styryl-1H-imidazole |
| 8 | 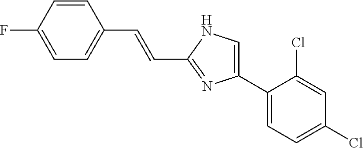 | 4-(2,4-dichloro-phenyl)-2- [2-(4-fluoro-phenyl)-(E)- vinyl]-1H-imidazole |
| 9 | 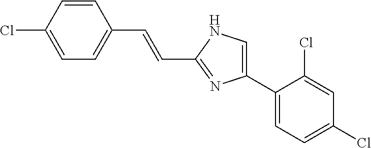 | 2-[2-(4-chloro-phenyl)-(E)- vinyl]-4-(2,4-dichloro- phenyl)-1H-imidazole |
| 10 | 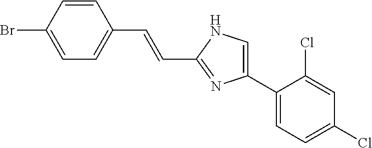 | 2-[2-(4-bromo-phenyl)-(E)- vinyl]-4-(2,4-dichloro- phenyl)-1H-imidazole |
| 11 | 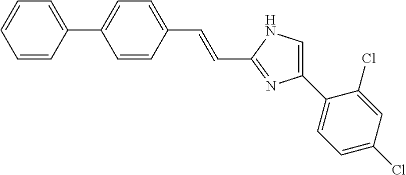 | 2-(2-biphenyl-4-yl-(E)- vinyl)-4-(2,4-dichloro- phenyl)-1H-imidazole |
| 12 | 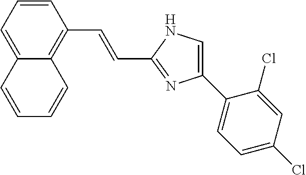 | 4-(2,4-dichloro-phenyl)-2- (2-naphthalen-1-yl-(E)- vinyl)-1H-imidazole |
| 13 | 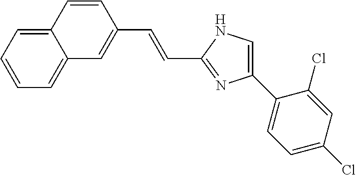 | 4-(2,4-dichloro-phenyl)-2- (2-naphthalen-2-yl-(E)- vinyl)-1H-imidazole |
| 14 | 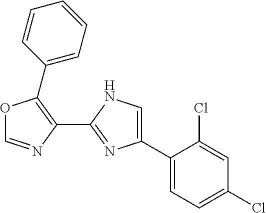 | 4-[4-(2,4-dichloro-phenyl)- 1H-imidazol-2-yl]-5-phenyl- oxazole |
| 15 | 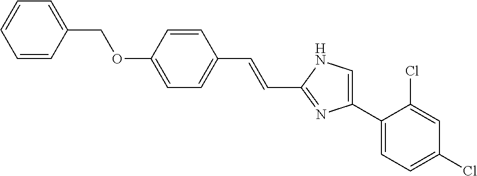 | 2-[2-(4-benzyloxy-phenyl)- (E)-vinyl]-4-(2,4-dichloro- phenyl)-1H-imidazole |
| 16 | 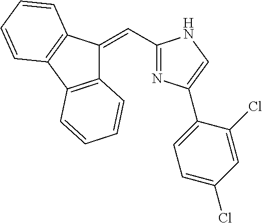 | 4-(2,4-dichloro-phenyl)-2- fluoren-9-ylidenemethyl- 1H-imidazole |
| 17 | 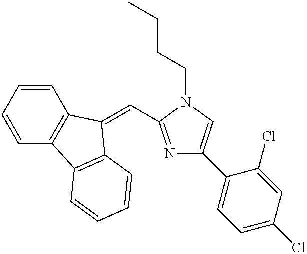 | 1-butyl-4-(2,4-dichloro- phenyl)-2-fluoren-9- ylidenemethyl-1H-imidazole |
| 18 | 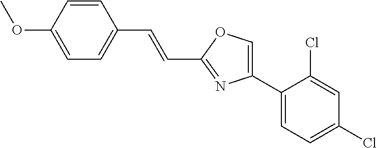 | 4-(2,4-dichloro-phenyl)-2- [2-(4-methoxy-phenyl)-(E)- vinyl]-oxazole |
| 19 | 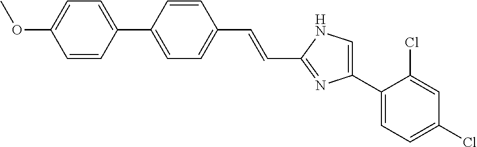 | 4-(2,4-dichloro-phenyl)-2- [2-(4′-methoxy-biphenyl-4- yl)-(E)-vinyl]-1H-imidazole |
| 20 | 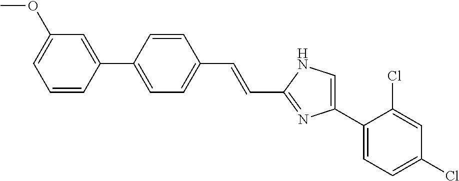 | 4-(2,4-dichloro-phenyl)-2- [2-(3′-methoxy-biphenyl-4- yl)-(E)-vinyl]-1H-imidazole |
| 21 | 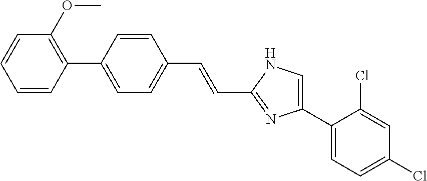 | 4-(2,4-dichloro-phenyl)-2- [2-(2′-methoxy-biphenyl-4- yl)-(E)-vinyl]-1H-imidazole |
| 22 | 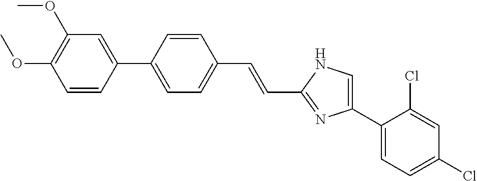 | 4-(2,4-dichloro-phenyl)-2- [2-(3′,4′-dimethoxy- biphenyl-4-yl)-(E)-vinyl]- 1H-imidazole |
| 23 | 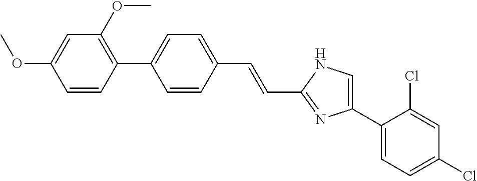 | 4-(2,4-dichloro-phenyl)-2- [2-(2′,4′-dimethoxy- biphenyl-4-yl)-(E)-vinyl]- 1H-imidazole |
| 24 | 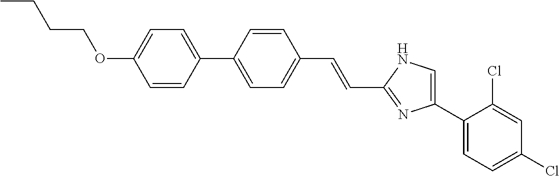 | 2-[2-(4′-butoxy-biphenyl- 4-yl)-(E)-vinyl]-4-(2,4- dichloro-phenyl)-1H- imidazole |
| 25 | 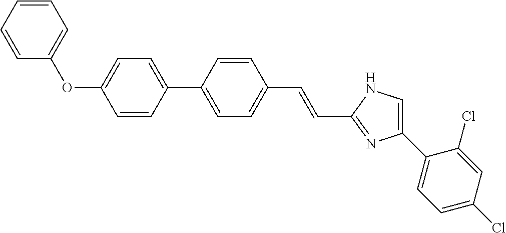 | 4-(2,4-dichloro-phenyl)-2- [2-(4′-phenoxy-biphenyl-4- yl)-(E)-vinyl]-1H-imidazole |
| 26 | 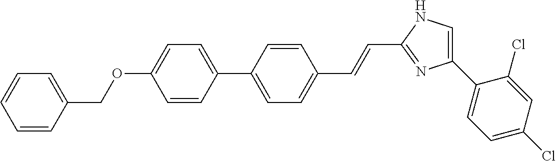 | 2-[2-(4′-benzyloxy- biphenyl-4-yl)-(E)-vinyl]-4- (2,4-dichloro-phenyl)-1H- imidazole |
| 27 | 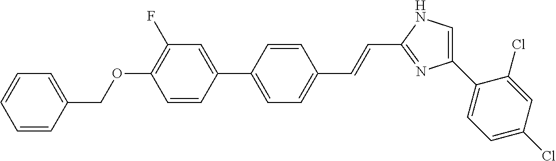 | 2-[2-(4′-benzyloxy-3′-fluoro- biphenyl-4-yl)-(E)-vinyl]-4- (2,4-dichloro-phenyl)-1H- imidazole |
| 28 | 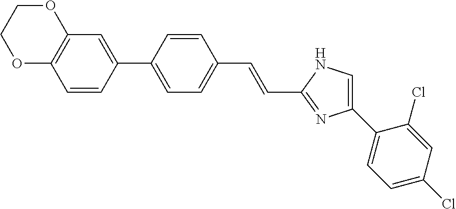 | 4-(2,4-dichloro-phenyl)-2- {2-[4-(2,3-dihydro- benzo[1,4]dioxin-6-yl)- phenyl]-(E)-vinyl}-1H- imidazole |
| 29 | 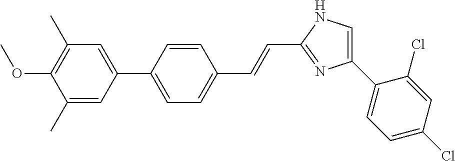 | 4-(2,4-dichloro-phenyl)-2- [2-(4′-methoxy-3′,5′- dimethyl-biphenyl-4-yl)-(E)- vinyl]-1H-imidazole |
| 30 | 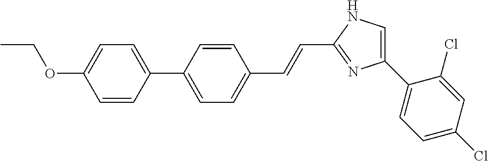 | 4-(2,4-Dichloro-phenyl)-2- [2-(4′-ethoxy-biphenyl-4-yl)- (E)-vinyl]-1H-imidazole |
| 31 | 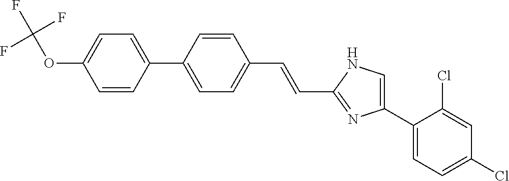 | 4-(2,4-Dichloro-phenyl)-2- [2-(4′-trifluoromethoxy- biphenyl-4-yl)-(E)-vinyl]-1H- imidazole |
| 32 | 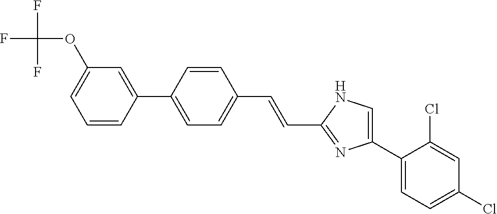 | 4-(2,4-dichloro-phenyl)-2- [2-(3′-trifluoromethoxy- biphenyl-4-yl)-(E)-vinyl]-1H- imidazole |
| 33 | 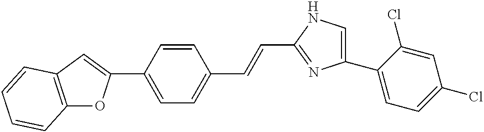 | 2-[2-(4-benzofuran-2-yl- phenyl)-(E)-vinyl]-4-(2,4- dichloro-phenyl)-1H- imidazole |
| 34 | 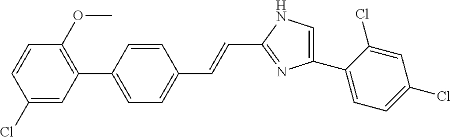 | 2-[2-(5′-chloro-2′-methoxy- biphenyl-4-yl)-(E)-vinyl]-4- (2,4-dichloro-phenyl)-1H- imidazole |
| 35 | 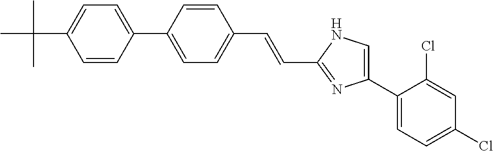 | 2-[2-(4′-tert-butyl-biphenyl- 4-yl)-(E)-vinyl]-4-(2,4- dichloro-phenyl)-1H- imidazole |
| 36 | 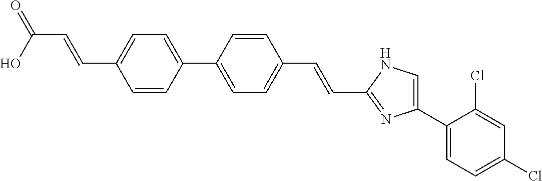 | 3-(4′-{2-[4-(2,4-dichloro- phenyl)-1H-imidazol-2-yl]- (E)-vinyl}-biphenyl-4-yl)- acrylic acid |
| 37 | 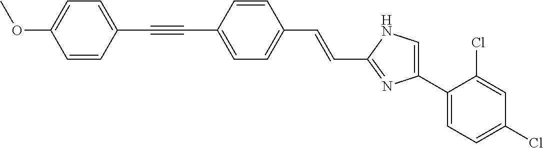 | 4-(2,4-dichloro-phenyl)-2- {2-[4-(4-methoxy- phenylethynyl)-phenyl]-(E)- vinyl}-1H-imidazole |
| 38 | 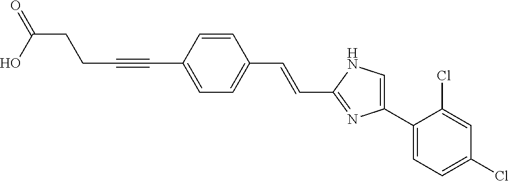 | 5-(4-{2-[4-(2,4-dichloro- phenyl)-1H-imidazol-2-yl]- (E)-vinyl}-phenyl)-pent-4- ynoic acid |
| 39 | 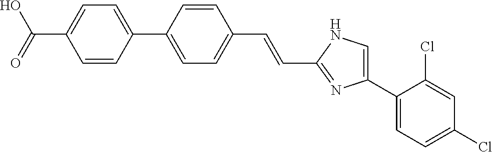 | 4′-{2-[4-(2,4-dichloro- phenyl)-1H-imidazol-2-yl]- (E)-vinyl}-biphenyl-4- carboxylic acid |
| 40 | 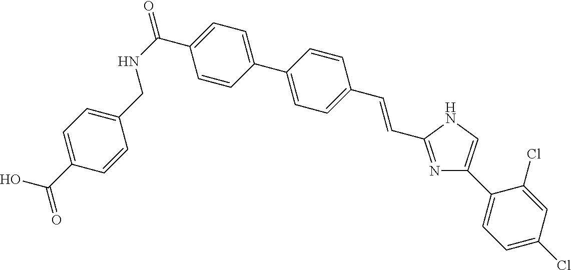 | 4-{[(4′-{2-[4-(2,4-dichloro- phenyl)-1H-imidazol-2-yl]- (E)-vinyl}-biphenyl-4- carbonyl)-amino]-methyl}- benzoic acid |
| 41 | 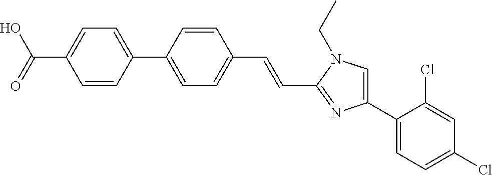 | 4′-{2-[4-(2,4-Dichloro- phenyl)-1-ethyl-1H- imidazol-2-yl]-(E)-vinyl}- biphenyl-4-carboxylic acid |
| 42 | 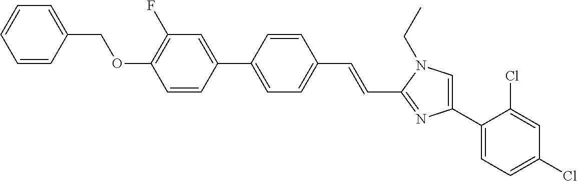 | 2-[2-(4′-benzyloxy-3′-fluoro- biphenyl-4-yl)-(E)-vinyl]-4- (2,4-dichloro-phenyl)-1- ethyl-1H-imidazole |
| 43 | 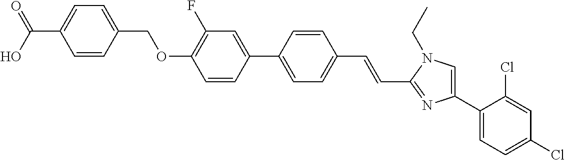 | 4-(4′-{2-[4-(2,4-dichloro- phenyl)-1-ethyl-1H- imidazol-2-yl]-(E)-vinyl}-3- fluoro-biphenyl-4- yloxymethyl)-benzoic acid |
| 44 | 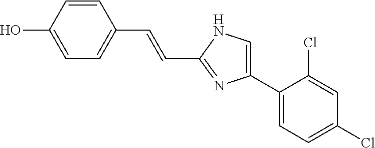 | 4-{2-[4-(2,4-Dichloro- phenyl)-1H-imidazol-2-yl]- (E)-vinyl}-phenol |
| 45 | 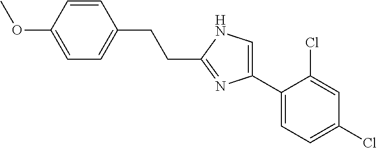 | 4-(2,4-dichloro-phenyl)-2- [2-(4-methoxy-phenyl)- ethyl]-1H-imidazole |
| 46 | 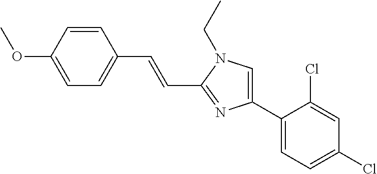 | 4-(2,4-dichloro-phenyl)-1- ethyl-2-[2-(4-methoxy- phenyl)-(E)-vinyl]-1H- imidazole |
| 47 | 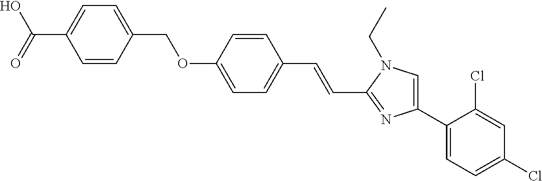 | 4-(4-{2-[4-(2,4-dichloro- phenyl)-1-ethyl-1H- imidazol-2-yl]-(E)-vinyl}- phenoxymethyl)-benzoic acid |
| 48 | 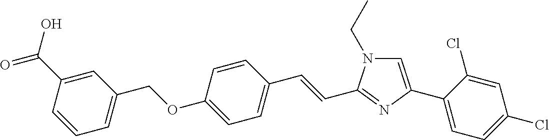 | 3-(4-{2-[4-(2,4-dichloro- phenyl)-1-ethyl-1H- imidazol-2-yl]-(E)-vinyl}- phenoxymethyl)-benzoic acid |
| 49 | 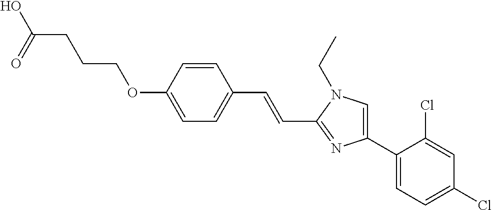 | 4-(4-{2-[4-(2,4-dichloro- phenyl)-1-ethyl-1H- imidazol-2-yl]-(E)-vinyl}- phenoxy)-butyric acid |
| 50 | 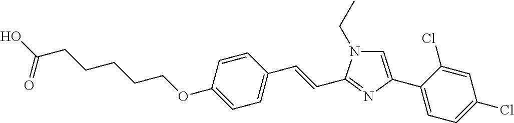 | 6-(4-{2-[4-(2,4-Dichloro- phenyl)-1-ethyl-1H- imidazol-2-yl]-(E)-vinyl}- phenoxy)-hexanoic acid |
| 51 | 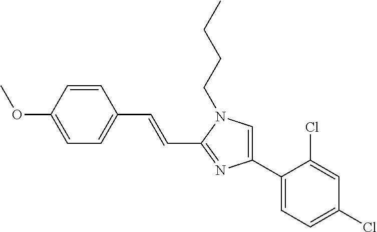 | 1-butyl-4-(2,4-dichloro- phenyl)-2-[2-(4-methoxy- phenyl)-(E)-vinyl]-1H- imidazole |
| 52 | 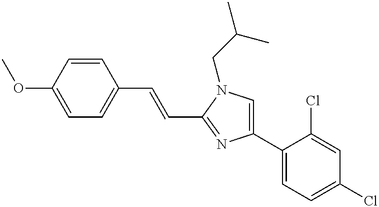 | 4-(2,4-dichloro-phenyl)-1- isobutyl-2-[2-(4-methoxy- phenyl)-(E)-vinyl]-1H- imidazole |
| 53 | 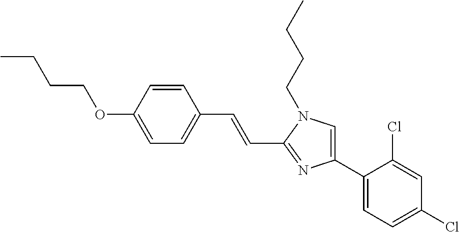 | 2-[2-(4-butoxy-phenyl)-(E)- vinyl]-1-butyl-4-(2,4- dichloro-phenyl)-1H- imidazole |
| 54 | 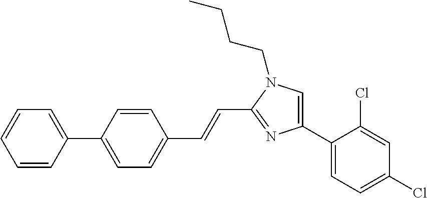 | 2-(2-biphenyl-4-yl-(E)- vinyl)-1-butyl-4-(2,4- dichloro-phenyl)-1H- imidazole |
| 55 | 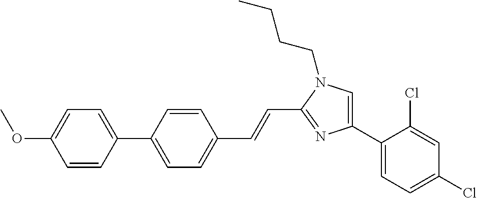 | 1-butyl-4-(2,4-dichloro- phenyl)-2-[2-(4′-methoxy- biphenyl-4-yl)-‘(E)-vinyl]- 1H-imidazole |
| 56 | 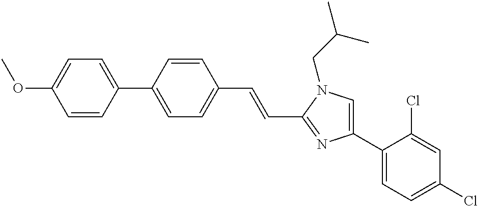 | 4-(2,4-dichloro-phenyl)-1- isobutyl-2-[2-(4′-methoxy- biphenyl-4-yl)-(E)-vinyl]-1H- imidazole |
| 57 | 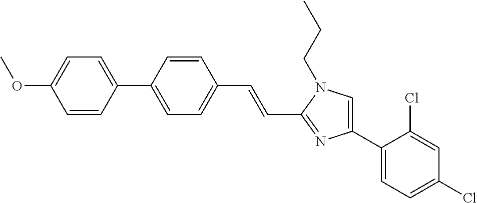 | 4-(2,4-dichloro-phenyl)-2- [2-(4′-methoxy-biphenyl-4- yl)-(E)-vinyl]-1-propyl-1H- imidazole |
| 58 | 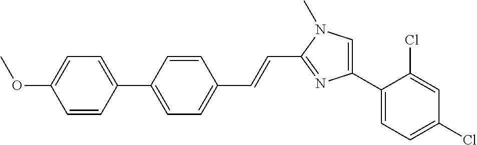 | 4-(2,4-dichloro-phenyl)-2- [2-(4′-methoxy-biphenyl-4- yl)-(E)-vinyl]-1-methyl-1H- imidazole |
| 59 | 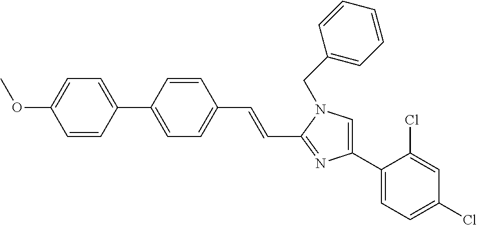 | 1-benzyl-4-(2,4-dichloro- phenyl)-2-[2-(4′-methoxy- biphenyl-4-yl)-(E)-vinyl]- 1H-imidazole |
| 60 | 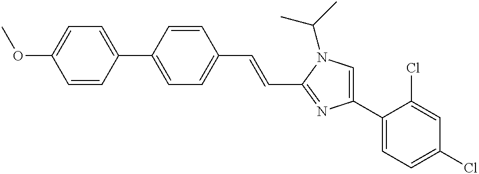 | 4-(2,4-dichloro-phenyl)-1- isopropyl-2-[2-(4′-methoxy- biphenyl-4-yl)-(E)-vinyl]- 1H-imidazole |
| 61 | 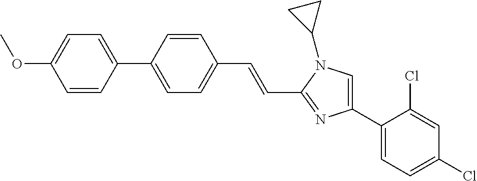 | 1-cyclopropyl-4-(2,4- dichloro-phenyl)-2-[2-(4′- methoxy-biphenyl-4-yl)-(E)- vinyl]-1H-imidazole |
| 62 | 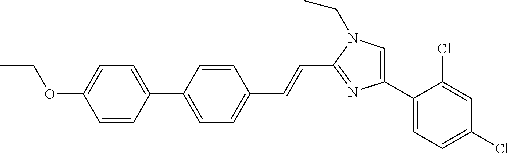 | 4-(2,4-dichloro-phenyl)-2- [2-(4′-ethoxy-biphenyl-4-yl)- (E)-vinyl]-1-ethyl-1H- imidazole |
| 63 | 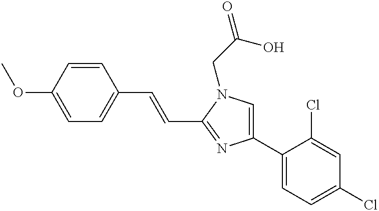 | {4-(2,4-dichloro-phenyl)-2- [2-(4-methoxy-phenyl)-(E)- vinyl]-imidazol-1-yl}-acetic acid |
| 64 | 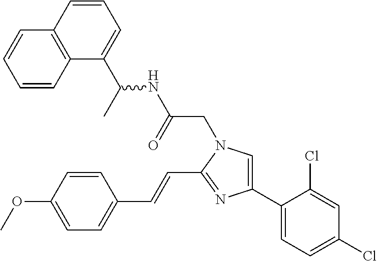 | 2-{4-(2,4-dichloro-phenyl)- 2-[2-(4-methoxy-phenyl)- (E)-vinyl]-imidazol-1-yl}-N- (1-naphthalen-1-yl-ethyl)- acetamide |
| 65 | 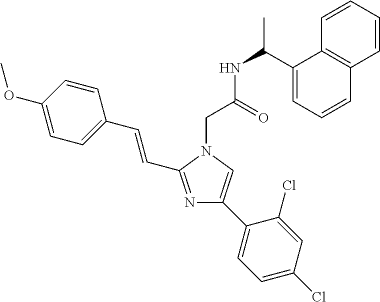 | 2-{4-(2,4-dichloro-phenyl)- 2-[2-(4-methoxy-phenyl)- (E)-vinyl]-imidazol-1-yl}-N- ((S)-1-naphthalen-1-yl- ethyl)-acetamide |
| 66 | 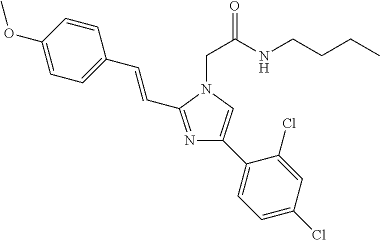 | N-butyl-2-{4-(2,4-dichloro- phenyl)-2-[2-(4-methoxy- phenyl)-(E)-vinyl]-imidazol- 1-yl}-acetamide |
| 67 | 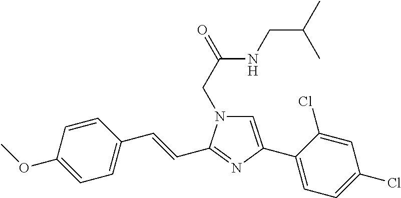 | 2-{4-(2,4-dichloro-phenyl)- 2-[2-(4-methoxy-phenyl)- (E)-vinyl]-imidazol-1-yl}-N- isobutyl-acetamide |
| 68 | 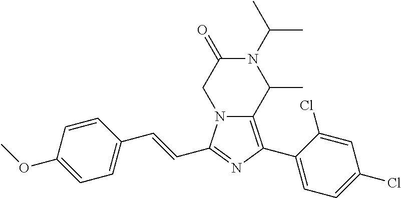 | 2-{4-(2,4-dichloro-phenyl)- 2-[2-(4-methoxy-phenyl)- (E)-vinyl]-imidazol-1-yl}- N,N-diisopropyl-acetamide |
| 69 | 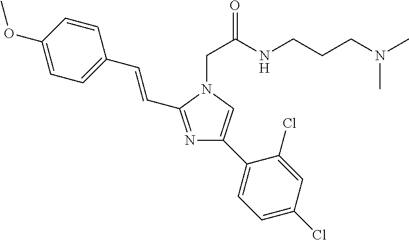 | 2-{4-(2,4-dichloro-phenyl)- 2-[2-(4-methoxy-phenyl)- (E)-vinyl]-imidazol-1-yl}-N- (3-dimethylamino-propyl)- acetamide |
| 70 | 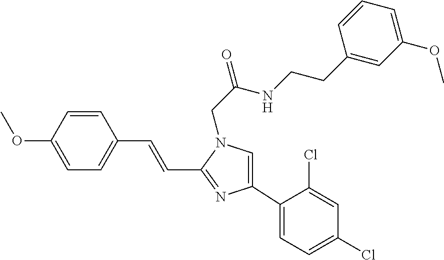 | 2-{4-(2,4-dichloro-phenyl)- 2-[2-(4-methoxy-phenyl)- (E)-vinyl]-imidazol-1-yl}-N- [2-(3-methoxy-phenyl)- ethyl]-acetamide |
| 71 | 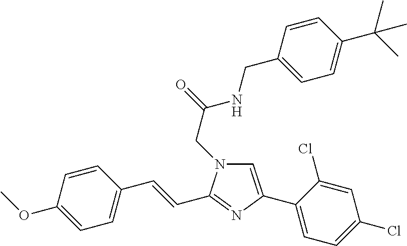 | N-(4-tert-butyl-benzyl)-2-{4- (2,4-dichloro-phenyl)-2-[2- (4-methoxy-phenyl)-(E)- vinyl]-imidazol-1-yl}- acetamide |
| 72 | 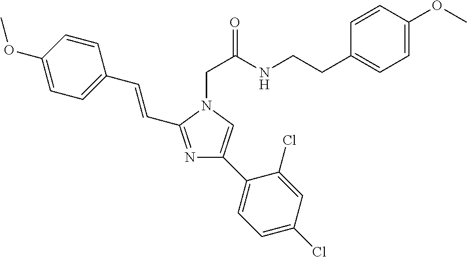 | 2-{4-(2,4-dichloro-phenyl)- 2-[2-(4-methoxy-phenyl)- (E)-vinyl]-imidazol-1-yl}-N- [2-(4-methoxy-phenyl)- ethyl]-acetamide |
| 73 | 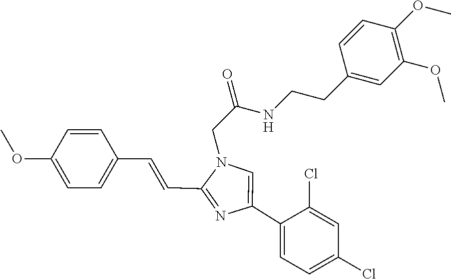 | 2-{4-(2,4-dichloro-phenyl)- 2-[2-(4-methoxy-phenyl)- (E)-vinyl]-imidazol-1yl}-N- [2-(3,4-dimethoxy-phenyl)- ethyl]-acetamid |
| 74 | 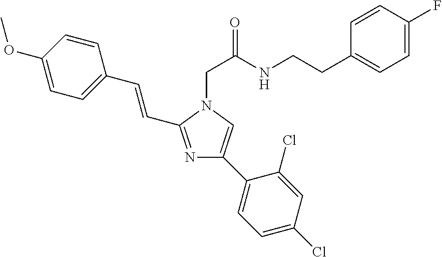 | 2-{4-(2,4-dichloro-phenyl)- 2-[2-(4-methoxy-phenyl)- (E)-vinyl]-imidazol-1-yl}-N- [2-(4-fluoro-phenyl)-ethyl]- acetamide |
| 75 | 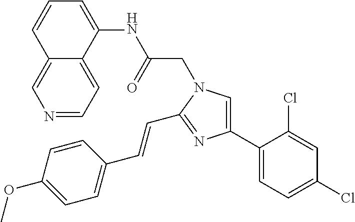 | 2-{4-(2,4-dichloro-phenyl)- 2-[2-(4-methoxy-phenyl)- (E)-vinyl]-imidazol-1-yl}-N- isoquinolin-5-yl-acetamide |
| 76 | 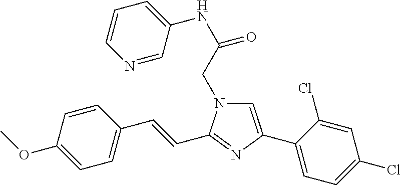 | 2-{4-(2,4-dichloro-phenyl)- 2-[2-(4-methoxy-phenyl)- (E)-vinyl]-imidazol-1-yl}-N- pyridin-4-yl-acetamide |
| 77 | 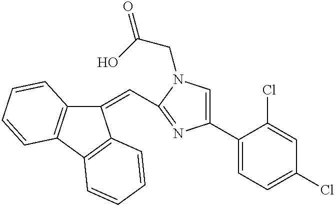 | [4-(2,4-dichloro-phenyl)-2- fluoren-9-ylidenemethyl- imidazol-1-yl]-acetic acid |
| 78 | 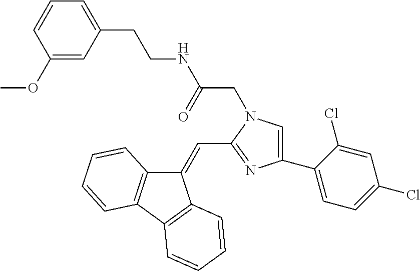 | 2-[4-(2,4-dichloro-phenyl)- 2-fluoren-9-ylidenemethyl- imidazol-1-yl]-N-[2-(3- methoxy-phenyl)-ethyl]- acetamide |
| 79 | 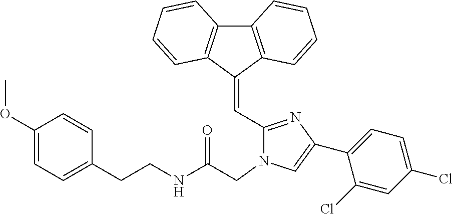 | 2-[4-(2,4-dichloro-phenyl)- 2-fluoren-9-ylidenemethyl- imidazol-1-yl]-N-[2-(4- methoxy-phenyl)-ethyl]- acetamide |
| 80 | 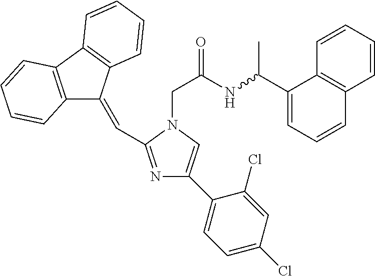 | 2-[4-(2,4-dichloro-phenyl)- 2-fluoren-9-ylidenemethyl- imidazol-1-yl]-N-(1- naphthalen-1-yl-ethyl)- acetamide |
| 81 | 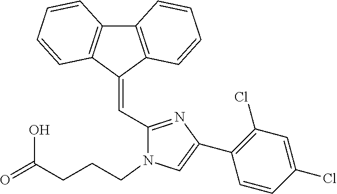 | 4-[4-(2,4-dichloro-phenyl)- 2-fluoren-9-ylidenemethyl- imidazol-1-yl]-butyric acid |
| 82 | 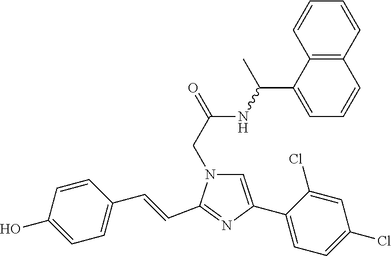 | 2-{4-(2,4-dichloro-phenyl)- 2-[2-(4-hydroxy-phenyl)- (E)-vinyl]-imidazol-1-yl}-N- (1-naphthalen-1-yl-ethyl)- acetamid |
| 83 | 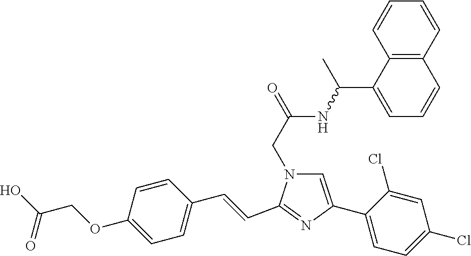 | [4-(2-{4-(2,4-dichloro- phenyl)-1-[(1-naphthalen-1- yl-ethylcarbamoyl)-methyl]- 1H-imidazol-2-yl}-(E)-vinyl)- phenoxy]-acetic acid |
| 84 | 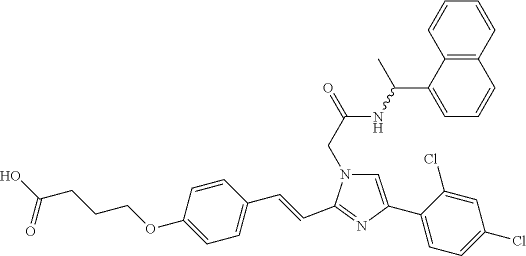 | 4-[4-(2-{4-(2,4-dichloro- phenyl)-1-[(1-naphthalen-1- yl-ethylcarbamoyl)-methyl]- 1H-imidazol-2-yl}-(E)-vinyl)- phenoxy]-butyric acid |
| 85 | 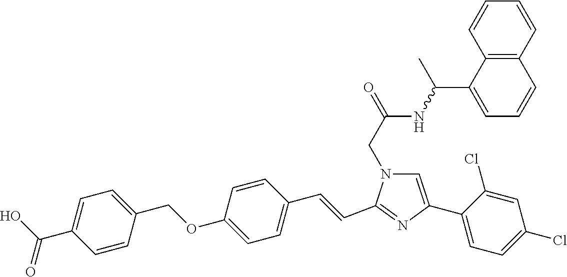 | 4-[4-(2-{4-(2,4-dichloro- phenyl)-1-[(1-naphthalen-1- yl-ethylcarbamoyl)-methyl]- 1H-imidazol-2-yl}-(E)-vinyl)- phenoxymethyl]-benzoic acid |
| 86 | 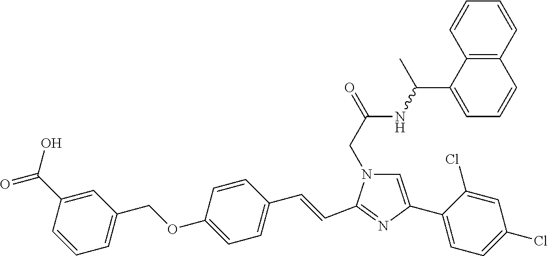 | 3-[4-(2-{4-(2,4-dichloro- phenyl)-1-[(1-naphthalen-1- yl-ethylcarbamoyl)-methyl]- 1H-imidazol-2-yl}-(E)-vinyl)- phenoxymethyl]-benzoic acid |
| 87 | 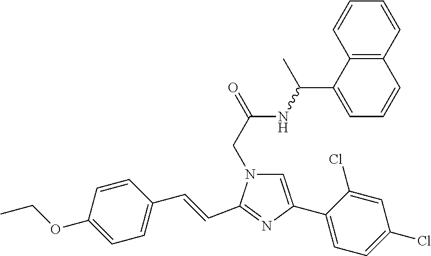 | 2-{4-(2,4-dichloro-phenyl)- 2-[2-(4-ethoxy-phenyl)-(E)- vinyl]-imidazol-1-yl}-N-(1- naphthalen-1-yl-ethyl)- acetamide |
| 88 | 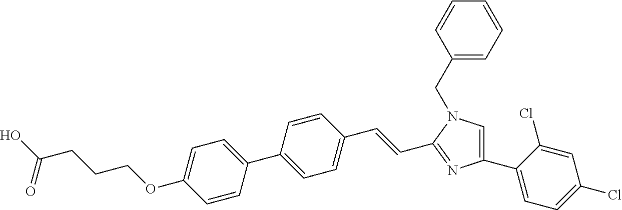 | 4-(4′-{2-[1-benzyl-4-(2,4- dichloro-phenyl)-1H- imidazol-2-yl]-(E)-vinyl}- biphenyl-4-yloxy)-butyric acid |
| 89 | 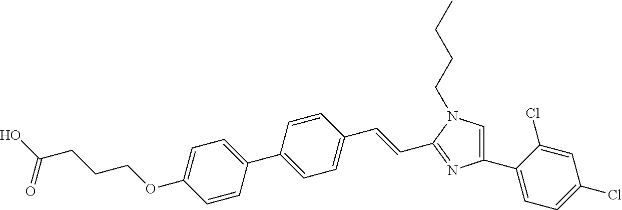 | 4-(4′-{2-[1-butyl-4-(2,4- dichloro-phenyl)-1H- imidazol-2-yl]-(E)-vinyl}- biphenyl-4-yloxy)-butyric acid |
| 90 | 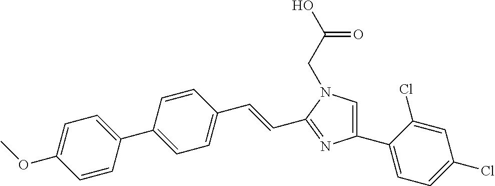 | {4-(2,4-dichloro-phenyl)-2- [2-(4′-methoxy-biphenyl-4- yl)-(E)-vinyl]-imidazol-1-yl}- acetic acid |
| 91 | 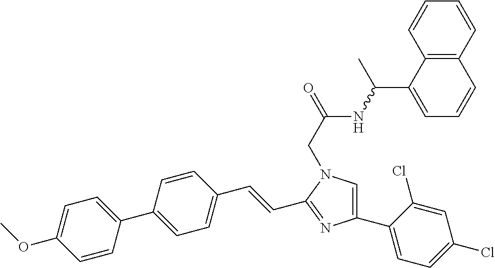 | 2-{4-(2,4-dichloro-phenyl)- 2-[2-(4′-methoxy-biphenyl- 4-yl)-(E)-vinyl]-imidazol-1- yl}-N-(1-naphthalen-1-yl- ethyl)-acetamide |
| 92 | 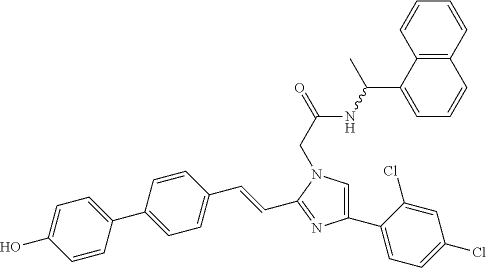 | 2-{4-(2,4-dichloro-phenyl)- 2-[2-(4′-hydroxy-biphenyl- 4-yl)-(E)-vinyl]-imidazol-1- yl}-N-(1-naphthalen-1-yl- ethyl)-acetamide |
| 93 | 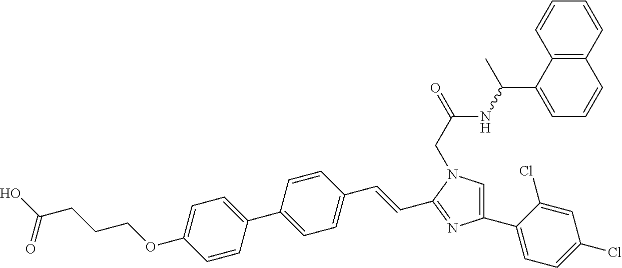 | 4-[4′-(2-{4-(2,4-dichloro- phenyl)-1-[(1-naphthalen-1- yl-ethylcarbamoyl)-methyl]- 1H-imidazol-2-yl}-(E)-vinyl)- biphenyl-4-yloxy]-butyric acid |
| 94 | 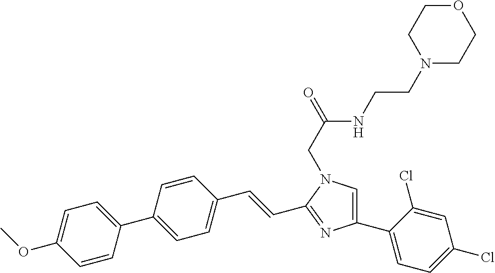 | 2-{4-(2,4-dichloro-phenyl)- 2-[2-(4′-methoxy-biphenyl- 4-yl)-(E)-vinyl]-imidazol-1- yl}-N-(2-morpholin-4-yl- ethyl)-acetamide |
| 95 | 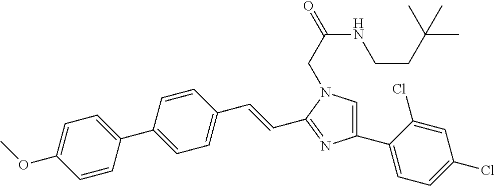 | 2-{4-(2,4-dichloro-phenyl)- 2-[2-(4′-methoxy-biphenyl- 4-yl)-(E)-vinyl]-imidazol-1- yl}-N-(3,3-dimethyl-butyl)- acetamide |
| 96 | 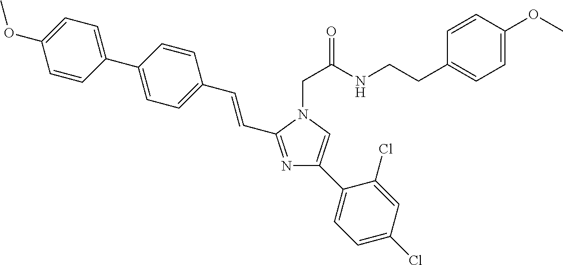 | 2-{4-(2,4-dichloro-phenyl)- 2-[2-(4′-methoxy-biphenyl- 4-yl)-(E)-vinyl]-imidazol-1- yl}-N-[2-(4-methoxy- phenyl)-ethyl]-acetamide |
| 97 | 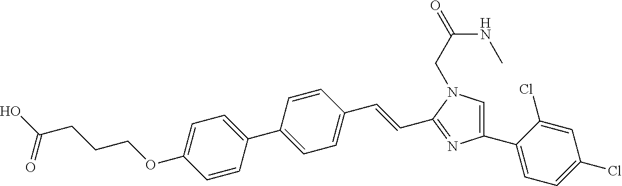 | 4-(4′-{2-[4-(2,4-dichloro- phenyl)-1- methylcarbamoylmethyl- 1H-imidazol-2-yl]-(E)-vinyl}- biphenyl-4-yloxy)-butyric acid |
| 98 | 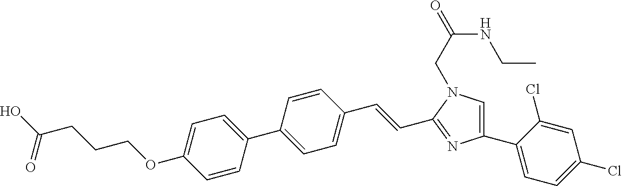 | 4-(4′-{2-[4-(2,4-dichloro- phenyl)-1- ethylcarbamoylmethyl-1H- imidazol-2-yl]-(E)-vinyl}- biphenyl-4-yloxy)-butyric acid |
| 99 | 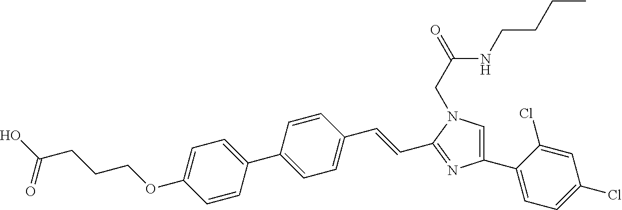 | 4-(4′-{2-[1- butylcarbamoylmethyl-4- (2,4-dichloro-phenyl)-1H- imidazol-2-yl]-(E)-vinyl}- biphenyl-4-yloxy)-butyric acid |
| 100 | 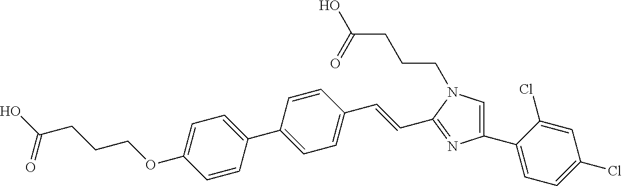 | 4-[2-{2-[4′-(3-carboxy- propoxy)-biphenyl-4-yl]-(E)- vinyl}-4-(2,4-dichloro- phenyl)-imidazol-1-yl]- butyric acid |
| 101 | 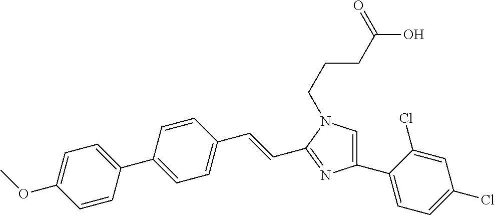 | 4-{4-(2,4-dichloro-phenyl)- 2-[2-(4′-methoxy-biphenyl- 4-yl)-(E)-vinyl]-imidazol-1- yl}-butyric acid |
| 102 | 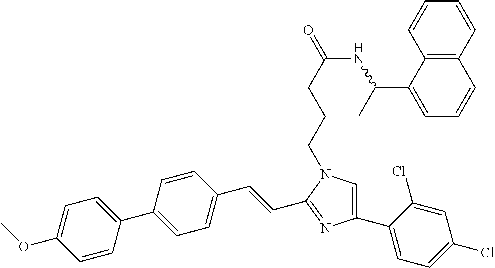 | 4-{4-(2,4-dichloro-phenyl)- 2-[2-(4′-methoxy-biphenyl- 4-yl)-(E)-vinyl]-imidazol-1- yl}-N-(1-naphthalen-1-yl- ethyl)-butyramide |
| 103 | 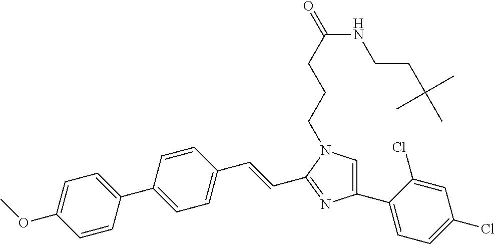 | 4-{4-(2,4-dichloro-phenyl)- 2-[2-(4′-methoxy-biphenyl- 4-yl)-(E)-vinyl]-imidazol-1- yl}-N-(3,3-dimethyl-butyl)- butyramide |
| 104 | 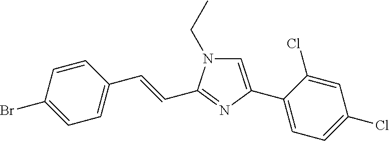 | 2-[2-(4-bromo-phenyl)-(E)- vinyl]-4-(2,4-dichloro- phenyl)-1-ethyl-1H- imidazole |
| 105 | 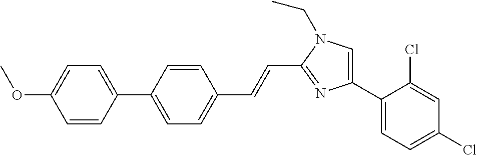 | 4-(2,4-dichloro-phenyl)-1- ethyl-2-[2-(4′-methoxy- biphenyl-4-yl)-(E)-vinyl]-1H- imidazole |
| 106 | 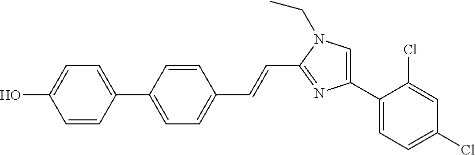 | 4′-{2-[4-(2,4-dichloro- phenyl)-1-ethyl-1H- imidazol-2-yl]-(E)-vinyl}- biphenyl-4-ol |
| 107 | 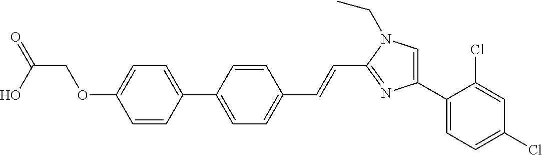 | (4′-{2-[4-(2,4-dichloro- phenyl)-1-ethyl-1H- imidazol-2-yl]-(E)-vinyl}- biphenyl-4-yloxy)-acetic acid |
| 108 | 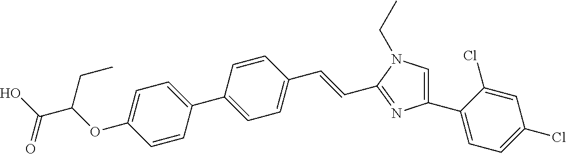 | 2-(4′-{2-[4-(2,4-dichloro- phenyl)-1-ethyl-1H- imidazol-2-yl]-(E)-vinyl}- biphenyl-4-yloxy)-butyric acid |
| 109 | 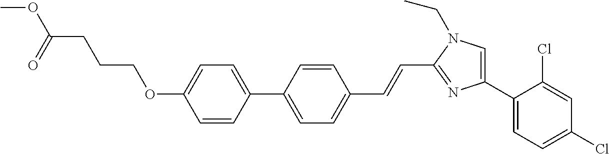 | 4-(4′-{2-[4-(2,4-dichloro- phenyl)-1-ethyl-1H- imidazol-2-yl]-(E)-vinyl}- biphenyl-4-yloxy)-butyric acid methyl ester |
| 110 | 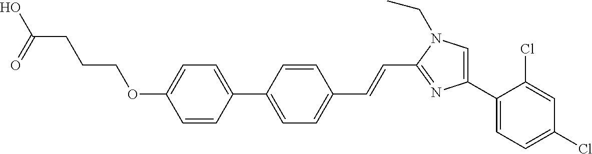 | 4-(4′-{2-[4-(2,4-dichloro- phenyl)-1-ethyl-1H- imidazol-2-yl]-(E)-vinyl}- biphenyl-4-yloxy)-butyric acid |
| 111 | 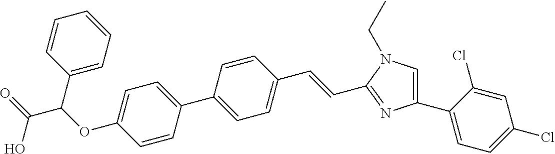 | (4′-{2-[4-(2,4-dichloro- phenyl)-1-ethyl-1H- imidazol-2-yl]-(E)-vinyl}- biphenyl-4-yloxy)-phenyl- acetic acid |
| 112 | 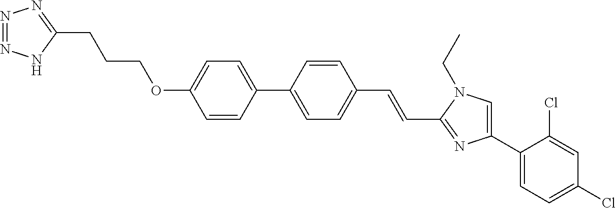 | 5-[3-(4′-{2-[4-(2,4-dichloro- phenyl)-1-ethyl-1H- imidazol-2-yl]-(E)-vinyl}- biphenyl-4-yloxy)-propyl]- 1H-tetrazole |
| 113 | 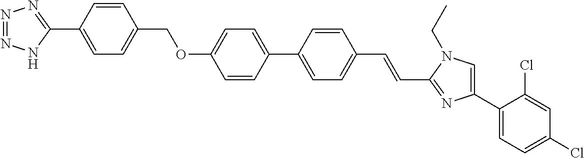 | 5-[4-(4′-{2-[4-(2,4-dichloro- phenyl)-1-ethyl-1H- imidazol-2-yl]-(E)-vinyl}- biphenyl-4-yloxymethyl)- phenyl]-1H-tetrazole |
| 114 | 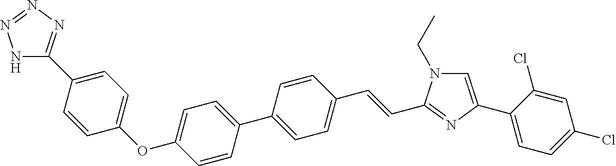 | 5-[4-(4′-{2-[4-(2,4-dichloro- phenyl)-1-ethyl-1H- imidazol-2-yl]-(E)-vinyl}- biphenyl-4-yloxy)-phenyl]- 1H-tetrazole |
| 115 | 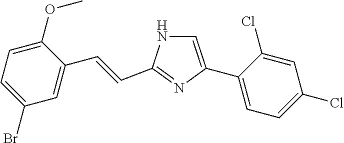 | 2-[2-(5-bromo-2-methoxy- phenyl)-(E)-vinyl]-4-(2,4- dichloro-phenyl)-1H- imidazole |
| 116 | 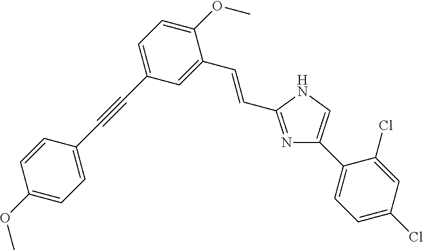 | 4-(2,4-dichloro-phenyl)-2-{2- [2-methoxy-5-(4-methoxy- phenylethynyl)-phenyl]-(E)- vinyl}-1H-imidazole |
| 117 | 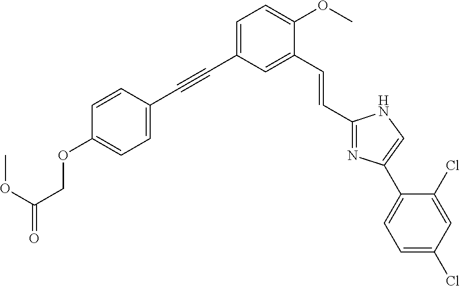 | [4-(3-{2-[4-(2,4-dichloro- phenyl)-1H-imidazol-2-yl]- (E)-vinyl}-4-methoxy- phenylethynyl)-phenoxy]- acetic acid methyl ester |
| 118 | 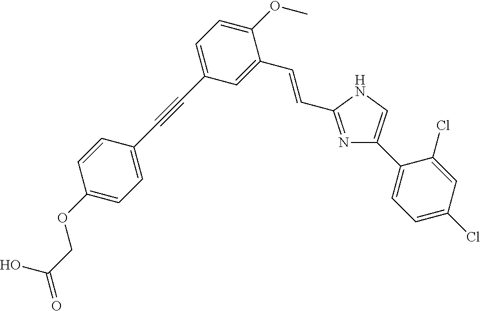 | [4-(3-{2-[4-(2,4-dichloro- phenyl)-1H-imidazol-2-yl]- (E)-vinyl}-4-methoxy- phenyl-ethynyl)-phenoxy]- acetic acid |
| 119 | 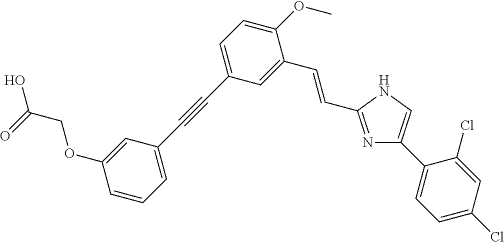 | [3-(3-{2-[4-(2,4-dichloro- phenyl)-1H-imidazol-2-yl]- (E)-vinyl}-4-methoxy- phenylethynyl)-phenoxy]- acetic acid |
| 120 | 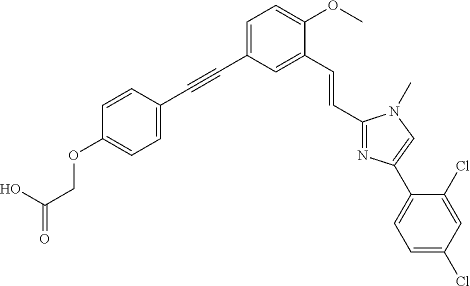 | [4-(3-{2-[4-(2,4-dichloro- phenyl)-1-methyl-1H- imidazol-2-yl]-(E)-vinyl}-4- methoxy-phenylethynyl)- phenoxy]-acetic acid |
| 121 | 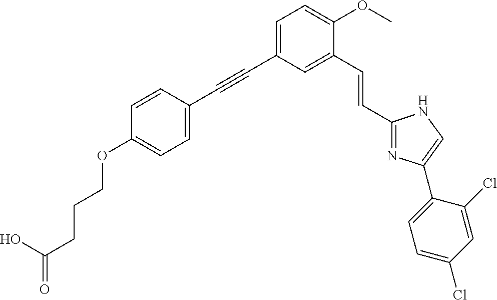 | 4-[4-(3-{2-[4-(2,4-dichloro- phenyl)-1H-imidazol-2-yl]- (E)-vinyl}-4-methoxy- phenylethynyl)-phenoxy]- butyric acid |
| 122 | 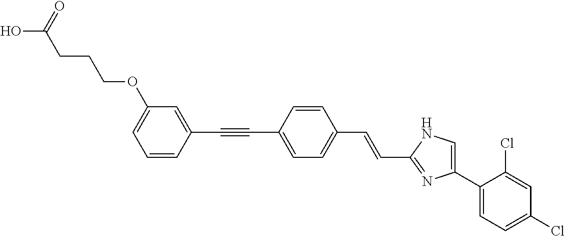 | 4-[3-(4-{2-[4-(2,4-dichloro- phenyl)-1H-imidazol-2-yl]- (E)-vinyl}-phenylethynyl)- phenoxy]-butyric acid |
| 123 | 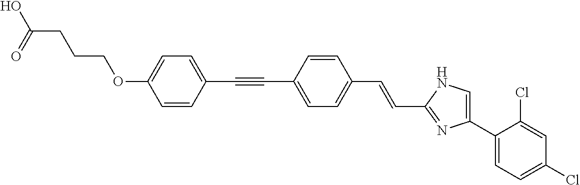 | 4-[4-(4-{2-[4-(2,4-dichloro- phenyl)-1H-imidazol-2-yl]- (E)-vinyl}-phenylethynyl)- phenoxy]-butyric acid |
| 124 | 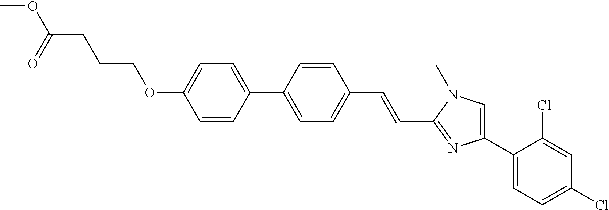 | 4-(4′-{2-[4-(2,4-dichloro- phenyl)-1-methyl-1H- imidazol-2-yl]-(E)-vinyl}- biphenyl-4-yloxy)-butyric acid methyl ester |
| 125 | 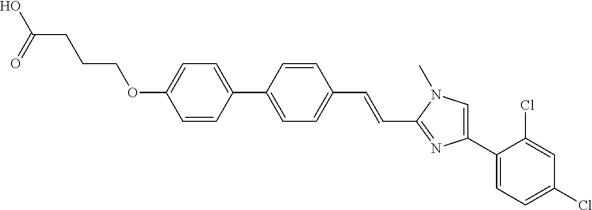 | 4-(4′-{2-[4-(2,4-dichloro- phenyl)-1-methyl-1H- imidazol-2-yl]-(E)-vinyl}- biphenyl-4-yloxy)-butyric acid |
| 126 | 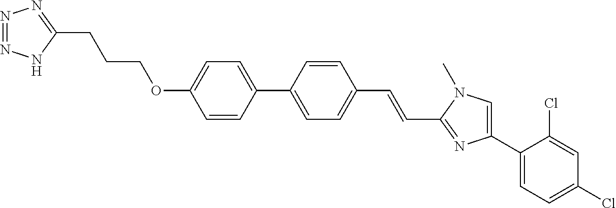 | 5-[3-(4′-{2-[4-(2,4-dichloro- phenyl)-1-methyl-1H- imidazol-2-yl]-(E)-vinyl}- biphenyl-4-yloxy)-propyl]- 1H-tetrazole |
| 127 | 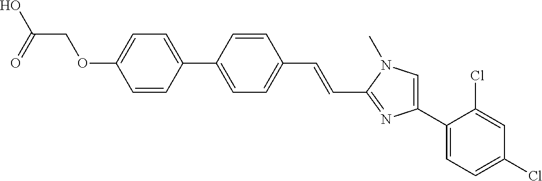 | (4′-{2-[4-(2,4-dichloro- phenyl)-1-methyl-1H- imidazol-2-yl]-(E)-vinyl}- biphenyl-4-yloxy)-acetic acid |
| 128 | 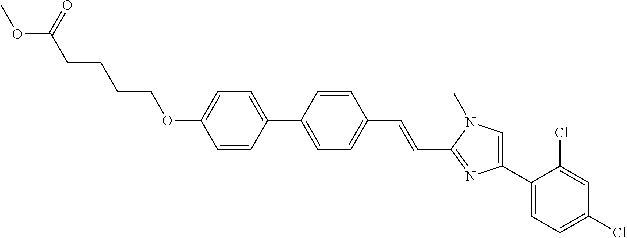 | 5-(4′-{2-[4-(2,4-dichloro- phenyl)-1-methyl-1H- imidazol-2-yl]-(E)-vinyl}- biphenyl-4-yloxy)-pentanoic acid methyl ester |
| 129 | 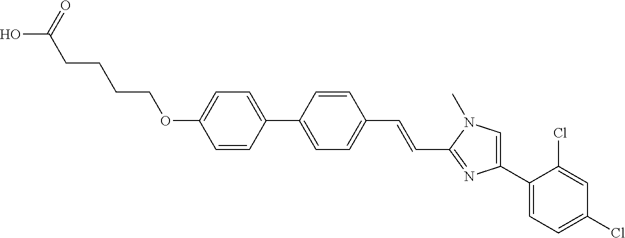 | 5-(4′-{2-[4-(2,4-dichloro- phenyl)-1-methyl-1H- imidazol-2-yl]-(E)-vinyl}- biphenyl-4-yloxy)-pentanoic acid |
| 130 | 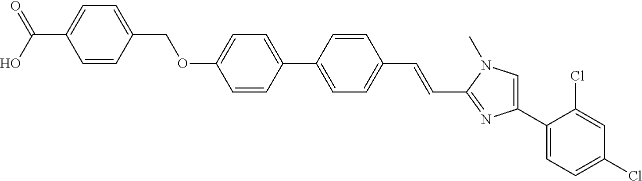 | 4-(4′-{2-[4-(2,4-dichloro- phenyl)-1-methyl-1H- imidazol-2-yl]-(E)-vinyl}- biphenyl-4-yloxymethyl)- benzoic acid |
| 131 | 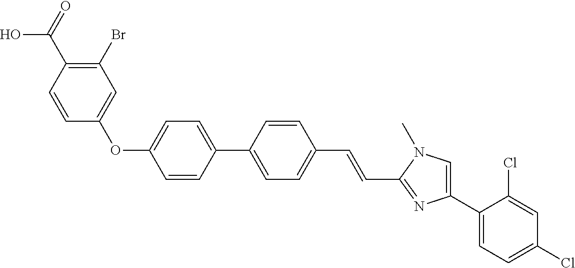 | 2-bromo-4-(4′-{2-[4-(2,4- dichloro-phenyl)-1-methyl- 1H-imidazol-2-yl]-(E)-vinyl}- biphenyl-4-yloxy)-benzoic acid |
| 132 | 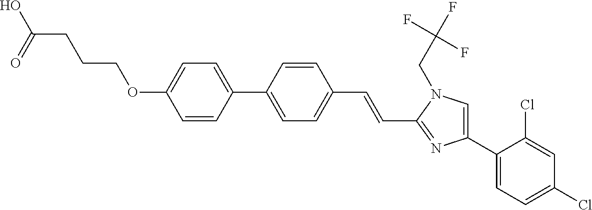 | 4-(4′-{2-[4-(2,4-dichloro- phenyl)-1-(2,2,2-trifluoro- ethyl)-1H-imidazol-2-yl]- (E)-vinyl}-biphenyl-4-yloxy)- butyric acid |
| 133 | 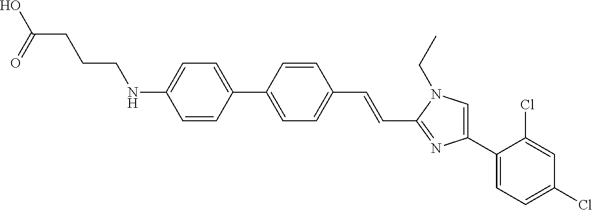 | 4-(4′-{2-[4-(2,4-dichloro- phenyl)-1-ethyl-1H- imidazol-2-yl]-(E)-vinyl}- biphenyl-4-ylamino)-butyric acid |
| 134 | 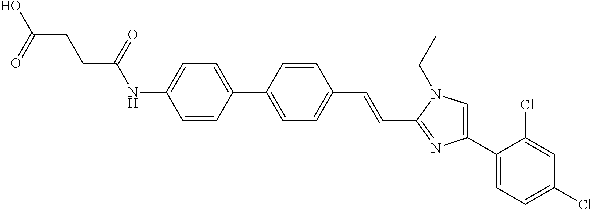 | N-(4′-{2-[4-(2,4-dichloro- phenyl)-1-ethyl-1H- imidazol-2-yl]-(E)-vinyl}- biphenyl-4-yl)-succinamic acid |
| 135 | 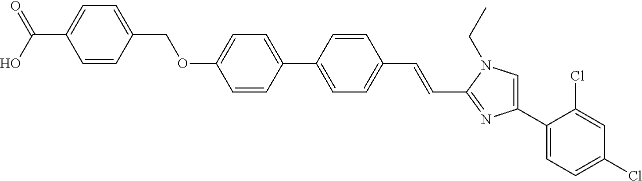 | 4-(4′-{2-[4-(2,4-dichloro- phenyl)-1-ethyl-1H- imidazol-2-yl]-(E)-vinyl}- biphenyl-4-yloxymethyl)- benzoic acid |
| 136 | 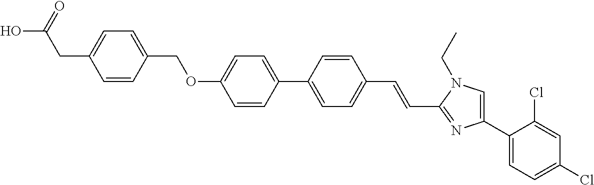 | [4-(4′-{2-[4-(2,4-dichloro- phenyl)-1-ethyl-1H- imidazol-2-yl]-(E)-vinyl}- biphenyl-4-yloxymethyl)- phenyl]-acetic acid |
| 137 | 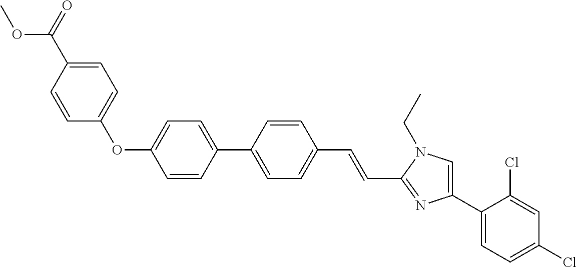 | 4-(4′-{2-[4-(2,4-dichloro- phenyl)-1-ethyl-1H- imidazol-2-yl]-(E)-vinyl}- biphenyl-4-yloxy)-benzoic acid methyl ester |
| 138 | 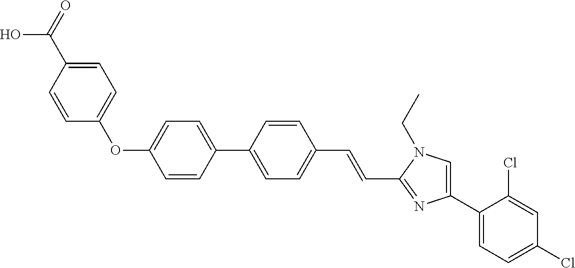 | 4-(4′-{2-[4-(2,4-dichloro- phenyl)-1-ethyl-1H- imidazol-2-yl]-(E)-vinyl}- biphenyl-4-yloxy)-benzoic acid |
| 139 | 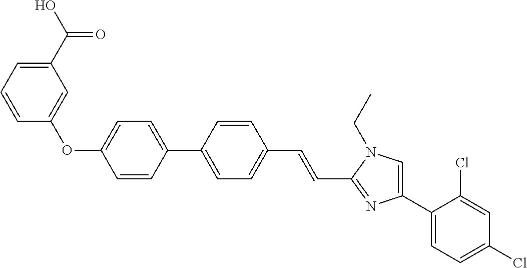 | 3-(4′-{2-[4-(2,4-dichloro- phenyl)-1-ethyl-1H- imidazol-2-yl]-(E)-vinyl}- biphenyl-4-yloxy)-benzoic acid |
| 140 | 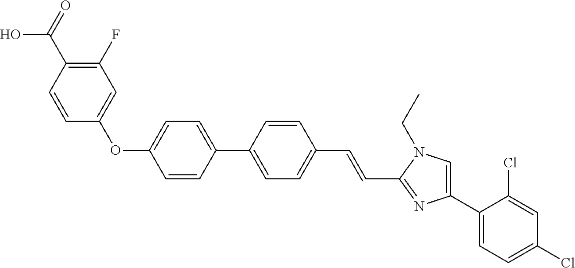 | 4-(4′-{2-[4-(2,4-dichloro- phenyl)-1-ethyl-1H- imidazol-2-yl]-(E)-vinyl}- biphenyl-4-yloxy)-2-fluoro- benzoic acid |
| 141 | 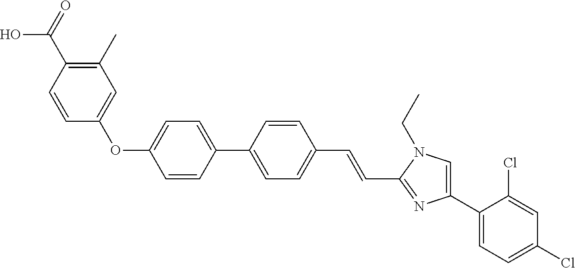 | 4-(4′-{2-[4-(2,4-dichloro- phenyl)-1-ethyl-1H- imidazol-2-yl]-(E)-vinyl}- biphenyl-4-yloxy)-2-methyl- benzoic acid |
| 142 | 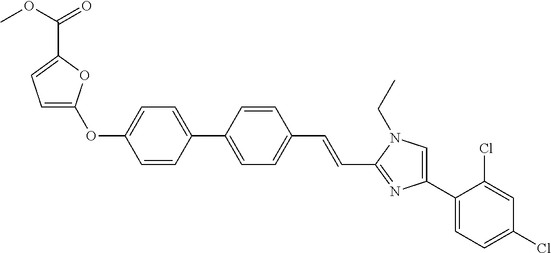 | 5-(4′-{2-[4-(2,4-dichloro- phenyl)-1-ethyl-1H- imidazol-2-yl]-(E)-vinyl}- biphenyl-4-yloxy)-furan-2- carboxylic acid methyl ester |
| 143 | 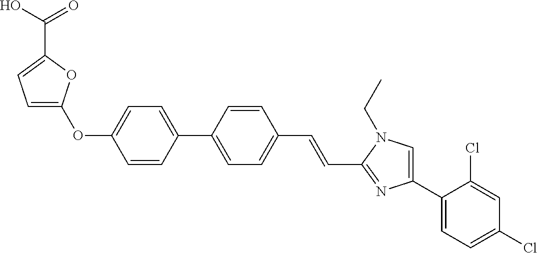 | 5-(4′-{2-[4-(2,4-dichloro- phenyl)-1-ethyl-1H- imidazol-2-yl]-(E)-vinyl}- biphenyl-4-yloxy)-furan-2- carboxylic aci |
| 144 | 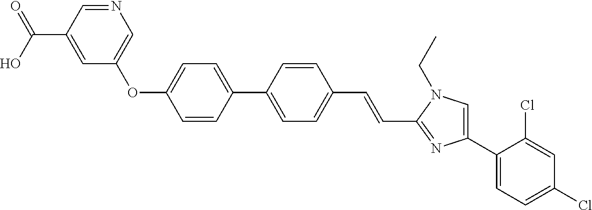 | 5-(4′-{2-[4-(2,4-dichloro- phenyl)-1-ethyl-1H- imidazol-2-yl]-(E)-vinyl}- biphenyl-4-yloxy)-nicotinic acid |
| 145 | 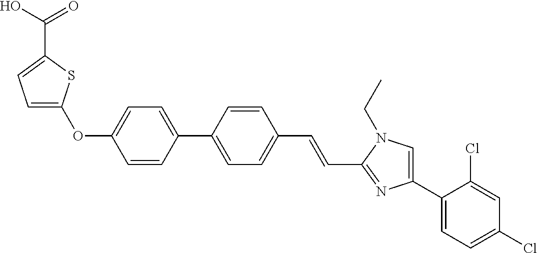 | 5-(4′-{2-[4-(2,4-dichloro- phenyl)-1-ethyl-1H- imidazol-2-yl]-(E)-vinyl}- biphenyl-4-yloxy)- thiophene-2-carboxylic acid |
| 146 | 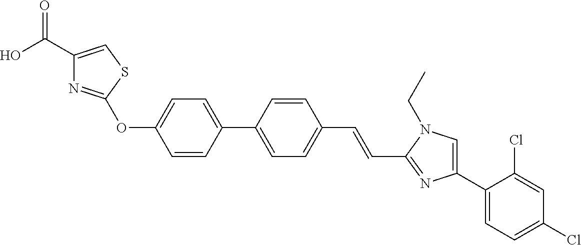 | 2-(4′-{2-[4-(2,4-dichloro- phenyl)-1-ethyl-1H- imidazol-2-yl]-(E)-vinyl}- biphenyl-4-yloxy)-thiazole- 4-carboxylic acid |
| 147 | 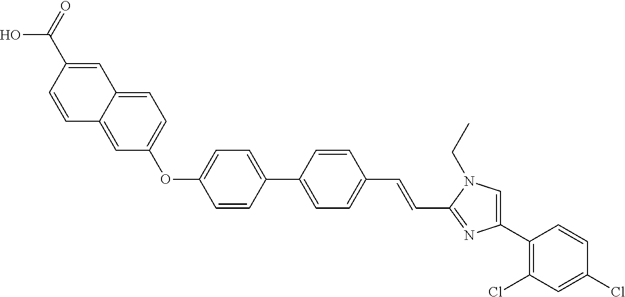 | 6-(4′-{2-[4-(2,4-dichloro- phenyl)-1-ethyl-1H- imidazol-2-yl]-(E)-vinyl}- biphenyl-4-yloxy)- naphthalene-2-carboxylic acid |
| 148 | 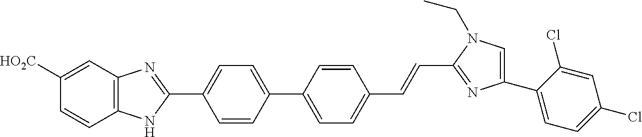 | 2-(4′-{2-[4-(2,4-dichloro- phenyl)-1-ethyl-1H- imidazol-2-yl]-(E)-vinyl}- biphenyl-4-yl)-1H- benzoimidazole-5-carboxylic acid |
| 149 | 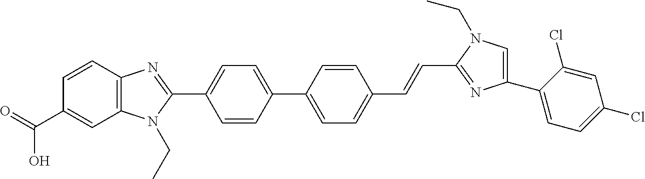 | 2-(4′-{2-[4-(2,4-dichloro- phenyl)-1-ethyl-1H- imidazol-2-yl]-(E)-vinyl}- biphenyl-4-yl)-3-ethyl-3H- benzoimidazole-5- carboxylic acid |
| 150 | 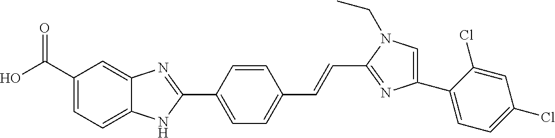 | 2-(4′-{2-[4-(2,4-dichloro- phenyl)-1-ethyl-1H- imidazol-2-yl]-(E)-vinyl}- phenyl)-1H-benzoimidazole-5- carboxylic acid |
| 151 | 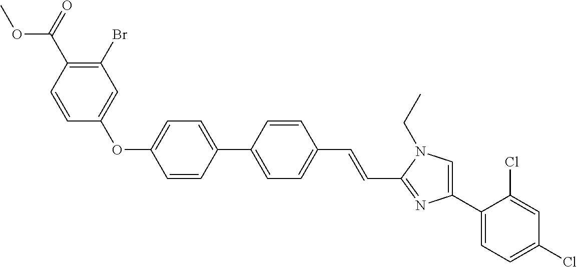 | 2-bromo-4-(4′-{2-[4-(2,4- dichloro-phenyl)-1-ethyl- 1H-imidazol-2-yl]-(E)-vinyl}- biphenyl-4-yloxy)-benzoic acid methyl este |
| 152 | 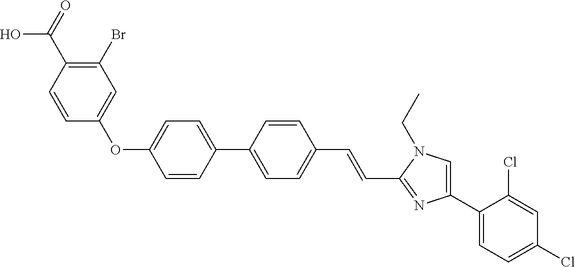 | 2-bromo-4-(4′-{2-[4-(2,4- dichloro-phenyl)-1-ethyl- 1H-imidazol-2-yl]-(E)-vinyl}- biphenyl-4-yloxy)-benzoic acid |
| 153 | 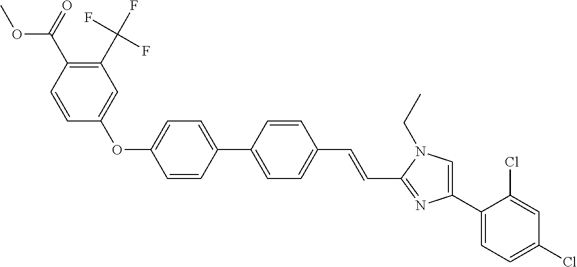 | 4-(4′-{2-[4-(2,4-dichloro- phenyl)-1-ethyl-1H- imidazol-2-yl]-(E)-vinyl}- biphenyl-4-yloxy)-2- trifluoromethyl-benzoic acid methyl ester |
| 154 | 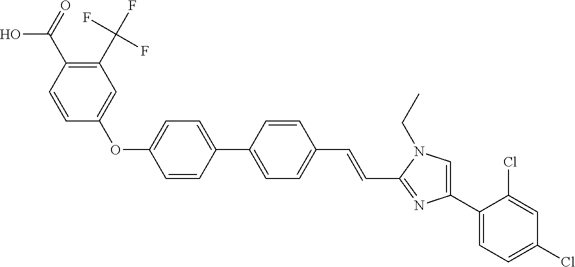 | 4-(4′-{2-[4-(2,4-dichloro- phenyl)-1-ethyl-1H- imidazol-2-yl]-(E)-vinyl}- biphenyl-4-yloxy)-2- trifluoromethyl-benzoic acid |
| 155 | 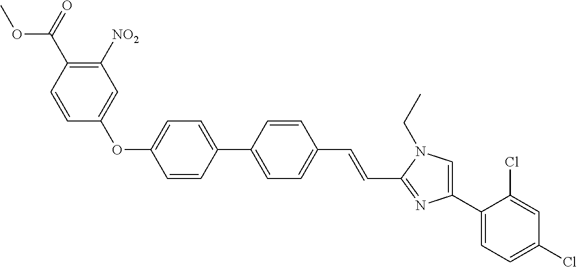 | 4-(4′-{2-[4-(2,4-dichloro- phenyl)-1-ethyl-1H- imidazol-2-yl]-(E)-vinyl}- biphenyl-4-yloxy)-2-nitro- benzoic acid methyl ester |
| 156 | 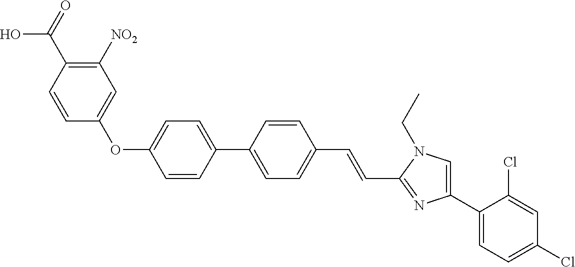 | 4-(4′-{2-[4-(2,4-dichloro- phenyl)-1-ethyl-1H- imidazol-2-yl]-(E)-vinyl}- biphenyl-4-yloxy)-2-nitro- benzoic acid |
| 157 | 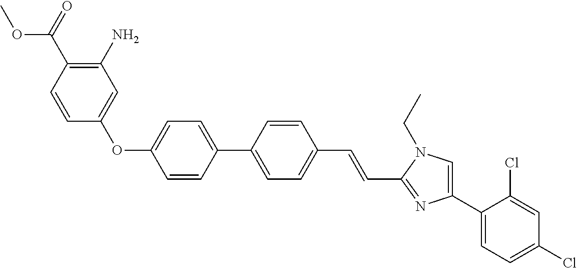 | 2-amino-4-(4′-{2-[4-(2,4- dichloro-phenyl)-1-ethyl- 1H-imidazol-2-yl]-(E)-vinyl}- biphenyl-4-yloxy)-benzoic acid methyl ester |
| 158 | 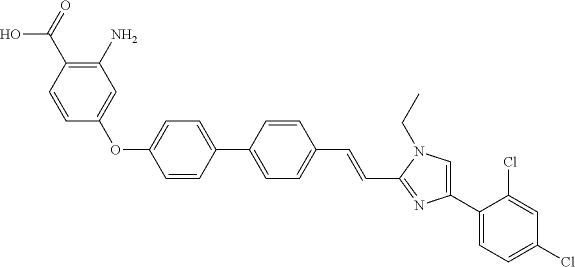 | 2-amino-4-(4′-{2-[4-(2,4- dichloro-phenyl)-1-ethyl- 1H-imidazol-2-yl]-(E)-vinyl}- biphenyl-4-yloxy)-benzoic acid |
| 159 | 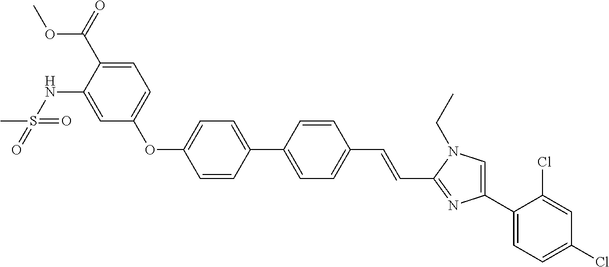 | 4-(4′-{2-[4-(2,4-dichloro- phenyl)-1-ethyl-1H- imidazol-2-yl]-(E)-vinyl}- biphenyl-4-yloxy)-2- methanesulfonylamino- benzoic acid methyl ester |
| 160 | 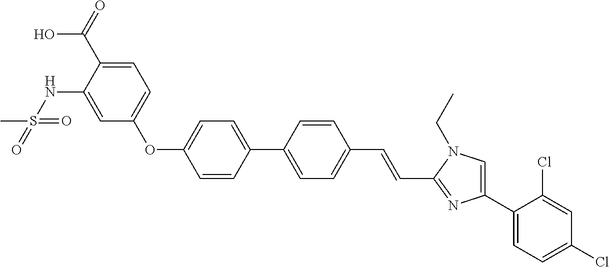 | 4-(4′-{2-[4-(2,4-dichloro- phenyl)-1-ethyl-1H- imidazol-2-yl]-(E)-vinyl}- biphenyl-4-yloxy)-2- methanesulfonylamino- benzoic acid |
| 161 | 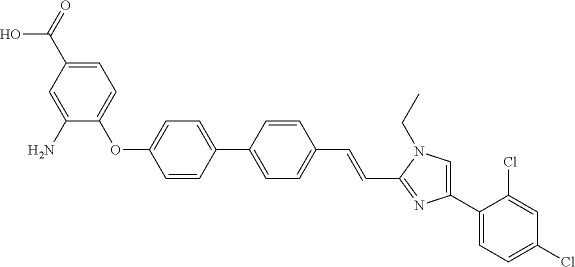 | 3-amino-4-(4′-{2-[4-(2,4- dichloro-phenyl)-1-ethyl- 1H-imidazol-2-yl]-(E)-vinyl}- biphenyl-4-yloxy)-benzoic acid |
| 162 | 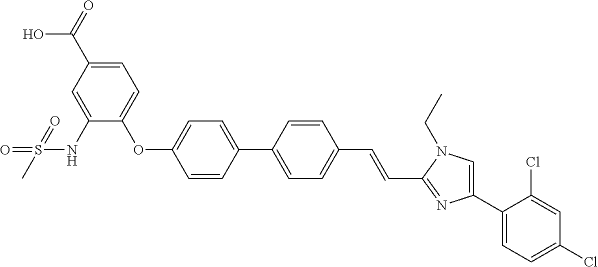 | 4-(4′-{2-[4-(2,4-dichloro- phenyl)-1-ethyl-1H- imidazol-2-yl]-(E)-vinyl}- biphenyl-4-yloxy)-3- methanesulfonylamino- benzoic acid |
| 163 | 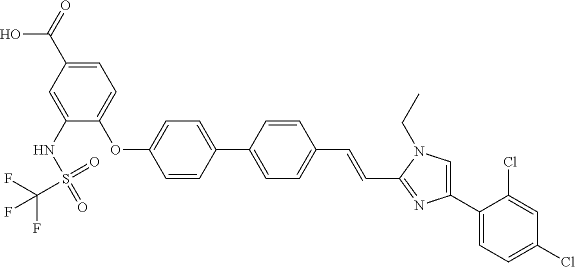 | 4-(4′-{2-[4-(2,4-dichloro- phenyl)-1-ethyl-1H- imidazol-2-yl]-(E)-vinyl}- biphenyl-4-yloxy)-3- trifluoromethanesulfonyl- amino-benzoic acid |
| 164 | 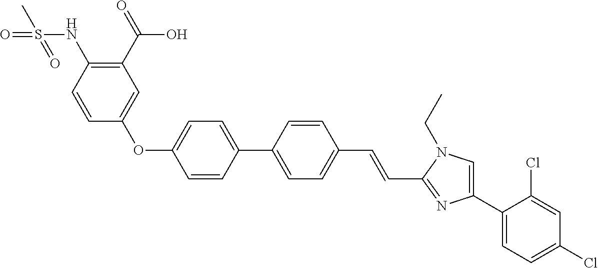 | 5-(4′-{2-[4-(2,4-dichloro- phenyl)-1-ethyl-1H- imidazol-2-yl]-(E)-vinyl}- biphenyl-4-yloxy)-2- methanesulfonylamino- benzoic acid |
| 165 | 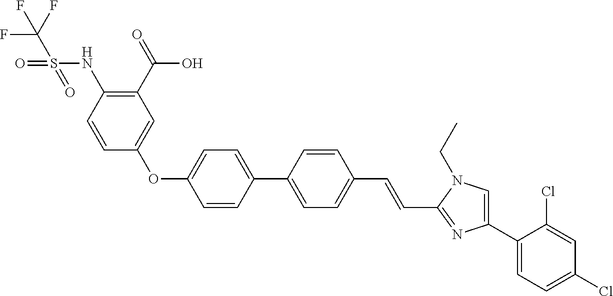 | 5-(4′-{2-[4-(2,4-dichloro- phenyl)-1-ethyl-1H- imidazol-2-yl]-(E)-vinyl}- biphenyl-4-yloxy)-2- trifluoromethane- sulfonylamino-benzoic acid |
| 166 | 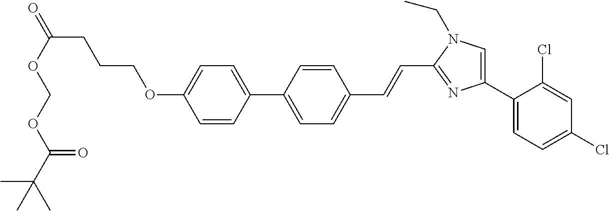 | 4-(4′-{2-[4-(2,4-Dichloro- phenyl)-1-ethyl-1H- imidazol-2-yl]-(E)-vinyl}- biphenyl-4-yloxy)-butyric acid 2,2-dimethyl- propionyloxymethyl ester |
| 167 | 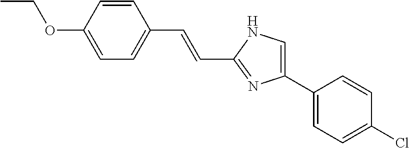 | 4-(4-chloro-phenyl)-2-[2-(4- ethoxy-phenyl)-(E)-vinyl- 1H-imidazole |
| 168 | 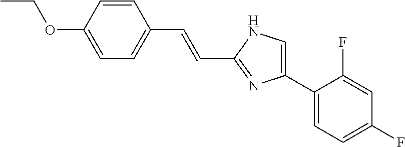 | 4-(2,4-difluoro-phenyl)-2-[2- (4-ethoxy-phenyl)-(E)- vinyl]-1H-imidazole |
| 169 | 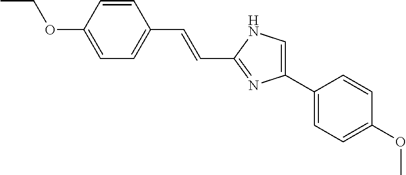 | 2-[2-(4-ethoxy-phenyl)-(E)- vinyl]-4(4-methoxy-phenyl)- 1H-imidazole |
| 170 | 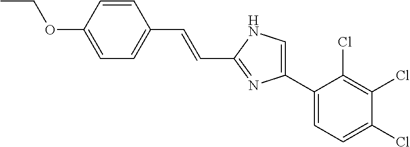 | 2-[2-(4-ethoxy-phenyl)-(E)- vinyl]-4-(2,3,4-trichloro- phenyl)-1H-imidazole |
| 171 | 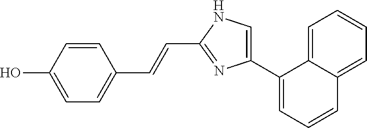 | 4-[2-(4-naphthalen-1yl-1H- imidazole-2-yl)-(E)-vinyl]- phenol |
| 172 | 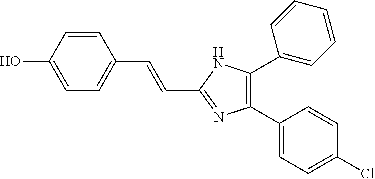 | 4-{2-[4-(4-chloro-phenyl)-5- phenyl-1H-imidazole-2-yl]- (E)-vinyl}-phenol |
| 173 | 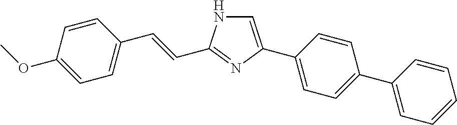 | 4-biphenyl-4-yl-2-[2-(4- methoxy-phenyl)-(E)-vinyl]- 1H-imidazole |
| 174 | 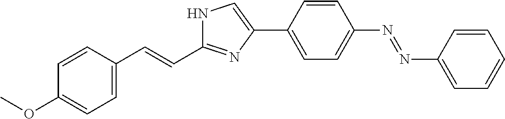 | (4-{2-[2-(4-methoxy- phenyl)-(E)-vinyl]-1H- imidazole-4-yl}-phenyl- diazene |
| 175 | 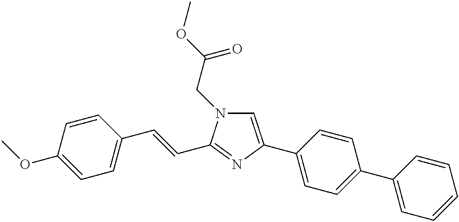 | {4-biphenyl-4-yl-2-[2-(4- methoxy-phenyl)-(E)-vinyl]- imidazole-1yl}-acetic acid methyl ester |
| 176 | 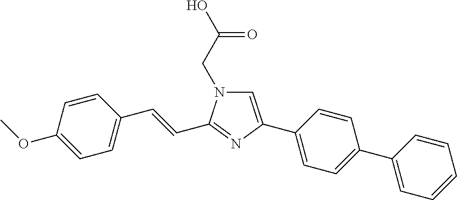 | {4-biphenyl-4-yl-2-[2-(4- methoxy-phenyl)-(E)-vinyl]- imidazole-1yl}-acetic acid |
| 177 | 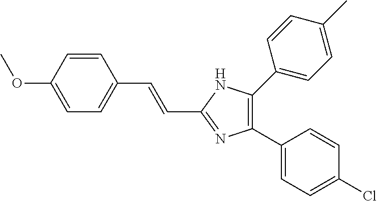 | 4-(4-chloro-phenyl)-2-[2-(4- methoxy-phenyl)-(E)-vinyl]- 5-p-tolyl-1H-imidazole |
| 178 | 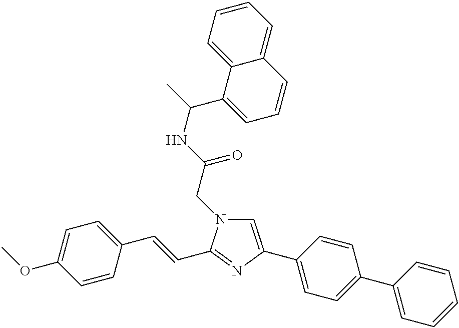 | 2-{4-biphenyl-4-yl-2-[2-(4- methoxy-phenyl)-(E)-vinyl]- imidazole-1yl}-N-(1- naphthalen-1-yl-ethyl)- acetamide |
| 179 | 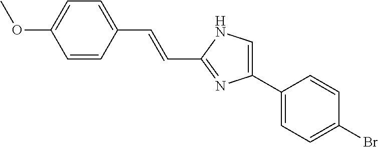 | 4-(4-bromo-phenyl)-2-[2-(4- methoxy-phenyl)-(E)-vinyl]- 1H-imidazole |
| 180 | 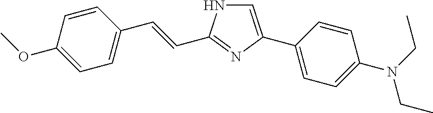 | diethyl-(4-{2-[2-(4-methoxy- phenyl)-(E)-vinyl]-1H- imidazol-4yl}-phenyl)- amine |
| 181 | 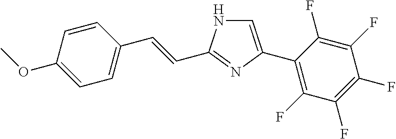 | 2-[2-(4-methoxy-phenyl)- (E)-vinyl]-4- pentafluorophenyl-1H- imidazole |
| 182 | 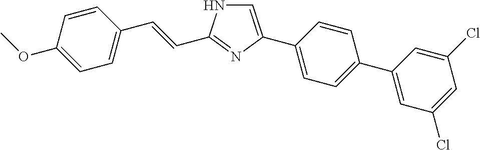 | 4-(3′,5′-dichloro-biphenyl- 4-yl)-2-[2-(4-methoxy- phenyl)-(E)-vinyl]-1H- imidazole |
| 183 | 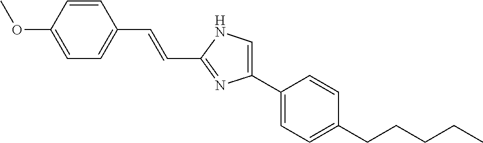 | 2-[2-(4-methoxy-phenyl)- (E)-vinyl]-4-(4-pentyl- phenyl)-1H-imidazole |
| 184 | 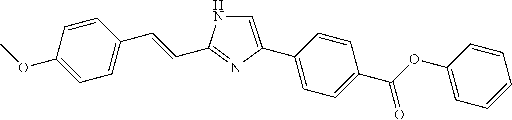 | 4-{2-[2-(4-methoxy-phenyl)- (E)-vinyl]-1H-imidazol-4-yl}- benzoic acid phenyl ester |
| 185 | 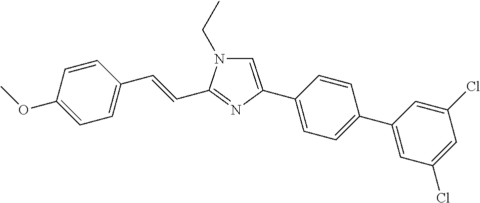 | 4-(3′,5′-dichloro-biphenyl- 4-yl)-1-ethyl-2-[-2-(4- methoxy-phenyl)-(E)-vinyl]- 1H-imidazole |
| 186 | 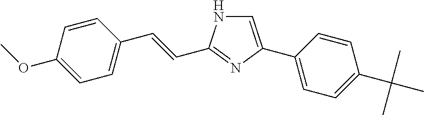 | 4-(4-tert-butyl-phenyl)-2-[2- (4-methoxy-phenyl)-(E)- vinyl]-1H-imidazole |
| 187 | 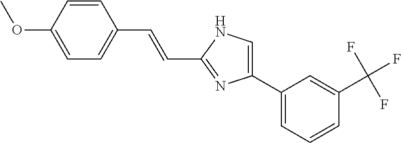 | 2-[2-(4-methoxy-phenyl)- (E)-vinyl]-4-(3- trifluoromethyl-phenyl)-1H- imidazole |
| 188 | 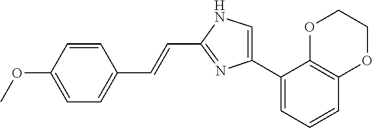 | 4-(2,3-dihydro- benzo[1,4]dioxin-5-yl)-2-[2- (4-methoxy-phenyl)-(E)- vinyl]-1H-imidazole |
| 189 | 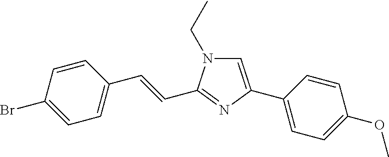 | 2-[2-(4-bromo-phenyl)-(E)- vinyl]-1-ethyl-4-(4-methoxy- phenyl)-1H-imidazole |
| 190 | 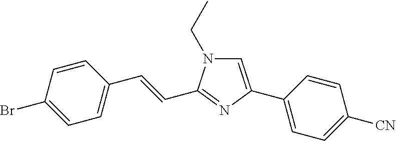 | 2-[2-(4-bromo-phenyl)-(E)- vinyl]-1-ethyl-4-(4-cyano- phenyl)-1H-imidazole |
| 191 | 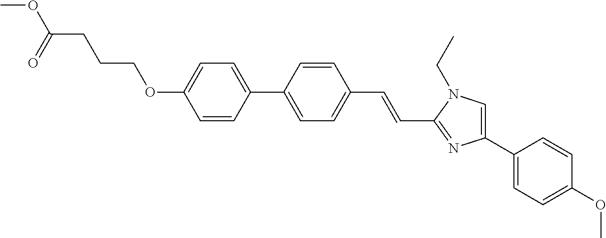 | 4-(4′-{2-[1-ethyl-4-(4- methoxy-phenyl)-1H- imidazol-2-yl]-(E)-vinyl}- biphenyl-4-yloxy)-butyric acid methyl ester |
| 192 | 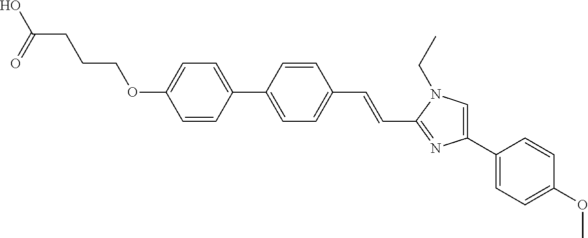 | 4-(4′-{2-[1-ethyl-4-(4- methoxy-phenyl)-1H- imidazol-2-yl]-(E)-vinyl}- biphenyl-4-yloxy)-butyric acid |
| 193 | 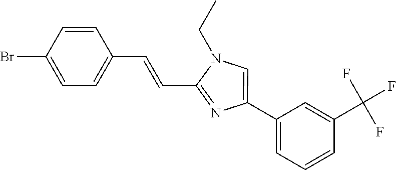 | 2-[2-(4-bromo-phenyl)-(E)- vinyl]-1-ethyl-4-(3- trifluoromethyl-phenyl)-1H- imidazole |
| 194 | 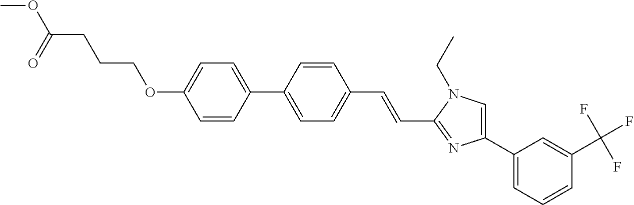 | 4-(4′-[2-[1-ethyl-4-(3- trifluoromethyl-phenyl)-1H- imidazol-2-yl]-(E)-vinyl}- biphenyl-4yloxy)-butyric acid methyl ester |
| 195 | 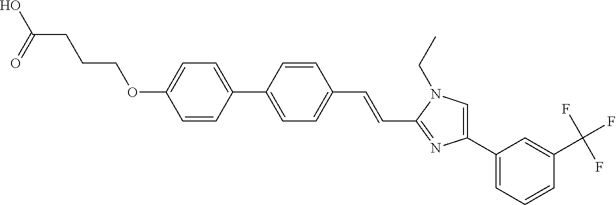 | 4-(4′-[2-[1-ethyl-4-(3- trifluoromethyl-phenyl)-1H- imidazol-2-yl]-(E)-vinyl}- biphenyl-4yloxy)-butyric acid |
| 196 | 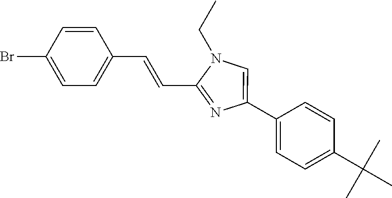 | 2-[2-(4-bromo-phenyl)-(E)- vinyl]-4-(4-tert-butyl- phenyl)-1-ethyl-1H- imidazole |
| 197 | 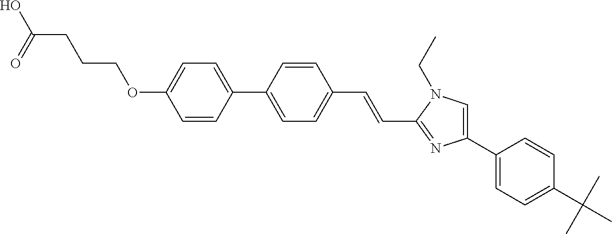 | 4-(4′-{2-[4-tert-butyl- phenyl)-1-ethyl-iH-imidazol- 2-yl]-(E)-vinyl}-biphenyl-4- yloxy)-butyric acid |
| 198 | 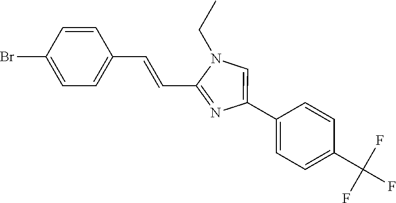 | 2-[2-(4-bromo-phenyl)-(E)- vinyl]-1-ethyl-4-(4- trifluoromethyl-phenyl)-1H- imidazole |
| 199 | 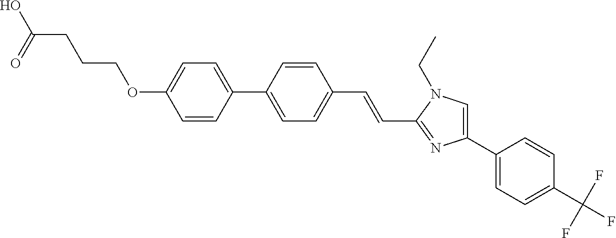 | 4-(-4′-{2-[1-ethyl-4-(4- trifluoromethyl-phenyl)-1H- imidazol-2-yl]-(E)-vinyl}- biphenyl-4-yloxy)-butyric acid |
| 200 | 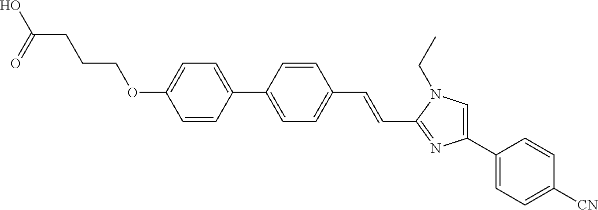 | 4-(-4′-{2-[1-ethyl-4-(4- cyano-phenyl)-1H-imidazol- 2-yl]-(E)-vinyl}-biphenyl-4- yloxy)-butyric acid |
| 201 | 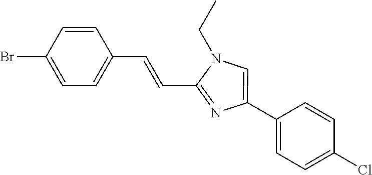 | 2-[2-(4-bromo-phenyl)-(E)- vinyl]-1-ethyl-4-(4-chloro- phenyl)-1H-imidazole |
| 202 | 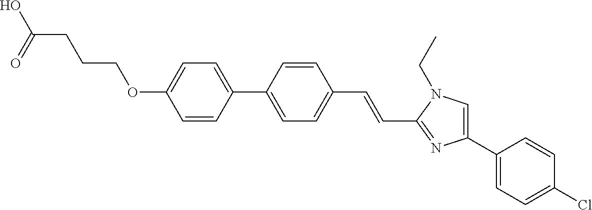 | 4-(-4′-{2-[1-ethyl-4-(4- chloro-phenyl)-1H- imidazol-2-yl]-(E)-vinyl}- biphenyl-4-yloxy)-butyric acid |
| 203 | 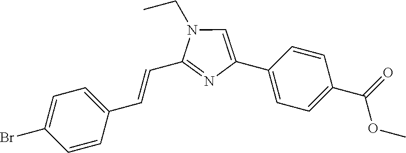 | 4-{2-[2-(4-bromo-phenyl)- (E)-vinyl]-1-ethyl-1H- imidazol-4-yl}-benzoic acid methyl ester |
| 204 | 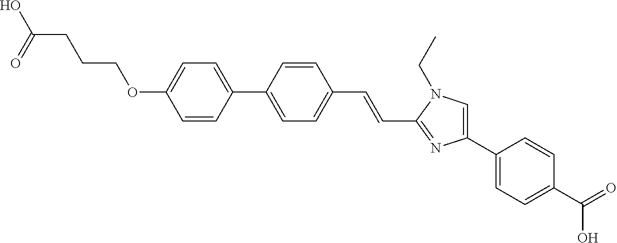 | 4-(1-ethyl-2-{2-[4′-(3- methoxycarbonyl-propoxy)- biphenyl-4-yl}-1H-imidazol- 4-yl)-benzoic acid |
| 205 | 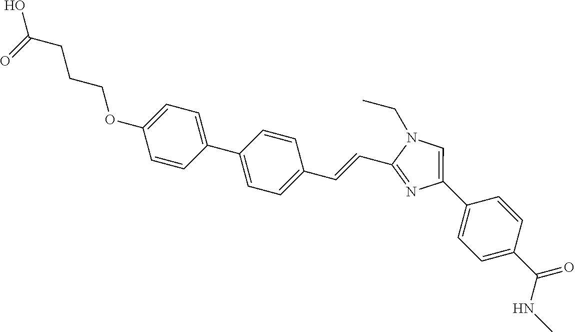 | 4-(-4′-{2-[1-ethyl-4-(4- methylcarbamoyl-phenyl)- 1H-imidazole-2yl]-(E)-vinyl}- biphenyl-4-yloxy)-butyric acid |
| 206 | 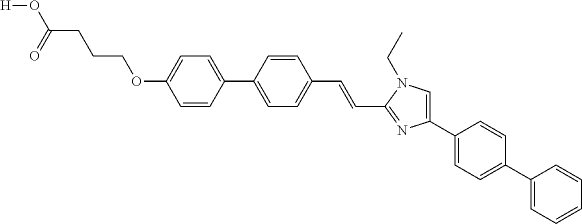 | 4-{4′-[2-(4-biphenyl-4-yl-1- ethyl-1H-imidazol-2-yl)-(E)- vinyl]-biphenyl-4-yloxy}- butyric acid |
| 207 | 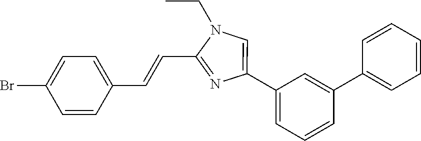 | 4-biphenyl-3-yl-2-[2-(4- bromo-phenyl)-(E)-vinyl]-1- ethyl-1H-imidazole |
| 208 | 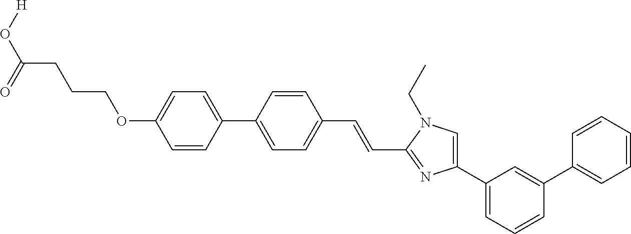 | 4-{-4′-[2-(4-biphenyl-3-yl-1- ethyl-1H-imidazol-2-yl)-(E)- vinyl]-biphenyl-4-yloxy}- butyric acid |
| 209 | 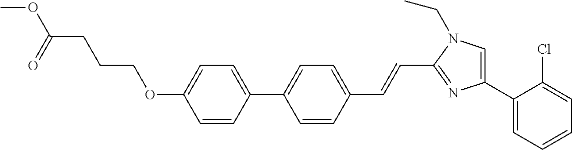 | 4-(4′-{2-[4-(2-chloro- phenyl)-1-ethyl-1H- imidazole-2-yl]-(E)-vinyl}- biphenyl-4-yloxy)-butyric acid methyl ester |
| 210 | 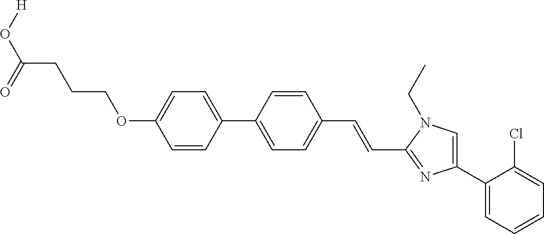 | 4-(4′-{2-[4-(2-chloro- phenyl)-1-ethyl-1H- imidazole-2-yl]-(E)-vinyl}- biphenyl-4-yloxy)-butyric acid |
| 211 | 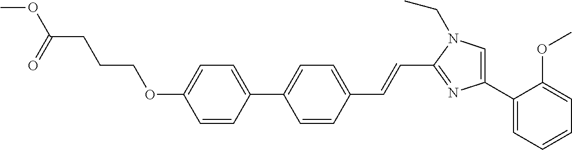 | 4-(4′-{2-[4-(2-methoxy- phenyl)-1-ethyl-1H- imidazole-2-yl]-(E)-vinyl}- biphenyl-4-yloxy)-butyric acid methyl ester |
| 212 | 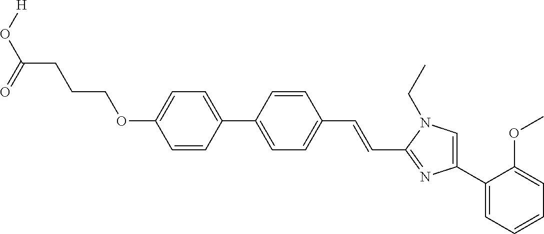 | 4-(4′-{2-[4-(2-methoxy- phenyl)-1-ethyl-1H- imidazole-2-yl]-(E)-vinyl}- biphenyl-4-yloxy)-butyric acid |
| 213 | 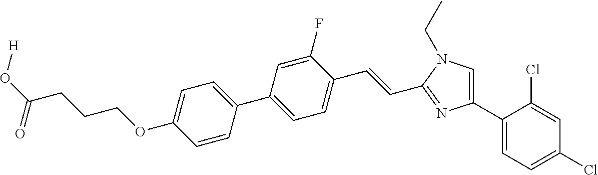 | 4-(4′-{2-[4-(2,4-dichloro- phenyl)-1-ethyl-1H- imidazol-2-yl]-(E)-vinyl}- 3′-fluoro-biphenyl-4-yloxy)- butyric acid |
| 214 | 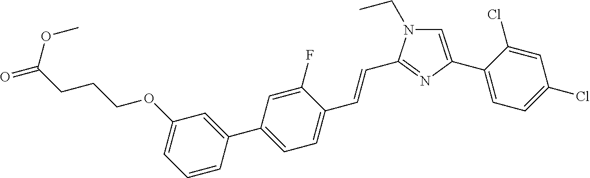 | 4-(4′-{2-[4-(2,4-dichloro- phenyl)-1-ethyl-1H- imidazol-2-yl]-(E)-vinyl}- 3′-fluoro-biphenyl-3-yloxy)- butyric acid methyl ester |
| 215 | 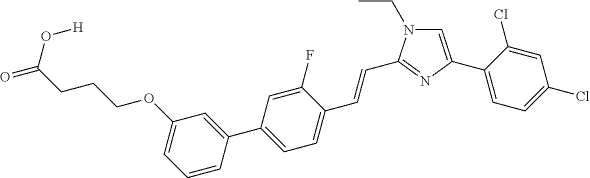 | 4-(4′-{2-[4-(2,4-dichloro- phenyl)-1-ethyl-1H- imidazol-2-yl]-(E)-vinyl}-3′- fluoro-biphenyl-3-yloxy)- butyric acid |
| 216 | 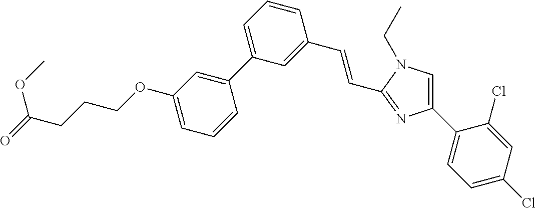 | 4-(3′-{2-[4-(2,4-dichloro- phenyl)-1-ethyl-1H- imidazol-2-yl]-(E)-vinyl}- biphenyl-3-yloxy)-butyric acid methyl ester |
| 217 | 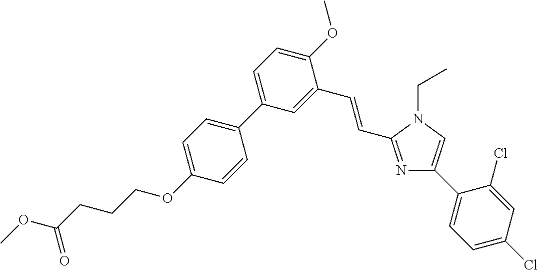 | 4-(3′-{2-[4-(2,4-dichloro- phenyl)-1-ethyl-1H- imidazol-2-yl]-(E)-vinyl}-4- methoxy-biphenyl-4-yloxy)- butyric acid methyl ester |
| 218 | 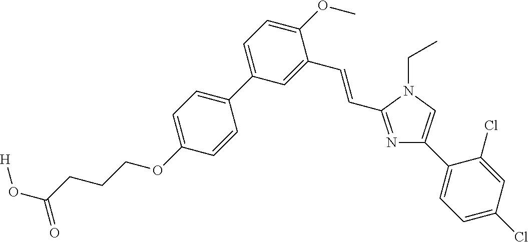 | 4-(3′-{2-[4-(2,4-dichloro- phenyl)-1-ethyl-1H- imidazol-2-yl]-(E)-vinyl}-4- methoxy-biphenyl-4-yloxy)- butyric acid |
| 219 | 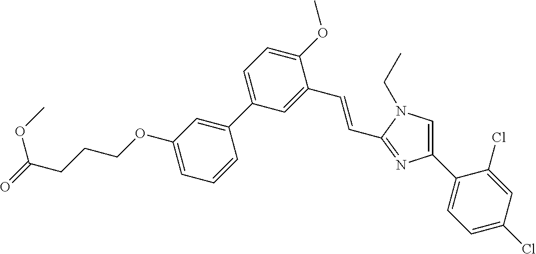 | 4-(3′-{2-[4-(2,4-Dichloro- phenyl)-1-ethyl-1H- imidazol-2-yl]-(E)-vinyl;}-4′- methoxy-biphenyl-3-yloxy)- butyric acid methyl ester |
| 220 | 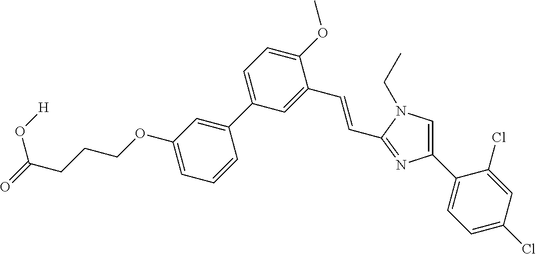 | 4-(3′-{2-[4-(2,4-dichloro- phenyl)-1-ethyl-1H- imidazol-2-yl]-(E)-vinyl;}-4′- methoxy-biphenyl-3-yloxy)- butyric acid |
| 221 | 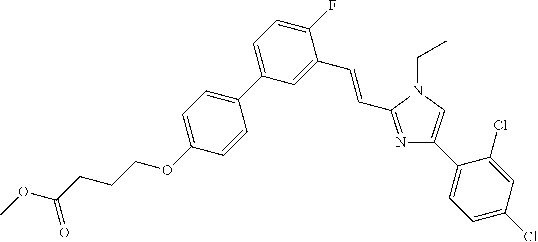 | 4-(3′-{2-[4-(2,4-dichloro- phenyl)-1-ethyl-1H- imidazol-2-yl]-(E)-vinyl}-4′- fluoro-biphenyl-4-yloxy)- butyric acid methyl ester |
| 222 | 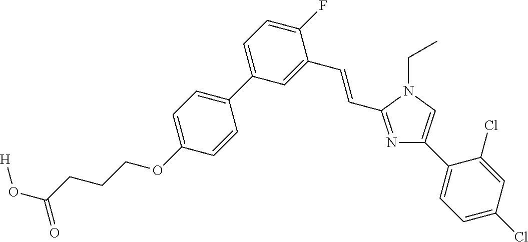 | 4-(3′-{2-[4-(2,4-dichloro- phenyl)-1-ethyl-1H- imidazol-2-yl]-(E)-vinyl}-4′- fluoro-biphenyl-4-yloxy)- butyric acid |
| 223 | 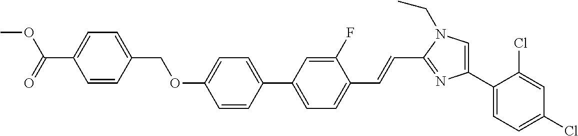 | 4-(4′-{2-[4-(2,4-dichloro- phenyl)-1-ethyl-1H- imidazol-2-yl]-(E)-vinyl-}- 3′-fluoro biphenyl-4- yloxymethyl)-benzoic acid methyl ester |
| 224 | 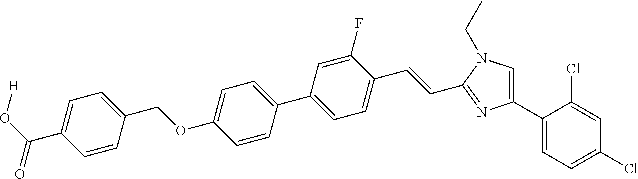 | 4-(4′-{2-[4-(2,4-dichloro- phenyl)-1-ethyl-1H- imidazol-2-yl]-(E)-vinyl-}- 3′-fluoro biphenyl-4- yloxymethyl)-benzoic acid |
| 225 | 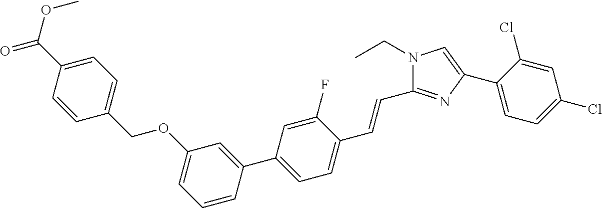 | 4-(4′-{2-[4-(2,4-dichloro- phenyl)-1-ethyl-1H- imidazol-2-yl]-(E)-vinyl-}- 3′-fluoro biphenyl-3- yloxymethyl)-benzoic acid methyl ester |
| 226 | 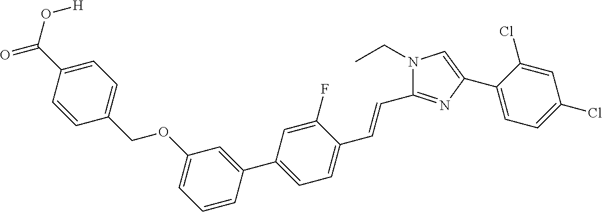 | 4-(4′-{2-[4-(2,4-dichloro- phenyl)-1-ethyl-1H- imidazol-2-yl]-(E)-vinyl-}- 3′-fluoro biphenyl-3- yloxymethyl)-benzoic acid |
| 227 | 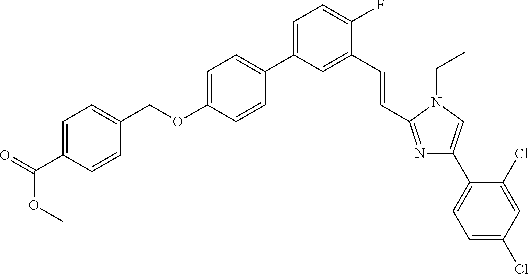 | 4-(3′-{2-[4-(2,4-dichloro- phenyl)-1-ethyl-1H- imidazol-2-yl]-(E)-vinyl-}- 4′-fluorobiphenyl-4- yloxymethyl)-benzoic acid methyl ester |
| 228 | 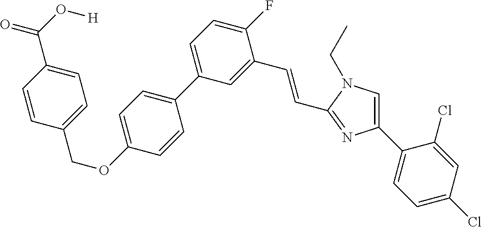 | 4-(3′-{2-[4-(2,4-dichloro- phenyl)-1-ethyl-1H- imidazol-2-yl]-(E)-vinyl-}- 4′-fluorobiphenyl-4- yloxymethyl)-benzoic acid |
| 229 | 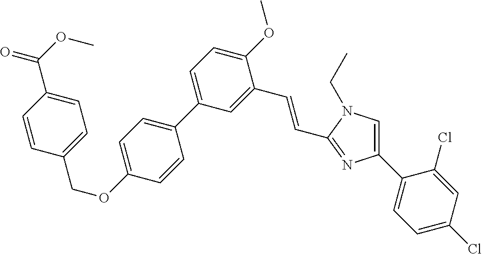 | 4-(3′-{2-[4-(2,4-dichloro- phenyl)-1-ethyl-1H- imidazol-2-yl]-(E)-vinyl-}- 4′-methoxy-biphenyl-4- yloxymethyl)-benzoic acid methyl ester |
| 230 | 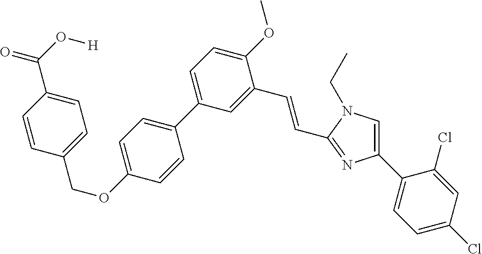 | 4-(3′-{2-[4-(2,4-dichloro- phenyl)-1-ethyl-1H- imidazol-2-yl]-(E)-vinyl-}- 4′-methoxy-biphenyl-4- yloxymethyl)-benzoic acid |
| 231 | 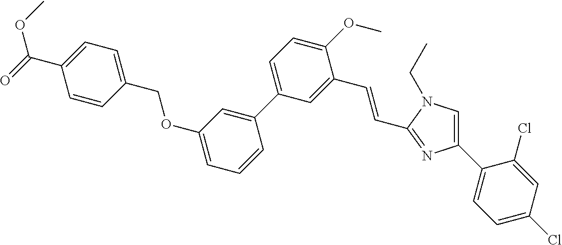 | 4-(3′-{2-[4-(2,4-dichloro- phenyl)-1-ethyl-1H- imidazol-2-yl]-(E)-vinyl-}- 4′-methoxy-biphenyl-3- yloxymethyl)-benzoic acid methyl ester |
| 232 | 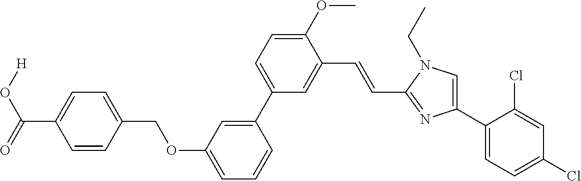 | 4-(3′-{2-[4-(2,4-dichloro- phenyl)-1-ethyl-1H- imidazol-2-yl]-(E)-vinyl-}- 4′-methoxy-biphenyl-3- yloxymethyl)-benzoic acid |
| 233 | 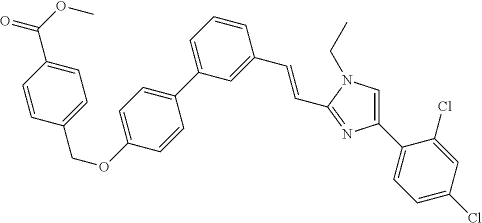 | 4-(3′-{2-[4-(2,4-dichloro- phenyl)-1-ethyl-1H- imidazol-2-yl]-(E)-vinyl-}- biphenyl-4-yloxymethyl)- benzoic acid methyl ester |
| 234 | 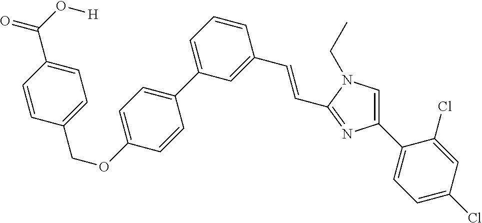 | 4-(3′-{2-[4-(2,4-dichloro- phenyl)-1-ethyl-1H- imidazol-2-yl]-(E)-vinyl-}- biphenyl-4-yloxymethyl)- benzoic acid |
| 235 | 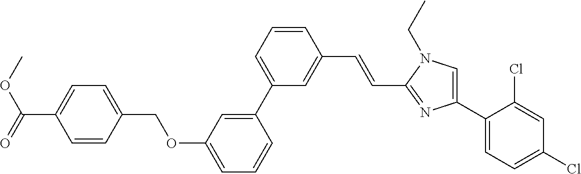 | 4-(3′-{2-[4-(2,4-dichloro- phenyl)-1-ethyl-1H- imidazol-2-yl]-(E)-vinyl-}- biphenyl-3-yloxymethyl)- benzoic acid methyl ester |
| 236 | 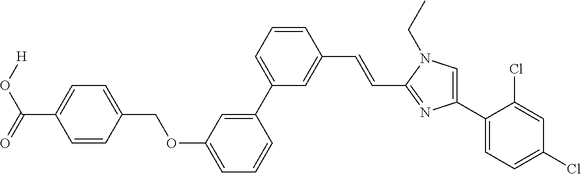 | 4-(3′-{2-[4-(2,4-dichloro- phenyl)-1-ethyl-1H- imidazol-2-yl]-(E)-vinyl-}- biphenyl-3-yloxymethyl)- benzoic acid |
| 237 | 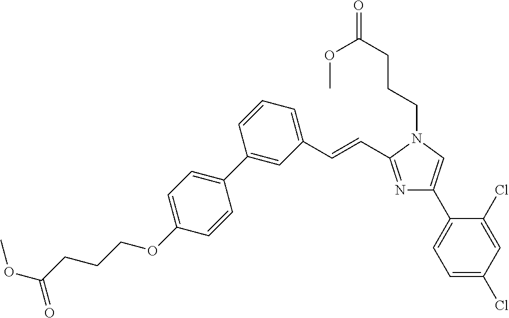 | 4-(4-(2,4-dichloro-phenyl)- 2-{2-[4′-(3- methoxycarbonyl-propoxy)- biphenyl-3yl]-(E)-vinyl}- imidazol-1yl)-butyric acid methyl ester |
| 238 | 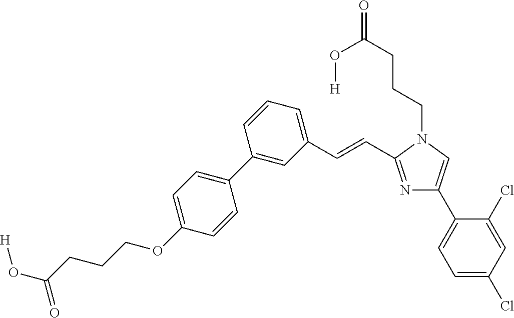 | 4-[2-{2-[4′-(3-carboxy- propoxy)-biphenyl-3-yl]-(E)- vinyl}-4-(2,4-dichloro- phenyl)-imidazol-1-yl]- butyric acid |
| 239 | 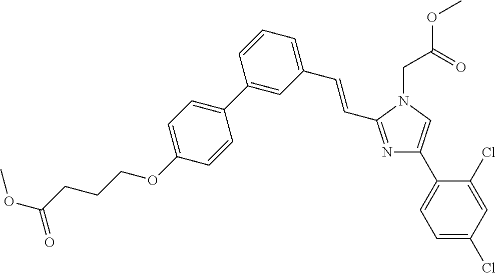 | 4-(3′-{2-[4-(2,4-dichloro- phenyl)-1- methoxycarbonylmethyl- 1H-imidazol-2-yl]-(E)-vinyl}- biphenyl-4yloxy)-butyric acid methyl ester |
| 240 | 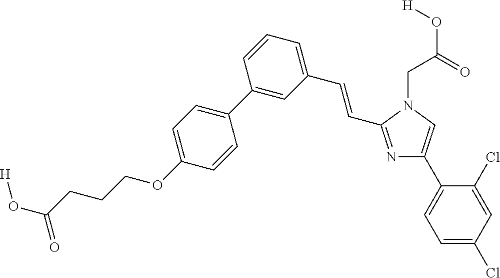 | 4-(3′-{2-[4-(2,4-dichloro- phenyl)-1- methoxycarbonylmethyl- 1H-imidazol-2-yl]-(E)-vinyl}- biphenyl-4yloxy)-butyric acid |
| 241 | 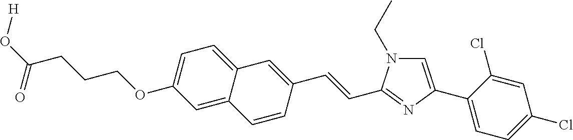 | 4-(6-{2-[4-(2,4-Dichloro- phenyl)-1-ethyl-1H- imidazol-2-yl]-(E)-vinyl}- naphthalen-2yloxy)-butyric acid |
| 242 | 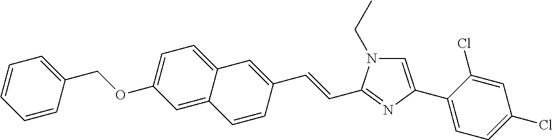 | 2-[2-(6-benzyloxy- naphthalen-2-yl)-(E)-vinyl]- 4-(2,4-dichloro-phenyl)-1- ethyl-1H-imidazole |
| 243 | 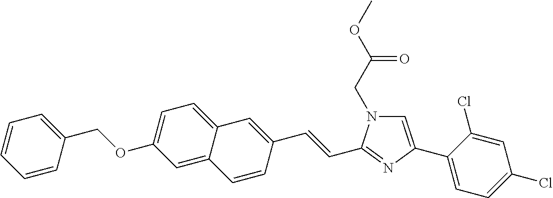 | 2-[2-(6-benzyloxy- naphthalen-2-yl)-(E)-vinyl]- 4-(2,4-dichloro-phenyl)- imidazol-1-yl]-acetic acid methyl ester |
| 244 | 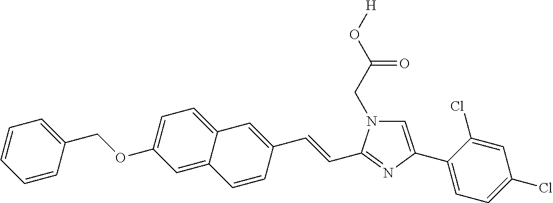 | 2-[2-(6-benzyloxy- naphthalen-2-yl)-(E)-vinyl]- 4-(2,4-dichloro-phenyl)- imidazol-1-yl]-acetic acid methyl ester |
| 245 | 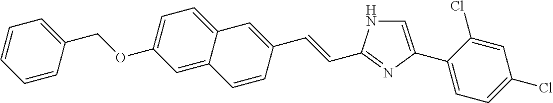 | 2-[2-(6-benzyloxy- naphthalen-2yl)-(E)-vinyl]- 4-(2,4-dichloro-phenyl)-1H- imidazole |
| 246 | 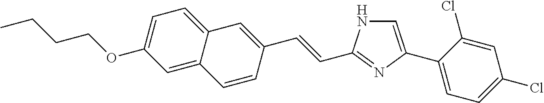 | 2-[2-(6-butoxy-naphthalen- 2yl)-(E)-vinyl]-4-(2,4- dichloro-phenyl)-1H- imidazole |
| 247 | 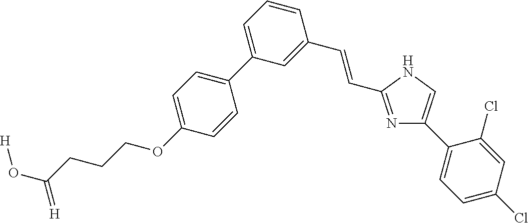 | 4-(3-{2-[-4-(2,4-dichloro- phenyl)-1H-imidazol-2-yl]- (E)-vinyl}-biphenyl-4-yloxy)- butyric acid |
| 248 | 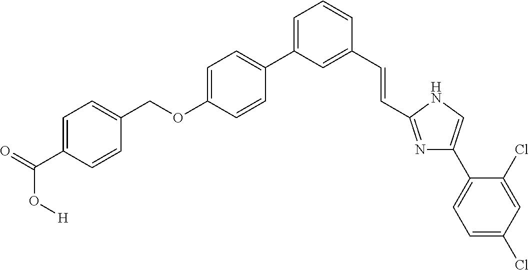 | 4-(3-{2-[-4-(2,4-dichloro- phenyl)-1H-imidazol-2-yl]- (E)-vinyl}-biphenyl-4- yloxymethyl)-benzoic acid |
| 249 | 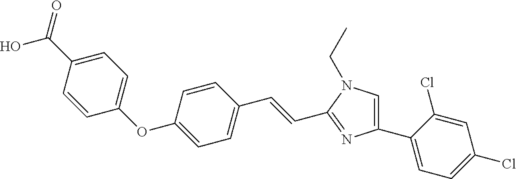 | 4-(4-{2-[4-(2,4-dichloro- phenyl)-1-ethyl-1H- imidazol-2-yl]-(E)-vinyl}- phenoxy)-benzoic acid |
| 250 | 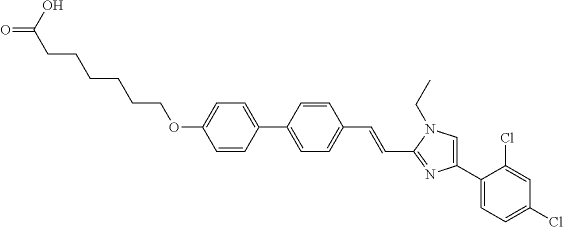 | 7-(4′-{2-[4-(2,4-dichloro- phenyl)-1-ethyl-1H- imidazol-2-yl]-(E)-vinyl}- biphenyl-4-yloxy)-heptanoic acid |
| 251 | 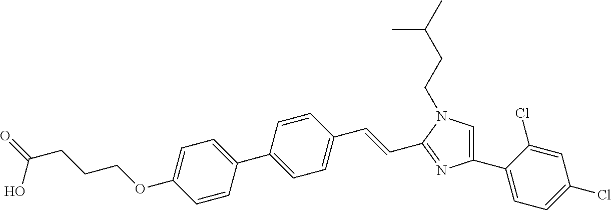 | 4-(4′-{2-[4-(2,4-dichloro- phenyl)-1-(3-methyl-butyl)- 1H-imidazol-2-yl]-(E)-vinyl}- biphenyl-4-yloxy)-butyric acid |
| 252 | 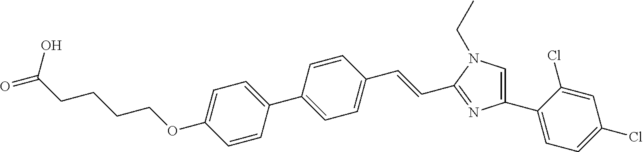 | 5-(4′-{2-[4-(2,4-dichloro- phenyl)-1-ethyl-1H- imidazol-2-yl]-(E)-vinyl}- biphenyl-4-yloxy)-pentanoic acid |
| 253 | 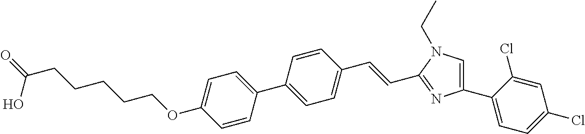 | 6-(4′-{2-[4-(2,4-dichloro- phenyl)-1-ethyl-1H- imidazol-2-yl]-(E)-vinyl}- biphenyl-4-yloxy)-hexanoic acid |
| 254 | 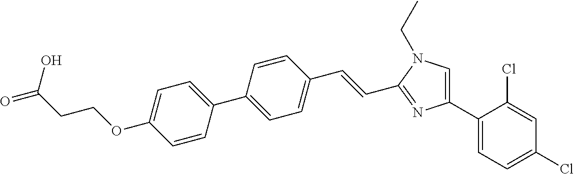 | 3-(4′-{2-[4-(2,4-dichloro- phenyl)-1-ethyl-1H- imidazol-2-yl]-(E)-vinyl}- biphenyl-4-yloxy)-propionic acid |
| 255 | 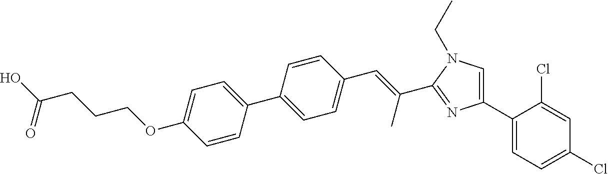 | 4-(4′-{2-[4-(2,4-dichloro- phenyl)-1-ethyl-1H- imidazol-2-yl]-(E)- propenyl}-biphenyl-4- yloxy)-butyric acid |
| 256 | 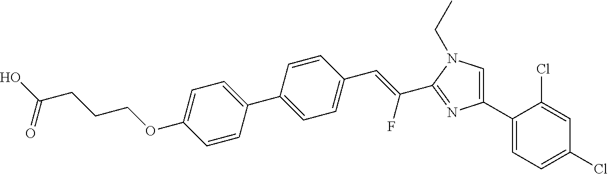 | 4-(4′-{2-[4-(2,4-dichloro- phenyl)-1-ethyl-1H- imidazol-2-yl]-(Z)-2-fluoro- vinyl}-biphenyl-4-yloxy)- butyric acid |
| 257 | 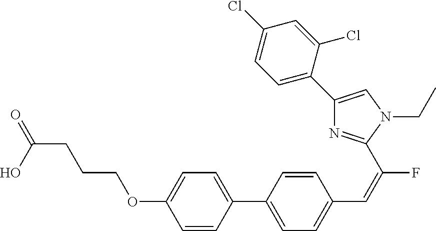 | 4-(4′-{2-[4-(2,4-dichloro- phenyl)-1-ethyl-1H- imidazol-2-yl]-(E)-2- fluorovinyl}-biphenyl-4- yloxy)-butyric acid |
| 258 | 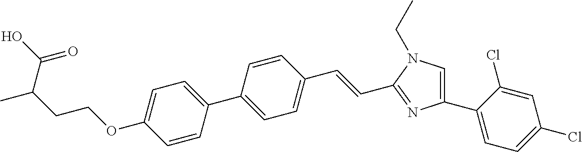 | 4-(4′-{2-[4-(2,4-dichloro- phenyl)-1-ethyl-1H- imidazol-2-yl]-(E)-vinyl}- biphenyl-4-yloxy)-2-methyl- butyric acid |
| 259 | 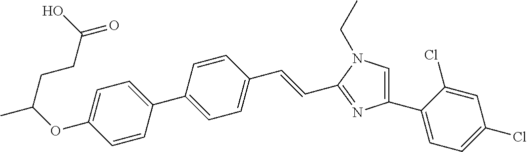 | 4-(4′-{2-[4-(2,4-dichloro- phenyl)-1-ethyl-1H- imidazol-2-yl]-(E)-vinyl}- biphenyl-4-yloxy)-pentanoic acid |
| 260 | 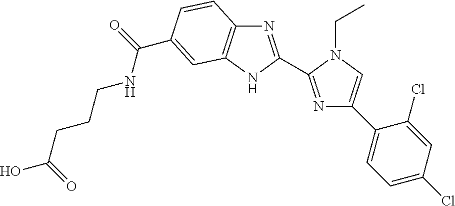 | 4-({2-[4-(2,4-dichloro- phenyl)-1-ethyl-1H- imidazol-2-yl]-3H- benzoimidazole-5- carbonyl}-amino)-butyric acid |
| 261 | 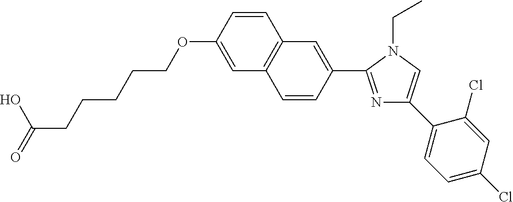 | 6-{6-[4-(2,4-dichloro- phenyl)-1-ethyl-1H- imidazol-2-yl]-naphthalen- 2-yloxy}-hexanoic acid |
| 262 | 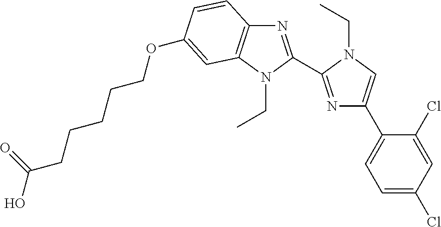 | 6-{2-[4-(2,4-dichloro- phenyl)-1-ethyl-1H- imidazol-2-yl]-3-ethyl-3H- benzoimidazol-5-yloxy}- hexanoic acid |
| 263 | 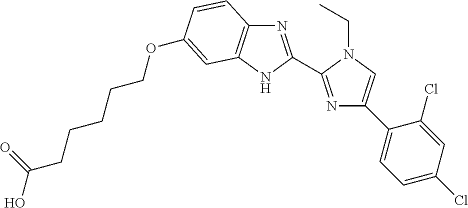 | 6-{2-[4-(2,4-dichloro- phenyl)-1-ethyl-1H- imidazol-2-yl]-3H- benzoimidazol-5-yloxy}- hexanoic acid |
| 264 | 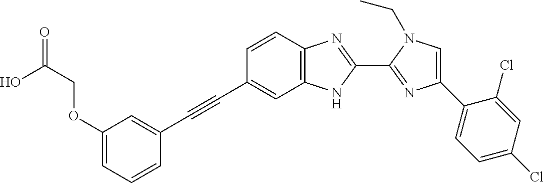 | (3-{2-[4-(2,4-dichloro- phenyl)-1-ethyl-1H- imidazol-2-yl]-3H- benzoimidazol-5-ylethynyl}- phenoxy)-acetic acid |
| 265 | 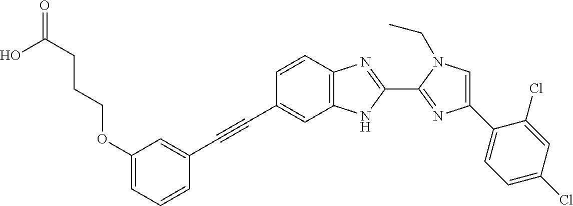 | 4-(3-{2-[4-(2,4-dichloro- phenyl)-1-ethyl-1H- imidazol-2-yl]-3H- benzoimidazol-5-ylethynyl}- phenoxy)-butyric acid |
| 266 | 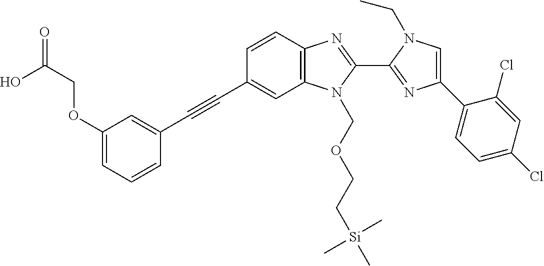 | {3-[2-[4-(2,4-dichloro- phenyl)-1-ethyl-1H- imidazol-2-yl]-3-(2- trimethylsilanyl- ethoxymethyl)-3H- benzoimidazol-5-ylethynyl]- phenoxy}-acetic acid |
| 267 | 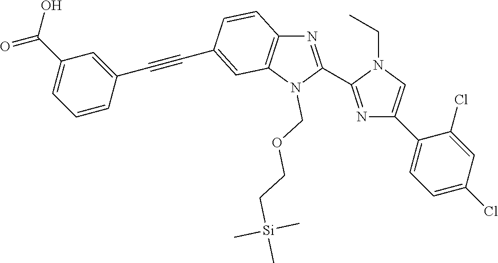 | 3-[2-[4-(2,4-dichloro- phenyl)-1-ethyl-1H- imidazol-2-yl]-3-(2- trimethylsilanyl- ethoxymethyl)-3H- benzoimidazol-5-ylethynyl]- benzoic acid |
| 268 | 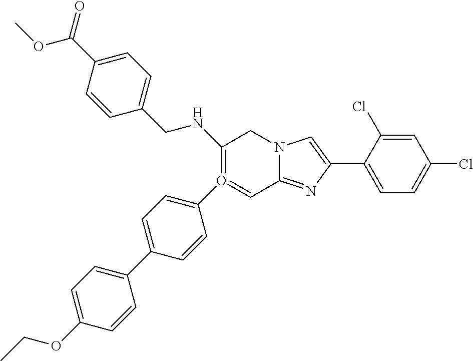 | 4-[(2-{4-(2,4-Dichloro- phenyl)-2-[2-(4′-ethoxy- biphenyl-4-yl)-(E)-vinyl]- imidazol-1-yl}- acetylamino)-methyl]- benzoic acid methyl ester |
| 269 | 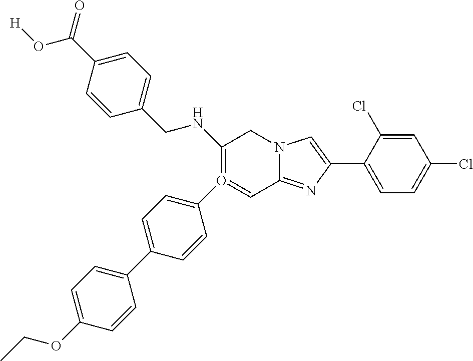 | 4-[(2-{4-(2,4-Dichloro- phenyl)-2-[2-(4′-ethoxy- biphenyl-4-yl)-(E)-vinyl]- imidazol-1-yl}- acetylamino)-methyl]- benzoic acid |
| 270 | 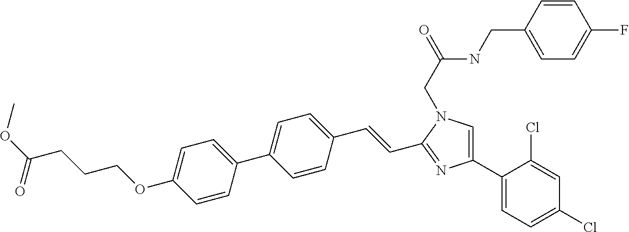 | 4-[4′-(2-{4-(2,4-Dichloro- phenyl)-1-[(4-fluoro- benzylcarbamoyl)-methyl]- 1H-imidazol-2-yl}-(E)-vinyl)- biphenyl-4-yloxy]-butyric acid methyl ester |
| 271 | 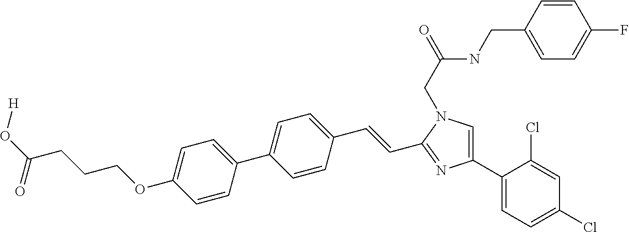 | 4-[4′-(2-{4-(2,4-Dichloro- phenyl)-1-[(4-fluoro- benzylcarbamoyl)-methyl]- 1H-imidazol-2-yl}-(E)-vinyl)- biphenyl-4-yloxy]-butyric acid |
| 272 | 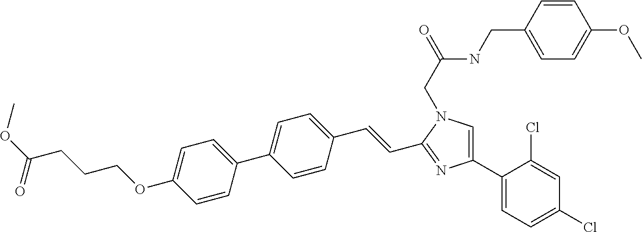 | 4-[4′-(2-{4-(2,4-Dichloro- phenyl)-1-[(4-methoxy- benzylcarbamoyl)-methyl]- 1H-imidazol-2-yl}-(E)-vinyl)- biphenyl-4-yloxy]-butyric acid methyl ester |
| 273 | 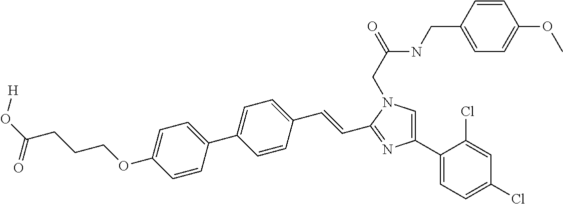 | 4-[4′-(2-{4-(2,4-Dichloro- phenyl)-1-[(4-methoxy- benzylcarbamoyl)-methyl]- 1H-imidazol-2-yl}-(E)-vinyl)- biphenyl-4-yloxy]-butyric acid |
| 274 | 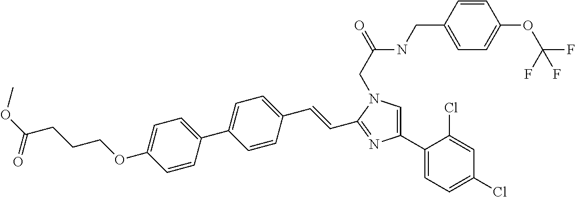 | 4-[4′-(2-{4-(2,4-Dichloro- phenyl)-1-[(4- trifluoromethoxy- benzylcarbamoyl)-methyl]- 1H-imidazol-2-yl}-(E)-vinyl)- biphenyl-4-yloxy]-butyric acid methyl ester |
| 275 | 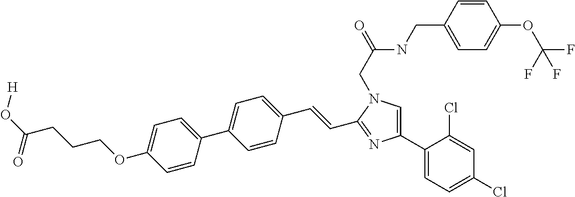 | 4-[4′-(2-{4-(2,4-Dichloro- phenyl)-1-[(4-- trifluoromethoxy- benzylcarbamoyl)-methyl]- 1H-imidazol-2-yl}-(E)-vinyl)- biphenyl-4-yloxy]-butyric acid |
| 276 | 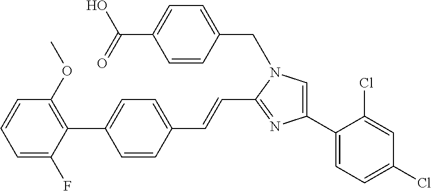 | 4-{4-(2,4-dichloro-phenyl)- 2-[2-(6′-fluoro-2′-methoxy- biphenyl-4-yl)-(E)-vinyl]- imidazol-1-ylmethyl}- benzoic acid |
| 277 | 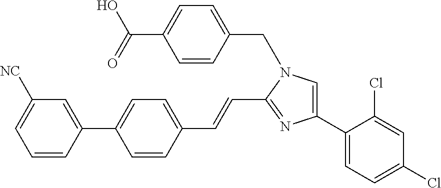 | 4-[2-[2-(3′-cyano-biphenyl- 4-yl)-(E)-vinyl]-4-(2,4- dichloro-phenyl)-imidazol- 1-ylmethyl]-benzoic acid |
| 278 | 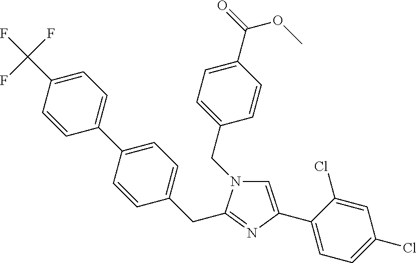 | 4-[4-(2,4-dichloro-phenyl)- 2-(4′-trifluoromethyl- biphenyl-4-ylmethyl)- imidazol-1-ylmethyl]- benzoic acid methyl ester |
| 279 | 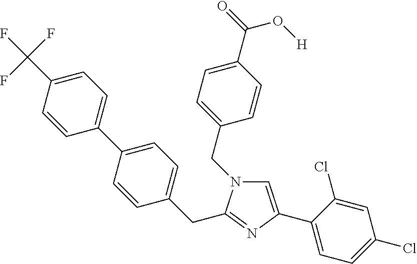 | 4-[4-(2,4-dichloro-phenyl)- 2-(4′-trifluoromethyl- biphenyl-4-ylmethyl)- imidazol-1-ylmethyl]- benzoic acid |
| 280 | 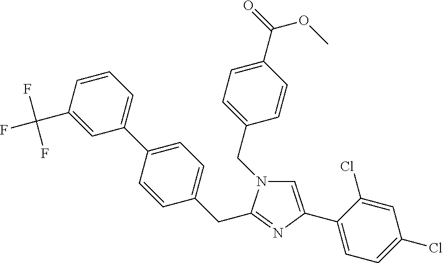 | 4-[4-(2,4-dichloro-phenyl)- 2-(3′-trifluoromethyl- biphenyl-4-ylmethyl)- imidazol-1-ylmethyl]- benzoic acid methyl ester |
| 281 | 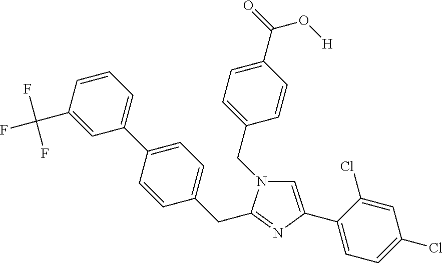 | 4-[4-(2,4-dichloro-phenyl)- 2-(3′-trifluoromethyl- biphenyl-4-ylmethyl)- imidazol-1-ylmethyl]- benzoic acid |
| 282 | 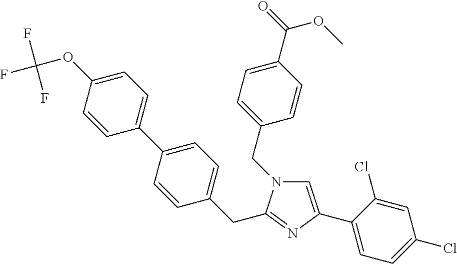 | 4-[4-(2,4-dichloro-phenyl)- 2-(4′-trifluoromethoxy- biphenyl-4-ylmethyl)- imidazol-1-ylmethyl]- benzoic acid methyl ester |
| 283 | 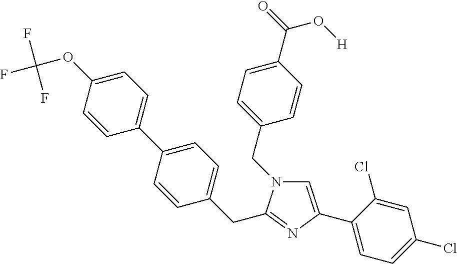 | 4-[4-(2,4-dichloro-phenyl)- 2-(4′-trifluoromethoxy- biphenyl-4-ylmethyl)- imidazol-1-ylmethyl]- benzoic acid |
| 284 | 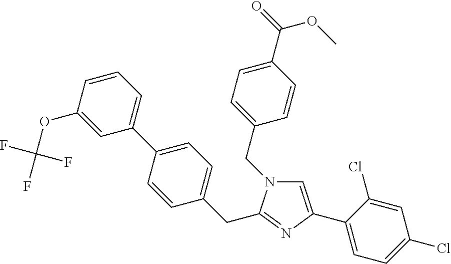 | 4-[4-(2,4-dichloro-phenyl)- 2-(3′-trifluoromethoxy- biphenyl-4-ylmethyl)- imidazol-1-ylmethyl]- benzoic acid methyl ester |
| 285 | 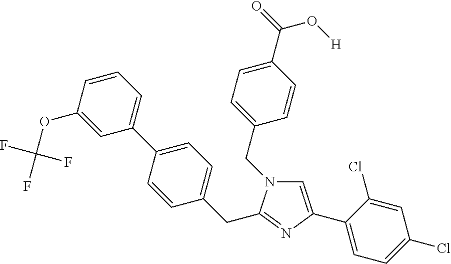 | 4-[4-(2,4-dichloro-phenyl)- 2-(3′-trifluoromethoxy- biphenyl-4-ylmethyl)- imidazol-1-ylmethyl]- benzoic acid |
| 286 | 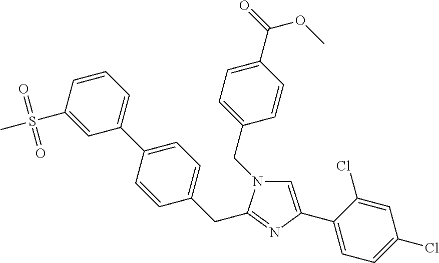 | 4-[4-(2,4-dichloro-phenyl)- 2-(3′-methanesulfonyl- biphenyl-4-ylmethyl)- imidazol-1-ylmethyl]- benzoic acid methyl ester |
| 287 | 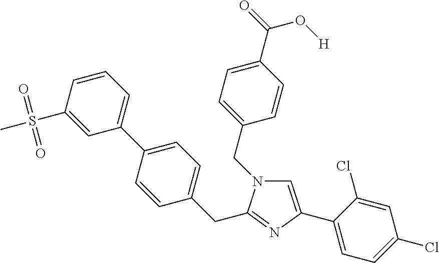 | 4-[4-(2,4-dichloro-phenyl)- 2-(3′-methanesulfonyl- biphenyl-4-ylmethyl)- imidazol-1-ylmethyl]- benzoic acid |
| 288 | 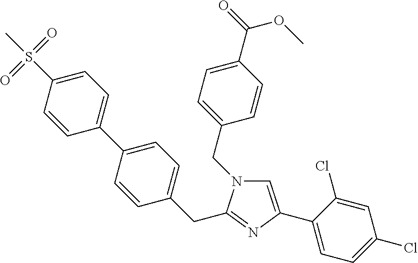 | 4-[4-(2,4-dichloro-phenyl)- 2-(4′-methanesulfonyl- biphenyl-4-ylmethyl)- imidazol-1-ylmethyl]- benzoic acid methyl ester |
| 289 | 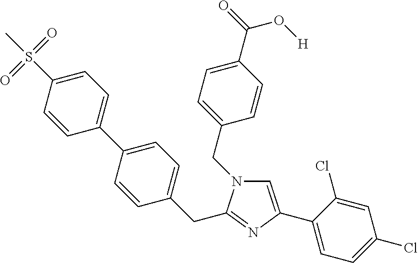 | 4-[4-(2,4-dichloro-phenyl)- 2-(4′-methanesulfonyl- biphenyl-4-ylmethyl)- imidazol-1-ylmethyl]- benzoic acid |
| 290 | 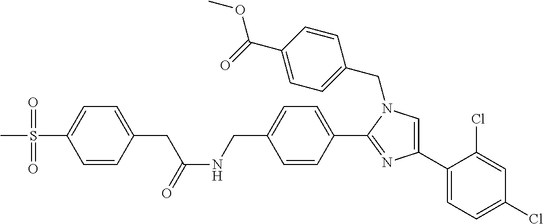 | 4-[4-(2,4-dichloro-phenyl)- 2-(4-{[2-(4- methanesulfonyl-phenyl)- acetylamino]-methyl}- phenyl)-imidazol-1- ylmethyl]-benzoic acid methyl ester |
| 291 | 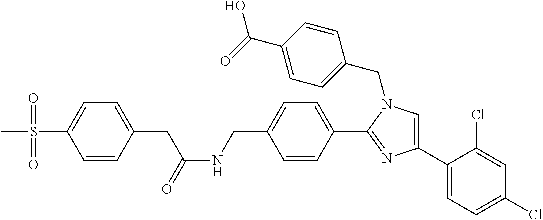 | 4-[4-(2,4-dichloro-phenyl)- 2-(4-{[2-(4- methanesulfonyl-phenyl)- acetylamino]-methyl}- phenyl)-imidazol-1- ylmethyl]-benzoic acid |
| 292 | 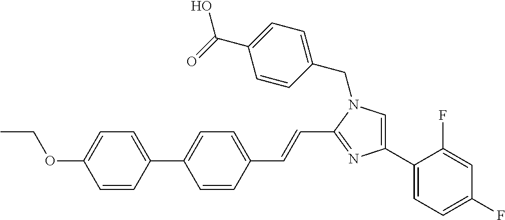 | 4-{4-(2,4-difluoro-phenyl)- 2-[2-(4′-ethoxy-biphenyl-4- yl)-(E)-vinyl]-imidazol-1- ylmethyl}-benzoic acid |
| 293 | 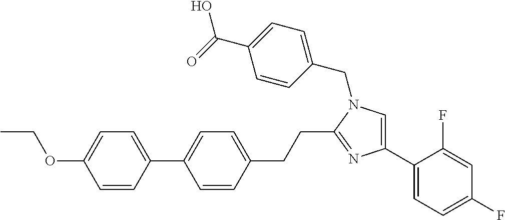 | 4-{4-(2,4-difluoro-phenyl)- 2-[2-(4′-ethoxy-biphenyl-4- yl)-ethyl]-imidazol-1- ylmethyl}-benzoic acid |
| 294 | 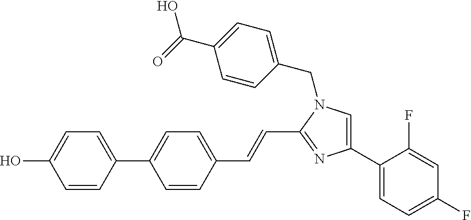 | 4-{4-(2,4-difluoro-phenyl)- 2-[2-(4′-hydroxy-biphenyl- 4-yl)-(E)-vinyl]-imidazol-1- ylmethyl}-benzoic acid |
| 295 | 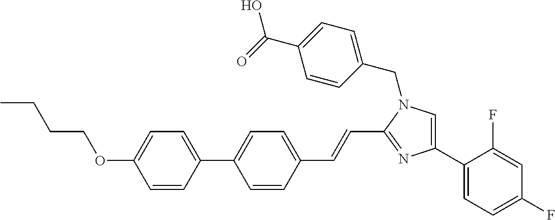 | 4-[2-[2-(4′-butoxy-biphenyl- 4-yl)-(E)-vinyl]-4-(2,4- difluoro-phenyl)-imidazol-1- ylmethyl]-benzoic acid |
| 296 | 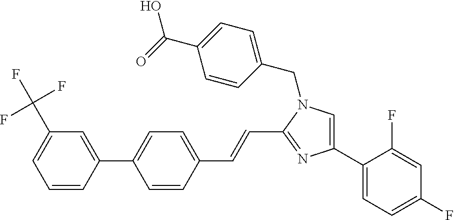 | 4-{4-(2,4-difluoro-phenyl)- 2-[2-(3′-trifluoromethyl- biphenyl-4-yl)-(E)-vinyl]- imidazol-1-ylmethyl}- benzoic acid |
| 297 | 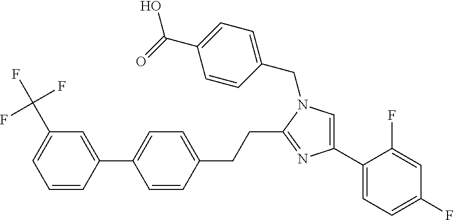 | 4-{4-(2,4-difluoro-phenyl)- 2-[2-(3′-trifluoromethyl- biphenyl-4-yl)-ethyl]- imidazol-1-ylmethyl}- benzoic acid |
| 298 | 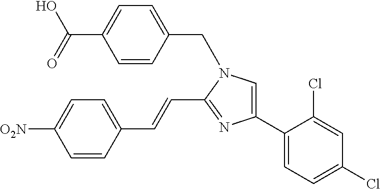 | 4-{4-(2,4-dichloro-phenyl)- 2-[2-(4-nitro-phenyl)-(E)- vinyl]-imidazol-1-ylmethyl}- benzoic acid |
| 299 | 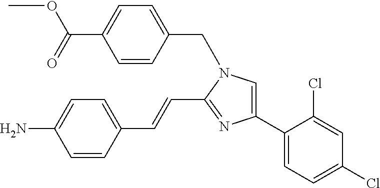 | 4-[2-[2-(4-amino-phenyl)- (E)-vinyl]-4-(2,4-dichloro- phenyl)-imidazol-1- ylmethyl]-benzoic acid methyl ester |
| 300 | 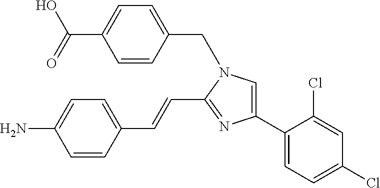 | 4-[2-[2-(4-amino-phenyl)- (E)-vinyl]-4-(2,4-dichloro- phenyl)-imidazol-1- ylmethyl]-benzoic acid |
| 301 | 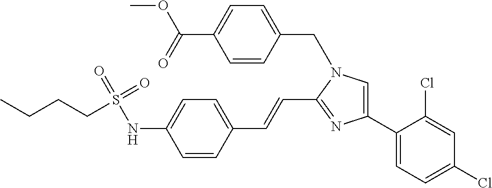 | 4-[2-{2-[4-(butane-1- sulfonylamino)-phenyl]-(E)- vinyl}-4-(2,4-dichloro- phenyl)-imidazol-1- ylmethyl]-benzoic acid methyl ester |
| 302 | 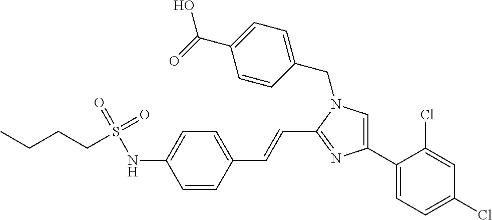 | 4-[2-{2-[4-(butane-1- sulfonylamino)-phenyl]-(E)- vinyl}-4-(2,4-dichloro- phenyl)-imidazol-1- ylmethyl]-benzoic acid |
| 303 | 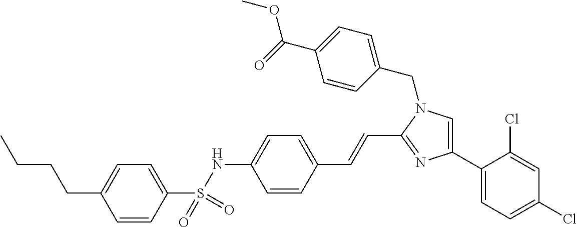 | 4-[2-{2-[4-(4-butyl- benzenesulfonylamino)- phenyl]-(E)-vinyl}-4-(2,4- dichloro-phenyl)-imidazol- 1-ylmethyl]-benzoic acid methyl ester |
| 304 | 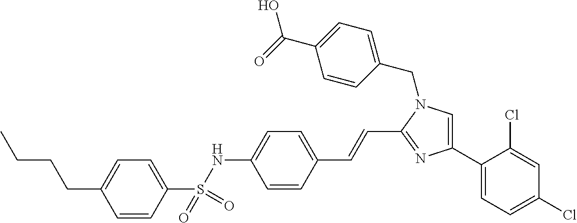 | 4-[2-{2-[4-(4-butyl- benzenesulfonylamino)- phenyl]-(E)-vinyl}-4-(2,4- dichloro-phenyl)-imidazol- 1-ylmethyl]-benzoic acid |
| 305 | 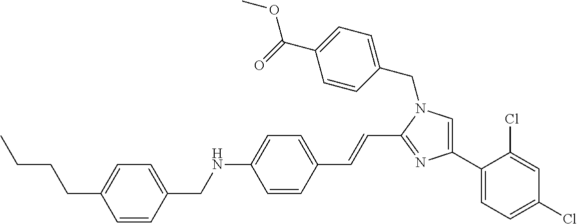 | 4-[2-{2-[4-(4-butyl- benzylamino)-phenyl]-(E)- vinyl}-4-(2,4-dichloro- phenyl)-imidazol-1- ylmethyl]-benzoic acid methyl ester |
| 306 | 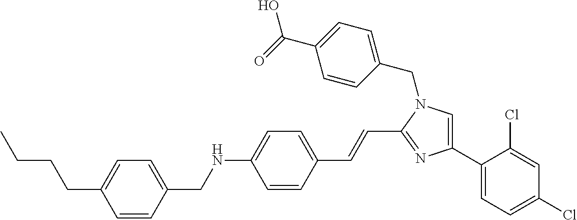 | 4-[2-{2-[4-(4-butyl- benzylamino)-phenyl]-(E)- vinyl}-4-(2,4-dichloro- phenyl)-imidazol-1- ylmethyl]-benzoic acid |
| 307 | 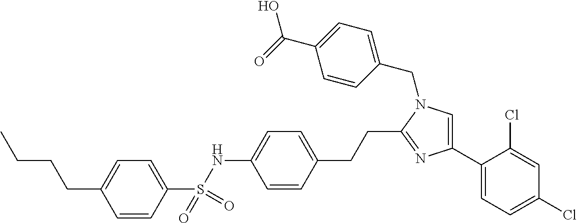 | 4-[2-{2-[4-(4-butyl- benzenesulfonylamino)- phenyl]-ethyl}-4-(2,4- dichloro-phenyl)-imidazol- 1-ylmethyl]-benzoic acid |
| 308 | 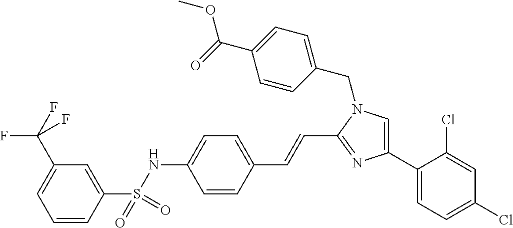 | 4-(4-(2,4-dichloro-phenyl)- 2-{2-[4-(3-trifluoromethyl- benzenesulfonylamino)- phenyl]-(E)-vinyl}-imidazol- 1-ylmethyl)-benzoic acid methyl ester |
| 309 | 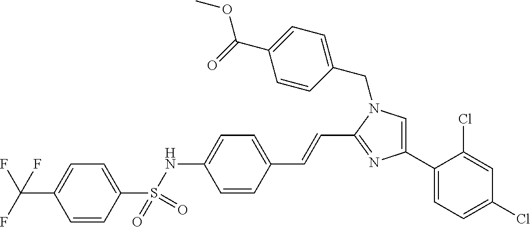 | 4-(4-(2,4-dichloro-phenyl)- 2-{2-[4-(4-trifluoromethyl- benzenesulfonylamino)- phenyl]-(E)-vinyl}-imidazol- 1-ylmethyl)-benzoic acid methyl ester |
| 310 | 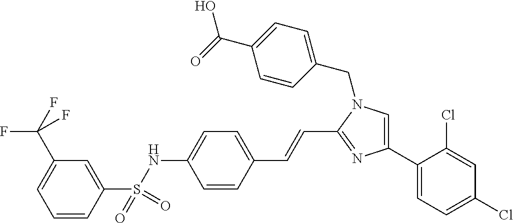 | 4-(4-(2,4-dichloro-phenyl)- 2-{2-[4-(3-trifluoromethyl- benzenesulfonylamino)- phenyl]-(E)-vinyl}-imidazol- 1-ylmethyl)-benzoic acid |
| 311 | 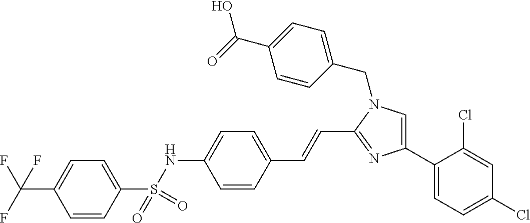 | 4-(4-(2,4-dichloro-phenyl)- 2-{2-[4-(4-trifluoromethyl- benzenesulfonylamino)- phenyl]-(E)-vinyl}-imidazol- 1-ylmethyl)-benzoic acid |
| 312 | 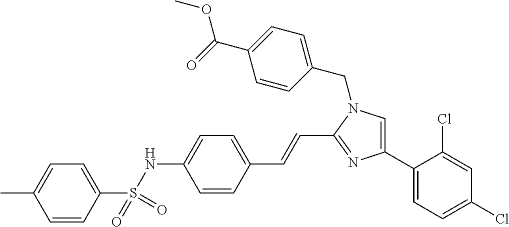 | 4-(4-(2,4-dichloro-phenyl)- 2-{2-[4-(toluene-4- sulfonylamino)-phenyl]-(E)- vinyl}-imidazol-1-ylmethyl)- benzoic acid methyl ester |
| 313 | 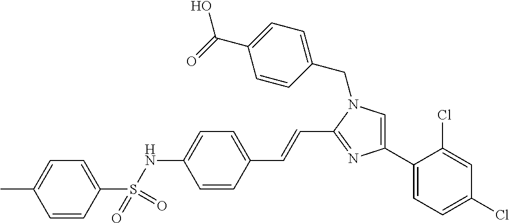 | 4-(4-(2,4-dichloro-phenyl)- 2-{2-[4-(toluene-4- sulfonylamino)-phenyl]-(E)- vinyl}-imidazol-1-ylmethyl)- benzoic acid |
| 314 | 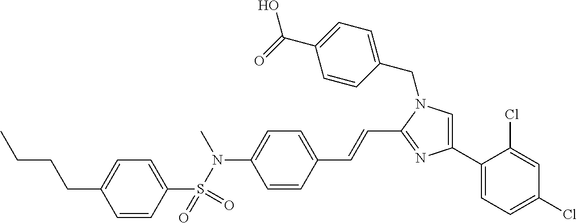 | 4-[2-(2-{4-[(4-butyl- benzenesulfonyl)-methyl- amino]-phenyl}-(E)-vinyl)-4- (2,4-dichloro-phenyl)- imidazol-1-ylmethyl]- benzoic acid |
| 315 | 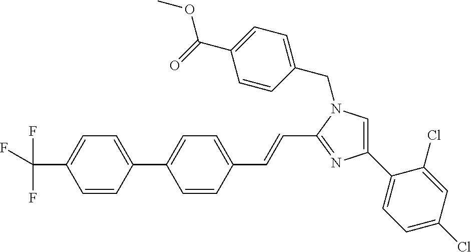 | 4-{4-(2,4-dichloro-phenyl)- 2-[2-(4′-trifluoromethyl- biphenyl-4-yl)-(E)-vinyl]- imidazol-1yl-methyl} benzoic acid methyl ester |
| 316 | 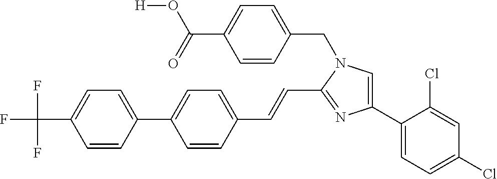 | 4-{4-(2,4-dichloro-phenyl)- 2[2-(4′-trifluoromethyl- biphenyl-4-yl)-(E)-vinyl]- imidazol-1-ylmethyl}- benzoic acid |
| 317 | 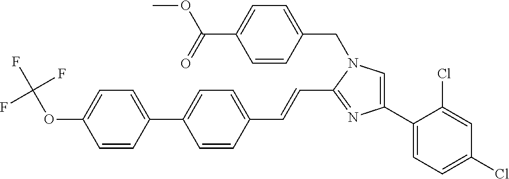 | 4-{4-(2,4-dichloro-phenyl)- 2-[2-(4′-trifluoromethoxy- biphenyl-4-yl)-(E)-vinyl]- imidazol-1yl-methyl} benzoic acid methyl ester |
| 318 | 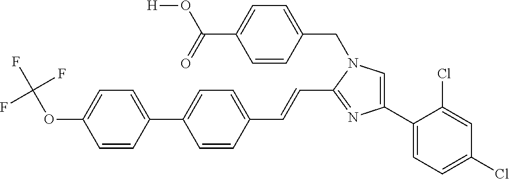 | 4-{4-(2,4-dichloro-phenyl)- 2[2-(4′-trifluoromethoxy- biphenyl-4-yl)-(E)-vinyl]- imidazol-1-ylmethyl}- benzoic acid |
| 319 | 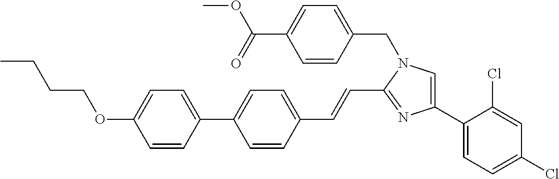 | 4-[2-[2-(4′-butoxy-biphenyl- 4-yl)-(E)-vinyl]-4-(2,4- dichloro-phenyl)-imidazol- 1-ylmethyl]-benzoic acid methyl ester |
| 320 | 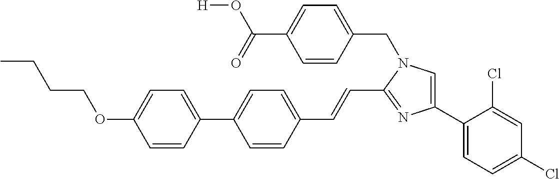 | 4-[2-[2-(4′-butoxy-biphenyl- 4-yl)-(E)-vinyl]-4-(2,4- dichloro-phenyl)-imidazol- 1-ylmethyl]-benzoic acid |
| 321 | 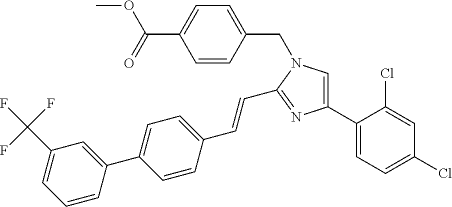 | 4-{4-(2,4-dichloro-phenyl)- 2-[2-(3′-trifluoromethyl- biphenyl-4-yl)-(E)-vinyl]- imidazol-1yl-methyl} benzoic acid methyl ester |
| 322 | 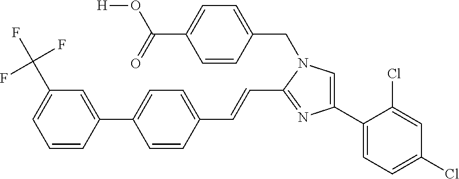 | 4-{4-(2,4-dichloro-phenyl)- 2[2-(3′-trifluoromethyl- biphenyl-4-yl)-(E)-vinyl]- imidazol-1-ylmethyl}- benzoic acid |
| 323 | 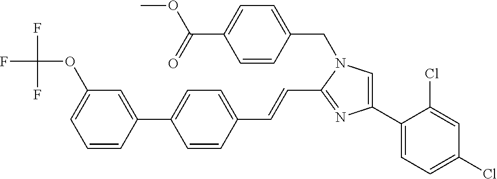 | 4-{4-(2,4-dichloro-phenyl)- 2-[2-(3′-trifluoromethoxy- biphenyl-4-yl)-(E)-vinyl]- imidazol-1yl-methyl} benzoic acid methyl ester |
| 324 | 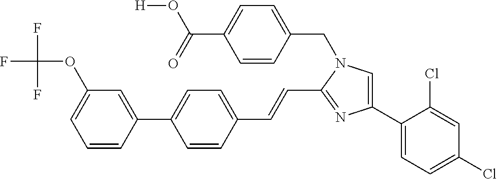 | 4-{4-(2,4-dichloro-phenyl)- 2-[2-(3′-trifluoromethoxy- biphenyl-4-yl)-(E)-vinyl]- imidazol-1-ylmethyl}- benzoic acid |
| 325 | 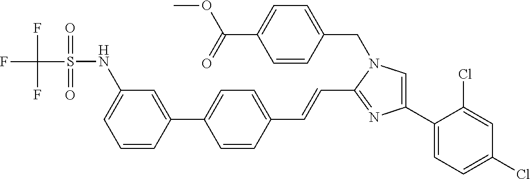 | 4-{4-(2,4-dichloro-phenyl)- 2[2-(3- trifluoromethanesulfonyl amino-biphenyl-4-yl)-(E)- vinyl]-imidazol-1-ylmethyl}- benzoic acid methyl ester |
| 326 | 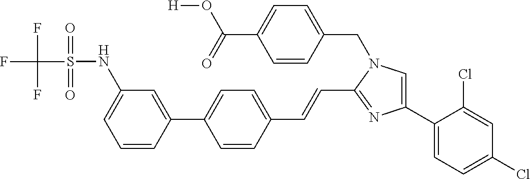 | 4-{4-(2,4-dichloro-phenyl)- 2-[2-(3′- trifluoromethanesulfonyl amino-biphenyl-4-yl)-(E)- vinyl]-imidazol-1-ylmethyl}- benzoic acid |
| 327 | 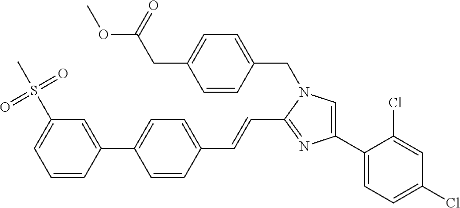 | (4-{4-(2,4-dichloro-phenyl)- 2-[2-(3′-methanesulfonyl- biphenyl-4-yl)-(E)-vinyl]- imidazol-1-ylmethyl}- phenyl)-acetic acid methyl ester |
| 328 | 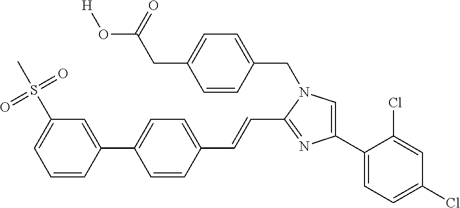 | (4-{4-(2,4-dichloro-phenyl)- 2-[2-(3′-methanesulfonyl- biphenyl-4-yl)-(E)-vinyl]- imidazol-1-ylmethyl}- phenyl)-acetic acid |
| 329 | 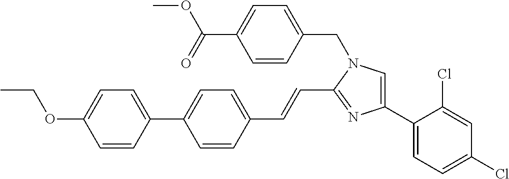 | 4-{4-(2,4-dichloro-phenyl)- 2-[2-(4′-ethoxy-biphenyl-4- yl)-(E)-vinyl]-imidazol-1- ylmethyl}-benzoic acid methyl ester |
| 330 | 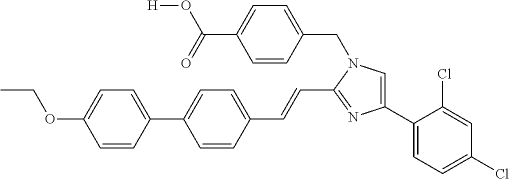 | 4-{4-(2,4-dichloro-phenyl)- 2-[2-(4′-ethoxy-biphenyl-4- yl)-(E)-vinyl]-imidazol-1- ylmethyl}-benzoic acid |
| 331 | 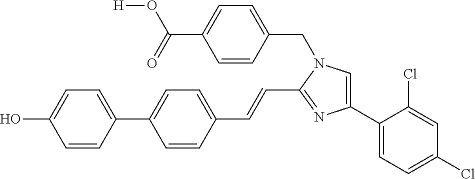 | 4-{4-(2,4-dichloro-phenyl)- 2-[2-(4′-hydroxy-biphenyl- 4-yl)-(E)-vinyl]-imidazol-1- ylmethyl}-benzoic acid |
| 332 | 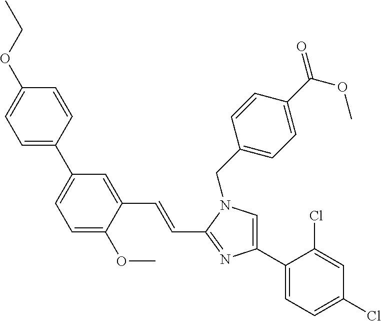 | 4-{4-(2,4-dichloro-phenyl)- 2-[2-(4′-ethoxy-4-methoxy- biphenyl-3-yl)-(E)-vinyl]- imidazol-1-ylmethyl}- benzoic acid methyl ester |
| 333 | 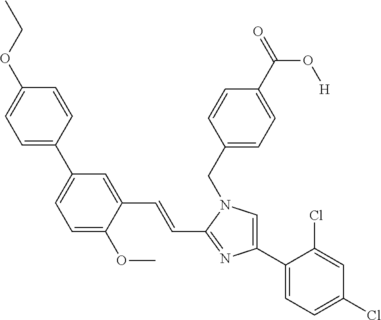 | 4-{4-(2,4-dichloro-phenyl)- 2-[2-(4′-ethoxy-4-methoxy- biphenyl-3-yl)-(E)-vinyl]- imidazol-1-ylmethyl}- benzoic acid |
| 334 | 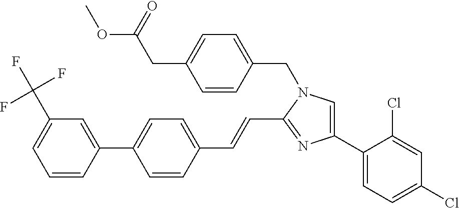 | (4-{4-(2,4-dichloro-phenyl)- 2-[2-(3′-trifluoromethyl- biphenyl-4-yl)-(E)-vinyl]- imidazol-1-ylmethyl}- phenyl)-acetic acid methyl ester |
| 335 | 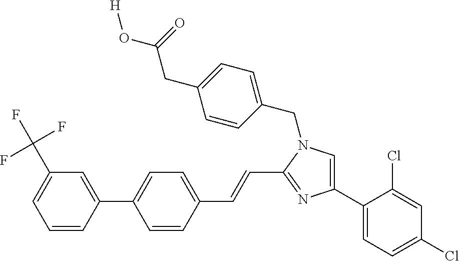 | (4-{4-(2,4-dichloro-phenyl)- 2-[2-(3′-trifluoromethyl- biphenyl-4-yl)-(E)-vinyl]- imidazol-1-ylmethyl}- phenyl)-acetic acid |
| 336 | 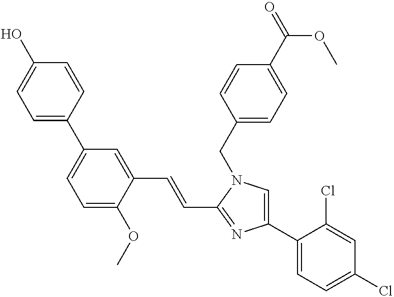 | 4-{4-(2,4-dichloro-phenyl)- 2-[2-(4′-hydroxy-4- methoxy-biphenyl-3-yl)-(E)- vinyl]-imidazol-1-ylmethyl}- benzoic acid methyl ester |
| 337 | 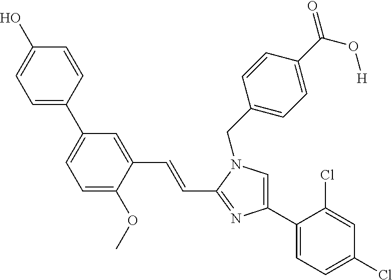 | 4-{4-(2,4-dichloro-phenyl)- 2-[2-(4′-hydroxy-4- methoxy-biphenyl-3-yl)-(E)- vinyl]-imidazol-1-ylmethyl}- benzoic acid |
| 338 | 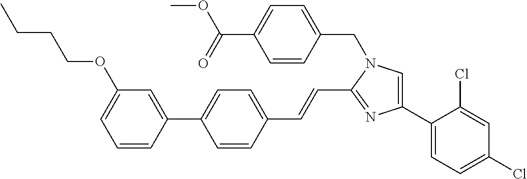 | 4-[2-[2-(3′-butoxy-biphenyl- 4-yl)-(E)-vinyl]-4-(2,4- dichloro-phenyl)-imidazol- 1-ylmethyl]-benzoic acid methyl ester |
| 339 | 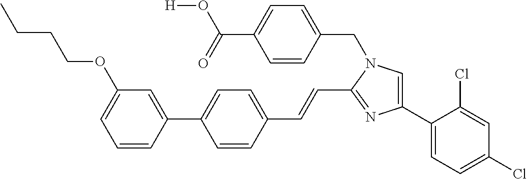 | 4-[2-[2-(3′-butoxy-biphenyl- 4-yl)-(E)-vinyl]-4-(2,4- dichloro-phenyl)-imidazol- 1-ylmethyl]-benzoic acid |
| 340 | 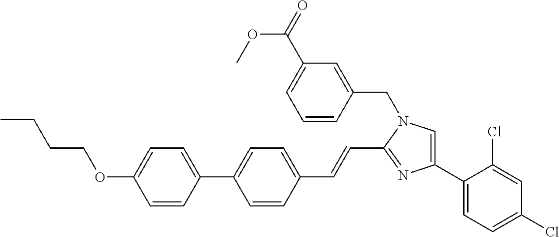 | 3-[2-[2-(4′-butoxy-biphenyl- 4-yl)-(E)-vinyl]-4-(2,4- dichloro-phenyl)-imidazol- 1-ylmethyl]-benzoic acid methyl ester |
| 341 | 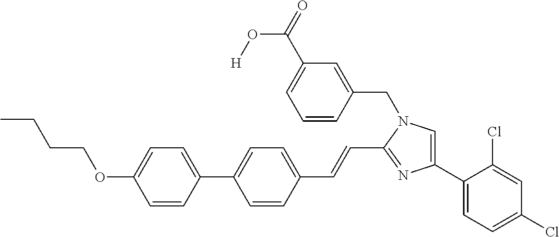 | 3-[2-[2-(4′-butoxy-biphenyl- 4-yl)-(E)-vinyl]-4-(2,4- dichloro-phenyl)-imidazol- 1-ylmethyl]-benzoic acid |
| 342 | 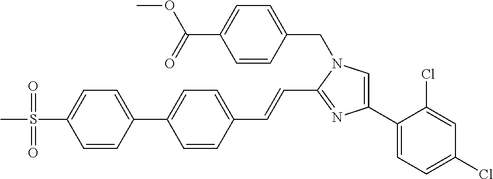 | 4-{4-(2,4-dichloro-phenyl)- 2-[2-(4′-methanesulfonyl- biphenyl-4-yl)-(E)-vinyl]- imidazol-1-ylmethyl}- benzoic acid methyl ester |
| 343 | 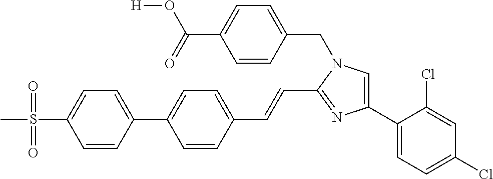 | 4-{4-(2,4-dichloro-phenyl)- 2-[2-(4′-methanesulfonyl- biphenyl-4-yl)-(E)-vinyl]- imidazol-1-ylmethyl}- benzoic acid |
| 344 | 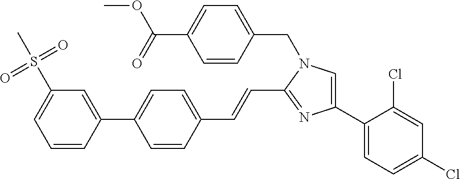 | 4-{4-(2,4-dichloro-phenyl)- 2-[2-(3′-methanesulfonyl- biphenyl-4-yl)-(E)-vinyl]- imidazol-1-ylmethyl}- benzoic acid methyl ester |
| 345 | 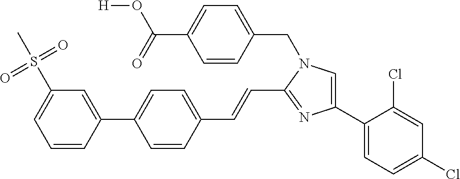 | 4-{4-(2,4-dichloro-phenyl)- 2-[2-(3′-methanesulfonyl- biphenyl-4-yl)-(E)-vinyl]- imidazol-1-ylmethyl}- benzoic acid |
| 346 | 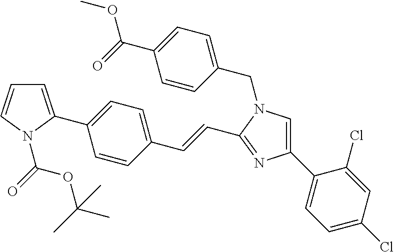 | 2-(4-{2-[4-(2,4-dichloro- phenyl)-1-(4- methoxycarbonyl-benzyl)- 1H-imidazol-2-yl]-(E)-vinyl}- phenyl)-pyrrole-1-carboxylic acid tert-butyl ester |
| 347 | 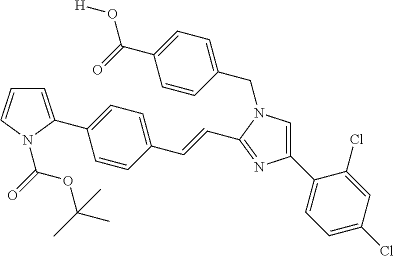 | 2-(4-{2-[1-(4-carboxy- benzyl)-4-(2,4-dichloro- phenyl)-1H-imidazol-2-yl]- (E)-vinyl}-phenyl)-pyrrole-1- carboxylic acid tert-butyl ester |
| 348 | 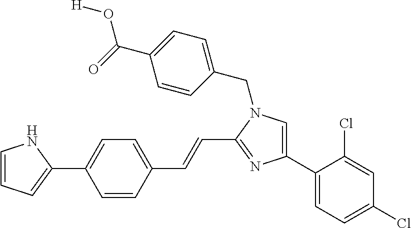 | 4-(4-(2,4-dichloro-phenyl)- 2-{2-[4-(1H-pyrrol-2-yl)- phenyl]-(E)-vinyl}-imidazol- 1-ylmethyl)-benzoic acid |
| 349 | 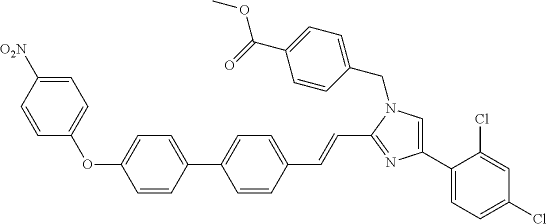 | 4-[2-{2-[4′-(4-nitro- phenoxy)-biphenyl-4-yl]- (E)-vinyl}-4-(2,4-dichloro- phenyl)-imidazol-1- ylmethyl]-benzoic acid methyl ester |
| 350 | 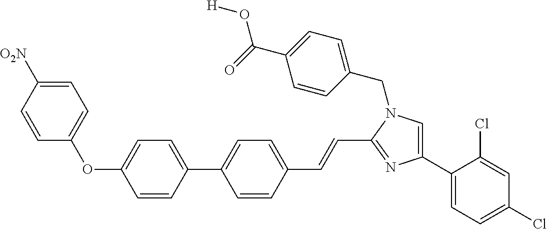 | 4-[2-{2-[4′-(4-nitro- phenoxy)-biphenyl-4-yl]- (E)-vinyl}-4-(2,4-dichloro- phenyl)-imidazol-1- ylmethyl]-benzoic acid |
| 351 | 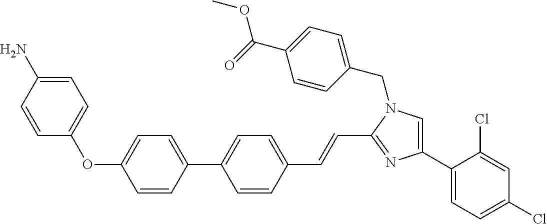 | 4-[2-{2-[4′-(4-amino- phenoxy)-biphenyl-4-yl]- (E)-vinyl}-4-(2,4-dichloro- phenyl)-imidazol-1- ylmethyl]-benzoic acid methyl ester |
| 352 | 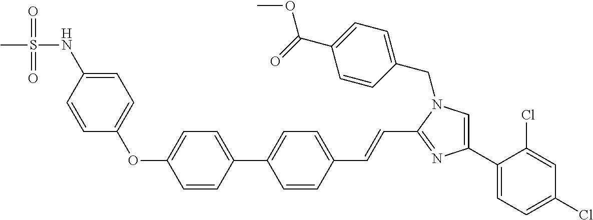 | 4-(4-(2,4-dichloro-phenyl)- 2-{2-[4′-(4- methanesulfonylamino- phenoxy)-biphenyl-4-yl]- (E)-vinyl}-imidazol-1- ylmethyl)-benzoic acid methyl ester |
| 353 | 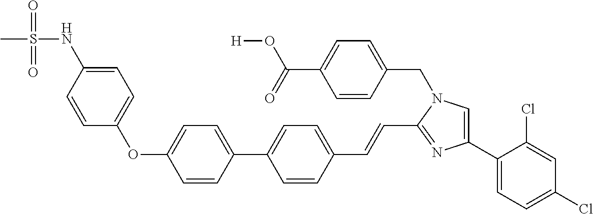 | 4-(4-(2,4-dichloro-phenyl)- 2-{2-[4′-(4- methanesulfonylamino- phenoxy)-biphenyl-4-yl]- (E)-vinyl}-imidazol-1- ylmethyl)-benzoic acid |
| 354 | 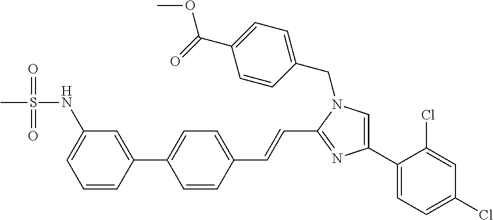 | 4-{4-(2,4-dichloro-phenyl)- 2-[2-(3′- methanesulfonylamino- biphenyl-4-yl)-(E)-vinyl]- imidazol-1-ylmethyl}- benzoic acid methyl ester |
| 355 | 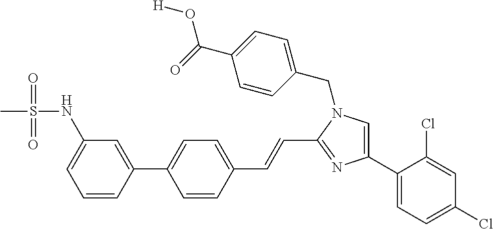 | 4-{4-(2,4-dichloro-phenyl)- 2-[2-(3′- methanesulfonylamino- biphenyl-4-yl)-(E)-vinyl]- imidazol-1-ylmethyl}- benzoic acid |
| 356 | 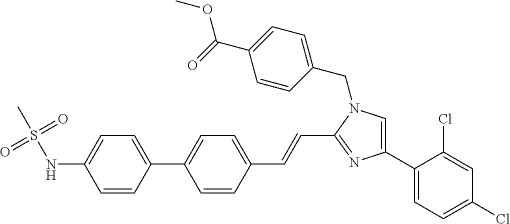 | 4-{4-(2,4-dichloro-phenyl)- 2-[2-(4′- methanesulfonylamino- biphenyl-4-yl)-(E)-vinyl]- imidazol-1-ylmethyl}- benzoic acid methyl ester |
| 357 | 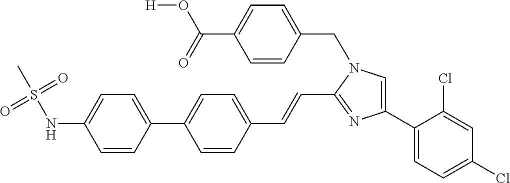 | 4-{4-(2,4-Dichloro-phenyl)- 2-[2-(4′- methanesulfonylamino- biphenyl-4-yl)-(E)-vinyl]- imidazol-1-ylmethyl}- benzoic acid |
| 358 | 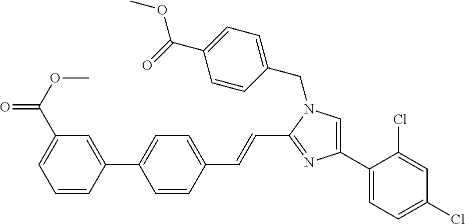 | 4′-{2-[4-(2,4-dichloro- phenyl)-1-(4- methoxycarbonyl-benzyl)- 1H-imidazol-2-yl]-(E)-vinyl}- biphenyl-3-carboxylic acid methyl ester |
| 359 | 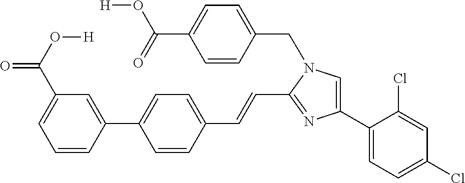 | 4′-{2-[1-(4-carboxy-benzyl)- 4-(2,4-dichloro-phenyl)-1H- imidazol-2-yl]-(E)-vinyl}- biphenyl-3-carboxylic acid |
| 360 | 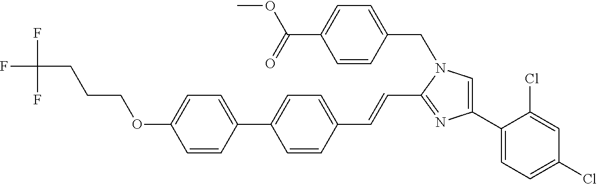 | 4-(4-(2,4-dichloro-phenyl)- 2-{2-[4′-(4,4,4-trifluoro- butoxy)-biphenyl-4-yl]-(E)- vinyl}-imidazol-1-ylmethyl)- benzoic acid methyl ester |
| 361 | 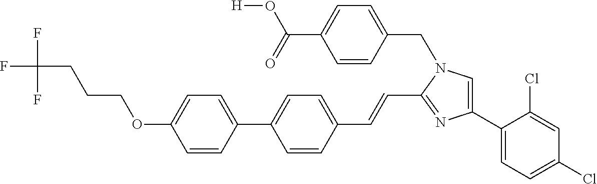 | 4-(4-(2,4-dichloro-phenyl)- 2-{2-[4′-(4,4,4-trifluoro- butoxy)-biphenyl-4-yl]-(E)- vinyl}-imidazol-1-ylmethyl)- benzoic acid |
| 362 | 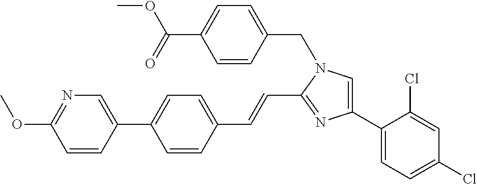 | 4-(4-(2,4-dichloro-phenyl)- 2-{2-[4-(6-methoxy-pyridin- 3-yl)-phenyl]-(E)-vinyl}- imidazol-1-ylmethyl)- benzoic acid methyl ester |
| 363 | 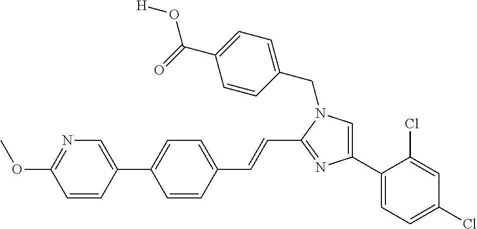 | 4-(4-(2,4-dichloro-phenyl)- 2-{2-[4-(6-methoxy-pyridin- 3-yl)-phenyl]-(E)-vinyl}- imidazol-1-ylmethyl)- benzoic acid |
| 364 | 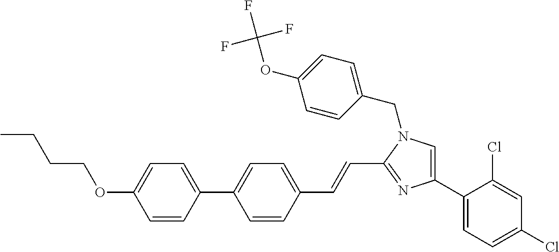 | 2-[2-(4′-butoxy-biphenyl-4- yl)-(E)-vinyl]-4-(2,4- dichloro-phenyl)-1-(4- trifluoromethoxy-benzyl)- 1H-imidazole |
| 365 | 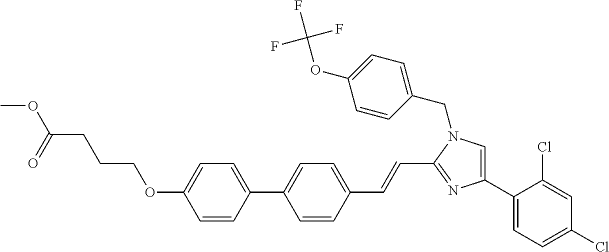 | 4-(4′-{2-[4-(2,4-dichloro- phenyl)-1-(4- trifluoromethoxy-benzyl)- 1H-imidazol-2-yl]-(E)-vinyl}- biphenyl-4-yloxy)-butyric acid methyl ester |
| 366 | 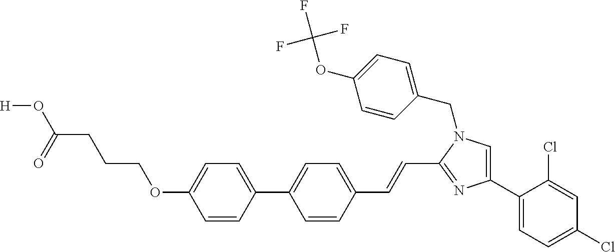 | 4-(4′-{2-[4-(2,4-dichloro- phenyl)-1-(4- trifluoromethoxy-benzyl)- 1H-imidazol-2-yl]-(E)-vinyl}- biphenyl-4-yloxy)-butyric acid |
| 367 | 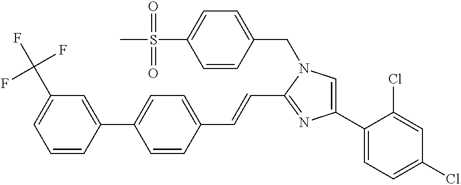 | 4-(2,4-dichloro-phenyl)-1- (4-methanesulfonyl- benzyl)-2-[2-(3′- trifluoromethyl-biphenyl-4- yl)-(E)-vinyl]-1H-imidazole |
| 368 | 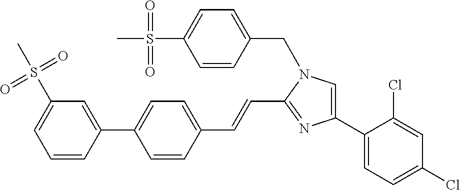 | 4-(2,4-dichloro-phenyl)-1- (4-methanesulfonyl- benzyl)-2-[2-(3′- methanesulfonyl-biphenyl- 4-yl)-(E)-vinyl]-1H- imidazole |
| 369 | 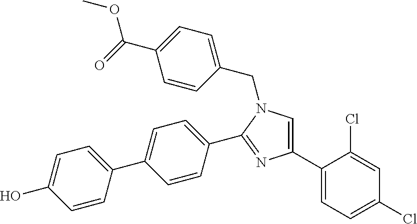 | 4-[4-(2,4-dichloro-phenyl)- 2-(4′-hydroxy-biphenyl-4- yl)-imidazol-1-ylmethyl]- benzoic acid methyl ester |
| 370 | 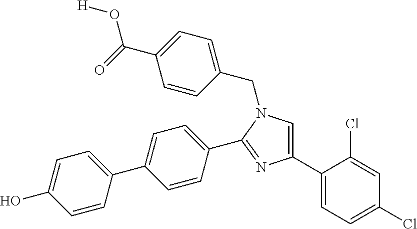 | 4-[4-(2,4-dichloro-phenyl)- 2-(4′-hydroxy-biphenyl-4- yl)-imidazol-1-ylmethyl]- benzoic acid |
| 371 | 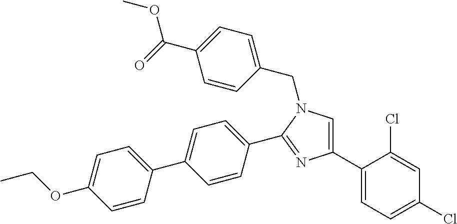 | 4-[4-(2,4-dichloro-phenyl)- 2-(4′-ethoxy-biphenyl-4- yl)-imidazol-1-ylmethyl]- benzoic acid methyl ester |
| 372 | 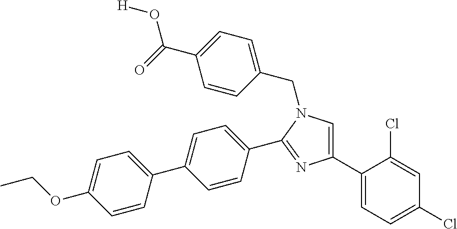 | 4-[4-(2,4-dichloro-phenyl)- 2-(4′-ethoxy-biphenyl-4- yl)-imidazol-1-ylmethyl]- benzoic acid |
| 373 | 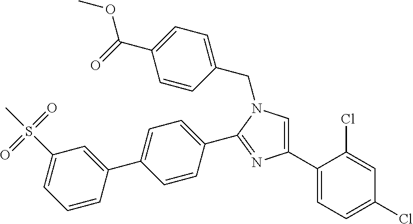 | 4-[4-(2,4-dichloro-phenyl)- 2-(3′-methanesulfonyl- biphenyl-4-yl)-imidazol-1- ylmethyl]-benzoic acid methyl ester |
| 374 | 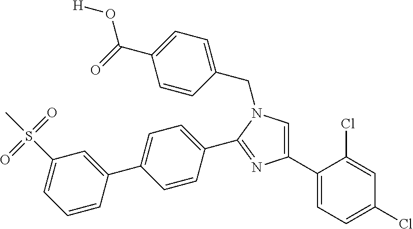 | 4-[4-(2,4-dichloro-phenyl)- 2-(3′-methanesulfonyl- biphenyl-4-yl)-imidazol-1- ylmethyl]-benzoic acid |
| 375 | 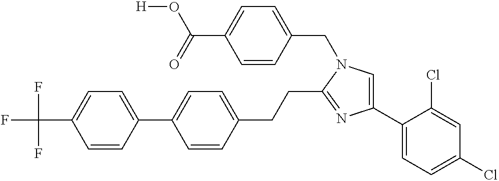 | 4-{4-(2,4-dichloro-phenyl)- 2-[2-(4′-trifluoromethyl- biphenyl-4-yl)-ethyl]- imidazol-1-ylmethyl}- benzoic acid |

In the structures listed above, it is understood that where a heteroatom such as nitrogen or oxygen has an unfilled valence, a covalent bond exists between a hydrogen and the heteroatom.

In another aspect, the present invention comprises a pharmaceutical composition comprising the compound of Formula (I) and one or more pharmaceutically acceptable carriers, excipients, or diluents.

As used herein, the term “lower” refers to a group having between one and six carbons.

As used herein, the term “alkyl” refers to a straight or branched chain hydrocarbon having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ atoms. Examples of “alkyl” as used herein include, but are not limited to, methyl, n-butyl, t-butyl, n-pentyl, isobutyl, and isopropyl, and the like.

As used herein, the term “alkylene” refers to a straight or branched chain divalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may containing one or more O, S, S(O), or S(O)₂ atoms. Examples of “alkylene” as used herein include, but are not limited to, methylene, ethylene, and the like.

As used herein, the term “alkenyl” refers to a hydrocarbon radical having from two to ten carbons and at least one carbon-carbon double bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may containing one or more O, S, S(O), or S(O)₂ atoms.

As used herein, the term “alkenylene” refers to a straight or branched chain divalent hydrocarbon radical having from two to ten carbon atoms and one or more carbon-carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may containing one or more O, S, S(O), or S(O)₂ atoms. Examples of “alkenylene” as used herein include, but are not limited to, ethene-1,2-diyl, propene-1,3-diyl, methylene-1,1-diyl, and the like.

As used herein, the term “alkynyl” refers to a hydrocarbon radical having from two to ten carbons and at least one carbon-carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may containing one or more O, S, S(O), or S(O)₂ atoms.

As used herein, the term “alkynylene” refers to a straight or branched chain divalent hydrocarbon radical having from two to ten carbon atoms and one or more carbon-carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may containing one or more O, S, S(O), or S(O)₂ atoms. Examples of “alkynylene” as used herein include, but are not limited to, ethyne-1,2-diyl, propyne-1,3-diyl, and the like.

As used herein, “cycloalkyl” refers to an alicyclic hydrocarbon group optionally possessing one or more degrees of unsaturation, having from three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “heterocyclic” or the term “heterocyclyl” refers to a three to twelve-membered heterocyclic ring optionally possessing one or more degrees of unsaturation, containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, 1,4-dioxane, 1,3-dioxane, piperidine, pyrrolidine, morpholine, piperazine, and the like.

As used herein, the term “heterocyclylene” refers to a three to twelve-membered heterocyclic ring diradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-diyl, and the like.

As used herein, the term “aryl” refers to a benzene ring or to an optionally substituted benzene ring system fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-naphthyl, 1-naphthyl, 1-anthracenyl, and the like.

As used herein, the term “arylene” refers to a benzene ring diradical or to a benzene ring system diradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

As used herein, the term “heteroaryl” refers to a five- to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, alkoxycarbonylamino optionally substituted by alkyl, acylamino optionally substituted by alkyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, aryloxycarbonyl, trialkylsilylalkyloxyalkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “fused cycloalkylaryl” refers to one or more cycloalkyl groups fused to an aryl group, the aryl and cycloalkyl groups having two atoms in common, and wherein the aryl group is the point of substitution. Examples of “fused cycloalkylaryl” used herein include 5-indanyl, 5,6,7,8-tetrahydro-2-naphthyl,

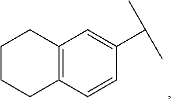

and the like.

As used herein, the term “fused cycloalkylarylene” refers to a fused cycloalkylaryl, wherein the aryl group is divalent. Examples include

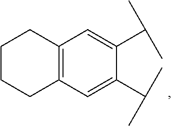

and the like.

As used herein, the term “fused arylcycloalkyl” refers to one or more aryl groups fused to a cycloalkyl group, the cycloalkyl and aryl groups having two atoms in common, and wherein the cycloalkyl group is the point of substitution. Examples of “fused arylcycloalkyl” used herein include 1-indanyl, 2-indanyl, 9-fluorenyl, 1-(1,2,3,4-tetrahydronaphthyl),

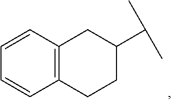

and the like.

As used herein, the term “fused arylcycloalkylene” refers to a fused arylcycloalkyl, wherein the cycloalkyl group is divalent. Examples include 9,1-fluorenylene,

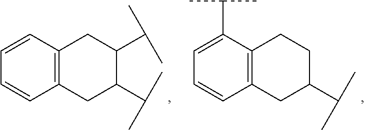

and the like.

As used herein, the term “fused heterocyclylaryl” refers to one or more heterocyclyl groups fused to an aryl group, the aryl and heterocyclyl groups having two atoms in common, and wherein the aryl group is the point of substitution. Examples of “fused heterocyclylaryl” used herein include 3,4-methylenedioxy-1-phenyl,

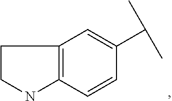

and the like

As used herein, the term “fused heterocyclylarylene” refers to a fused heterocyclylaryl, wherein the aryl group is divalent. Examples include

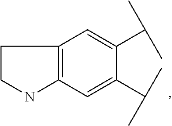

and the like.

As used herein, the term “fused arylheterocyclyl” refers to one or more aryl groups fused to a heterocyclyl group, the heterocyclyl and aryl groups having two atoms in common, and wherein the heterocyclyl group is the point of substitution. Examples of “fused arylheterocyclyl” used herein include 2-(1,3-benzodioxolyl),

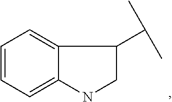

and the like.

As used herein, the term “fused arylheterocyclylene” refers to a fused arylheterocyclyl, wherein the heterocyclyl group is divalent. Examples include

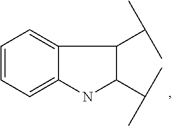

and the like.

As used herein, the term “fused cycloalkylheteroaryl” refers to one or more cycloalkyl groups fused to a heteroaryl group, the heteroaryl and cycloalkyl groups having two atoms in common, and wherein the heteroaryl group is the point of substitution. Examples of “fused cycloalkylheteroaryl” used herein include 5-aza-6-indanyl,

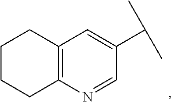

and the like.

As used herein, the term “fused cycloalkylheteroarylene” refers to a fused cycloalkylheteroaryl, wherein the heteroaryl group is divalent. Examples include

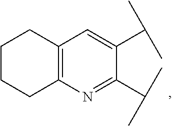

and the like.

As used herein, the term “fused heteroarylcycloalkyl” refers to one or more heteroaryl groups fused to a cycloalkyl group, the cycloalkyl and heteroaryl groups having two atoms in common, and wherein the cycloalkyl group is the point of substitution. Examples of “fused heteroarylcycloalkyl” used herein include 5-aza-1-indanyl,

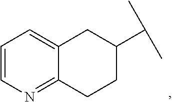

and the like.

As used herein, the term “fused heteroarylcycloalkylene” refers to a fused heteroarylcycloalkyl, wherein the cycloalkyl group is divalent. Examples include

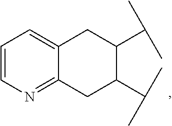

and the like.

As used herein, the term “fused heterocyclylheteroaryl” refers to one or more heterocyclyl groups fused to a heteroaryl group, the heteroaryl and heterocyclyl groups having two atoms in common, and wherein the heteroaryl group is the point of substitution. Examples of “fused heterocyclylheteroaryl” used herein include 1,2,3,4-tetrahydro-beta-carbolin-8-yl,

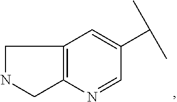

and the like.

As used herein, the term “fused heterocyclylheteroarylene” refers to a fused heterocyclylheteroaryl, wherein the heteroaryl group is divalent. Examples include

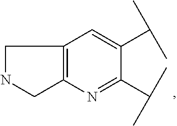

and the like.

As used herein, the term “fused heteroarylheterocyclyl” refers to one or more heteroaryl groups fused to a heterocyclyl group, the heterocyclyl and heteroaryl groups having two atoms in common, and wherein the heterocyclyl group is the point of substitution. Examples of “fused heteroarylheterocyclyl” used herein include -5-aza-2,3-dihydrobenzofuran-2-yl,

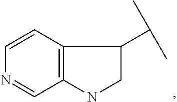

and the like.

As used herein, the term “fused heteroarylheterocyclylene” refers to a fused heteroarylheterocyclyl, wherein the heterocyclyl group is divalent. Examples include

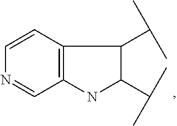

and the like.

As used herein, the term “acid isostere” refers to a substituent group which will ionize at physiological pH to bear a net negative charge. Examples of such “acid isosteres” include but are not limited to heteroaryl groups such as but not limited to isoxazol-3-ol-5-yl, 1H-tetrazole-5-yl, or 2H-tetrazole-5-yl. Such acid isosteres include but are not limited to heterocyclyl groups such as but not limited to imidazolidine-2,4-dione-5-yl, imidazolidine-2,4-dione-1-yl, 1,3-thiazolidine-2,4-dione-5-yl, or 5-hydroxy-4H-pyran-4-on-2-yl.

As used herein, the term “direct bond”, where part of a structural variable specification, refers to the direct joining of the substituents flanking (preceding and succeeding) the variable taken as a “direct bond”. Where two or more consecutive variables are specified each as a “direct bond”, those substituents flanking (preceding and succeeding) those two or more consecutive specified “direct bonds” are directly joined.

As used herein, the term “alkoxy” refers to the group RₐO—, where Rₐ is alkyl.

As used herein, the term “alkenyloxy” refers to the group RₐO—, where Rₐ is alkenyl.

As used herein, the term “alkynyloxy” refers to the group RₐO—, where Rₐ is alkynyl.

As used herein, the term “alkylsulfanyl” refers to the group RₐS—, where Rₐ is alkyl.

As used herein, the term “alkenylsulfanyl” refers to the group RₐS—, where Rₐ is alkenyl.

As used herein, the term “alkynylsulfanyl” refers to the group RₐS—, where Rₐ is alkynyl.

As used herein, the term “alkylsulfenyl” refers to the group RₐS(O)—, where Rₐ is alkyl.

As used herein, the term “alkenylsulfenyl” refers to the group RₐS(O)—, where Rₐ is alkenyl.

As used herein, the term “alkynylsulfenyl” refers to the group RₐS(O)—, where Rₐ is alkynyl.

As used herein, the term “alkylsulfonyl” refers to the group RₐSO₂—, where Rₐ is alkyl.

As used herein, the term “alkenylsulfonyl” refers to the group RₐSO₂—, where Rₐ is alkenyl.

As used herein, the term “alkynylsulfonyl” refers to the group RₐSO₂—, where Rₐ is alkynyl.

As used herein, the term “acyl” refers to the group RₐC(O)—, where Rₐ is alkyl, alkenyl, alkynyl, cycloalkyl, cycloalkenyl, or heterocyclyl.

As used herein, the term “aroyl” refers to the group RₐC(O)—, where Rₐ is aryl.

As used herein, the term “heteroaroyl” refers to the group RₐC(O)—, where Rₐ is heteroaryl.

As used herein, the term “alkoxycarbonyl” refers to the group RₐOC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “aroyloxy” refers to the group RₐC(O)O—, where Rₐ is aryl.

As used herein, the term “heteroaroyloxy” refers to the group RₐC(O)O—, where Rₐ is heteroaryl.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Alkyl or cycloalkyl substituents shall be recognized as being functionally equivalent to those having one or more degrees of unsaturation. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “oxo” shall refer to the substituent ═O.

As used herein, the term “halogen” or “halo” shall include iodine, bromine, chlorine and fluorine.

As used herein, the term “mercapto” shall refer to the substituent —SH.

As used herein, the term “carboxy” shall refer to the substituent —COOH.

As used herein, the term “cyano” shall refer to the substituent —CN.

As used herein, the term “aminosulfonyl” shall refer to the substituent —SO₂NH₂.

As used herein, the term “carbamoyl” shall refer to the substituent —C(O)NH₂.

As used herein, the term “sulfanyl” shall refer to the substituent —S—.

As used herein, the term “sulfenyl” shall refer to the substituent —S(O)—.

As used herein, the term “sulfonyl” shall refer to the substituent —S(O)₂—.

The compounds can be prepared readily according to the following reaction Schemes (in which variables are as defined before or are defined) using readily available starting materials, reagents and conventional synthesis procedures. In these reactions, it is also possible to make use of variants which are themselves known to those of ordinary skill in this art, but are not mentioned in greater detail.

The present invention also provides a method for the synthesis of compounds useful as intermediates in the preparation of compounds of Formula (I) along with methods for the preparation of compounds of Formula (I). Unless otherwise specified, structural variables are as defined for Formula (I).

An unsaturated carboxylic acid (Scheme 1) can be reacted with aryl acyl bromides in the presence of base such as DIEA, triethyl amine, or DBU in a polar solvents such as THF, or DMF to afford intermediate keto-ester (2), which can be treated with ammonium acetate in acetic acid at temperatures ranging from 60-120° C., which leads to the corresponding mixture of oxazole (W=O) and imidazole (W=N) (3) (Strzybny, P. P. E; van Es, T.; Backeberg, O. G. J. Org. Chem. 1963, 25, 1151). The ratio of oxazole and imidazole may vary depending on the substitution and reaction conditions and the two compounds were separated through silica gel column. Alternatively other conditions may also be employed for cyclization of keto-esters (2), such as BF₃/Et₂O, methanolic ammonia, at temperatures ranging from room temperature to 120° C.

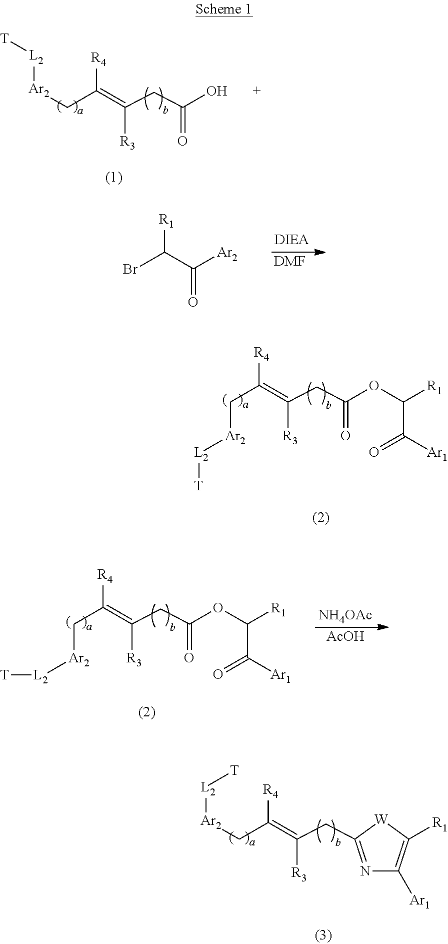

In another embodiment, a bromo or iodo aryl compound (4) (Scheme 2) can be subjected to palladium catalyzed coupling (Syn. Commu. 1981, 11, 513-574) with an optionally substituted heteroaryl or aryl boronic acid. Ar₃ is a group such as but not limited to a heteroaryl or aryl group. Typical conditions used to carry out the coupling reaction include the use of boronic acid or ester as the coupling partner, a palladium catalyst (2 to 20 mole %) such as Pd(PPh₃)₄ or [1,1-bis(diphenylphosphino)-ferrocene]dichloro-palladium (II) and base such as potassium carbonate, sodium carbonate, barium hydroxide, potassium phosphate or triethyl amine in a suitable solvent such as aqueous dimethoxyethane, THF, acetone, DMF or toluene at temperatures ranging from 25° C. to 125° C. In this instance, Ar₃ is a group such as, but not limited to, an aryl or heteroaryl group.

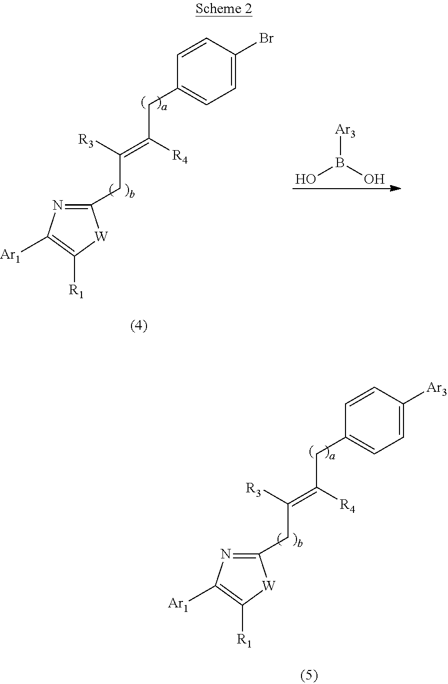

In another embodiment (Scheme 3), the O-alkyl, or O-aryl group in compound (5) can be dealkylated or dearylated using reagents such as boron tribromide or PhSMe, in a solvent such as dichloromethane or TFA, at temperatures ranging from −20° C. to room temperature to afford hydroxy biphenyls (6). In this instance, Ar₄ is a group such as, but not limited to, heteroarylene or arylene, and R₃₀ is a group such as, but not limited to, lower alkyl.

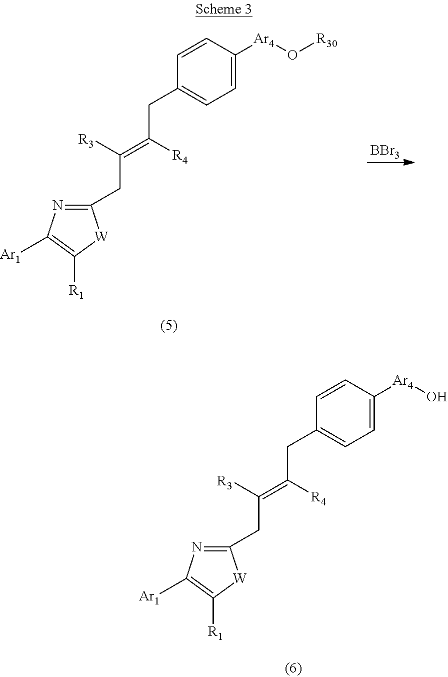

In Scheme 4, the biphenyl alcohols (5) were alkylated with bromo or chloro alkyl carboxylates [(Br or Cl)(CH₂)ₙ—CO₂—R₃₀] [where n=1 to 6] in the presence of base such as sodium hydride, potassium tert-butoxide, or potassium carbonate using DMF, THF, acetonitrile as the solvent at temperatures ranging from 50° C. to 100° C. Subsequent saponification of esters (6) with bases such as sodium hydroxide, lithium hydroxide in aqueous and organic solvents such as THF, methanol, at temperatures ranging from room temperature to 60° C. produces carboxylic acid (8). In this instance, R₃₀ is a group such as, but not limited to, lower alkyl. In this instance, Arₐ is a group such as, but not limited to, an arylene or heteroarylene group.

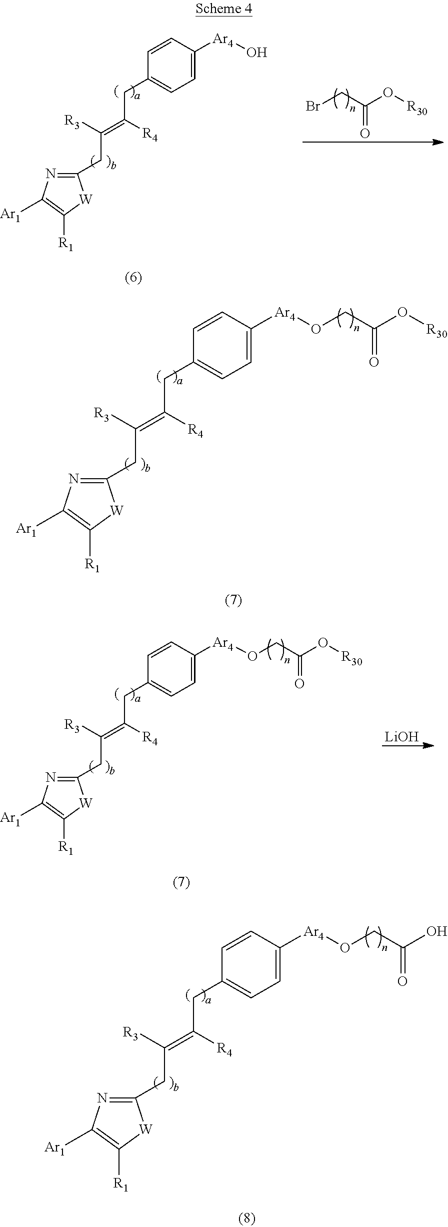

In another embodiment (Scheme 5), the imidazole nitrogen in compound (9) can be alkylated with bromo or chloro alkyl carboxylates [(Br or Cl)(CH₂)ₙCO₂R₃₀] in the presence of base such as sodium hydride, potassium tert-butoxide, or potassium carbonate using DMF, THF, or acetonitrile as the solvent at temperatures ranging from 50° C. to 100° C. Subsequent saponification of esters (10) with base such as sodium hydroxide, lithium hydroxide in aqueous and organic solvents such as THF, or methanol at temperatures ranging from room temperature to 60° C. produces carboxylic acid (11). In this instance, R₃₀ is a group such as, but not limited to, lower alkyl.

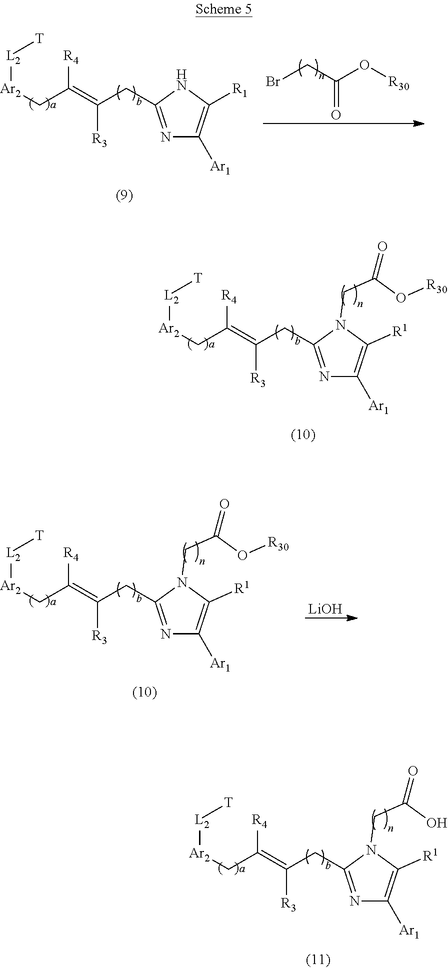

In Scheme 6 the carboxylic acids (12) can be transformed into their carboxylic acid amide analogs. This transformation can be accomplished using standard methods to effect carboxylic acid to carboxylic acid amide transformations. These methods include converting the acid to an activated acid, reacting with one or more molar equivalents of the desired amine. Methods to activate the carboxylic acid include reacting the acid with one or more molar equivalents of DIC or DIEA, with or without one or more molar equivalents of HOBt or HBTU in a suitable solvent such as dichloromethane or DMF at temperatures ranging from 0° C. to 40° C. to afford amides (13). In this instance, R₃₁ is a group such as, but not limited to, -alkyl or -alkylene-aryl.

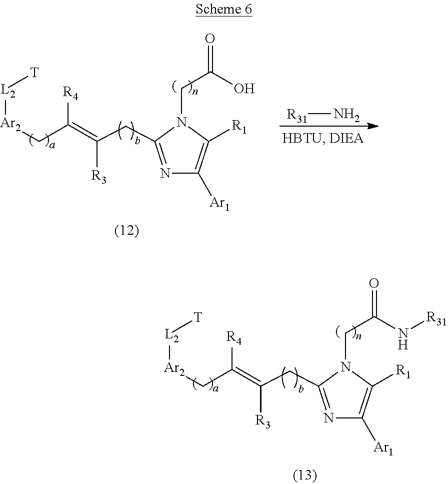

In another embodiment (Scheme 7), an imidazole nitrogen in compound (14) was alkylated with alkyl halides [(Br or Cl)(CH₂)ₙ—R₃₂] [n=1 to 6] in the presence of base such as sodium hydride, potassium tert-butoxide, or potassium carbonate using DMF, THF, or acetonitrile as the solvent at temperatures ranging from 0° C. to 80° C. afford N-alkylated products (15). In this instance R₃₂ is a group such as, but not limited to, -alkyl, aryl, or -alkenylene-aryl.

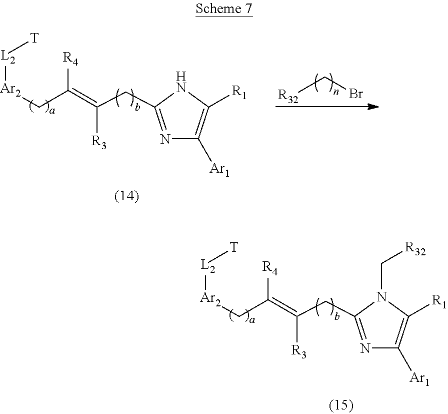

The term “amino protecting group” as used herein refers to substituents of the amino group commonly employed to block or protect the amino functionality while reacting other functional groups on the compound. Examples of such amino-protecting groups include the formyl group, the trityl group, the phthalimido group, the trichloroacetyl group, the chloroacetyl, bromoacetyl and iodoacetyl groups, urethane-type blocking groups such as benzyloxycarbonyl, 4-phenyl benzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxy-carbonyl, 2-(4-xenyl)iso-propoxycarbonyl, 1,1-diphenyleth-1-yloxycarbonyl, 1,1-diphenylprop-1-yloxycarbonyl, 2-phenylprop-2-yloxycarbonyl, 2-(p-toluoyl)prop-2-yloxycarbonyl, cyclopentanyloxycarbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfonypethoxycarbonyl, 2(methylsulfonyl)ethoxycarbonyl, 2-(triphenylphosphino)ethoxycarbonyl, 9-fluorenylmethoxycarbonyl (“FMOC”), t-butoxycarbonyl (“BOC”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonyl and the like; the benzoylmethylsulfonyl group, the 2-(nitro)phenylsulfenyl group, the diphenylphosphine oxide group and like amino-protecting groups. The species of amino-protecting group employed is not critical so long as the derivatized amino group is stable to the condition of subsequent reaction(s) on other positions of the compound of Formula (I) and can be removed at the desired point without disrupting the remainder of the molecule. In an embodiment, amino-protecting groups are the allyloxycarbonyl, the t-butoxycarbonyl, 9-fluorenylmethoxycarbonyl, and the trityl groups. Similar amino-protecting groups used in the cephalosporin, penicillin and peptide art are also embraced by the above terms. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected amino” or “protected amino group” defines an amino group substituted with an amino-protecting group discussed above.

The term “hydroxyl protecting group” as used herein refers to substituents of the alcohol group commonly employed to block or protect the alcohol functionality while reacting other functional groups on the compound. Examples of such alcohol-protecting groups include the 2-tetrahydropyranyl group, 2-ethoxyethyl group, the trityl group, the trichloroacetyl group, urethane-type blocking groups such as benzyloxycarbonyl, and the trialkylsilyl group, examples of such being trimethylsilyl, tert-butyldimethylsilyl, phenyldimethylsilyl, triisopropylsilyl and thexyldimethylsilyl. The choice of alcohol-protecting group employed is not critical so long as the derivatized alcohol group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected hydroxyl” or “protected alcohol” defines a hydroxyl group substituted with a hydroxyl-protecting group as discussed above.

The term “carboxyl protecting group” as used herein refers to substituents of the carboxyl group commonly employed to block or protect the —OH functionality while reacting other functional groups on the compound. Examples of such alcohol-protecting groups include the 2-tetrahydropyranyl group, 2-ethoxyethyl group, the trityl group, the allyl group, the trimethylsilylethoxymethyl group, the 2,2,2-trichloroethyl group, the benzyl group, and the trialkylsilyl group, examples of such being trimethylsilyl, tert-butyldimethylsilyl, phenyldimethylsilyl, triisopropylsilyl and thexyldimethylsilyl. The choice of carboxyl protecting group employed is not critical so long as the derivatized alcohol group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected carboxyl” defines a carboxyl group substituted with a carboxyl-protecting group as discussed above.

The general procedures used in the methods of the present invention are described below.

### GENERAL EXPERIMENTAL

LC-MS data was obtained using gradient elution on a Waters 600 controller equipped with a 2487 dual wavelength detector and a Leap Technologies HTS PAL Autosampler using an YMC Combiscreen ODS-A 50×4.6 mm column. A three minute gradient was run from 25% B (97.5% acetonitrile, 2.5% water, 0.05% TFA) and 75% A (97.5% water, 2.5% acetonitrile, 0.05% TFA) to 100% B. The mass spectrometer used was a Micromass ZMD instrument. All data was obtained in the positive mode unless otherwise noted. ¹H NMR data was obtained on a Varian 400 MHz spectrometer. Abbreviations used in the Examples are as follows:

APCI=atmospheric pressure chemical ionization
BOC=tert-butoxycarbonyl
BOP=(1-benzotriazolyloxy)tris(dimethylamino)phosphonium hexafluorophosphate
d=day
DIAD=diisopropyl azodicarboxylate
DCC=dicyclohexylcarbodiimide
DCM=dichloromethane
DIC=diisopropylcarbodiimide
DIEA=diisopropylethylamine

### DMA=N,N-dimethylacetamide

DMAP=dimethylaminopyridine
DME=1,2 dimethoxyethane

### DMF=N,N-dimethylformamide

DMPU=1,3-dimethylpropylene urea
DMSO=dimethylsulfoxide
EDC=1-ethyl-3-(3-dimethylaminopropyl)-carbodiimide hydrochloride
EDTA=ethylenediamine tetraacetic acid
ELISA=enzyme-linked immunosorbent assay
ESI=electrospray ionization
ether=diethyl ether
EtOAc=ethyl acetate
FBS=fetal bovine serum
g=gram
h=hour
HBTU=O-benzotriazol-1-yl-N,N,N′,N′-tetramethyluronium hexafluorophosphate
HMPA=hexamethylphosphoric triamide
HOBt=1-hydroxybenzotriazole
Hz=hertz
i.v.=intravenous
kD=kiloDalton
L=liter
LAH=lithium aluminum hydride
LDA=lithium diisopropylamide
LPS=lipopolysaccharide
M=molar
m/z=mass to charge ratio
mbar=millibar
MeOH=methanol
mg=milligram
min=minute
mL=milliliter
mM=millimolar
mmol=millimole
mol=mole
mp=melting point
MS=mass spectrometry
N=normal
NMM=N-methylmorpholine, 4-methylmorpholine
NMR=nuclear magnetic resonance spectroscopy
p.o.=per oral
PBS=phosphate buffered saline solution
PMA=phorbol myristate acetate
ppm=parts per million
psi=pounds per square inch
R_{f}=relative TLC mobility
rt=room temperature
s.c.=subcutaneous
SPA=scintillation proximity assay
TEA=triethylamine
TFA=trifluoroacetic acid
THF=tetrahydrofuran
THP=tetrahydropyranyl
TLC=thin layer chromatography
TMSBr=bromotrimethylsilane, trimethylsilylbromide
Tᵣ=retention time

### Insert New Experimental

### General Procedure A

### Imidazole Formation

To a mixture of a carboxylic acid (1 eq) and an aromatic acyl bromide (2 eq) in anhydrous DMF (0.1-0.5 M) was added DIEA (3 eq). The reaction mixture was stirred at room temperature under nitrogen for 6 to 8 hours. After that, it was poured into water, acidified with 10% citric acid and extracted with ethyl acetate. The organic extract was washed with water and brine, dried over Na₂SO₄. After evaporation of the solvent, the pale-brown residue was recrystallized from EtOAc-Hexanes, dried and used directly in the next step.

The intermediate obtained above was dissolved in glacial acetic acid (0.1-0.5 M), and ammonium acetate (20 eq) was added. The mixture was then heated at 120° C. under nitrogen for 8 to 10 hours. At completion, it was poured into water, neutralized with saturated sodium bicarbonate and extracted with ethyl acetate. The organic extract was washed with water and brine, and dried over Na₂SO₄. After removal of the solvent in vacuo, the residue was purified by flash column chromatography to afford the desired product.

### General Procedure B

### Boronic Acid Coupling

To a solution of the bromo compound (1 eq) in a 2:1 mixture of toluene and ethanol (0.1-0.5 M) was added the appropriate boronic acid (1.2 eq) and a catalytic amount of tetrakis(triphenylphosphine)palladium(0) (0.05 eq), followed by 2 M sodium carbonate solution in water (30 eq). The reaction mixture was stirred at 90° C. under nitrogen for 6 hours. After cooling, the reaction mixture was diluted with water and extracted with ethyl acetate. The organic extract was washed with water and brine, and dried over Na₂SO₄. After removal of the solvent in vacuo, the residue was purified by flash column chromatography to afford the desired compound.

### General Procedure C

### Dealkylation

To the solution of alkyl phenolic ether (1 eq) in anhydrous DCM (0.1-0.5 M) at −20° C. was added dropwise BBr₃ (2 eq, solution in anhydrous DCM). The solution was warmed to room temperature over 30 minutes, and the reaction mixture quenched with ice water. The reaction mixture was then diluted with water/EtOAc and the layers were separated. The aqueous layer was further extracted with EtOAc, and the organic layers combined, washed with water and brine, and dried over Na₂SO₄. The solvent was removed in vacuo, and the residue subjected to silica gel chromatography to yield the final product.

### General Procedure D

### Hydrogenation of Double Bond

To 1 equivalent of the desired alkene suspension in ethyl acetate (0.1-0.5 M) was added a catalytic amount of platinum(IV) oxide (wet). After degassing and introducing of nitrogen and degassing again, hydrogen was introduced through a hydrogen balloon. The reaction mixture was stirred at room temperature for 0.5 hour. The reaction mixture was then filtered through celite, the celite cake was washed three times with ethyl acetate, and the filtrates combined. The solvent was then removed in vacuo, and the residue was purified by silica gel chromatography to afford the desired compound.

### General Procedure E

### Alkylation of Imidazole Nitrogen or Phenolic Oxygen

To a solution of imidazole or phenol (1 eq) in anhydrous DMF (0.1-0.5 M) was added an alkyl or aryl halide (2 eq) followed by freshly ground K₂CO₃ (4 eq). The reaction mixture was heated at 100° C. under nitrogen for 2 hours. The mixture was then diluted with water/EtOAc and the layers separated. The aqueous layer was further extracted with EtOAc, and the organic layers combined and dried over Na₂SO₄. The solvent was removed in vacuo and the residue was purified by silica gel chromatography to yield the final product.

### General Procedure F

### Hydrolysis of Ester

The ester (1 eq) was suspended in a mixture of MeOH:THF:H₂O (1:1:1; 0.1-0.2 M). LiOH (10-15 eq) was added and the mixture stirred at 40° C. for 3 hours. The solution was acidified with 10% citric acid solution, and extracted with ethyl acetate. The organic extracts were combined, washed with brine, dried over Na₂SO₄, and the solvent removed in vacuo. The residue was purified by silica gel chromatography to yield the final compound.

### General Procedure G

### Coupling of Carboxylic Acid and Amine

To a solution of carboxylic acid (1.1 eq) in DMF (0.1-0.5 M), HBTU (1.1 eq) was added followed by DIEA (1.2 eq) and the appropriate protected amine (1 eq.). The reaction mixture was then stirred at room temperature for 4 hours. At completion, the reaction mixture was diluted with water/EtOAc, acidified with 10% citric acid, and the layers were separated. The combined organic layer was washed with water, saturated NaHCO₃ and brine, dried over Na₂SO₄ and filtered. The filtrate was concentrated and purified by silica gel chromatography to afford the amide derivative.

### General Procedure H

### Sonogashira Coupling

To a solution of aryl bromide or aryl iodide (1 eq) in anhydrous DMF (0.1-0.5 M) was added the appropriate terminal acetylene (1.2 eq) followed by tetrakis (triphenylphosphine)palladium(0) (0.05 eq), CuI (0.1 eq), and DIEA (2 eq). The reaction mixture was then heated at 120° C. under nitrogen for 6-8 hours. At completion, the reaction mixture was diluted with water/EtOAc, acidified with 10% citric acid, and the layers separated. The combined organic layers was washed with water and brine, dried over Na₂SO₄ and filtered. The filtrate was concentrated and purified by silica gel chromatography to afford the acetylene derivative.

### General Procedure I

### Diaryl Ether Formation Using Aryl Fluoride

To a solution of phenol compound (1 eq) in anhydrous DMF (0.1-0.5 M), the appropriate activated aryl fluoride (1.5 eq) was added followed by Cs₂CO₃ (3 eq). The reaction mixture was then heated at 120° C. under nitrogen for 2 hours. At completion, the reaction mixture was diluted with water/EtOAc and the layers separated. The aqueous layer was reextracted with EtOAc and the organic layers combined, washed with water and brine. The organic phase was then dried over Na₂SO₄, filtered, and the filtrate was concentrated and purified by silica gel chromatography to afford the diaryl ether derivative.

### General Procedure J

### Ullmann Diaryl Ether Coupling

To a solution of phenol compound (1 eq) in anhydrous NMP (0.1-0.5 M), the appropriate aryl bromide or iodide (1.5 eq) was added followed by CuCl (0.2 eq), 2,2,6,6-tetramethyl-3,5-heptanedione (0.2 eq) and Cs₂CO₃ (3 eq). The reaction mixture was then heated at 120° C. under nitrogen for 6 to 8 hours. At completion, the reaction mixture was diluted with water/EtOAc and the layers separated. The aqueous layer was reextracted with EtOAc and the organic layers combined, washed with water and brine. The organic phase was then dried over Na₂SO₄, filtered, and the filtrate was concentrated and purified by silica gel chromatography to afford the diaryl ether derivative.

### General Procedure K

### Reduction of Aryl Nitro Group

To a suspension of aryl nitro compound (1 eq) in HOAc (0.1-0.5 M), iron powder (−325 mesh, 4 eq) was added and the mixture was then heated at 120° C. under nitrogen for 3 to 4 hours. At completion, the reaction mixture was diluted with water/EtOAc and the leftover iron powder was filtered and washed with EtOAc. The combined organic layer was washed with water, saturated NaHCO₃ and brine. The organic phase was then dried over Na₂SO₄, filtered, and the filtrate was concentrated and purified by silica gel chromatography to afford the aniline derivative.

### General Procedure L

### Coupling of Aniline with Sulfonyl Chloride or Sulfonic Anhydride

To a suspension of aniline compound (1 eq) in anhydrous DCM (0.1-0.5 M) at 0° C. was added DIEA (1.2 eq) followed by the appropriate sulfonyl chloride or sulfonic anhydride (1.1 eq, diluted in anhydrous DCM). The reaction mixture was then warmed up and stirred at room temperature under nitrogen for 3 to 4 hours. At completion, the reaction mixture was diluted with water/EtOAc and the layers separated. The aqueous layer was reextracted with EtOAc and the organic layers combined, washed with 10% citric acid, water and brine. The organic phase was then dried over Na₂SO₄, filtered, and the filtrate was concentrated and purified by silica gel chromatography to afford the sulfonamide derivative.

### General Procedure M

### Formation of Tetrazole

To a solution of phenol compound (1 eq) in anhydrous DMF (0.1-0.5 M) was added an appropriate bromoalkylnitrile (2 eq) followed by freshly ground K₂CO₃ (4 eq). The reaction mixture was heated at 100° C. under nitrogen for 2 hours. The mixture was then diluted with water/EtOAc and the layers separated. The aqueous layer was further extracted with EtOAc, and the organic layers combined and dried over Na₂SO₄. The solvent was removed in vacuo and the residue purified by silica gel chromatography to yield the nitrile intermediate.

The nitrile intermediate (1 eq) obtained above was dissolved in anhydrous DMF (0.1-0.5 M) and sodium azide (10 eq) and ammonium chloride (10 eq) were added. The reaction mixture was heated at 120° C. under nitrogen for 8 to 10 hours. At completion, the reaction mixture was diluted with water/EtOAc and the layers separated. The aqueous layer was further extracted with EtOAc, and the organic layers combined and dried over Na₂SO₄. The solvent was removed in vacuo and the residue was purified by silica gel chromatography to afford the final product.

### General Procedure N

### Protection of Imidazole Nitrogen

1 equivalent of an imidazole was suspended in anhydrous THF (0.1-0.5 M), to which was added 1.4 equivalents of TEA and 1.5 equivalents of di-tert-butyl-dicarbonate. The mixture was stirred for 2 hours and diluted with water and the layers were separated. The aqueous layer was further extracted with EtOAc, the organic layers combined, washed with brine, and the organic layer dried over sodium sulfate. The solvent was removed in vacuo, and the crude product purified by flash chromatography on silica gel to give the final product.

### General Procedure O

### Removal of the T-Butyl Carbamate Group

The protected compound was stirred in 4N HCl/dioxane for 1 hour. The solvent removed, and the product triturated several times with ether to afford the desired compound.

### General Procedure P

### Alkylation

To a solution of imidazole or phenol (1 eq) in anhydrous DMF (0.1-0.5M) was added 1-2 eq sodium hydride, either solid or as a suspension in DMF or THF. The mixture was stirred at room temperature for 20 min and a solution of alkyl or aryl halide (1-3 eq) was added in DMF or THF. Stirring continued for 1 hour, then the mixture was diluted with water/EtOAc and neutralized with 10% aqueous citric acid. The organic layer was washed with brine, dried over Na₂SO₄, and evaporated in vacuo. The residue was purified by silica gel chromatography to provide the final product.

### General Procedure Q

### Benzimidazole Formation

To a solution of an aldehyde (1 eq) in ethanol (0.1-0.5 M) was added 1.5 eq of a benzenediamine. The mixture was sealed in a heavy walled glass tube with stir bar and stirred at 100° C. for 2 hours to overnight. The mixture was then evaporated and taken up in water/EtOAc and layers were separated. The aqueous layer was further extracted with EtOAc and the combined organic extracts were washed with brine, dried over Na₂SO₄, and evaporated in vacuo. The residue was purified by silica gel chromatography to give the product.

### General Procedure R

### Catalytic Reduction of Aryl Nitro Group

To a solution of aryl nitro compound (1 eq) in methanol (0.1-0.5 M) was added 0.1 eq of 10% Pd/C catalyst. The flask was flushed with H₂ and stirred under H₂ pressure (balloon) overnight at room temperature. The mixture was then filtered on a celite pad and evaporated, and the residue was purified by silica gel column chromatography to provide the desired product.

### General Procedure S

### Silyl Group Deprotection

To a solution of O— or N— silyl compound (1 eq) in THF (0.1-0.5 M) was added 5 eq of tetrabutylammonium fluoride as a solution in THF. The mixture was stirred at 65° C. for 1-3 hours, then was evaporated to a small volume and taken up in water/EtOAc. Layers were separated and the aqueous layer was further extracted with EtOAc. The combined organic extracts were washed with brine, dried over Na₂SO₄, and evaporated in vacuo. The residue was purified by silica gel column chromatography to give the desired product.

### General Procedure T

### Selective Trimethylsilyl Group Deprotection

To a solution of trimethylsilyl compound (1 eq) in anhydrous methanol (0.1-0.5 M) was added 10 eq anhydrous K₂CO₃ under nitrogen. The mixture was stirred under nitrogen at room temperature for 3 hours, then diluted with water/EtOAc and layers were separated. The aqueous layer was further extracted with EtOAc and the combined organic layers were washed with brine, dried over Na₂SO₄ and evaporated in vacuo. The residue was purified by silica gel column chromatography to provide the desired product.

### General Procedure U

### Reductive Amination

To a solution of amine (1 eq) in 1,2-dichloroethane (0.1-0.5 M) was added an aldehyde (1.2 eq) and a catalytic amount of acetic acid. The mixture was stirred at room temperature for 30 minutes under nitrogen, then sodium triacetoxyborohydride (3 eq) was added and the mixture was allowed to stir for 12-16 hours at room temperature. The mixture was then diluted with water/EtOAc and layers were separated. The aqueous layer was extracted additionally with EtOAc and the combined organic extracts were washed with water, brine, dried over Na₂SO₄ and evaporated in vacuo. The residue was purified by silica gel column chromatography to provide the desired product.

### General Procedure V

### Saturation of Double Bond

To a suspension of double bond containing compound (1 eq) in HOAc (0.1-0.5 M) was added iron powder (−325 mesh, 10-20 eq) and the mixture was stirred and heated at 120° C. for 18-24 hours. The mixture was then diluted with water/EtOAc and filtered to remove excess iron powder, then layers were separated and the aqueous layer was washed again with EtOAc. The combined organic extracts were washed with water, saturated NaHCO₃, and brine, then dried over Na₂SO₄. After evaporation in vacuo, the residue was purified by silica gel column chromatography to provide the desired product.

### Example 1

### 4-(2,4-Dichloro-phenyl)-2-[2-(4-methoxy-phenyl)-(E)-vinyl]-1H-imidazole

Trans-4-methoxycinnamic acid (178 mg, 1 mmol) was treated according to general procedure A using 2,4-dichlorophenacyl bromide to give 4-(2,4-dichloro-phenyl)-2-[2-(4-methoxy-phenyl)-(E)-vinyl]-1H-imidazole (193 mg, 56% yield).

LCMS: m/z 345 (M+H)⁺; ¹H NMR (CD₃OD, 400 MHz): δ 3.82 (s, 3H), 6.88 (d, 1H), 6.95 (d, 2H), 7.33 (d, 1H), 7.51 (d, 2H), 7.52 (d, 1H), 7.54 (s, 1H), 7.66 (d, 1H), 7.93 (s, 1H) ppm.

### Example 2

### 4-(2,4-Dichloro-phenyl)-2-[2-(3-methoxy-phenyl)-(E)-vinyl]-1H-imidazole

Trans-3-methoxycinnamic acid (178 mg, 1 mmol) was treated according to general procedure A using 2,4-dichlorophenacyl bromide to give 4-(2,4-dichloro-phenyl)-2-[2-(3-methoxy-phenyl)-(E)-vinyl]-1H-imidazole (176 mg, 51% yield).

LCMS: m/z 345 (M+H)⁺; ¹H NMR (CD₃OD, 400 MHz): δ 3.81 (s, 3H), 6.88 (d, 1H), 7.04 (m, 3H), 7.32 (d, 1H), 7.41 (s, 1H), 7.50 (d, 1H), 7.54 (s, 1H), 7.67 (d, 1H), 7.92 (s, 1H) ppm.

### Example 3

### 4-(2,4-Dichloro-phenyl)-2-[2-(2-methoxy-phenyl)-(E)-vinyl]-1H-imidazole

Trans-2-methoxycinnamic acid (178 mg, 1 mmol) was treated according to general procedure A using 2,4-dichlorophenacyl bromide to give 4-(2,4-dichloro-phenyl)-2-[2-(2-methoxy-phenyl)-(E)-vinyl]-1H-imidazole (207 mg, 60% yield).

LCMS: m/z 345 (M+H)⁺; ¹H NMR (CD₃OD, 400 MHz): δ 3.82 (s, 3H), 6.88 (d, 1H), 7.04-7.15 (m, 4H), 7.32 (d, 1H), 7.50 (d, 1H), 7.54 (s, 1H), 7.67 (d, 1H), 7.93 (s, 1H) ppm.

### Example 4

### 4-(2,4-Dichloro-phenyl)-2-[2-(3,4-dimethoxy-phenyl)-(E)-vinyl]-1H-imidazole

Trans-3,4-dimethoxycinnamic acid (208 mg, 1 mmol) was treated according to general procedure A using 2,4-dichlorophenacyl bromide to give 4-(2,4-dichloro-phenyl)-2-[2-(3,4-dimethoxy-phenyl)-(E)-vinyl]-1H-imidazole (176 mg, 47% yield).

LCMS: m/z 375 (M+H)⁺; ¹H NMR (CD₃OD, 400 MHz): δ 3.89 (s, 3H), 3.91 (s, 3H), 7.00 (d, 1H), 7.05 (d, 1H), 7.24-7.28 (m, 2H), 7.56 (dd, 1H), 7.66 (d, 1H), 7.69 (d, 1H), 7.75 (d, 1H), 7.89 (s, 1H) ppm.

### Example 5

### 4-(2,4-Dichloro-phenyl)-2-[2-(2,3,4-trimethoxy-phenyl-(E)-vinyl]-1H-imidazole

Trans-2,3,4-trimethoxycinnamic acid (238 mg, 1 mmol) was treated according to general procedure A using 2,4-dichlorophenacyl bromide to give 4-(2,4-dichloro-phenyl)-2-[2-(2,3,4-trimethoxy-phenyl)-vinyl]-1H-imidazole (170 mg, 42% yield).

LCMS: m/z 405 (M+H)⁺; ¹H NMR (CD₃OD, 400 MHz): δ 3.85 (s, 3H), 3.91 (s, 3H), 3.98 (s, 3H), 6.91 (d, 1H), 7.12 (d, 1H), 7.44 (d, 1H), 7.55 (dd, 1H), 7.69 (d, 1H), 7.74 (d, 1H), 7.87 (s, 1H), 7.92 (d, 1H) ppm.

### Example 6

### 4-(2,4-Dichloro-phenyl)-2-[2-(4-ethoxy-phenyl)-(E)-vinyl]-1H-imidazole

Trans-4-ethoxycinnamic acid (192 mg, 1 mmol) was treated according to general procedure A using 2,4-dichlorophenacyl bromide to give 4-(2,4-dichloro-phenyl)-2-[2-(4-ethoxy-phenyl)-(E)-vinyl]-1H-imidazole (222 mg, 64% yield).

LCMS: m/z 359 (M+H)⁺; ¹H NMR (CD₃OD, 400 MHz): δ 1.41 (t, 3H), 4.10 (q, 2H), 6.97 (d, 1H), 7.01 (d, 2H), 7.55 (dd, 1H), 7.63 (d, 2H), 7.68 (d, 1H), 7.69 (d, 1H), 7.74 (d, 1H), 7.88 (s, 1H) ppm.

### Example 7

### 4-(2,4-Dichloro-phenyl)-2-styryl-1H-imidazole

Trans-cinnamic acid (148 mg, 1 mmol) was treated according to general procedure A using 2,4-dichlorophenacyl bromide to give 4-(2,4-dichloro-phenyl)-2-styryl-1H-imidazole (202 mg, 64% yield).

LCMS: m/z 315 (M+H)⁺; ¹H NMR (CD₃OD, 400 MHz): δ 7.13 (d, 1H), 7.49 (m, 3H), 7.68-7.73 (m, 4H), 7.77 (d, 1H), 8.03 (m, 2H) ppm.

### Example 8

### 4-(2,4-Dichloro-phenyl)-2-[2-(4-fluoro-phenyl)-(E)-vinyl]-1H-imidazole

Trans-4-fluorocinnamic acid (166 mg, 1 mmol) was treated according to general procedure A using 2,4-dichlorophenacyl bromide to give 4-(2,4-dichloro-phenyl)-2-[2-(4-fluoro-phenyl)-(E)-vinyl]-1H-imidazole (236 mg, 71% yield).

LCMS: m/z 333 (M+H)⁺; ¹H NMR (CD₃OD, 400 MHz): δ 7.12 (d, 1H), 7.51 (d, 2H), 7.68 (d, 2H), 7.70 (m, 2H), 7.72 (d, 1H), 8.03 (m, 1H), 8.04 (s, 1H) ppm.

### Example 9

### 2-[2-(4-Chloro-phenyl)-(E)-vinyl]-4-(2,4-dichloro-phenyl)-1H-imidazole

Trans-4-chlorocinnamic acid (182 mg, 1 mmol) was treated according to general procedure A using 2,4-dichlorophenacyl bromide to give 2-[2-(4-chloro-phenyl)-(E)-vinyl]-4-(2,4-dichloro-phenyl)-1H-imidazole (227 mg, 65% yield).

LCMS: m/z 349 (M+H)⁺; ¹H NMR (CD₃OD, 400 MHz): δ 7.14 (d, 1H), 7.52 (d, 2H), 7.69 (d, 2H), 7.72-7.73 (m, 2H), 7.74 (d, 1H), 8.03 (m, 1H), 8.05 (s, 1H) ppm.

### Example 10

### 2-[2-(4-Bromo-phenyl)-(E)-vinyl]-4-(2,4-dichloro-phenyl)-1H-imidazole

Trans-4-bromocinnamic acid (2.27 g, 10 mmol) was treated according to general procedure A using 2,4-dichlorophenacyl bromide to give 2-[2-(4-bromo-phenyl)-(E)-vinyl]-4-(2,4-dichloro-phenyl)-1H-imidazole (2.24 g, 57% yield).

LCMS: m/z 394 (M+H)⁺; ¹H NMR (CD₃OD, 400 MHz): δ 7.14 (d, 1H), 7.51 (d, 2H), 7.69 (d, 2H), 7.71 (m, 2H), 7.74 (d, 1H), 8.02 (m, 1H), 8.04 (s, 1H) ppm.

### Example 11

### 2-(2-Biphenyl-4-yl-(E)-vinyl)-4-(2,4-dichloro-phenyl)-1H-imidazole

Trans-4-phenylcinnamic acid (224 mg, 1 mmol) was treated according to general procedure A using 2,4-dichlorophenacyl bromide to give 2-(2-biphenyl-4-yl-(E)-vinyl)-4-(2,4-dichloro-phenyl)-1H-imidazole (227 mg, 58% yield).

LCMS: m/z 391 (M+H)⁺; ¹H NMR (CDCl₃, 400 MHz): δ 6.94 (d, 1H), 7.31-7.39 (m, 2H), 7.43-7.48 (m, 3H), 7.61-7.64 (m, 6H), 7.66 (s, 1H), 7.74 (d, 1H), 8.26 (d, 1H) ppm.

### Example 12

### 4-(2,4-Dichloro-phenyl)-2-(2-naphthalen-1-yl-(E)-vinyl)-1H-imidazole

Trans-3-(1-naphthyl)acrylic acid (198 mg, 1 mmol) was treated according to general procedure A using 2,4-dichlorophenacyl bromide to give 4-(2,4-dichloro-phenyl)-2-(2-naphthalen-1-yl-(E)-vinyl)-1H-imidazole (201 mg, 55% yield).

LCMS: m/z 365 (M+H)⁺; ¹H NMR (CD₃OD, 400 MHz): δ 7.25 (d, 1H), 7.58-7.69 (m, 4H), 7.75 (d, 1H), 7.78 (d, 1H), 7.97-8.04 (m, 4H), 8.35 (d, 1H), 8.70 (d, 1H) ppm.

### Example 13

### 4-(2,4-Dichloro-phenyl)-2-(2-naphthalen-2-yl-(E)-vinyl)-1H-imidazole

Trans-3-(2-naphthyl)acrylic acid (198 mg, 1 mmol) was treated according to general procedure A using 2,4-dichlorophenacyl bromide to give 4-(2,4-dichloro-phenyl)-2-(2-naphthalen-2-yl-(E)-vinyl)-1H-imidazole (248 mg, 68% yield).

LCMS: m/z 365 (M+H)⁺; ¹H NMR (CD₃OD, 400 MHz): δ 7.27 (d, 1H), 7.57-7.69 (m, 4H), 7.75 (d, 1H), 7.76 (d, 1H), 7.96-8.02 (m, 4H), 8.33 (d, 1H), 8.71 (d, 1H) ppm.

### Example 14

### 4-[4-(2,4-Dichloro-phenyl)-1H-imidazol-2-yl]-5-phenyl-oxazole

5-Phenyl-1,3-oxazole-4-carboxylic acid (189 mg, 1 mmol) was treated according to general procedure A using 2,4-dichlorophenacyl bromide to give 4-[4-(2,4-dichloro-phenyl)-1H-imidazol-2-yl]-5-phenyl-oxazole (135 mg, 38% yield).

LCMS: m/z 356 (M+H)⁺.

### Example 15

### 2-[2-(4-Benzyloxy-phenyl)-(E)-vinyl]-4-(2,4-dichloro-phenyl)-1H-imidazole

Trans-4-benzyloxycinnamic acid (254 mg, 1 mmol) was treated according to general procedure A using 2,4-dichlorophenacyl bromide to give 2-[2-(4-benzyloxy-phenyl)-(E)-vinyl]-4-(2,4-dichloro-phenyl)-1H-imidazole (185 mg, 44% yield).

LCMS: m/z 421 (M+H)⁺; ¹H NMR (CD₃OD, 400 MHz): δ 5.16 (s, 2H), 7.48 (d, 2H), 7.51 (s, 5H), 7.61 (d, 2H), 7.65 (d, 2H), 7.69 (d, 2H), 7.74 (s, 1H), 7.81 (d, 1H) ppm.

### Example 16

### 4-(2,4-Dichloro-phenyl)-2-fluoren-9-ylidenemethyl-1H-imidazole

9-Fluorenylideneacetic acid (222 mg, 1 mmol) was treated according to general procedure A using 2,4-dichlorophenacyl bromide to give 4-(2,4-dichloro-phenyl)-2-fluoren-9-ylidenemethyl-1H-imidazole (245 mg, 63% yield).

LCMS: m/z 389 (M+H)⁺. ¹H NMR (CD₃OD, 400 MHz): δ 7.25 (m, 1H), 7.37-7.51 (m, 5H), 7.57 (dd, 1H), 7.73 (d, 1H), 7.77-7.82 (m, 3H), 7.93 (d, 1H), 8.08 (s, 1H) ppm.

### Example 17

### 1-Butyl-4-(2,4-dichloro-phenyl)-2-fluoren-9-ylidenemethyl-1H-imidazole

4-(2,4-Dichloro-phenyl)-2-fluoren-9-ylidenemethyl-1H-imidazole (39 mg, 0.1 mmol) was treated according to general procedure E using 1-bromobutane to give 1-butyl-4-(2,4-dichloro-phenyl)-2-fluoren-9-ylidenemethyl-1H-imidazole (35 mg, 78% yield).

LCMS: m/z 445 (M+H)⁺.

### Example 18

### 4-(2,4-Dichloro-phenyl)-2-[2-(4-methoxy-phenyl)-(E)-vinyl]-oxazole

Trans-4-methoxycinnamic acid (178 mg, 1 mmol) was treated according to general procedure A using 2,4-dichlorophenacyl bromide to afford 4-(2,4-dichloro-phenyl)-2-[2-(4-methoxy-phenyl)-(E)-vinyl]-oxazole as a less polar by-product (38 mg, 11% yield) along with 4-(2,4-dichloro-phenyl)-2-[2-(4-methoxy-phenyl)-(E)-vinyl]-1H-imidazole (193 mg, 56% yield).

LCMS: m/z 346 (M+H)⁺; ¹H NMR (CD₃OD, 400 MHz): δ 3.81 (s, 3H), 6.89 (d, 1H), 6.95 (d, 2H), 7.34 (d, 1H), 7.51 (d, 2H), 7.52 (d, 1H), 7.58 (s, 1H), 7.67 (d, 1H), 7.94 (s, 1H) ppm.

### Example 19

### 4-(2,4-Dichloro-phenyl)-2-[2-(4′-methoxy-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 4-methoxyphenylboronic acid to give 4-(2,4-dichloro-phenyl)-2-[2-(4′-methoxy-biphenyl-4-yl)-(E)-vinyl]-1H-imidazole (30 mg, 72% yield).

LCMS: m/z 421 (M+H)⁺; ¹H NMR (CD₃OD, 400 MHz): δ 3.82 (s, 3H), 7.03 (d, 2H), 7.15 (d, 1H), 7.54 (dd, 1H), 7.62 (d, 2H), 7.70 (s, 1H), 7.71 (m, 5H), 7.73 (d, 1H), 7.91 (s, 1H) ppm.

### Example 20

### 4-(2,4-Dichloro-phenyl)-2-[2-(3′-methoxy-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 3-methoxyphenylboronic acid to give 4-(2,4-dichloro-phenyl)-2-[2-(3′-methoxy-biphenyl-4-yl)-(E)-vinyl]-1H-imidazole (28 mg, 67% yield).

LCMS: m/z 421 (M+H)⁺; ¹H NMR (CD₃OD, 400 MHz): δ 3.81 (s, 3H), 7.03 (d, 2H), 7.15 (d, 1H), 7.58-7.61 (m, 3H), 7.68-7.70 (m, 6H), 7.73 (d, 1H), 7.90 (s, 1H) ppm.

### Example 21

### 4-(2,4-Dichloro-phenyl)-2-[2-(2′-methoxy-biphenyl-4-yl)-(E)-vinyl]-1H-imidazole

2-[2-(4-bromo-phenyl)-(E)-vinyl]-4-(2,4-dichloro-phenyl)-1H-imidazole (40 mg, 0.1 mmol) was treated as described in general procedure B using 2-methoxyphenylboronic acid to give 4-(2,4-dichloro-phenyl)-2-[2-(2′-methoxy-biphenyl-4-yl)-(E)-vinyl]-1H-imidazole (24 mg, 57% yield).

LCMS: m/z 421 (M+H)⁺; ¹H NMR (CD₃OD, 400 MHz): δ 3.83 (s, 3H), 7.03 (d, 2H), 7.15 (d, 1H), 7.55-7.60 (m, 3H), 7.66-7.71 (m, 6H), 7.73 (d, 1H), 7.92 (s, 1H) ppm.

### Example 22

### 4-(2,4-Dichloro-phenyl)-2-[2-(3′,4′-dimethoxy-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 3,4-dimethoxyphenylboronic acid to give 4-(2,4-dichloro-phenyl)-2-[2-(3′,4′-dimethoxy-biphenyl-4-yl)-(E)-vinyl]-1H-imidazole (24 mg, 54% yield).

LCMS: m/z 451 (M+H)⁺; ¹H NMR (CD₃OD, 400 MHz): δ 3.84 (s, 3H), 3.87 (s, 3H), 7.03 (d, 2H), 7.15 (d, 1H), 7.58-7.61 (m, 3H), 7.68-7.71 (m, 5H), 7.73 (d, 1H), 7.90 (s, 1H) ppm.

### Example 23

### 4-(2,4-Dichloro-phenyl)-2-[2-(2′,4′-dimethoxy-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 2,4-dimethoxyphenylboronic acid to give 4-(2,4-dichloro-phenyl)-2-[2-(2′,4′-dimethoxy-biphenyl-4-yl)-(E)-vinyl]-1H-imidazole (22 mg, 49% yield).

LCMS: m/z 451 (M+H)⁺.

### Example 24

### 2-[2-(4′-Butoxy-biphenyl-4-yl)-(E)-vinyl]-4-(2,4-dichloro-phenyl)-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 4-n-butoxyphenylboronic acid to give 2-[2-(4′-butoxy-biphenyl-4-yl)-(E)-vinyl]-4-(2,4-dichloro-phenyl)-1H-imidazole (24 mg, 52% yield).

LCMS: m/z 463 (M+H)⁺; ¹H NMR (CD₃OD, 400 MHz): δ 1.15 (t, 3H), 1.43 (m, 2H), 1.84 (m, 2H), 4.18 (t, 2H), 7.03 (d, 2H), 7.15 (d, 1H), 7.54 (dd, 1H), 7.62 (d, 2H), 7.70 (s, 1H), 7.71 (m, 5H), 7.73 (d, 1H), 7.91 (s, 1H) ppm.

### Example 25

### 4-(2,4-Dichloro-phenyl)-2-[2-(4′-phenoxy-biphenyl-4-yl)-(E)-vinyl]-1H-imidazole

2-[2-(4-Bromo-phenyl)-(E)-vinyl]-4-(2,4-dichloro-phenyl)-1H-imidazole (39 mg, 0.1 mmol) was treated with 4-phenoxyphenyl boronic acid as described in general procedure B to give 4-(2,4-dichloro-phenyl)-2-[2-(4′-phenoxy-biphenyl-4-yl)-(E)-vinyl]-1H-imidazole (30 mg, 63% yield).

LCMS: m/z 483 (M+H)⁺; ¹H NMR (CDCl₃, 400 MHz): δ 7.03 (d, 1H), 7.06 (d, 1H), 7.08 (m, 3H), 7.15 (d, 1H), 7.35 (m, 2H), 7.37 (d, 1H), 7.45 (s, 1H), 7.58 (m, 7H), 7.78 (s, 1H), 8.20 (d, 1H), 9.38 (bs, 1H) ppm.

### Example 26

### 2-[2-(4′-Benzyloxy-biphenyl-4-yl)-(E)-vinyl]-4-(2,4-dichloro-phenyl)-1H-imidazole

2-[2-(4-Bromo-phenyl)-(E)-vinyl]-4-(2,4-dichloro-phenyl)-1H-imidazole (39 mg, 0.1 mmol) was treated with 4-benzyloxy benzene boronic acid as described in general procedure B to give 2-[2-(4′-benzyloxy-biphenyl-4-yl)-(E)-vinyl]-4-(2,4-dichloro-phenyl)-1H-imidazole (39 mg, 78% yield).

LCMS: m/z 497 (M+H)⁺; ¹H NMR (CD₃OD, 400 MHz): δ 5.16 (s, 2H), 7.10 (d, 1H), 7.12 (d, 1H), 7.42 (m, 2H), 7.48 (d, 2H), 7.51 (s, 5H), 7.61 (d, 2H), 7.65 (d, 2H), 7.69 (d, 2H), 7.74 (s, 1H), 7.81 (d, 1H) ppm.

### Example 27

### 2-[2-(4′-Benzyloxy-3′-fluoro-biphenyl-4-yl)-(E)-vinyl]-4-(2,4-dichloro-phenyl)-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 4-benzyloxy-3-fluorobenzeneboronic acid to give 2-[2-(4′-benzyloxy-3′-fluoro-biphenyl-4-yl)-(E)-vinyl]-4-(2,4-dichloro-phenyl)-1H-imidazole (36 mg, 71% yield).

LCMS: m/z 515 (M+H)⁺; ¹H NMR (CD₃OD, 400 MHz): δ 5.22 (s, 2H), 7.13 (d, 1H), 7.20 (t, 1H), 7.38-7.49 (m, 6H), 7.54 (m, 1H), 7.66 (d, 1H), 7.69-7.72 (m, 5H), 7.74 (s, 1H), 7.75 (d, 1H), 7.86 (s, 1H) ppm.

### Example 28

### 4-(2,4-Dichloro-phenyl)-2-{2-[4-(2,3-dihydro-benzo[1,4]dioxin-6-yl)-phenyl]-(E)-vinyl}-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 2,3-dihydro-1,4-benzodioxin-6-ylboronic acid to give 4-(2,4-dichloro-phenyl)-2-{2-[4-(2,3-dihydro-benzo[1,4]dioxin-6-yl)-phenyl]-(E)-vinyl}-1H-imidazole (27 mg, 61% yield).

LCMS: m/z 449 (M+H)⁺; ¹H NMR (CD₃OD, 400 MHz): δ 4.28 (s, 4H), 6.91 (d, 1H), 7.12 (d, 1H), 7.15 (m, 2H), 7.51 (m, 1H), 7.62 (d, 1H), 7.64-7.70 (m, 6H), 7.78 (d, 1H) ppm.

### Example 29

### 4-(2,4-Dichloro-phenyl)-2-[2-(4′-methoxy-3′,5′-dimethyl-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 4-methoxy-3,5-dimethylbenzeneboronic acid to give 4-(2,4-dichloro-phenyl)-2-[2-(4′-methoxy-3′,5′-dimethyl-biphenyl-4-yl)-(E)-vinyl]-1H-imidazole (28 mg, 63% yield).

LCMS: m/z 449 (M+H)⁺; ¹H NMR (CDCl₃, 400 MHz): δ 2.36 (s, 6H), 3.77 (s, 3H), 7.13 (d, 1H), 7.54 (m, 1H), 7.67 (d, 1H), 7.70-7.73 (m, 5H), 7.76 (d, 1H), 7.78 (s, 2H), 7.87 (s, 1H) ppm.

### Example 30

### 4-(2,4-Dichloro-phenyl)-2-[2-(4′-ethoxy-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 4-ethoxybenzeneboronic acid to give 4-(2,4-dichloro-phenyl)-2-[2-(4′-ethoxy-biphenyl-4-yl)-(E)-vinyl]-1H-imidazole (29 mg, 68% yield).

LCMS: m/z 435 (M+H)⁺; ¹H NMR (CDCl₃, 400 MHz): δ 1.57 (t, 3H), 4.30 (q, 2H), 6.93 (d, 1H), 6.97 (d, 2H), 7.45 (d, 1H), 7.50-7.56 (m, 6H), 7.75 (d, 2H), 8.59 (d, 1H), 8.94 (d, 1H) ppm.

### Example 31

### 4-(2,4-Dichloro-phenyl)-2-[2-(4′-trifluoromethoxy-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 4-trifluoromethoxyphenyl boronic acid to give 4-(2,4-dichloro-phenyl)-2-[2-(4′-trifluoromethoxy-biphenyl-4-yl)-(E)-vinyl]-1H-imidazole (20 mg, 42% yield).

LCMS: m/z 475 (M+H)⁺; ¹H NMR (CD₃OD, 400 MHz): δ 7.03 (d, 2H), 7.15 (d, 1H), 7.54 (dd, 1H), 7.62 (d, 2H), 7.68-7.71 (m, 6H), 7.73 (d, 1H), 7.91 (s, 1H) ppm.

### Example 32

### 4-(2,4-Dichloro-phenyl)-2-[2-(3′-trifluoromethoxy-biphenyl-4-yl)-(E)-vinyl]-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 3-trifluoromethoxyphenylboronic acid to give 4-(2,4-dichloro-phenyl)-2-[2-(3′-trifluoromethoxy-biphenyl-4-yl)-(E)-vinyl]-1H-imidazole (23 mg, 48% yield).

LCMS: m/z 475 (M+H)⁺; ¹H NMR (CD₃OD, 400 MHz): δ 7.04 (d, 2H), 7.15 (d, 1H), 7.54 (dd, 1H), 7.62 (d, 2H), 7.68-7.74 (m, 7H), 7.92 (s, 1H) ppm.

### Example 33

### 2-[2-(4-Benzofuran-2-yl-phenyl)-(E)-vinyl]-4-(2,4-dichloro-phenyl)-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using benzo[B]furan-2-boronic acid to give 2-[2-(4-benzofuran-2-yl-phenyl)-(E)-vinyl]-4-(2,4-dichloro-phenyl)-1H-imidazole (15 mg, 34% yield).

LCMS: m/z 431 (M+H)⁺.

### Example 34

### 2-[2-(5′-Chloro-2′-methoxy-biphenyl-4-(E)-vinyl]-(E)-vinyl-4-(2,4-dichloro-phenyl)-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 5-chloro-2-methoxyphenylboronic acid to give 2-[2-(5′-chloro-2′-methoxy-biphenyl-4-yl)-(E)-vinyl]-4-(2,4-dichloro-phenyl)-1H-imidazole (22 mg, 47% yield).

LCMS: m/z 455 (M+H)⁺; ¹H NMR (CD₃OD, 400 MHz): δ 3.81 (s, 3H), 7.03 (d, 2H), 7.15 (d, 1H), 7.58-7.61 (m, 3H), 7.68-7.70 (m, 5H), 7.73 (d, 1H), 7.90 (s, 1H) ppm.

### Example 35

### 2-[2-(4′-tert-Butyl-biphenyl-4-yl)-(E)-vinyl]-4-(2,4-dichloro-phenyl)-1H-imidazole

2-[2-(4-Bromo-phenyl)-(E)-vinyl]-4-(2,4-dichloro-phenyl)-1H-imidazole (40 mg, 0.1 mmol) was treated as described in general procedure B using 4-tert-butylbenzeneboronic acid to give 2-[2-(4′-tert-butyl-biphenyl-4-yl)-(E)-vinyl]-4-(2,4-dichloro-phenyl)-1H-imidazole (19 mg, 42% yield).

LCMS: m/z 447 (M+H)⁺; ¹H NMR (CD₃OD, 400 MHz): δ 1.22 (s 9H), 7.03 (d, 2H), 7.15 (d, 1H), 7.54 (dd, 1H), 7.62 (d, 2H), 7.68-7.71 (m, 6H), 7.73 (d, 1H), 7.92 (s, 1H) ppm.

### Example 36

### 3-(4′-{2-[4-(2,4-Dichloro-phenyl)-1H-imidazol-2-yl]-(E)-vinyl}-biphenyl-4-yl)-acrylic acid

2-[2-(4-Bromo-phenyl)-(E)-vinyl]-4-(2,4-dichloro-phenyl)-1H-imidazole (79 mg, 0.2 mmol) was treated as described in general procedure B using 4-(2-carboxy(E)-vinyl)benzene boronic acid to give 3-(4′-{2-[4-(2,4-dichloro-phenyl)-1H-imidazol-2-yl]-(E)-vinyl}-biphenyl-4-yl)-acrylic acid (21 mg, 22% yield).

LCMS: m/z 461 (M+H)⁺; ¹H NMR (CD₃OD, 400 MHz): δ 6.53 (d, 1H), 7.14 (d, 1H), 7.54 (dd, 1H), 7.62 (d, 1H), 7.68-7.79 (m, 10H), 7.89 (d, 1H), 7.94 (s, 1H) ppm.

### Example 37

### 4-(2,4-Dichloro-phenyl-2-{2-[4-(4-methoxy-phenylethynl)-phenyl]-(E)-vinyl}-1H-imidazole

2-[2-(4-Bromo-phenyl)-(E)-vinyl]-4-(2,4-dichloro-phenyl)-1H-imidazole (40 mg, 0.1 mmol) was treated as described in general procedure H using 1-ethynyl-4-methoxybenzene to give 4-(2,4-dichloro-phenyl)-2-{2-[4-(4-methoxy-phenylethynyl)-phenyl]-(E)-vinyl}-1H-imidazole (23 mg, 51% yield).

LCMS: m/z 445 (M+H)⁺; ¹H NMR (CD₃OD, 400 MHz): δ 3.81 (s, 3H), 7.03 (d, 2H), 7.15 (d, 1H), 7.58-7.61 (m, 3H), 7.68-7.70 (m, 6H), 7.73 (d, 1H), 7.90 (s, 1H) ppm.

### Example 38

### 5-(4-{2-[4-(2,4-Dichloro-phenyl)-1H-imidazol-2-yl]-(E)-vinyl}-phenyl)-pent-4-ynoic acid

2-[2-(4-Bromo-phenyl)-(E)-vinyl]-4-(2,4-dichloro-phenyl)-1H-imidazole (40 mg, 0.1 mmol) was treated as described in general procedure H using 4-pentynoic acid methyl ester followed by ester hydrolysis as described in general procedure F to give 5-(4-{2-[4-(2,4-dichloro-phenyl)-1H-imidazol-2-yl]-(E)-vinyl}-phenyl)-pent-4-ynoic acid (12 mg, 29% yield).

LCMS: m/z 411 (M+H)⁺; ¹H NMR (CD₃OD, 400 MHz): δ 2.53 (m, 2H), 2.64 (m, 2H), 7.03 (d, 2H), 7.15 (d, 1H), 7.58-7.61 (m, 3H), 7.68 (m, 2H), 7.73 (d, 1H), 7.90 (s, 1H) ppm.

### Example 39

### 4′-{2-[4-(2,4-Dichloro-phenyl)-1H-imidazol-2-yl]-(E)-vinyl}-biphenyl-4-carboxylic acid

2-[2-(4-Bromo-phenyl)-(E)-vinyl]-4-(2,4-dichloro-phenyl)-1H-imidazole (394 mg, 1 mmol) was treated as described in general procedure B using 4-carboxybenzeneboronic acid to give 4′-{2-[4-(2,4-dichloro-phenyl)-1H-imidazol-2-yl]-(E)-vinyl}-biphenyl-4-carboxylic acid (105 mg, 24% yield).

LCMS: m/z 435 (M+H)⁺; ¹H NMR (CD₃OD, 400 MHz): δ 7.03 (d, 2H), 7.15 (d, 1H), 7.54 (dd, 1H), 7.62 (d, 2H), 7.68-7.71 (m, 6H), 7.73 (d, 1H), 7.92 (s, 1H) ppm.

### Example 40

### 4-{[(4′-{2-[4-(2,4-Dichloro-phenyl)-1H-imidazol-2-yl]-(E)-vinyl}-biphenyl-4-carbonyl)-amino]-methyl}-benzoic acid

4′-{2-[4-(2,4-Dichloro-phenyl)-1H-imidazol-2-yl]-(E)-vinyl}-biphenyl-4-carboxylic acid (44 mg, 0.1 mmol) was treated as described in general procedure G using methyl 4-(aminomethyl)benzoate hydrochloride followed by ester hydrolysis as described in general procedure F to give 4-{[(4′-{2-[4-(2,4-dichloro-phenyl)-1H-imidazol-2-yl]-(E)-vinyl}-biphenyl-4-carbonyl)-amino]-methyl}-benzoic acid (25 mg, 44% yield).

LCMS: m/z 568 (M+H)⁺; ¹H NMR (CD₃OD, 400 MHz): δ 5.03 (d, 2H), 7.03 (d, 2H), 7.15 (d, 1H), 7.23 (d, 2H), 7.35 (d, 2H), 7.54 (dd, 1H), 7.62 (d, 2H), 7.68-7.71 (m, 6H), 7.73 (d, 1H), 7.92 (s, 1H) ppm.

### Example 41

### 4′-{2-[4-(2,4-Dichloro-phenyl)-1-ethyl-1H-imidazol-2-yl]-(E)-vinyl}-biphenyl-4-carboxylic acid

2-[2-(4-Bromo-phenyl)-(E)-vinyl]-4-(2,4-dichloro-phenyl)-1-ethyl-1H-imidazole (44 mg, 0.1 mmol) was treated as described in general procedure B using 4-carboxybenzeneboronic acid to give 4′-{2-[4-(2,4-dichloro-phenyl)-1-ethyl-1H-imidazol-2-yl]-(E)-vinyl}-biphenyl-4-carboxylic acid (29 mg, 63% yield).

LCMS: m/z 463 (M+H)⁺; ¹H NMR (CD₃OD, 400 MHz): δ 1.45 (t, 2H), 4.28 (q, 2H), 7.03 (d, 2H), 7.15 (d, 1H), 7.54 (dd, 1H), 7.62 (d, 2H), 7.68-7.71 (m, 6H), 7.73 (d, 1H), 7.92 (s, 1H) ppm.

### Example 42

### 2-[2-(4′-Benzyloxy-3′-fluoro-biphenyl-4-yl)-(E)-vinyl]-4-(2,4-dichloro-phenyl)-1-ethyl-1H-imidazole

2-[2-(4′-Benzyloxy-3′-fluoro-biphenyl-4-yl)-(E)-vinyl]-4-(2,4-dichloro-phenyl)-1H-imidazole (52 mg, 0.1 mmol) was treated as described in general procedure E using ethyl bromide to give 2-[2-(4′-benzyloxy-3′-fluoro-biphenyl-4-yl)-(E)-vinyl]-4-(2,4-dichloro-phenyl)-1-ethyl-1H-imidazole (39 mg, 71% yield).

LCMS: m/z 543 (M+H)⁺; ¹H NMR (CD₃OD, 400 MHz): δ 1.46 (t, 3H), 4.30 (q, 2H), 5.22 (s, 2H), 7.13 (d, 1H), 7.20 (t, 1H), 7.38-7.49 (m, 6H), 7.54 (m, 1H), 7.66 (d, 1H), 7.69-7.72 (m, 5H), 7.74 (s, 1H), 7.75 (d, 1H), 7.86 (s, 1H) ppm.

### Example 43

### 4-(4′-{2-[4-(2,4-Dichloro-phenyl)-1-ethyl-1H-imidazol-2-yl]-(E)-vinyl}-3-fluoro-biphenyl-4-yloxymethyl)-benzoic acid

2-[2-(4′-Benzyloxy-3′-fluoro-biphenyl-4-yl)-(E)-vinyl]-4-(2,4-dichloro-phenyl)-1-ethyl-1H-imidazole (55 mg, 0.1 mmol) was treated as described in general procedure C and the resulting phenol was treated with methyl 4-(bromomethyl)benzoate as described in the general procedure E followed by ester hydrolysis as described in the general procedure F to give 4-(4′-{2-[4-(2,4-dichloro-phenyl)-1-ethyl-1H-imidazol-2-yl]-(E)-vinyl}-3-fluoro-biphenyl-4-yloxymethyl)-benzoic acid (18 mg, 31% yield).

LCMS: m/z 587 (M+H)⁺; ¹H NMR (CD₃OD, 400 MHz): δ 1.46 (t, 3H), 4.30 (q, 2H), 5.22 (s, 2H), 7.13 (d, 1H), 7.20 (t, 1H), 7.38-7.49 (m, 5H), 7.54 (m, 1H), 7.66 (d, 1H), 7.69-7.72 (m, 5H), 7.74 (s, 1H), 7.75 (d, 1H), 7.86 (s, 1H) ppm.

### Example 44

### 4-{2-[4-(2,4-Dichloro-phenyl)-1H-imidazol-2-yl]-(E)-vinyl}-phenol

4-(2,4-Dichloro-phenyl)-2-[2-(4-methoxy-phenyl)-(E)-vinyl]-1H-imidazole (34 mg, 0.1 mmol) was treated as described in general procedure C to give 4-{2-[4-(2,4-dichloro-phenyl)-1H-imidazol-2-yl]-(E)-vinyl}-phenol (20 mg, 61% yield).

LCMS: m/z 331 (M+H)⁺; ¹H NMR (CD₃OD, 400 MHz): δ 6.88 (d, 1H), 6.95 (d, 2H), 7.33 (d, 1H), 7.51 (d, 2H), 7.52 (d, 1H), 7.54 (s, 1H), 7.66 (d, 1H), 7.93 (s, 1H) ppm.

### Example 45

### 4-(2,4-Dichloro-phenyl)-2-[2-(4-methoxy-phenyl)-ethyl]-1H-imidazole

4-(2,4-Dichloro-phenyl)-2-[2-(4-methoxy-phenyl)-(E)-vinyl]-1H-imidazole (34 mg, 0.1 mmol) was treated as described in general procedure D to give 4-(2,4-dichloro-phenyl)-2-[2-(4-methoxy-phenyl)-ethyl]-1H-imidazole (17 mg, 51% yield).

LCMS: m/z 347 (M+H)⁺; ¹H NMR (CD₃OD, 400 MHz): δ 3.00 (s, 4H), 3.77 (s, 3H), 6.82 (d, 2H), 7.10 (d, 2H), 7.32 (m, 1H), 7.46 (m, 2H), 7.74 (s, 1H) ppm.

### Example 46

### 4-(2,4-Dichloro-phenyl)-1-ethyl-2-[2-(4-methoxy-phenyl)-(E)-vinyl]-1H-imidazole

4-(2,4-Dichloro-phenyl)-2-[2-(4-methoxy-phenyl)-(E)-vinyl]-1H-imidazole (34 mg, 0.1 mmol) was treated with ethyl bromide as described in general procedure E to give 4-(2,4-dichloro-phenyl)-1-ethyl-2-[2-(4-methoxy-phenyl)-(E)-vinyl]-1H-imidazole (32 mg, 84% yield).

LCMS: m/z 373 (M+H)⁺.

### Example 47

### 4-(4-{2-[4-(2,4-Dichloro-phenyl)-1-ethyl-1H-imidazol-2-yl]-(E)-vinyl}-phenoxymethyl)-benzoic acid

4-(2,4-Dichloro-phenyl)-1-ethyl-2-[2-(4-methoxy-phenyl)-(E)-vinyl]-1H-imidazole (38 mg, 0.1 mmol) was treated as described in general procedure C and the resulting phenol was treated with methyl 4-(bromomethyl)benzoate as described in the general procedure E followed by ester hydrolysis as described in the general procedure F to give 4-(4-{2-[4-(2,4-dichloro-phenyl)-1-ethyl-1H-imidazol-2-yl]-(E)-vinyl}-phenoxymethyl)-benzoic acid (17 mg, 34% yield)

LCMS: m/z 493 (M+H)⁺.

### Example 48

### 3-(4-{2-[4-(2,4-Dichloro-phenyl)-1-ethyl-1H-imidazol-2-yl]-(E)-vinyl}-phenoxymethyl)-benzoic acid

4-(2,4-Dichloro-phenyl)-1-ethyl-2-[2-(4-methoxy-phenyl)-(E)-vinyl]-1H-imidazole (38 mg, 0.1 mmol) was treated as described in general procedure C and the resulting phenol was treated with methyl 3-(bromomethyl)benzoate as described in the general procedure E followed by ester hydrolysis as described in the general procedure F to give 3-(4-{2-[4-(2,4-dichloro-phenyl)-1-ethyl-1H-imidazol-2-yl]-(E)-vinyl}-phenoxymethyl)-benzoic acid (15 mg, 30% yield)

LCMS: m/z 493 (M+H)⁺.

### Example 49

### 4-(4-{2-[4-(2,4-Dichloro-phenyl)-1-ethyl-1H-imidazol-2-yl]-(E)-vinyl}-phenoxy)-butyric acid

4-(2,4-Dichloro-phenyl)-1-ethyl-2-[2-(4-methoxy-phenyl)-(E)-vinyl]-1H-imidazole (38 mg, 0.1 mmol) was treated as described in general procedure C and the resulting phenol was treated with methyl 4-bromobutyrate as described in the general procedure E followed by ester hydrolysis as described in the general procedure F to give 4-(4-{2-[4-(2,4-dichloro-phenyl)-1-ethyl-1H-imidazol-2-yl]-(E)-vinyl}-phenoxy)-butyric acid (15 mg, 33% yield).

LCMS: m/z 445 (M+H)⁺; ¹H NMR (CDCl₃, 400 MHz): δ 1.21 (t, 3H), 2.15 (m, 2H), 2.56 (t, 2H), 3.94 (q, 2H), 4.06 (t, 2H), 6.95 (d, 1H), 6.97 (d, 2H), 7.30 (m, 1H), 7.42 (d, 1H), 7.55 (m, 2H), 7.71 (s, 1H), 7.73 (d, 1H), 8.25 (d, 1H) ppm.

### Example 50

### 6-(4-{2-[4-(2,4-Dichloro-phenyl)-1-ethyl-1H-imidazol-2-yl]-(E)-vinyl}-phenoxy)-hexanoic acid

4-(2,4-Dichloro-phenyl)-1-ethyl-2-[2-(4-methoxy-phenyl)-(E)-vinyl]-1H-imidazole (38 mg, 0.1 mmol) was treated as described in general procedure C and the resulting phenol was treated with ethyl 6-bromohexanoate as described in the general procedure E followed by ester hydrolysis as described in the general procedure F to give 6-(4-{2-[4-(2,4-dichloro-phenyl)-1-ethyl-1H-imidazol-2-yl]-(E)-vinyl}-phenoxy)-hexanoic acid (18 mg, 38% yield).

LCMS: m/z 473 (M+H)⁺.

### Example 51

### 1-Butyl-4-(2,4-dichloro-phenyl)-2-[2-(4-methoxy-phenyl)-(E)-vinyl]-1H-imidazole

4-(2,4-Dichloro-phenyl)-2-[2-(4-methoxy-phenyl)-(E)-vinyl]-1H-imidazole (34 mg, 0.1 mmol) was treated with 1-bromobutane as described in general procedure E to give 1-butyl-4-(2,4-dichloro-phenyl)-2-[2-(4-methoxy-phenyl)-(E)-vinyl]-1H-imidazole (32 mg, 81% yield)

LCMS: m/z 401 (M+H)⁺; ¹H NMR (CD₃OD, 400 MHz): δ 1.01 (t, 3H), 1.46 (m, 2H), 1.90 (m, 2H), 3.87 (s, 3H), 4.31 (t, 2H), 7.04 (d, 2H), 7.16 (d, 1H), 7.71-7.74 (m, 4H), 7.78 (d, 1H), 8.05 (m, 2H) ppm.

### Example 52

### 4-(2,4-Dichloro-phenyl)-1-isobutyl-2-[2-(4-methoxy-phenyl)-(E)-vinyl]-1H-imidazole

4-(2,4-Dichloro-phenyl)-2-[2-(4-methoxy-phenyl)-(E)-vinyl]-1H-imidazole (34 mg, 0.1 mmol) was treated with isobutyl bromide as described in general procedure E to give 4-(2,4-dichloro-phenyl)-1-isobutyl-2-[2-(4-methoxy-phenyl)-(E)-vinyl]-1H-imidazole (29 mg, 72% yield).

LCMS: m/z 401 (M+H)⁺; ¹H NMR (CD₃OD, 400 MHz): δ 1.03 (d, 6H), 1.87 (m, 1H), 3.87 (s, 3H), 4.24 (d, 2H), 7.04 (d, 2H), 7.16 (d, 1H), 7.71-7.74 (m, 4H), 7.78 (d, 1H), 8.05 (m, 2H) ppm.

### Example 53

### 2-[2-(4-Butoxy-phenyl)-(E)-vinyl]-1-butyl-4-(2,4-dichloro-phenyl)-1H-imidazole

4-{2-[4-(2,4-Dichloro-phenyl)-1H-imidazol-2-yl]-(E)-vinyl}-phenol (33 mg, 0.1 mmol) was treated with 1-bromobutane as described in general procedure E to give 2-[2-(4-butoxy-phenyl)-(E)-vinyl]-1-butyl-4-(2,4-dichloro-phenyl)-1H-imidazole (34 mg, 76% yield)

LCMS: m/z 443 (M+H)⁺; ¹H NMR (CDCl₃, 400 MHz): δ 1.02 (dt, 6H), 1.43 (m, 4H), 1.88 (m, 4H), 4.08 (t, 2H), 4.34 (t, 2H), 7.04 (d, 2H), 7.16 (d, 1H), 7.71-7.74 (m, 4H), 7.78 (d, 1H), 8.05 (m, 2H) ppm.

### Example 54

### 2-(2-Biphenyl-4-yl-(E)-vinyl)-1-butyl-4-(2,4-dichloro-phenyl)-1H-imidazole

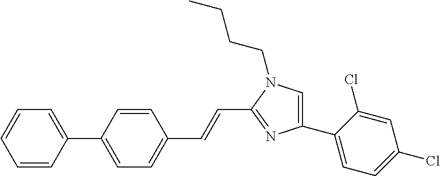

2-(2-Biphenyl-4-yl-(E)-vinyl)-4-(2,4-dichloro-phenyl)-1H-imidazole (20 mg, 0.05 mmol) was treated with 1-bromobutane as described in general procedure E to give 2-(2-biphenyl-4-yl-(E)-vinyl)-1-butyl-4-(2,4-dichloro-phenyl)-1H-imidazole (16 mg, 73% yield)

LCMS: m/z 447 (M+H)⁺; ¹H NMR (CDCl₃, 400 MHz): δ 1.00 (t, 3H), 1.43 (m, 2H), 1.84 (m, 2H), 4.08 (t, 2H), 6.94 (d, 1H), 7.31-7.39 (m, 2H), 7.43-7.48 (m, 3H), 7.61-7.64 (m, 6H), 7.66 (s, 1H), 7.74 (d, 1H), 8.26 (d, 1H) ppm.

### Example 55

### 1-Butyl-4-(2,4-dichloro-phenyl)-2-[2-(4′-methoxy-biphenyl-4-yl)-(E)-vinyl]-1H-imidazole

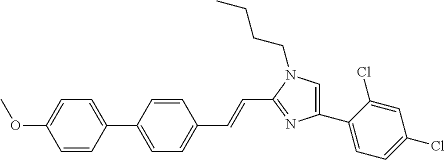

4-(2,4-Dichloro-phenyl)-2-[2-(4′-methoxy-biphenyl-4-yl)-(E)-vinyl]-1H-imidazole (21 mg, 0.05 mmol) was treated with 1-bromobutane as described in general procedure E to give 1-butyl-4-(2,4-dichloro-phenyl)-2-[2-(4′-methoxy-biphenyl-4-yl)-(E)-vinyl]-1H-imidazole (18 mg, 76% yield).

LCMS: m/z 477 (M+H)⁺; ¹H NMR (CDCl₃, 400 MHz): δ 1.00 (t, 3H), 1.43 (m, 2H), 1.84 (m, 2H), 3.85 (s, 3H), 4.08 (t, 2H), 6.90 (d, 1H), 7.00 (d, 2H), 7.32 (dd, 1H), 7.42 (d, 1H), 7.55-7.61 (m, 6H), 7.63 (s, 1H), 7.74 (d, 1H), 8.26 (d, 1H) ppm.

### Example 56

### 4-(2,4-Dichloro-phenyl)-1-isobutyl-2-[2-(4′-methoxy-biphenyl-4-yl)-(E)-vinyl]-1H-imidazole

4-(2,4-Dichloro-phenyl)-2-[2-(4′-methoxy-biphenyl-4-yl)-(E)-vinyl]-1H-imidazole

(21 mg, 0.05 mmol) was treated with isobutyl bromide as described in general procedure E to give 4-(2,4-dichloro-phenyl)-1-isobutyl-2-[2-(4′-methoxy-biphenyl-4-yl)-(E)-vinyl]-1H-imidazole (15 mg, 62% yield).

LCMS: m/z 477 (M+H)⁺.

### Example 57

### 4-(2,4-Dichloro-phenyl)-2-[2-(4′-methoxy-biphenyl-4-yl)-(E)-vinyl]-1-propyl-1H-imidazole

4-(2,4-Dichloro-phenyl)-2-[2-(4′-methoxy-biphenyl-4-yl)-(E)-vinyl]-1H-imidazole (21 mg, 0.05 mmol) was treated with 1-bromopropane as described in general procedure E to give 4-(2,4-dichloro-phenyl)-2-[2-(4′-methoxy-biphenyl-4-yl)-(E)-vinyl]-1-propyl-1H-imidazole (16 mg, 68% yield).

LCMS: m/z 463 (M+H)⁺.

### Example 58

### 4-(2,4-Dichloro-phenyl)-2-[2-(4′-methoxy-biphenyl-4-yl)-(E)-vinyl]-1-methyl-1H-imidazole

4-(2,4-Dichloro-phenyl)-2-[2-(4′-methoxy-biphenyl-4-yl)-(E)-vinyl]-1H-imidazole (42 mg, 0.1 mmol) was treated with methyl iodide as described in general procedure E to give 4-(2,4-dichloro-phenyl)-2-[2-(4′-methoxy-biphenyl-4-yl)-(E)-vinyl]-1-methyl-1H-imidazole (18 mg, 76% yield).

LCMS: m/z 435 (M+H)⁺; ¹H NMR (CDCl₃, 400 MHz): δ 3.81 (s, 3H), 3.86 (s, 3H), 6.90 (d, 1H), 7.00 (d, 2H), 7.32 (dd, 1H), 7.42 (d, 1H), 7.55-7.61 (m, 6H), 7.63 (s, 1H), 7.74 (d, 1H), 8.26 (d, 1H) ppm.

### Example 59

### 1-Benzyl-4-(2,4-dichloro-phenyl)-2-[2-(4′-methoxy-biphenyl-4-yl)-(E)-vinyl]-1H-imidazole

4-(2,4-Dichloro-phenyl)-2-[2-(4′-methoxy-biphenyl-4-yl)-(E)-vinyl]-1H-imidazole (42 mg, 0.1 mmol) was treated with benzyl bromide as described in general procedure E to give 1-benzyl-4-(2,4-dichloro-phenyl)-2-[2-(4′-methoxy-biphenyl-4-yl)-(E)-vinyl]-1H-imidazole (32 mg, 63% yield).

LCMS: m/z 511 (M+H)⁺; ¹H NMR (CD₃OD, 400 MHz): δ 3.83 (s, 3H), 5.36 (s, 2H), 7.10 (d, 1H), 7.12 (d, 1H), 7.42 (m, 2H), 7.48 (d, 2H), 7.51 (m, 5H), 7.61 (d, 2H), 7.65 (d, 2H), 7.69 (d, 2H), 7.74 (s, 1H), 7.81 (d, 1H) ppm.

### Example 60

### 4-(2,4-Dichloro-phenyl)-1-isopropyl-2-[2-(4′-methoxy-biphenyl-4-yl)-(E)-vinyl]-1H-imidazole

4-(2,4-Dichloro-phenyl)-2-[2-(4′-methoxy-biphenyl-4-yl)-(E)-vinyl]-1H-imidazole (42 mg, 0.1 mmol) was treated with 2-bromopropane as described in general procedure E to give 4-(2,4-dichloro-phenyl)-1-isopropyl-2-[2-(4′-methoxy-biphenyl-4-yl)-(E)-vinyl]-1H-imidazole (16 mg, 33% yield).

LCMS: m/z 463 (M+H)⁺.

### Example 61

### 1-Cyclopropyl-4-(2,4-dichloro-phenyl)-2-[2-(4′-methoxy-biphenyl-4-yl)-(E)-vinyl]-1H-imidazole

4-(2,4-Dichloro-phenyl)-2-[2-(4′-methoxy-biphenyl-4-yl)-(E)-vinyl]-1H-imidazole (42 mg, 0.1 mmol) was treated with cyclopropyl bromide as described in general procedure E to give 1-cyclopropyl-4-(2,4-dichloro-phenyl)-2-[2-(4′-methoxy-biphenyl-4-yl)-(E)-vinyl]-1H-imidazole (14 mg, 30% yield).

LCMS: m/z 461 (M+H)⁺.

### Example 62

### 4-(2,4-Dichloro-phenyl)-2-[2-(4′-ethoxy-biphenyl-4-yl)-(E)-vinyl]-1-ethyl-1H-imidazole

4′-{2-[4-(2,4-Dichloro-phenyl)-1H-imidazol-2-yl]-(E)-vinyl}-biphenyl-4-ol (44 mg, 0.1 mmol) was treated as described in general procedure E using ethyl bromide to give 4-(2,4-dichloro-phenyl)-2-[2-(4′-ethoxy-biphenyl-4-yl)-(E)-vinyl]-1-ethyl-1H-imidazole (36 mg, 79% yield).

LCMS: m/z 463 (M+H)⁺; ¹H NMR (CDCl₃, 400 MHz): δ 1.46 (t, 3H), 1.57 (t, 3H), 4.09 (q, 2H), 4.30 (q, 2H), 6.94 (d, 1H), 6.97 (d, 2H), 7.45 (d, 1H), 7.50-7.56 (m, 6H), 7.75 (d, 2H), 8.59 (d, 1H), 8.93 (d, 1H) ppm.

### Example 63

### {4-(2,4-Dichloro-phenyl)-2-[2-(4-methoxy-phenyl)-(E)-vinyl]-imidazol-1-yl}-acetic acid

4-(2,4-Dichloro-phenyl)-2-[2-(4-methoxy-phenyl)-(E)-vinyl]-1H-imidazole (3.45 g, mmol) was treated with methyl bromoacetate as described in general procedure E followed by ester hydrolysis as described in general procedure F to afford {4-(2,4-dichloro-phenyl)-2-[2-(4-methoxy-phenyl)-(E)-vinyl]-imidazol-1-yl}-acetic acid (2.26 g, 56% yield).

LCMS: m/z 403 (M+H)⁺; ¹H NMR (CD₃OD, 400 MHz): δ 3.82 (s, 3H), 4.97 (s, 2H), 6.88 (d, 1H), 6.95 (d, 2H), 7.33 (d, 1H), 7.51 (d, 2H), 7.52 (d, 1H), 7.54 (s, 1H), 7.66 (d, 1H), 7.93 (s, 1H) ppm.

### Example 64

### 2-{4-(2,4-Dichlorophenyl)-2-[2-(4-methoxy-phenyl)-(E)-vinyl]-imidazol-1-yl}-N-(1-naphthalen-1-yl-ethyl)-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with DL-1-(1-naphthyl)ethylamine following the general procedure G to afford 2-{4-(2,4-dichloro-phenyl)-2-[2-(4-methoxy-phenyl)-(E)-vinyl]-imidazol-1-yl}-N-(1-naphthalen-1-yl-ethyl)-acetamide (42 mg, 78% yield).

LCMS: m/z 556 (M+H)⁺; ¹H NMR (CD₃OD, 400 MHz): δ 1.59 (d, 3H), 3.86 (s, 3H), 4.83 (s, 2H), 5.77 (m, 1H), 5.98 (m, 1H), 6.59 (d, 1H), 6.89 (d, 2H), 7.28-7.50 (m, 6H), 7.56 (s, 1H), 7.60 (d, 1H), 7.62 (d, 1H), 7.72 (d, 1H), 7.82 (d, 1H), 8.03 (d, 1H), 8.18 (d, 1H) ppm.

### Example 65

### 2-{4-(2,4-Dichlorophenyl)-2-[2-(4-methoxy-phenyl)-(E)-vinyl]-imidazol-1-yl}-N-(1-naphthalen-1-yl-ethyl)-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with (S)-1-(1-naphthyl)ethylamine following the general procedure G to afford 2-{4-(2,4-dichloro-phenyl)-2-[2-(4-methoxy-phenyl)-(E)-vinyl]-imidazol-1-yl}-N-(1-naphthalen-1-yl-ethyl)-acetamide (41 mg, 73% yield).

LCMS: m/z 556 (M+H)⁺; ¹H NMR (CD₃OD, 400 MHz): δ 1.61 (d, 3H), 3.83 (s, 3H), 4.78 (s, 2H), 5.77 (m, 1H), 5.98 (m, 1H), 6.59 (d, 1H), 6.89 (d, 2H), 7.28-7.50 (m, 6H), 7.56 (s, 1H), 7.60 (d, 1H), 7.62 (d, 1H), 7.72 (d, 1H), 7.82 (d, 1H), 8.03 (d, 1H), 8.19 (d, 1H) ppm.

### Example 66

### N-Butyl-2-{4-(2,4-dichloro-phenyl)-2-[2-(4-methoxy-phenyl-(E)-vinyl]-imidazol-1-yl}-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with n-butylamine following the general procedure G to afford N-butyl-2-{4-(2,4-dichloro-phenyl)-2-[2-(4-methoxy-phenyl)-(E)-vinyl]-imidazol-1-yl}-acetamide (39 mg, 85% yield).

LCMS: m/z 458 (M+H)⁺; ¹H NMR (CD₃OD, 400 MHz): δ 1.24 (t, 3H), 1.43 (m, 2H), 1.84 (m, 2H), 3.08 (d, 2H), 3.83 (s, 3H), 4.89 (s, 2H), 6.87 (d, 1H), 6.94 (d, 2H), 7.33 (d, 1H), 7.51 (d, 2H), 7.52 (d, 1H), 7.54 (s, 1H), 7.66 (d, 1H), 7.93 (s, 1H) ppm.

### Example 67

### 2-{4-(2,4-Dichloro-phenyl)-2-[2-(4-methoxy-phenyl)-(E)-vinyl]-imidazol-1-yl}-N-isobutyl-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with isobutylamine following the general procedure G to afford 2-{4-(2,4-dichloro-phenyl)-2-[2-(4-methoxy-phenyl)-(E)-vinyl]-imidazol-1-yl}-N-isobutyl-acetamide (36 mg, 78% yield).

LCMS: m/z 458 (M+H)⁺; ¹H NMR (CD₃OD, 400 MHz): δ 0.90 (d, 6H), 1.80 (m, 1H), 3.07 (d, 2H), 3.82 (s, 3H), 4.87 (s, 2H), 6.87 (d, 1H), 6.94 (d, 2H), 7.33 (d, 1H), 7.51 (d, 2H), 7.52 (d, 1H), 7.54 (s, 1H), 7.66 (d, 1H), 7.93 (s, 1H) ppm.

### Example 68

### 2-{4-(2,4-Dichloro-phenyl)-2-[2-(4-methoxy-phenyl)-(E)-vinyl]-imidazol-1-yl}-N,N-diisopropyl-acetamide

{4-(2,4-Dichloro-phenyl)-2-[2-(4-methoxy-phenyl)-(E)-vinyl]-imidazol-1-yl}-acetic acid (20 mg, 0.05 mmol) was coupled with diisopropylamine following the general procedure G to afford 2-{4-(2,4-dichloro-phenyl)-2-[2-(4-methoxy-phenyl)-(E)-vinyl]-imidazol-1-yl}-N,N-diisopropyl-acetamide (14 mg, 58% yield).

LCMS: m/z 486 (M+H)⁺; ¹H NMR (CD₃OD, 400 MHz): δ 1.32 (d, 6H), 1.38 (d, 6H), 3.61 (m, 1H), 3.82 (s, 3H), 4.13 (m, 1H), 5.12 (s, 2H), 6.81 (d, 1H), 6.94 (d, 2H), 7.45 (d, 1H), 7.50-7.52 (m, 4H), 7.68 (dd, 1H), 7.96 (d, 1H) ppm.

### Example 69

### 2-{4-(2,4-Dichloro-phenyl)-2-[2-(4-methoxy-phenyl)-(E)-vinyl]-imidazol-1-yl}-N-(3-dimethylamino-propyl)-acetamide

{4-(2,4-Dichloro-phenyl)-2-[2-(4-methoxy-phenyl)-(E)-vinyl]-imidazol-1-yl}-acetic acid (20 mg, 0.05 mmol) was coupled with 3-(dimethylamino)-propylamine following the general procedure G to afford 2-{4-(2,4-dichloro-phenyl)-2-[2-(4-methoxy-phenyl)-(E)-vinyl]-imidazol-1-yl}-N-(3-dimethylamino-propyl)-acetamide (19 mg, 78% yield).

LCMS: m/z 487 (M+H)⁺.

### Example 70

### 2-{4-(2,4-Dichloro-phenyl)-2-[2-(4-methoxy-phenyl)-(E)-vinyl]-imidazol-1-yl}-N-[2-(3-methoxy-phenyl)-ethyl]-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with 3-methoxyphenethyl-amine following the general procedure G to afford 2-{4-(2,4-dichloro-phenyl)-2-[2-(4-methoxy-phenyl)-(E)-vinyl]-imidazol-1-yl}-N-[2-(3-methoxy-phenyl)-ethyl]-acetamide (43 mg, 80% yield).

LCMS: m/z 536 (M+H)⁺; ¹H NMR (CD₃OD, 400 MHz): δ 2.82 (t, 2H), 3.53 (m, 2H), 3.73 (s, 3H), 3.86 (s, 3H), 5.11 (s, 2H), 6.71-6.80 (m, 3H), 7.01 (d, 1H), 7.04 (d, 2H), 7.15 (m, 1H), 7.57 (dd, 1H), 7.66 (d, 2H), 7.71 (d, 1H), 7.73 (d, 1H), 7.76 (d, 1H), 7.83 (s, 1H) ppm.

### Example 71

### N-(4-tert-Butyl-benzyl)-2-{4-(2,4-dichloro-phenyl)-2-[2-(4-methoxy-phenyl)-(E)-vinyl]-imidazol-1-yl}-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with 4-tert-butyl-benzylamine following the general procedure G to afford N-(4-tert-butyl-benzyl)-2-{4-(2,4-dichloro-phenyl)-2-[2-(4-methoxy-phenyl)-(E)-vinyl]-imidazol-1-yl}-acetamide (46 mg, 83% yield).

LCMS: m/z 548 (M+H)⁺; ¹H NMR (CDCl₃, 400 MHz): δ 1.22 (s, 9H), 3.85 (s, 3H), 4.43 (d, 2H), 4.82 (s, 2H), 5.82 (m, 1H), 6.69 (d, 1H), 6.93 (d, 2H), 7.08 (d, 2H), 7.17 (d, 2H), 7.33 (dd, 1H), 7.43 (d, 1H), 7.49 (d, 2H), 7.65 (s, 1H), 7.67 (d, 1H), 8.23 (d, 1H) ppm.

### Example 72

### 2-{4-(2,4-Dichloro-phenyl)-2-[2-(4-methoxy-phenyl)-(E)-vinyl]-imidazol-1-yl}-N-[2-(4-methoxy-phenyl)-ethyl]-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with 4-methoxyphenethyl-amine following the general procedure G to afford 2-{4-(2,4-dichloro-phenyl)-2-[2-(4-methoxy-phenyl)-(E)-vinyl]-imidazol-1-yl}-N-[2-(4-methoxy-phenyl)-ethyl]-acetamide (47 mg, 87% yield).

LCMS: m/z 536 (M+H)⁺; ¹H NMR (CD₃OD, 400 MHz): δ 2.84 (t, 2H), 3.53 (m, 2H), 3.73 (s, 3H), 3.86 (s, 3H), 5.11 (s, 2H), 6.71-6.80 (m, 3H), 7.04 (d, 2H), 7.10 (d, 2H), 7.57 (dd, 1H), 7.66 (d, 2H), 7.71 (d, 1H), 7.73 (d, 1H), 7.76 (d, 1H), 7.81 (s, 1H) ppm.

### Example 73

### 2-{4-(2,4-Dichloro-phenyl)-2-[2-(4-methoxy-phenyl)-(E)-vinyl]-imidazol-1-yl}-N-[2-(3,4-dimethoxy-phenyl)-ethyl]-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with 3,4-dimethoxyphenethylamine following the general procedure G to afford 2-{4-(2,4-dichloro-phenyl)-2-[2-(4-methoxy-phenyl)-(E)-vinyl]-imidazol-1-yl}-N-[2-(3,4-dimethoxy-phenyl)-ethyl]-acetamide (48 mg, 84% yield).

LCMS: m/z 566 (M+H)⁺; ¹H NMR (CD₃OD, 400 MHz): δ 2.84 (t, 2H), 3.53 (m, 2H), 3.73 (s, 3H), 3.82 (s, 3H), 3.86 (s, 3H), 5.11 (s, 2H), 6.71-6.80 (m, 3H), 7.04 (d, 2H), 7.10 (d, 2H), 7.57 (dd, 1H), 7.66 (d, 2H), 7.71 (d, 1H), 7.73 (d, 1H), 7.76 (d, 1H), 7.81 (s, 1H) ppm.

### Example 74

### 2-{4-(2,4-Dichloro-phenyl)-2-[2-(4-methoxy-phenyl)-(E)-vinyl]-imidazol-1-yl}-N-[2-(4-fluoro-phenyl)-ethyl]-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with 4-fluorophenethylamine following the general procedure G to afford 2-{4-(2,4-dichloro-phenyl)-2-[2-(4-methoxy-phenyl)-(E)-vinyl]-imidazol-1-yl}-N-[2-(4-fluoro-phenyl)-ethyl]-acetamide (48 mg, 91% yield).

LCMS: m/z 524 (M+H)⁺; ¹H NMR (CD₃OD, 400 MHz): δ 2.83 (t, 2H), 3.52 (m, 2H), 3.83 (s, 3H), 5.11 (s, 2H), 6.71-6.80 (m, 3H), 7.04 (d, 2H), 7.10 (d, 2H), 7.57 (dd, 1H), 7.66 (d, 2H), 7.71 (d, 1H), 7.73 (d, 1H), 7.76 (d, 1H), 7.81 (s, 1H) ppm.

### Example 75

### 2-{4-(2,4-Dichloro-phenyl)-2-[2-(4-methoxy-phenyl)-(E)-vinyl]-imidazol-1-yl}-N-wasoquinolin-5-yl-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with 5-aminoisoquinoline following the general procedure G to afford 2-{4-(2,4-dichloro-phenyl)-2-[2-(4-methoxy-phenyl)-(E)-vinyl]-imidazol-1-yl}-N-isoquinolin-5-yl-acetamide (39 mg, 74% yield).

LCMS: m/z 529 (M+H)⁺; ¹H NMR (CD₃OD, 400 MHz): δ 3.83 (s, 3H), 5.12 (s, 2H), 6.73-6.87 (m, 5H), 7.04 (d, 2H), 7.10 (d, 2H), 7.57 (dd, 1H), 7.66 (d, 2H), 7.71 (d, 1H), 7.73 (d, 1H), 7.76 (d, 1H), 7.81 (s, 1H) ppm.

### Example 76

### 2-{4-(2,4-Dichloro-phenyl)-2-[2-(4-methoxy-phenyl)-(E)-vinyl]-imidazol-1-yl}-N-pyridin-4-yl-acetamide

{4-(2,4-Dichloro-phenyl)-2-[2-(4-methoxy-phenyl)-(E)-vinyl]-imidazol-1-yl}-acetic acid (41 mg, 0.1 mmol) was coupled with 4-aminopyridine following the general procedure G to afford 2-{4-(2,4-dichloro-phenyl)-2-[2-(4-methoxy-phenyl)-(E)-vinyl]-imidazol-1-yl}-N-pyridin-4-yl-acetamide (33 mg, 68% yield).

LCMS: m/z 479 (M+H)⁺; ¹H NMR (CD₃OD, 400 MHz): δ 3.80 (s, 3H), 5.11 (s, 2H), 6.73-6.81 (m, 3H), 7.04 (d, 2H), 7.10 (d, 2H), 7.57 (dd, 1H), 7.66 (d, 2H), 7.71 (d, 1H), 7.73 (d, 1H), 7.76 (d, 1H), 7.83 (s, 1H) ppm.

### Example 77

### [4-(2,4-Dichloro-phenyl)-2-fluoren-9-ylidenemethyl-imidazol-1-yl]-acetic acid

4-(2,4-Dichloro-phenyl)-2-fluoren-9-ylidenemethyl-1H-imidazole (389 mg, 1 mmol) was treated with methyl bromoacetate as described in general procedure E followed by ester hydrolysis as described in general procedure F to afford [4-(2,4-dichloro-phenyl)-2-fluoren-9-ylidenemethyl-imidazol-1-yl]-acetic acid (260 mg, 58% yield).

LCMS: m/z 447 (M+H)⁺; ¹H NMR (CD₃OD, 400 MHz): δ 5.02 (s, 2H), 7.25 (m, 1H), 7.37-7.51 (m, 5H), 7.57 (dd, 1H), 7.73 (d, 1H), 7.77-7.82 (m, 3H), 7.93 (d, 1H), 8.08 (s, 1H) ppm.

### Example 78

### 2-[4-(2,4-Dichloro-phenyl)-2-fluoren-9-ylidenemethyl-imidazol-1-yl]-N-[2-(3-methoxy-phenyl)-ethyl]-acetamide

[4-(2,4-Dichloro-phenyl)-2-fluoren-9-ylidenemethyl-imidazol-1-yl]-acetic acid (45 mg, 0.1 mmol) was coupled with 3-methoxyphenethylamine following the general procedure G to afford 2-[4-(2,4-dichloro-phenyl)-2-fluoren-9-ylidenemethyl-imidazol-1-yl]-N-[2-(3-methoxy-phenyl)-ethyl]-acetamide (47 mg, 81% yield).

LCMS: m/z 580 (M+H)⁺; ¹H NMR (CD₃OD, 400 MHz): δ 2.82 (t, 2H), 3.53 (m, 2H), 3.73 (s, 3H), 5.08 (s, 2H), 6.71-6.80 (m, 3H), 7.01 (d, 1H), 7.25 (m, 1H), 7.37-7.51 (m, 5H), 7.57 (dd, 1H), 7.73 (d, 1H), 7.77-7.82 (m, 3H), 7.93 (d, 1H), 8.08 (s, 1H) ppm.

### Example 79

### 2-[4-(2,4-Dichloro-phenyl)-2-fluoren-9-ylidenemethyl-imidazol-1-yl]-N-[2-(4-methoxy-phenyl)-ethyl]-acetamide

[4-(2,4-Dichloro-phenyl)-2-fluoren-9-ylidenemethyl-imidazol-1-yl]-acetic acid (45 mg, 0.1 mmol) was coupled with 4-methoxyphenethyl-amine following the general procedure G to afford 2-[4-(2,4-dichloro-phenyl)-2-fluoren-9-ylidenemethyl-imidazol-1-yl]-N-[2-(4-methoxy-phenyl)-ethyl]-acetamide (51 mg, 88% yield).

LCMS: m/z 580 (M+H)⁺; ¹H NMR (CD₃OD, 400 MHz): δ 2.83 (t, 2H), 3.53 (m, 2H), 3.73 (s, 3H), 5.08 (s, 2H), 6.77 (d, 2H), 7.03 (d, 2H), 7.25 (m, 1H), 7.37-7.51 (m, 5H), 7.57 (dd, 1H), 7.73 (d, 1H), 7.77-7.82 (m, 3H), 7.93 (d, 1H), 8.09 (s, 1H) ppm.

### Example 80

### 2-[4-(2,4-Dichloro-phenyl)-2-fluoren-9-ylidenemethyl-imidazol-1-yl]-N-(1-naphthalen-1-yl-ethyl)-acetamide

[4-(2,4-Dichloro-phenyl)-2-fluoren-9-ylidenemethyl-imidazol-1-yl]-acetic acid (45 mg, 0.1 mmol) was coupled with DL-1-(1-naphthyl)ethylamine following the general procedure G to afford 2-[4-(2,4-dichloro-phenyl)-2-fluoren-9-ylidenemethyl-imidazol-1-yl]-N-(1-naphthalen-1-yl-ethyl)-acetamide (53 mg, 88% yield).

LCMS: m/z 600 (M+H)⁺.

### Example 81

### 4-[4-(2,4-Dichloro-phenyl)-2-fluoren-9-ylidenemethyl-imidazol-1-yl]-butyric acid

4-(2,4-Dichloro-phenyl)-2-fluoren-9-ylidenemethyl-1H-imidazole (39 mg, 0.1 mmol) was treated with methyl 1-bromobutyrate as described in general procedure E followed by ester hydrolysis as described in general procedure F to afford 4-[4-(2,4-dichloro-phenyl)-2-fluoren-9-ylidenemethyl-imidazol-1-yl]-butyric acid (23 mg, 48% yield).

LCMS: m/z 475 (M+H)⁺; ¹H NMR (CD₃OD, 400 MHz): δ 2.14 (m, 2H), 2.40 (t, 2H), 4.32 (t, 2H), 7.26 (m, 1H), 7.33 (m, 1H), 7.39 (t, 2H), 7.44 (dd, 1H), 7.53 (s 1H), 7.56 (dd, 1H), 7.75 (t, 2H), 7.97 (s, 1H), 8.02 (d, 1H), 8.12 (d, 1H), 8.83 (d, 1H) ppm.

### Example 82

### 2-{4-(2,4-Dichloro-phenyl)-2-[2-(4-hydroxy-phenyl)-(E)-vinyl]-imidazol-1-yl}-N-(1-naphthalen-1-yl-ethyl)-acetamide

2-{4-(2,4-Dichloro-phenyl)-2-[2-(4-methoxy-phenyl)-(E)-vinyl]-imidazol-1-yl}-N-(1-naphthalen-1-yl-ethyl)-acetamide (556 mg, 1 mmol) was treated according to the general procedure C to afford 2-{4-(2,4-dichloro-phenyl)-2-[2-(4-hydroxy-phenyl)-(E)-vinyl]-imidazol-1-yl}-N-(1-naphthalen-1-yl-ethyl)-acetamide (412 mg, 76% yield).

LCMS: m/z 542 (M+H)⁺; ¹H NMR (CD₃OD, 400 MHz): δ 1.59 (d, 3H), 4.78 (s, 2H), 5.77 (m, 1H), 5.98 (m, 1H), 6.59 (d, 1H), 6.89 (d, 2H), 7.28-7.50 (m, 6H), 7.56 (s, 1H), 7.60 (d, 1H), 7.62 (d, 1H), 7.72 (d, 1H), 7.82 (d, 1H), 8.03 (d, 1H), 8.18 (d, 1H) ppm.

### Example 83

### [4-(2-{4-(2,4-Dichloro-phenyl)-1-[(1-naphthalen-1-yl-ethylcarbamoyl)-methyl]-1H-imidazol-2-yl}-(E)-vinyl)-phenoxy]-acetic acid

2-{4-(2,4-Dichloro-phenyl)-2-[2-(4-hydroxy-phenyl)-(E)-vinyl]-imidazol-1-yl}-N-(1-naphthalen-1-yl-ethyl)-acetamide (54 mg, 0.1 mmol) was treated with methyl bromoacetate as described in the general procedure E followed by ester hydrolysis as described in the general procedure F to give [4-(2-{4-(2,4-Dichloro-phenyl)-1-[(1-naphthalen-1-yl-ethylcarbamoyl)-methyl]-1H-imidazol-2-yl}-(E)-vinyl)-phenoxy]-acetic acid (21 mg, 35% yield).

LCMS: m/z 600 (M+H)⁺; ¹H NMR (CD₃OD, 400 MHz): δ 1.59 (d, 3H), 4.21 (s, 2H), 4.78 (s, 2H), 5.77 (m, 1H), 5.98 (m, 1H), 6.59 (d, 1H), 6.89 (d, 2H), 7.28-7.50 (m, 6H), 7.56 (s, 1H), 7.60 (d, 1H), 7.62 (d, 1H), 7.72 (d, 1H), 7.82 (d, 1H), 8.03 (d, 1H), 8.18 (d, 1H) ppm.

### Example 84

### 4-[4-(2-{4-(2,4-Dichloro-phenyl)-1-[(1-naphthalen-1-yl-ethylcarbamoyl)-methyl]-1H-imidazol-2-yl}-(E)-vinyl)-phenoxy]-butyric acid

2-{4-(2,4-Dichloro-phenyl)-2-[2-(4-hydroxy-phenyl)-(E)-vinyl]-imidazol-1-yl}-N-(1-naphthalen-1-yl-ethyl)-acetamide (54 mg, 0.1 mmol) was treated with methyl 4-bromobutyrate as described in the general procedure E followed by ester hydrolysis as described in the general procedure F to give 4-[4-(2-{4-(2,4-dichloro-phenyl)-1-[(1-naphthalen-1-yl-ethylcarbamoyl)-methyl]-1H-imidazol-2-yl}-(E)-vinyl)-phenoxy]-butyric acid (25 mg, 39% yield).

LCMS: m/z 628 (M+H)⁺.

### Example 85

### 4-[4-(2-{4-(2,4-Dichloro-phenyl)-1-[(1-naphthalen-1-yl-ethylcarbamoyl)-methyl]-1H-imidazol-2-yl}-(E)-vinyl)-phenoxymethyl]-benzoic acid

2-{4-(2,4-Dichloro-phenyl)-2-[2-(4-hydroxy-phenyl)-(E)-vinyl]-imidazol-1-yl}-N-(1-naphthalen-1-yl-ethyl)-acetamide (54 mg, 0.1 mmol) was treated with methyl 4-(bromomethyl)benzoate as described in the general procedure E followed by ester hydrolysis as described in the general procedure F to give 4-[4-(2-{4-(2,4-dichloro-phenyl)-1-[(1-naphthalen-1-yl-ethylcarbamoyl)-methyl]-1H-imidazol-2-yl}-(E)-vinyl)-phenoxymethyl]-benzoic acid (29 mg, 42% yield).

LCMS: m/z 676 (M+H)⁺; ¹H NMR (CD₃OD, 400 MHz): δ 1.59 (d, 3H), 4.78 (s, 2H), 5.21 (s, 2H), 5.77 (m, 1H), 5.98 (m, 1H), 6.59 (d, 1H), 6.89 (d, 2H), 7.28-7.50 (m, 10H), 7.56 (s, 1H), 7.60 (d, 1H), 7.62 (d, 1H), 7.72 (d, 1H), 7.82 (d, 1H), 8.03 (d, 1H), 8.18 (d, 1H) ppm.

### Example 86

### 3-[4-(2-{4-(2,4-Dichloro-phenyl)-1-[(1-naphthalen-1-yl-ethylcarbamoyl)-methyl]-1H-imidazol-2-yl}-(E)-vinyl)-phenoxymethyl]-benzoic acid

2-{4-(2,4-Dichloro-phenyl)-2-[2-(4-hydroxy-phenyl)-(E)-vinyl]-imidazol-1-yl}-N-(1-naphthalen-1-yl-ethyl)-acetamide (54 mg, 0.1 mmol) was treated with methyl 3-(bromomethyl)benzoate as described in the general procedure E followed by ester hydrolysis as described in the general procedure F to give 3-[4-(2-{4-(2,4-dichloro-phenyl)-1-[(1-naphthalen-1-yl-ethylcarbamoyl)-methyl]-1H-imidazol-2-yl}-(E)-vinyl)-phenoxymethyl]-benzoic acid (26 mg, 38% yield).

LCMS: m/z 676 (M+H)⁺; ¹H NMR (CD₃OD, 400 MHz): δ 1.61 (d, 3H), 4.81 (s, 2H), 5.21 (s, 2H), 5.77 (m, 1H), 5.98 (m, 1H), 6.59 (d, 1H), 6.89 (d, 2H), 7.29-7.52 (m, 10H), 7.56 (s, 1H), 7.60 (d, 1H), 7.62 (d, 1H), 7.72 (d, 1H), 7.82 (d, 1H), 8.03 (d, 1H), 8.19 (d, 1H) ppm.

### Example 87

### 2-{4-(2,4-Dichloro-phenyl)-2-[2-(4-ethoxy-phenyl)-(E)-vinyl]-imidazol-1-yl}-N-(1-naphthalen-1-yl-ethyl)-acetamide

2-{4-(2,4-Dichloro-phenyl)-2-[2-(4-hydroxy-phenyl)-(E)-vinyl]-imidazol-1-yl}-N-(1-naphthalen-1-yl-ethyl)-acetamide (54 mg, 0.1 mmol) was treated with ethyl bromide as described in the general procedure E to give 2-{4-(2,4-dichloro-phenyl)-2-[2-(4-ethoxy-phenyl)-(E)-vinyl]-imidazol-1-yl}-N-(1-naphthalen-1-yl-ethyl)-acetamide (47 mg, 82% yield).

LCMS: m/z 570 (M+H)⁺; ¹H NMR (CD₃OD, 400 MHz): δ 1.43 (t, 3H), 1.59 (d, 3H), 4.22 (q, 2H), 4.78 (s, 2H), 5.77 (m, 1H), 5.98 (m, 1H), 6.59 (d, 1H), 6.89 (d, 2H), 7.28-7.50 (m, 6H), 7.56 (s, 1H), 7.60 (d, 1H), 7.62 (d, 1H), 7.72 (d, 1H), 7.82 (d, 1H), 8.03 (d, 1H), 8.18 (d, 1H) ppm.

### Example 88

### 4-(4′-{2-[1-Benzyl-4-(2,4-dichloro-phenyl)-1H-imidazol-2-yl]-(E)-vinyl}-biphenyl-4-yloxy)-butyric acid

1-Benzyl-4-(2,4-dichloro-phenyl)-2-[2-(4′-methoxy-biphenyl-4-yl)-(E)-vinyl]-1H-imidazole (51 mg, 0.1 mmol) was treated as described in general procedure C and the resulting phenol was treated with methyl 4-bromobutyrate as described in the general procedure E followed by ester hydrolysis as described in the general procedure F to give 4-(4′-{2-[1-benzyl-4-(2,4-dichloro-phenyl)-1H-imidazol-2-yl]-(E)-vinyl}-biphenyl-4-yloxy)-butyric acid (20 mg, 34% yield).

LCMS: m/z 583 (M+H)⁺; ¹H NMR (CD₃OD, 400 MHz): δ 1.95 (m, 2H), 2.38 (t, 2H), 4.12 (t, 2H), 5.33 (s, 2H), 7.10 (d, 1H), 7.12 (d, 1H), 7.42 (m, 2H), 7.48 (d, 2H), 7.51 (m, 5H), 7.61 (d, 2H), 7.65 (d, 2H), 7.69 (d, 2H), 7.74 (s, 1H), 7.81 (d, 1H) ppm.

### Example 89

### 4-(4′-{2-[1-Butyl-4-(2,4-dichloro-phenyl)-1H-imidazol-2-yl]-(E)-vinyl}-biphenyl-4-yloxy)-butyric acid

1-Butyl-4-(2,4-dichloro-phenyl)-2-[2-(4′-methoxy-biphenyl-4-yl)-(E)-vinyl]-1H-imidazole (48 mg, 0.1 mmol) was treated as described in general procedure C and the resulting phenol was treated with methyl 4-bromobutyrate as described in the general procedure E followed by ester hydrolysis as described in the general procedure F to give 4-(4′-{2-[1-butyl-4-(2,4-dichloro-phenyl)-1H-imidazol-2-yl]-(E)-vinyl}-biphenyl-4-yloxy)-butyric acid (22 mg, 39% yield).

LCMS: m/z 549 (M+H)⁺.

### Example 90

### {4-(2,4-Dichloro-phenyl)-2-[2-(4′-methoxy-biphenyl-4-yl)-(E)-vinyl]-imidazol-1-yl}-acetic acid

4-(2,4-Dichloro-phenyl)-2-[2-(4′-methoxy-biphenyl-4-yl)-(E)-vinyl]-1H-imidazole (421 mg, 1 mmol) was treated with methyl bromoacetate as described in general procedure E followed by ester hydrolysis as described in general procedure F to afford {4-(2,4-dichloro-phenyl)-2-[2-(4′-methoxy-biphenyl-4-yl)-(E)-vinyl]-imidazol-1-yl}-acetic acid (268 mg, 56% yield).

LCMS: m/z 479 (M+H)⁺; ¹H NMR (CD₃OD, 400 MHz): δ 3.82 (s, 3H), 4.95 (s, 2H), 7.03 (d, 2H), 7.15 (d, 1H), 7.58-7.61 (m, 3H), 7.68-7.70 (m, 6H), 7.73 (d, 1H), 7.90 (s, 1H) ppm.

### Example 91

### 2-{4-(2,4-Dichloro-phenyl)-2-[2-(4′-methoxy-biphenyl-4-yl)-(E)-vinyl]-imidazol-1-yl}-N-(1-naphthalen-1-yl-ethyl)-acetamide

{4-(2,4-Dichloro-phenyl)-2-[2-(4′-methoxy-biphenyl-4-yl)-(E)-vinyl]-imidazol-1-yl}-acetic acid (24 mg, 0.05 mmol) was coupled with DL-1-(1-naphthyl)ethylamine following the general procedure G to afford 2-{4-(2,4-dichloro-phenyl)-2-[2-(4′-methoxy-biphenyl-4-yl)-(E)-vinyl]-imidazol-1-yl}-N-(1-naphthalen-1-yl-ethyl)-acetamide (21 mg, 67% yield).

LCMS: m/z 632 (M+H)⁺; ¹H NMR (CD₃OD, 400 MHz): δ 1.61 (d, 3H), 3.83 (s, 3H), 4.78 (s, 2H), 5.77 (m, 1H), 5.98 (m, 1H), 6.59 (d, 1H), 6.89 (d, 2H), 7.29-7.52 (m, 10H), 7.56 (s, 1H), 7.60 (d, 1H), 7.62 (d, 1H), 7.72 (d, 1H), 7.82 (d, 1H), 8.03 (d, 1H), 8.19 (d, 1H) ppm.

### Example 92

### 2-{4-(2,4-Dichloro-phenyl)-2-[2-(4′-hydroxy-biphenyl-4-yl)-(E)-vinyl]-imidazol-1-yl}-N-(1-naphthalen-1-yl-ethyl)-acetamide

2-{4-(2,4-Dichloro-phenyl)-2-[2-(4′-methoxy-biphenyl-4-yl)-(E)-vinyl]-imidazol-1-yl}-N-(1-naphthalen-1-yl-ethyl)-acetamide (64 mg, 0.1 mmol) was treated as described in the general procedure C to afford 2-{4-(2,4-dichloro-phenyl)-2-[2-(4′-hydroxy-biphenyl-4-yl)-(E)-vinyl]-imidazol-1-yl}-N-(1-naphthalen-1-yl-ethyl)-acetamide (52 mg, 83% yield).

LCMS: m/z 618 (M+H)⁺; ¹H NMR (CD₃OD, 400 MHz): δ 1.63 (d, 3H), 4.80 (s, 2H), 5.77 (m, 1H), 5.98 (m, 1H), 6.59 (d, 1H), 6.89 (d, 2H), 7.29-7.52 (m, 10H), 7.56 (s, 1H), 7.60 (d, 1H), 7.62 (d, 1H), 7.72 (d, 1H), 7.82 (d, 1H), 8.03 (d, 1H), 8.17 (d, 1H) ppm.

### Example 93

### 4-[4′-(2-{4-(2,4-Dichloro-phenyl)-1-[(1-naphthalen-1-yl-ethylcarbamoyl)-methyl]-1H-imidazol-2-yl}-(E)-vinyl)-biphenyl-4-yloxy]1-butyric acid

2-{4-(2,4-Dichloro-phenyl)-2-[2-(4′-hydroxy-biphenyl-4-yl)-(E)-vinyl]-imidazol-1-yl}-N-(1-naphthalen-1-yl-ethyl)-acetamide (62 mg, 0.1 mmol) was treated with methyl 4-bromobutyrate as described in the general procedure E followed by ester hydrolysis as described in the general procedure F to afford 4-[4′-(2-{4-(2,4-dichloro-phenyl)-1-[(1-naphthalen-1-yl-ethylcarbamoyl)-methyl]-1H-imidazol-2-yl}-(E)-vinyl)-biphenyl-4-yloxy]-butyric acid (38 mg, 53% yield).

LCMS: m/z 704 (M+H)⁺; ¹H NMR (CD₃OD, 400 MHz): δ 1.63 (d, 3H), 1.97 (m, 2H), 2.41 (t, 2H), 4.12 (t, 2H), 4.80 (s, 2H), 5.77 (m, 1H), 5.98 (m, 1H), 6.59 (d, 1H), 6.89 (d, 2H), 7.29-7.52 (m, 10H), 7.56 (s, 1H), 7.60 (d, 1H), 7.62 (d, 1H), 7.72 (d, 1H), 7.82 (d, 1H), 8.03 (d, 1H), 8.17 (d, 1H) ppm.

### Example 94

### 2-{4-(2,4-Dichloro-phenyl)-2-[2-(4′-methoxy-biphenyl-4-yl)-(E)-vinyl]-imidazol-1-yl}-N-(2-morpholin-4-yl-ethyl)-acetamide

{4-(2,4-Dichloro-phenyl)-2-[2-(4′-methoxy-biphenyl-4-yl)-(E)-vinyl]-imidazol-1-yl}-acetic acid (24 mg, 0.05 mmol) was coupled with 4-(2-aminoethyl)-morpholine following the general procedure G to afford 2-{4-(2,4-dichloro-phenyl)-2-[2-(4′-methoxy-biphenyl-4-yl)-(E)-vinyl]-imidazol-1-yl}-N-(2-morpholin-4-yl-ethyl)-acetamide (23 mg, 76% yield).

LCMS: m/z 591 (M+H)⁺.

### Example 95

### 2-{4-(2,4-Dichloro-phenyl)-2-[2-(4′-methoxy-biphenyl-4-yl)-(E)-vinyl]-imidazol-1-yl}-N-(3,3-dimethyl-butyl)-acetamide

{4-(2,4-Dichloro-phenyl)-2-[2-(4′-methoxy-biphenyl-4-yl)-(E)-vinyl]-imidazol-1-yl}-acetic acid (24 mg, 0.05 mmol) was coupled with 3,3-dimethylbutylamine following the general procedure G to afford 2-{4-(2,4-dichloro-phenyl)-2-[2-(4′-methoxy-biphenyl-4-yl)-(E)-vinyl]-imidazol-1-yl}-N-(3,3-dimethyl-butyl)-acetamide (23 mg, 82% yield).

LCMS: m/z 562 (M+H)⁺.

### Example 96

### 2-{4-(2,4-Dichloro-phenyl)-2-[2-(4′-methoxy-biphenyl-4-yl)-(E)-vinyl]-imidazol-1-yl}-N-[2-(4-methoxy-phenyl)-ethyl]-acetamide

{4-(2,4-Dichloro-phenyl)-2-[2-(4′-methoxy-biphenyl-4-yl)-(E)-vinyl]-imidazol-1-yl}-acetic acid (24 mg, 0.05 mmol) was coupled with 4-methoxyphenethyl-amine following the general procedure G to afford 2-{4-(2,4-dichloro-phenyl)-2-[2-(4′-methoxy-biphenyl-4-yl)-(E)-vinyl]-imidazol-1-yl}-N-[2-(4-methoxy-phenyl)-ethyl]-acetamide (25 mg, 83% yield).

LCMS: m/z 612 (M+H)⁺; ¹H NMR (CD₃OD, 400 MHz): δ 2.84 (t, 2H), 3.53 (m, 2H), 3.73 (s, 3H), 3.86 (s, 3H), 5.02 (s, 2H), 6.71-6.80 (m, 3H), 7.04 (d, 2H), 7.10 (d, 2H), 7.23 (d, 2H), 7.36 (d, 2H), 7.57 (dd, 1H), 7.66 (d, 2H), 7.71 (d, 1H), 7.73 (d, 1H), 7.76 (d, 1H), 7.81 (s, 1H) ppm.

### Example 97

### 4-(4′-{2-[4-(2,4-Dichloro-phenyl)-1-methylcarbamoylmethyl-1H-imidazol-2-yl]-(E)-vinyl}-biphenyl-4-yloxy)-butyric acid

{4-(2,4-Dichloro-phenyl)-2-[2-(4′-methoxy-biphenyl-4-yl)-(E)-vinyl]-imidazol-1-yl}-acetic acid (48 mg, 0.1 mmol) was coupled with methylamine as described in the general procedure G and then demethylated as described in the general procedure C. The resulting phenol was treated with methyl 4-bromobutyrate as described in the general procedure E followed by ester hydrolysis as described in the general procedure F to afford 4-(4′-{2-[4-(2,4-dichloro-phenyl)-1-methylcarbamoylmethyl-1H-imidazol-2-yl]-(E)-vinyl}-biphenyl-4-yloxy)-butyric acid (13 mg, 23% yield).

LCMS: m/z 564 (M+H)⁺; ¹H NMR (CD₃OD, 400 MHz): δ 1.95 (m, 2H), 2.38 (t, 2H), 2.88 (d, 3H), 4.12 (t, 2H), 4.88 (s, 2H), 7.10 (d, 1H), 7.12 (d, 1H), 7.42 (m, 2H), 7.48 (d, 2H), 7.61 (d, 2H), 7.65 (d, 2H), 7.69 (d, 2H), 7.74 (s, 1H), 7.81 (d, 1H) ppm.

### Example 98

### 4-(4′-{2-[4-(2,4-Dichloro-phenyl)-1-ethylcarbamoylmethyl-1H-imidazol-2-yl]-(E)-vinyl}-biphenyl-4-yloxy)-butyric acid

{4-(2,4-Dichloro-phenyl)-2-[2-(4′-methoxy-biphenyl-4-yl)-(E)-vinyl]-imidazol-1-yl}-acetic acid (48 mg, 0.1 mmol) was coupled with ethylamine as described in the general procedure G and then demethylated as described in the general procedure C. The resulting phenol was treated with methyl 4-bromobutyrate as described in the general procedure E followed by ester hydrolysis as described in the general procedure F to afford 4-(4′-{2-[4-(2,4-dichloro-phenyl)-1-ethylcarbamoylmethyl-1H-imidazol-2-yl]-(E)-vinyl}-biphenyl-4-yloxy)-butyric acid (15 mg, 26% yield).

LCMS: m/z 578 (M+H)⁺.

### Example 99

### 4-(4′-{2-[1-Butylcarbamoylmethyl-4-(2,4-dichloro-phenyl)-1H-imidazol-2-yl]-(E)-vinyl}-biphenyl-4-yloxy)-butyric acid

{4-(2,4-Dichloro-phenyl)-2-[2-(4′-methoxy-biphenyl-4-yl)-(E)-vinyl]-imidazol-1-yl}-acetic acid (48 mg, 0.1 mmol) was coupled with n-butylamine as described in the general procedure G and then demethylated as described in the general procedure C. The resulting phenol was treated with methyl 4-bromobutyrate as described in the general procedure E followed by ester hydrolysis as described in the general procedure F to afford 4-(4′-{2-[1-butylcarbamoylmethyl-4-(2,4-dichloro-phenyl)-1H-imidazol-2-yl]-(E)-vinyl}-biphenyl-4-yloxy)-butyric acid (19 mg, 31% yield).

LCMS: m/z 606 (M+H)⁺.

### Example 100

### 4-[2-{2-[4′-(3-Carboxy-propoxy)-biphenyl-4-yl]-(E)-vinyl}-4-(2,4-dichloro-phenyl)-imidazol-1-yl]-butyric acid

4-(2,4-Dichloro-phenyl)-2-[2-(4′-methoxy-biphenyl-4-yl)-(E)-vinyl]-1H-imidazole (42 mg, 0.1 mmol) was demethylated as described in the general procedure C and the resulting intermediate was treated with 2 equivalents of methyl 4-bromobutyrate as described in the general procedure E followed by ester hydrolysis as described in the general procedure F to afford 4-[2-{2-[4′-(3-carboxy-propoxy)-biphenyl-4-yl]-(E)-vinyl}-4-(2,4-dichloro-phenyl)-imidazol-1-yl]-butyric acid (16 mg, 27% yield).

LCMS: m/z 579 (M+H)⁺.

### Example 101

### 4-{4-(2,4-Dichloro-phenyl)-2-[2-(4′-methoxy-biphenyl-4-yl)-(E)-vinyl]-imidazol-1-yl}-butyric acid

4-(2,4-Dichloro-phenyl)-2-[2-(4′-methoxy-biphenyl-4-yl)-(E)-vinyl]-1H-imidazole (42 mg, 0.1 mmol) was treated with methyl 1-bromobutyrate as described in general procedure E followed by ester hydrolysis as described in general procedure F to provide 4-{4-(2,4-dichloro-phenyl)-2-[2-(4′-methoxy-biphenyl-4-yl)-(E)-vinyl]-imidazol-1-yl}-butyric acid (27 mg, 53% yield).

LCMS: m/z 507 (M+H)⁺.

### Example 102

### 4-{4-(2,4-Dichloro-phenyl)-2-[2-(4′-methoxy-biphenyl-4-yl)-(E)-vinyl]-imidazol-1-yl}-N-(1-naphthalen-1-yl-ethyl)-butyramide

4-{4-(2,4-Dichloro-phenyl)-2-[2-(4′-methoxy-biphenyl-4-yl)-(E)-vinyl]-imidazol-1-yl}-butyric acid (26 mg, 0.05 mmol) was coupled with DL-1-(1-naphthyl)ethylamine following the general procedure G to afford 4-{4-(2,4-dichloro-phenyl)-2-[2-(4′-methoxy-biphenyl-4-yl)-(E)-vinyl]-imidazol-1-yl}-N-(1-naphthalen-1-yl-ethyl)-butyramide (15 mg, 45% yield).

LCMS: m/z 660 (M+H)⁺.

### Example 103

### 4-{4-(2,4-Dichloro-phenyl)-2-[2-(4′-methoxy-biphenyl-4-yl)-(E)-vinyl]-imidazol-1-yl}-N-(3,3-dimethyl-butyl)-butyramide

4-{4-(2,4-Dichloro-phenyl)-2-[2-(4′-methoxy-biphenyl-4-yl)-(E)-vinyl]-imidazol-1-yl}-butyric acid (26 mg, 0.05 mmol) was coupled with 3,3-dimethylbutylamine following the general procedure G to afford 4-{4-(2,4-dichloro-phenyl)-2-[2-(4′-methoxy-biphenyl-4-yl)-(E)-vinyl]-imidazol-1-yl}-N-(3,3-dimethyl-butyl)-butyramide (22 mg, 75% yield).

LCMS: m/z 590 (M+H)⁺.

### Example 104

### 2-[2-(4-Bromo-phenyl)-(E)-vinyl]-4-(2,4-dichloro-phenyl)-1-ethyl-1H-imidazole

2-[2-(4-Bromo-phenyl)-(E)-vinyl]-4-(2,4-dichloro-phenyl)-1H-imidazole (394 mg, 1 mmol) was treated as described in general procedure E using ethyl bromide to give 2-[2-(4-bromo-phenyl)-(E)-vinyl]-4-(2,4-dichloro-phenyl)-1-ethyl-1H-imidazole (367 mg, 87% yield).

LCMS: m/z 422 (M+H)⁺; ¹H NMR (CDCl₃, 400 MHz): δ 1.51 (t, 3H), 4.14 (q, 2H), 7.14 (d, 1H), 7.51 (d, 2H), 7.70 (d, 2H), 7.72 (m, 2H), 7.75 (d, 1H), 8.02 (m, 1H), 8.05 (s, 1H) ppm.

### Example 105

### 4-(2,4-Dichloro-phenyl)-1-ethyl-2-[2-(4′-methoxy-biphenyl-4-yl)-(E)-vinyl]-1H-imidazole

2-[2-(4-Bromo-phenyl)-(E)-vinyl]-4-(2,4-dichloro-phenyl)-1-ethyl-1H-imidazole (300 mg, 0.71 mmol) was treated as described in general procedure B using 4-methoxyphenylboronic acid to give 4-(2,4-dichloro-phenyl)-1-ethyl-2-[2-(4′-methoxy-biphenyl-4-yl)-(E)-vinyl]-1H-imidazole (210 mg, 66% yield).

LCMS: m/z 449 (M+H)⁺; ¹H NMR (CDCl₃, 400 MHz): δ 1.52 (t, 3H), 3.86 (s, 3H), 4.14 (q, 2H), 6.94 (d, 1H), 6.99 (d, 2H), 7.32 (m, 1H), 7.42 (d, 1H), 7.55-7.63 (m, 6H), 7.67 (s, 1H), 7.73 (d, 1H), 8.25 (d, 1H) ppm.

### Example 106

### 4′-{2-[4-(2,4-Dichloro-phenyl)-1-ethyl-1H-imidazol-2-yl]-(E)-vinyl}-biphenyl-4-ol

4-(2,4-Dichloro-phenyl)-1-ethyl-2-[2-(4′-methoxy-biphenyl-4-yl)-(E)-vinyl]-1H-imidazole (200 mg, 0.44 mmol) was treated as described in general procedure C to give 4′-{2-[4-(2,4-dichloro-phenyl)-1-ethyl-1H-imidazol-2-yl]-(E)-vinyl}-biphenyl-4-ol (153 mg, 79% yield).

LCMS: m/z 435 (M+H)⁺; ¹H NMR (DMSO-d₆, 400 MHz): δ 1.42 (t, 3H), 4.10 (q, 2H), 6.86 (d, 2H), 7.46 (d, 1H), 7.58 (d, 2H), 7.66 (dd, 1H), 7.70 (d, 2H), 7.82 (d, 2H), 7.85-7.92 (m, 3H), 8.19 (s, 1H) ppm.

### Example 107

### (4′-{2-[4-(2,4-Dichloro-phenyl)-1-ethyl-1H-imidazol-2-yl]-(E)-vinyl}-biphenyl-4-yloxy)-acetic acid

4′-{2-[4-(2,4-Dichloro-phenyl)-1-ethyl-1H-imidazol-2-yl]-(E)-vinyl}-biphenyl-4-ol (44 mg, 0.1 mmol) was treated with methyl bromoacetate according to the general procedure E followed by ester hydrolysis according to the general procedure F to give (4′-{2-[4-(2,4-dichloro-phenyl)-1-ethyl-1H-imidazol-2-yl]-(E)-vinyl}-biphenyl-4-yloxy)-acetic acid (23 mg, 47% yield).

LCMS: m/z 493 (M+H)⁺; ¹H NMR (CDCl₃, 400 MHz): δ 1.50 (t, 3H), 4.35 (q, 2H), 4.79 (s, 2H), 6.94 (d, 1H), 6.99 (d, 2H), 7.32 (m, 1H), 7.42 (d, 1H), 7.55-7.63 (m, 6H), 7.67 (s, 1H), 7.73 (d, 1H), 8.25 (d, 1H) ppm.

### Example 108

### 2-(4′-{2-[4-(2,4-Dichloro-phenyl)-1-ethyl-1H-imidazol-2-yl]-(E)-vinyl}-biphenyl-4-yloxy)-butyric acid

4′-{2-[4-(2,4-Dichloro-phenyl)-1-ethyl-1H-imidazol-2-yl]-(E)-vinyl}-biphenyl-4-ol (44 mg, 0.1 mmol) was treated with (DL-)-methyl 2-bromobutyrate as described in the general procedure E followed by ester hydrolysis as described in the general procedure F to give 2-(4′-{2-[4-(2,4-dichloro-phenyl)-1-ethyl-1H-imidazol-2-yl]-(E)-vinyl}-biphenyl-4-yloxy)-butyric acid (17 mg, 32% yield).

LCMS: m/z 521 (M+H)⁺.

### Example 109

### 4-(4′-{2-[4-(2,4-Dichloro-phenyl)-1-ethyl-1H-imidazol-2-yl]-(E)-vinyl}-biphenyl-4-yloxy)-butyric acid methyl ester

4′-{2-[4-(2,4-Dichloro-phenyl)-1-ethyl-1H-imidazol-2-yl]-(E)-vinyl}-biphenyl-4-ol (87 mg, 0.2 mmol) was treated with methyl 4-bromobutyrate following the general procedure E to give 4-(4′-{2-[4-(2,4-dichloro-phenyl)-1-ethyl-1H-imidazol-2-yl]-(E)-vinyl}-biphenyl-4-yloxy)-butyric acid methyl ester (86 mg, 81% yield).

LCMS: m/z 535 (M+H)⁺; ¹H NMR (CDCl₃, 400 MHz): δ 1.21 (t, 3H), 2.15 (m, 2H), 2.56 (t, 2H), 3.71 (s, 3H), 3.94 (q, 2H), 4.06 (t, 2H), 6.95 (d, 1H), 6.97 (d, 2H), 7.30 (m, 1H), 7.42 (d, 1H), 7.55-7.61 (m, 6H), 7.71 (s, 1H), 7.73 (d, 1H), 8.25 (d, 1H) ppm.

### Example 110

### 4-(4′-{2-[4-(2,4-Dichloro-phenyl)-1-ethyl-1H-imidazol-2-yl]-(E)-vinyl}-biphenyl-4-yloxy)-butyric acid

4-(4′-{2-[4-(2,4-Dichloro-phenyl)-1-ethyl-1H-imidazol-2-yl]-(E)-vinyl}-biphenyl-4-yloxy)-butyric acid methyl ester (54 mg, 0.1 mmol) was treated as described in general procedure F to give 4-(4′-{2-[4-(2,4-dichloro-phenyl)-1-ethyl-1H-imidazol-2-yl]-(E)-vinyl}-biphenyl-4-yloxy)-butyric acid (45 mg, 86% yield).

LCMS: m/z 521 (M+H)⁺; ¹H NMR (DMSO-d₆, 400 MHz): δ 1.37 (t, 3H), 1.96 (m, 2H), 2.41 (t, 2H), 4.04 (t, 2H), 4.27 (q, 2H), 7.04 (d, 2H), 7.32 (d, 1H), 7.50 (dd, 1H), 7.57 (d, 1H), 7.64-7.67 (m, 5H), 7.79 (d, 2H), 7.96 (s, 1H), 8.25 (d, 1H) ppm.

### Example 111

(4′-{2-[4-(2,4-Dichloro-phenyl)-1-ethyl-1H-imidazol-2-yl]-(E)-vinyl}-biphenyl-4-yloxy)-phenyl-acetic acid

4′-{2-[4-(2,4-Dichloro-phenyl)-1-ethyl-1H-imidazol-2-yl]-(E)-vinyl}-biphenyl-4-ol (44 mg, 0.1 mmol) was treated with methyl α-bromophenylacetate according to the general procedure E followed by ester hydrolysis according to the general procedure F to give (4′-{2-[4-(2,4-dichloro-phenyl)-1-ethyl-1H-imidazol-2-yl]-(E)-vinyl}-biphenyl-4-yloxy)-phenyl-acetic acid (21 mg, 37% yield).

LCMS: m/z 569 (M+H)⁺; ¹H NMR (CD₃OD, 400 MHz): δ 1.50 (t, 3H), 4.35 (q, 2H), 5.79 (s, 1H), 6.94 (d, 1H), 6.99 (d, 2H), 7.32 (m, 1H), 7.42 (d, 1H), 7.49 (m, 5H), 7.55-7.63 (m, 6H), 7.67 (s, 1H), 7.73 (d, 1H), 8.25 (d, 1H) ppm.

### Example 112

### 5-[3-(4′-{2-[4-(2,4-Dichloro-phenyl)-1-ethyl-1H-imidazol-2-yl]-(E)-vinyl}-biphenyl-4-yloxy)-propyl]-1H-tetrazole

4′-{2-[4-(2,4-Dichloro-phenyl)-1-ethyl-1H-imidazol-2-yl]-(E)-vinyl}-biphenyl-4-ol (44 mg, 0.1 mmol) was treated with 4-bromobutyronitrile as described in the general procedure E followed by tetrazole formation as described in the general procedure M to give 5-[3-(4′-{2-[4-(2,4-dichloro-phenyl)-1-ethyl-1H-imidazol-2-yl]-(E)-vinyl}-biphenyl-4-yloxy)-propyl]-1H-tetrazole (22 mg, 41% total yield).

LCMS: m/z 545 (M+H)⁺; ¹H NMR (CDCl₃, 400 MHz): δ 1.22 (t, 3H), 2.08 (m, 2H), 2.55 (t, 2H), 3.95 (q, 2H), 4.09 (t, 2H), 6.94 (d, 1H), 6.97 (d, 2H), 7.12 (s, 1H), 7.41 (d, 1H), 7.47-7.57 (m, 6H), 7.62 (s, 1H), 7.78 (d, 1H), 8.14 (d, 1H) ppm.

### Example 113

### 5-[4-(4′-{2-[4-(2,4-Dichloro-phenyl)-1-ethyl-1H-imidazol-2-yl]-(E)-vinyl}-biphenyl-4-yloxymethyl)-phenyl]-1H-tetrazole

4′-{2-[4-(2,4-Dichloro-phenyl)-1-ethyl-1H-imidazol-2-yl]-(E)-vinyl}-biphenyl-4-ol (44 mg, 0.1 mmol) was treated with α-bromo-p-tolunitrile as described in the general procedure E followed by tetrazole formation as described in the general procedure M to give 5-[4-(4′-{2-[4-(2,4-dichloro-phenyl)-1-ethyl-1H-imidazol-2-yl]-(E)-vinyl}-biphenyl-4-yloxymethyl)-phenyl]-1H-tetrazole (22 mg, 37% total yield).

LCMS: m/z 593 (M+H)⁺.

### Example 114

### 5-[4-(4′-{2-[4-(2,4-Dichloro-phenyl)-1-ethyl-1H-imidazol-2-yl]-(E)-vinyl}-biphenyl-4-yloxy)-phenyl]-1H-tetrazole

4′-{2-[4-(2,4-Dichloro-phenyl)-1-ethyl-1H-imidazol-2-yl]-(E)-vinyl}-biphenyl-4-ol (44 mg, 0.1 mmol) was treated with 4-iodobenzonitrile as described in the general procedure J followed by tetrazole formation as described in the general procedure M to give 5-[4-(4′-{2-[4-(2,4-dichloro-phenyl)-1-ethyl-1H-imidazol-2-yl]-(E)-vinyl}-biphenyl-4-yloxy)-phenyl]-1H-tetrazole (13 mg, 22% total yield).

LCMS: m/z 579 (M+H)⁺; ¹H NMR (CD₃OD, 400 MHz): δ 1.37 (t, 3H), 4.30 (q, 2H), 7.06 (dd, 1H), 7.24 (d, 2H), 7.32 (d, 1H), 7.37 (d, 1H), 7.52 (dd, 1H), 7.62 (d, 1H), 7.67 (d, 1H), 7.74 (d, 2H), 7.79-7.86 (m, 6H), 7.99 (s, 1H), 8.17 (d, 1H) ppm.

### Example 115

### 2-[2-(5-Bromo-2-methoxy-phenyl)-(E)-vinyl]-4-(2,4-dichloro-phenyl)-1H-imidazole

Trans-5-bromo-2-methoxycinnamic acid (257 mg, 1 mmol) was treated according to general procedure A using 2,4-dichlorophenacyl bromide to give 2-[2-(5-bromo-2-methoxy-phenyl)-(E)-vinyl]-4-(2,4-dichloro-phenyl)-1H-imidazole (195 mg, 46% yield).

LCMS: m/z 424 (M+H)⁺; ¹H NMR (CD₃OD, 400 MHz): δ 3.98 (s, 3H), 6.99 (d, 1H), 7.26 (d, 1H), 7.49-7.56 (m, 2H), 7.61-7.66 (m, 2H), 7.75 (d, 1H), 7.79 (s, 1H), 7.95 (d, 1H) ppm.

### Example 116

### 4-(2,4-Dichloro-phenyl)-2-{2-[2-methoxy-5-(4-methoxy-phenylethynyl)-phenyl]-(E)-vinyl}-1H-imidazole

2-[2-(5-Bromo-2-methoxy-phenyl)-(E)-vinyl]-4-(2,4-dichloro-phenyl)-1H-imidazole (43 mg, 0.1 mmol) was treated as described in general procedure H using 1-ethynyl-4-methoxybenzene to give 4-(2,4-dichloro-phenyl)-2-{2-[2-methoxy-5-(4-methoxy-phenylethynyl)-phenyl]-(E)-vinyl}-1H-imidazole (19 mg, 39% yield).

LCMS: m/z 475 (M+H)⁺; ¹H NMR (CD₃OD, 400 MHz): δ 3.81 (s, 3H), 3.88 (s, 3H), 6.94 (dd, 1H), 6.99 (d, 1H), 7.07 (m, 1H), 7.11 (d, 1H), 7.16 (d, 1H), 7.26 (m, 2H), 7.35 (dd, 1H), 7.44-7.48 (m, 2H), 7.63 (s, 1H), 7.72 (d, 1H), 7.83 (d, 1H) ppm.

### Example 117

### [4-(3-{2-[4-(2,4-Dichloro-phenyl)-1H-imidazol-2-yl]-(E)-vinyl}-4-methoxy-phenylethynyl)-phenoxy]-acetic acid methyl ester

2-[2-(5-Bromo-2-methoxy-phenyl)-(E)-vinyl]-4-(2,4-dichloro-phenyl)-1H-imidazole (43 mg, 0.1 mmol) was treated as described in general procedure H using 4-(methoxy-carbonyl-methoxy)-phenylacetylene to give [4-(3-{2-[4-(2,4-dichloro-phenyl)-1H-imidazol-2-yl]-(E)-vinyl}-4-methoxy-phenylethynyl)-phenoxy]-acetic acid methyl ester (26 mg, 49% yield).

LCMS: m/z 533 (M+H)⁺; ¹H NMR (CD₃OD, 400 MHz): δ 3.78 (s, 3H), 3.98 (s, 3H), 4.50 (s, 2H), 6.94 (dd, 1H), 6.99 (d, 1H), 7.07 (m, 1H), 7.11 (d, 1H), 7.16 (d, 1H), 7.26 (m, 2H), 7.35 (dd, 1H), 7.44-7.48 (m, 2H), 7.63 (s, 1H), 7.72 (d, 1H), 7.83 (d, 1H) ppm.

### Example 118

### [4-(3-{2-[4-(2,4-Dichloro-phenyl)-1H-imidazol-2-yl]-(E)-vinyl}-4-methoxy-phenylethynyl)-phenoxy]-acetic acid

[4-(3-{2-[4-(2,4-Dichloro-phenyl)-1H-imidazol-2-yl]-(E)-vinyl}-4-methoxy-phenylethynyl)-phenoxy]-acetic acid methyl ester (20 mg, 0.037 mmol) was treated as described in general procedure F to give [4-(3-{2-[4-(2,4-dichloro-phenyl)-1H-imidazol-2-yl]-(E)-vinyl}-4-methoxy-phenyl-ethynyl)-phenoxy]-acetic acid (17 mg, 88% yield).

LCMS: m/z 519 (M+H)⁺; ¹H NMR (CD₃OD, 400 MHz): δ 3.97 (s, 3H), 4.51 (s, 2H), 6.94 (dd, 1H), 6.99 (d, 1H), 7.07 (m, 1H), 7.11 (d, 1H), 7.16 (d, 1H), 7.26 (m, 2H), 7.35 (dd, 1H), 7.44-7.49 (m, 2H), 7.64 (s, 1H), 7.74 (d, 1H), 7.85 (d, 1H) ppm.

### Example 119

### [3-(3-{2-[4-(2,4-Dichloro-phenyl)-1H-imidazol-2-yl]-(E)-vinyl}-4-methoxy-phenylethynyl)-phenoxy]-acetic acid

2-[2-(5-Bromo-2-methoxy-phenyl)-(E)-vinyl]-4-(2,4-dichloro-phenyl)-1H-imidazole (43 mg, 0.1 mmol) was treated with 3-(methoxy-carbonyl-methoxy)-phenyl acetylene as described in general procedure H followed by ester hydrolysis as described in general procedure F to give [3-(3-{2-[4-(2,4-dichloro-phenyl)-1H-imidazol-2-yl]-(E)-vinyl}-4-methoxy-phenylethynyl)-phenoxy]-acetic acid (15 mg, 29% yield).

LCMS: m/z 519 (M+H)⁺; ¹H NMR (CD₃OD, 400 MHz): δ 3.81 (s, 3H), 4.59 (s, 2H), 6.94 (dd, 1H), 6.99 (d, 1H), 7.07 (m, 1H), 7.11 (d, 1H), 7.16 (d, 1H), 7.26 (m, 2H), 7.35 (dd, 1H), 7.44-7.48 (m, 2H), 7.63 (s, 1H), 7.72 (d, 1H), 7.83 (d, 1H) ppm.

### Example 120

### [4-(3-{2-[4-(2,4-Dichloro-phenyl)-1-methyl-1H-imidazol-2-yl]-(E)-vinyl}-4-methoxy-phenylethynyl)-phenoxy]1-acetic acid

[4-(3-{2-[4-(2,4-Dichloro-phenyl)-1H-imidazol-2-yl]-(E)-vinyl}-4-methoxy-phenylethynyl)-phenoxy]-acetic acid methyl ester (25 mg, 0.05 mmol) was treated with methyl iodide as described in general procedure E followed by ester hydrolysis as described in general procedure F to give [4-(3-{2-[4-(2,4-dichloro-phenyl)-1-methyl-1H-imidazol-2-yl]-(E)-vinyl}-4-methoxy-phenylethynyl)-phenoxy]-acetic acid (18 mg, 68% yield).

LCMS: m/z 533 (M+H)⁺; ¹H NMR (CD₃OD, 400 MHz): δ 3.84 (s, 3H), 3.87 (s, 3H), 4.69 (s, 2H), 6.94 (dd, 1H), 6.99 (d, 1H), 7.07 (m, 1H), 7.11 (d, 1H), 7.16 (d, 1H), 7.26 (m, 2H), 7.35 (dd, 1H), 7.44-7.49 (m, 2H), 7.64 (s, 1H), 7.74 (d, 1H), 7.85 (d, 1H) ppm.

### Example 121

### 4-[4-(3-{2-[4-(2,4-Dichloro-phenyl)-1H-imidazol-2-yl]-(E)-vinyl}-4-methoxy-phenylethynyl)-phenoxy]-butyric acid

2-[2-(5-Bromo-2-methoxy-phenyl)-(E)-vinyl]-4-(2,4-dichloro-phenyl)-1H-imidazole (43 mg, 0.1 mmol) was treated as described in general procedure H using 4-(4-methoxy-carbonyl-propyloxy)-phenyl acetylene followed by ester hydrolysis as described in general procedure F to give 4-[4-(3-{2-[4-(2,4-dichloro-phenyl)-1H-imidazol-2-yl]-(E)-vinyl}-4-methoxy-phenylethynyl)-phenoxy]-butyric acid (16 mg, 29% yield).

LCMS: m/z 547 (M+H)⁺; ¹H NMR (CD₃OD, 400 MHz): δ 2.18 (m, 2H), 2.53 (t, 2H), 3.80 (s, 3H), 4.10 (t, 2H), 6.95 (d, 1H), 6.97 (d, 2H), 7.13 (s, 1H), 7.42 (d, 1H), 7.47-7.59 (m, 5H), 7.64 (s, 1H), 7.78 (d, 1H), 8.19 (d, 1H) ppm.

### Example 122

### 4-[3-(4-{2-[4-(2,4-Dichloro-phenyl)-1H-imidazol-2-yl]-(E)-vinyl}-phenylethynyl)-phenoxy]-butyric acid

2-[2-(4-Bromo-phenyl)-(E)-vinyl]-4-(2,4-dichloro-phenyl)-1H-imidazole (40 mg, 0.1 mmol) was treated as described in general procedure H using 3-(4-methoxy-carbonyl-propyloxy)-phenyl acetylene followed by ester hydrolysis as described in general procedure F to give 4-[3-(4-{2-[4-(2,4-dichloro-phenyl)-1H-imidazol-2-yl]-(E)-vinyl}-phenylethynyl)-phenoxy]-butyric acid (14 mg, 27% yield).

LCMS: m/z 517 (M+H)⁺; ¹H NMR (CD₃OD, 400 MHz): δ 2.12 (m, 2H), 2.53 (t, 2H), 4.08 (t, 2H), 6.93 (m, 1H), 7.06-7.13 (m, 3H), 7.27 (m, 1H), 7.36 (dd, 1H), 7.38 (d, 1H), 7.49 (d, 1H), 7.52-7.58 (m, 4H), 7.65 (s, 1H), 7.85 (d, 1H) ppm.

### Example 123

### 4-[4-(4-{2-[4-(2,4-Dichloro-phenyl)-1H-imidazol-2-yl]-(E)-vinyl}-phenylethynyl)-phenoxy]-butyric acid

2-[2-(4-Bromo-phenyl)-(E)-vinyl]-4-(2,4-dichloro-phenyl)-1H-imidazole (40 mg, 0.1 mmol) was treated as described in general procedure H using 4-(4-methoxy-carbonyl-propyloxy)-phenylacetylene followed by ester hydrolysis as described in general procedure F to give 4-[4-(4-{2-[4-(2,4-dichloro-phenyl)-1H-imidazol-2-yl]-(E)-vinyl}-phenylethynyl)-phenoxy]-butyric acid (15 mg, 29% yield).

LCMS: m/z 517 (M+H)⁺; ¹H NMR (CDCl₃, 400 MHz): δ 2.18 (m, 2H), 2.53 (t, 2H), 4.10 (t, 2H), 6.95 (d, 1H), 6.97 (d, 2H), 7.13 (s, 1H), 7.42 (d, 1H), 7.47-7.59 (m, 6H), 7.64 (s, 1H), 7.78 (d, 1H), 8.19 (d, 1H) ppm.

### Example 124

### 4-(4′-{2-[4-(2,4-Dichloro-phenyl)-1-methyl-1H-imidazol-2-yl]-(E)-vinyl}-biphenyl-4-yloxy)-butyric acid methyl ester

4-(2,4-Dichloro-phenyl)-2-[2-(4′-methoxy-biphenyl-4-yl)-(E)-vinyl]-1-methyl-1H-imidazole (44 mg, 0.1 mmol) was demethylated as described in general procedure C and the resulting phenol intermediate was treated with methyl 4-bromobutyrate as described in the general procedure E to give 4-(4′-{2-[4-(2,4-dichloro-phenyl)-1-methyl-1H-imidazol-2-yl]-(E)-vinyl}-biphenyl-4-yloxy)-butyric acid methyl ester (32 mg, 61% total yield).

LCMS: m/z 521 (M+H)⁺; ¹H NMR (CD₃OD, 400 MHz): δ 2.15 (m, 2H), 2.56 (t, 2H), 3.78 (s, 3H), 3.86 (s, 3H), 4.09 (t, 2H), 7.00 (d, 2H), 7.06 (d, 1H), 7.35 (dd, 1H), 7.48 (d, 1H), 7.55-7.67 (m, 8H), 8.01 (d, 1H) ppm.

### Example 125

### 4-(4′-{2-[4-(2,4-Dichloro-phenyl)-1-methyl-1H-imidazol-2-yl]-(E)-vinyl}-biphenyl-4-yloxy)-butyric acid

4-(4′-{2-[4-(2,4-Dichloro-phenyl)-1-methyl-1H-imidazol-2-yl]-(E)-vinyl}-biphenyl-4-yloxy)-butyric acid methyl ester (26 mg, 0.05 mmol) was treated as described in general procedure F to give 4-(4′-{2-[4-(2,4-dichloro-phenyl)-1-methyl-1H-imidazol-2-yl]-(E)-vinyl}-biphenyl-4-yloxy)-butyric acid (21 mg, 84% yield).

LCMS: m/z 507 (M+H)⁺; ¹H NMR (CD₃OD, 400 MHz): δ 2.14 (m, 2H), 2.55 (t, 2H), 3.87 (s, 3H), 4.09 (t, 2H), 7.00 (d, 2H), 7.06 (d, 1H), 7.35 (dd, 1H), 7.47 (d, 1H), 7.56-7.66 (m, 8H), 7.99 (d, 1H) ppm.

### Example 126

### 5-[3-(4′-{2-[4-(2,4-Dichloro-phenyl)-1-methyl-1H-imidazol-2-yl]-(E)-vinyl}-biphenyl-4-yloxy)-propyl]-1H-tetrazole

4-(2,4-Dichloro-phenyl)-2-[2-(4′-methoxy-biphenyl-4-yl)-(E)-vinyl]-1-methyl-1H-imidazole (44 mg, 0.1 mmol) was demethylated as described in general procedure C and the resulting phenol intermediate was treated with 4-bromobutyronitrile as described in the general procedure E followed by tetrazole formation as described in the general procedure L to give 5-[3-(4′-{2-[4-(2,4-dichloro-phenyl)-1-methyl-H-imidazol-2-yl]-(E)-vinyl}-biphenyl-4-yloxy)-propyl]-1H-tetrazole (11 mg, 21% total yield).

LCMS: m/z 531 (M+H)⁺; ¹H NMR (CD₃OD, 400 MHz): δ 2.15 (m, 2H), 2.56 (t, 2H), 3.86 (s, 3H), 4.09 (t, 2H), 7.00 (d, 2H), 7.06 (d, 1H), 7.35 (dd, 1H), 7.48 (d, 1H), 7.55-7.67 (m, 8H), 8.01 (d, 1H) ppm.

### Example 127

(4′-{2-[4-(2,4-Dichloro-phenyl)-1-methyl-1H-imidazol-2-yl]-(E)-vinyl}-biphenyl-4-yloxy)-acetic acid

4-(2,4-Dichloro-phenyl)-2-[2-(4′-methoxy-biphenyl-4-yl)-(E)-vinyl]-1-methyl-1H-imidazole (44 mg, 0.1 mmol) was demethylated as described in general procedure C and the resulting phenol intermediate was treated with methyl bromoacetate as described in the general procedure E followed by ester hydrolysis as described in the general procedure F to give (4′-{2-[4-(2,4-dichloro-phenyl)-1-methyl-1H-imidazol-2-yl]-(E)-vinyl}-biphenyl-4-yloxy)-acetic acid (32 mg, 61% total yield).

LCMS: m/z 479 (M+H)⁺; ¹H NMR (CD₃OD, 400 MHz): δ 3.87 (s, 3H), 4.81 (s, 2H), 7.00 (d, 2H), 7.06 (d, 1H), 7.35 (dd, 1H), 7.47 (d, 1H), 7.56-7.66 (m, 8H), 7.99 (d, 1H) ppm.

### Example 128

### 5-(4′-{2-[4-(2,4-Dichloro-phenyl)-1-methyl-1H-imidazol-2-yl]-(E)-vinyl}-biphenyl-4-yloxy)-pentanoic acid methyl ester

4-(2,4-Dichloro-phenyl)-2-[2-(4′-methoxy-biphenyl-4-yl)-(E)-vinyl]-1-methyl-1H-imidazole (44 mg, 0.1 mmol) was treated as described in general procedure C to give the phenolic intermediate. The intermediate was treated with methyl 5-bromovalerate following the general procedure E to give 5-(4′-{2-[4-(2,4-dichloro-phenyl)-1-methyl-1H-imidazol-2-yl]-(E)-vinyl}-biphenyl-4-yloxy)-pentanoic acid methyl ester (31 mg, 58% total yield).

LCMS: m/z 535 (M+H)⁺; ¹H NMR (DMSO-d₆, 400 MHz): δ 1.69 (m, 2H), 1.77 (m, 2H), 2.31 (t, 2H), 3.74 (s, 3H), 3.86 (s, 3H), 4.02 (t, 2H), 7.00 (d, 2H), 7.06 (d, 1H), 7.35 (dd, 1H), 7.48 (d, 1H), 7.55-7.67 (m, 8H), 8.01 (d, 1H) ppm.

### Example 129

### 5-(4′-{2-[4-(2,4-Dichloro-phenyl)-1-methyl-1H-imidazol-2-yl]-(E)-vinyl}-biphenyl-4-yloxy)-pentanoic acid

5-(4′-{2-[4-(2,4-Dichloro-phenyl)-1-methyl-1H-imidazol-2-yl]-(E)-vinyl}-biphenyl-4-yloxy)-pentanoic acid methyl ester (27 mg, 0.05 mmol) was treated as described in general procedure F to give 5-(4′-{2-[4-(2,4-dichloro-phenyl)-1-methyl-1H-imidazol-2-yl]-(E)-vinyl}-biphenyl-4-yloxy)-pentanoic acid (21 mg, 82% yield).

LCMS: m/z 521 (M+H)⁺; ¹H NMR (DMSO-d₆, 400 MHz): δ 1.67 (m, 2H), 1.74 (m, 2H), 2.30 (t, 2H), 3.85 (s, 3H), 4.02 (t, 2H), 7.02 (d, 2H), 7.31 (d, 1H), 7.49 (dd, 1H), 7.57 (d, 1H), 7.63-7.67 (m, 5H), 7.78 (d, 2H), 7.96 (s, 1H), 8.25 (d, 1H) ppm.

### Example 130

### 4-(4′-{2-[4-(2,4-Dichloro-phenyl)-1-methyl-1H-imidazol-2-yl]-(E)-vinyl}-biphenyl-4-yloxymethyl)-benzoic acid

4-(2,4-Dichloro-phenyl)-2-[2-(4′-methoxy-biphenyl-4-yl)-(E)-vinyl]-1-methyl-1H-imidazole (44 mg, 0.1 mmol) was demethylated as described in general procedure C and the resulting phenol intermediate was treated with methyl 4-(bromomethyl)benzoate as described in the general procedure E followed by ester hydrolysis as described in the general procedure F to give 4-(4′-{2-[4-(2,4-dichloro-phenyl)-1-methyl-1H-imidazol-2-yl]-(E)-vinyl}-biphenyl-4-yloxymethyl)-benzoic acid (25 mg, 44% total yield).

LCMS: m/z 555 (M+H)⁺; ¹H NMR (CD₃OD, 400 MHz): δ 3.87 (s, 3H), 5.25 (s, 2H), 7.00 (d, 2H), 7.06 (d, 1H), 7.27 (d, 2H), 7.35 (dd, 1H), 7.47 (d, 1H), 7.56-7.66 (m, 8H), 7.74 (d, 2H), 7.99 (d, 1H) ppm.

### Example 131

### 2-Bromo-4-(4′-{2-[4-(2,4-dichloro-phenyl)-1-methyl-1H-imidazol-2-yl]-(E)-vinyl}-biphenyl-4-yloxy)-benzoic acid

4-(2,4-Dichloro-phenyl)-2-[2-(4′-methoxy-biphenyl-4-yl)-(E)-vinyl]-1-methyl-1H-imidazole (44 mg, 0.1 mmol) was demethylated as described in general procedure C and the resulting phenol intermediate was treated with methyl methyl 4-fluoro-2-bromobenzoate as described in the general procedure E followed by ester hydrolysis as described in the general procedure F to give 2-bromo-4-(4′-{2-[4-(2,4-dichloro-phenyl)-1-methyl-1H-imidazol-2-yl]-(E)-vinyl}-biphenyl-4-yloxy)-benzoic acid (24 mg, 39% total yield).

LCMS: m/z 620 (M+H)⁺; ¹H NMR (CD₃OD, 400 MHz): δ 3.87 (s, 3H), 7.00 (d, 2H), 7.06 (d, 1H), 7.27 (d, 2H), 7.35 (dd, 1H), 7.47 (d, 1H), 7.56-7.66 (m, 7H), 7.74 (d, 2H), 8.02 (d, 1H) ppm.

### Example 132

### 4-(4′-{2-[4-(2,4-Dichloro-phenyl)-1-(2,2,2-trifluoro-ethyl)-1H-imidazol-2-yl]-(E)-vinyl}-biphenyl-4-yloxy)-butyric acid

2-[2-(4-Bromo-phenyl)-(E)-vinyl]-4-(2,4-dichloro-phenyl)-1H-imidazole (79 mg, 0.2 mmol) was treated with 1-iodo-2,2,2-trifluoroethane as described in general procedure E followed by Suzuki coupling with 4-methoxybenzeneboronic acid as described in general procedure B. The resulting intermediate was demethylated as described in general procedure C, treated with methyl 4-bromobutyrate as described in general procedure E followed by ester hydrolysis as described in general procedure F to give 4-(4′-{2-[4-(2,4-dichloro-phenyl)-1-(2,2,2-trifluoro-ethyl)-1H-imidazol-2-yl]-(E)-vinyl}-biphenyl-4-yloxy)-butyric acid (19 mg, 16% yield).

LCMS: m/z 575 (M+H)⁺; ¹H NMR (DMSO-d₆, 400 MHz): δ 1.96 (m, 2H), 2.41 (t, 2H), 4.04 (t, 2H), 4.72 (q, 2H), 7.04 (d, 2H), 7.32 (d, 1H), 7.50 (dd, 1H), 7.57 (d, 1H), 7.64-7.67 (m, 5H), 7.79 (d, 2H), 7.96 (s, 1H), 8.27 (d, 1H) ppm.

### Example 133

### 4-(4′-{2-[4-(2,4-Dichloro-phenyl)-1-ethyl-1H-imidazol-2-yl]-(E)-vinyl}-biphenyl-4-ylamino)-butyric acid

2-[2-(4-Bromo-phenyl)-(E)-vinyl]-4-(2,4-dichloro-phenyl)-1-ethyl-1H-imidazole (43 mg, 0.1 mmol) was treated with 4-aminobenzeneboronic acid as described in general procedure B. The resulting intermediate was treated with methyl 4-bromobutyrate as described in general procedure E followed by ester hydrolysis as described in general procedure F to give 4-(4′-{2-[4-(2,4-dichloro-phenyl)-1-ethyl-1H-imidazol-2-yl]-(E)-vinyl}-biphenyl-4-ylamino)-butyric acid (19 mg, 36% total yield).

LCMS: m/z 520 (M+H)⁺; ¹H NMR (DMSO-d₆, 400 MHz): δ 1.40 (t, 3H), 1.96 (m, 2H), 2.41 (t, 2H), 4.04 (m, 2H), 4.36 (q, 2H), 7.04 (d, 2H), 7.32 (d, 1H), 7.50 (dd, 1H), 7.57 (d, 1H), 7.64-7.67 (m, 5H), 7.79 (d, 2H), 7.96 (s, 1H), 8.27 (d, 1H) ppm.

### Example 134

### N-(4′-{2-[4-(2,4-Dichloro-phenyl)-1-ethyl-1H-imidazol-2-yl]-(E)-vinyl}-biphenyl-4-yl)-succinamic acid

2-[2-(4-Bromo-phenyl)-(E)-vinyl]-4-(2,4-dichloro-phenyl)-1-ethyl-1H-imidazole (43 mg, 0.1 mmol) was treated with 4-aminobenzeneboronic acid as described in general procedure B. The resulting intermediate was heated in anhydrous DMF (0.1-0.5 M) with 2 equivalents of succinic anhydride and 2 equivalents of DIEA at 100° C. for 2 hours. At completion, the reaction mixture was worked up with EtOAc and water. The combined organic layer was washed, condensed and purified by silica gel chromatography to afford N-(4′-{2-[4-(2,4-dichloro-phenyl)-1-ethyl-1H-imidazol-2-yl]-(E)-vinyl}-biphenyl-4-yl)-succinamic acid (18 mg, 33% total yield).

LCMS: m/z 534 (M+H)⁺; ¹H NMR (DMSO-d₆, 400 MHz): δ 1.40 (t, 3H), 2.45-2.58 (m, 4H), 4.36 (q, 2H), 7.04 (d, 2H), 7.32 (d, 1H), 7.50 (dd, 1H), 7.57 (d, 1H), 7.64-7.67 (m, 5H), 7.79 (d, 2H), 7.96 (s, 1H), 8.27 (d, 1H) ppm.

### Example 135

### 4-(4′-{2-[4-(2,4-Dichloro-phenyl)-1-ethyl-1H-imidazol-2-yl]-(E)-vinyl}-biphenyl-4-yloxymethyl)-benzoic acid

4′-{2-[4-(2,4-Dichloro-phenyl)-1-ethyl-1H-imidazol-2-yl]-(E)-vinyl}-biphenyl-4-ol (44 mg, 0.1 mmol) was treated with methyl 4-(bromomethyl)benzoate as described in the general procedure E followed by ester hydrolysis as described in the general procedure F to give 4-(4′-{2-[4-(2,4-dichloro-phenyl)-1-ethyl-1H-imidazol-2-yl]-(E)-vinyl}-biphenyl-4-yloxymethyl)-benzoic acid (31 mg, 54% total yield).

LCMS: m/z 569 (M+H)⁺; ¹H NMR (DMSO-d₆, 400 MHz): δ 1.37 (t, 3H), 4.30 (q, 2H), 5.25 (s, 2H), 7.06 (dd, 1H), 7.24 (d, 2H), 7.32 (d, 1H), 7.37 (d, 1H), 7.52 (dd, 1H), 7.62 (d, 1H), 7.67 (d, 1H), 7.74 (d, 2H), 7.79-7.86 (m, 6H), 7.99 (s, 1H), 8.17 (d, 1H) ppm.

### Example 136

### [4-(4′-{2-[4-(2,4-Dichloro-phenyl)-1-ethyl-1H-imidazol-2-yl]-(E)-vinyl}-biphenyl-4-yloxymethyl)-phenyl]-acetic acid

4′-{2-[4-(2,4-Dichloro-phenyl)-1-ethyl-1H-imidazol-2-yl]-(E)-vinyl}-biphenyl-4-ol (44 mg, 0.1 mmol) was treated with methyl 4-(bromomethyl)phenylacetate as described in the general procedure E followed by ester hydrolysis as described in the general procedure F to give [4-(4′-{2-[4-(2,4-dichloro-phenyl)-1-ethyl-1H-imidazol-2-yl]-(E)-vinyl}-biphenyl-4-yloxymethyl)-phenyl]-acetic acid (22 mg, 37% total yield).

LCMS: m/z 583 (M+H)⁺; ¹H NMR (DMSO-d₆, 400 MHz): δ 1.39 (t, 3H), 3.21 (s, 2H), 4.32 (q, 2H), 5.25 (s, 2H), 7.06 (dd, 1H), 7.24 (d, 2H), 7.32 (d, 1H), 7.37 (d, 1H), 7.52 (dd, 1H), 7.62 (d, 1H), 7.67 (d, 1H), 7.74 (d, 2H), 7.79-7.86 (m, 6H), 7.99 (s, 1H), 8.19 (d, 1H) ppm.

### Example 137

### 4-(4′-{2-[4-(2,4-Dichloro-phenyl)-1-ethyl-1H-imidazol-2-yl]-(E)-vinyl}-biphenyl-4-yloxy)-benzoic acid methyl ester

4′-{2-[4-(2,4-Dichloro-phenyl)-1-ethyl-1H-imidazol-2-yl]-(E)-vinyl}-biphenyl-4-ol (44 mg, 0.1 mmol) was treated as described in general procedure J using methyl 4-iodobenzoate to give 4-(4′-{2-[4-(2,4-dichloro-phenyl)-1-ethyl-1H-imidazol-2-yl]-(E)-vinyl}-biphenyl-4-yloxy)-benzoic acid methyl ester (26 mg, 46% yield).

LCMS: m/z 569 (M+H)⁺; ¹H NMR (DMSO-d₆, 400 MHz): δ 1.40 (t, 3H), 3.81 (s, 3H), 4.31 (q, 2H), 7.07 (dd, 1H), 7.25 (d, 2H), 7.33 (d, 1H), 7.38 (d, 1H), 7.52 (dd, 1H), 7.63 (d, 1H), 7.68 (d, 1H), 7.74 (d, 2H), 7.80-7.87 (m, 6H), 8.00 (s, 1H), 8.19 (d, 1H) ppm.

### Example 138

### 4-(4′-{2-[4-(2,4-Dichloro-phenyl)-1-ethyl-1H-imidazol-2-yl]-(E)-vinyl}-biphenyl-4-yloxy)-benzoic acid

4-(4′-{2-[4-(2,4-Dichloro-phenyl)-1-ethyl-1H-imidazol-2-yl]-(E)-vinyl}-biphenyl-4-yloxy)-benzoic acid methyl ester (18 mg, 0.03 mmol) was treated as described in general procedure F to give 4-(4′-{2-[4-(2,4-dichloro-phenyl)-1-ethyl-1H-imidazol-2-yl]-(E)-vinyl}-biphenyl-4-yloxy)-benzoic acid (14 mg, 81% yield).

LCMS: m/z 555 (M+H)⁺; ¹H NMR (DMSO-d₆, 400 MHz): δ 1.37 (t, 3H), 4.30 (q, 2H), 7.06 (dd, 1H), 7.24 (d, 2H), 7.32 (d, 1H), 7.37 (d, 1H), 7.52 (dd, 1H), 7.62 (d, 1H), 7.67 (d, 1H), 7.74 (d, 2H), 7.79-7.86 (m, 6H), 7.99 (s, 1H), 8.17 (d, 1H) ppm.

### Example 139

### 3-(4′-{2-[4-(2,4-Dichloro-phenyl)-1-ethyl-1H-imidazol-2-yl]-(E)-vinyl}-biphenyl-4-yloxy)-benzoic acid

4′-{2-[4-(2,4-Dichloro-phenyl)-1-ethyl-1H-imidazol-2-yl]-(E)-vinyl}-biphenyl-4-ol (44 mg, 0.1 mmol) was treated as described in general procedure J using methyl 3-iodobenzoate followed by ester hydrolysis as described in general procedure F to give 3-(4′-{2-[4-(2,4-dichloro-phenyl)-1-ethyl-1H-imidazol-2-yl]-(E)-vinyl}-biphenyl-4-yloxy)-benzoic acid (21 mg, 38% yield).

LCMS: m/z 555 (M+H)⁺; ¹H NMR (DMSO-d₆, 400 MHz): δ 1.38 (t, 3H), 4.31 (q, 2H), 7.06 (dd, 1H), 7.24 (d, 2H), 7.32 (d, 1H), 7.37 (d, 1H), 7.52 (dd, 1H), 7.62 (d, 1H), 7.67 (m, 1H), 7.74 (d, 2H), 7.81-7.89 (m, 6H), 7.99 (s, 1H), 8.17 (d, 1H) ppm.

### Example 140

### 4-(4′-{2-[4-(2,4-Dichloro-phenyl)-1-ethyl-1H-imidazol-2-yl]-(E)-vinyl}-biphenyl-4-yloxy)-2-fluoro-benzoic acid

4′-{2-[4-(2,4-Dichloro-phenyl)-1-ethyl-1H-imidazol-2-yl]-(E)-vinyl}-biphenyl-4-ol (44 mg, 0.1 mmol) was treated as described in general procedure J using methyl 2-fluoro-4-bromobenzoate followed by ester hydrolysis as described in general procedure F to give 4-(4′-{2-[4-(2,4-dichloro-phenyl)-1-ethyl-1H-imidazol-2-yl]-(E)-vinyl}-biphenyl-4-yloxy)-2-fluoro-benzoic acid (20 mg, 34% yield).

LCMS: m/z 573 (M+H)⁺; ¹H NMR (DMSO-d₆, 400 MHz): δ 1.37 (t, 3H), 4.32 (q, 2H), 7.06 (dd, 1H), 7.24 (d, 2H), 7.32 (d, 1H), 7.37 (d, 1H), 7.52 (dd, 1H), 7.62 (d, 1H), 7.67 (m, 1H), 7.74 (d, 2H), 7.81-7.89 (m, 5H), 8.01 (s, 1H), 8.19 (d, 1H) ppm.

### Example 141

### 4-(4′-{2-[4-(2,4-Dichloro-phenyl)-1-ethyl-1H-imidazol-2-yl]-(E)-vinyl}-biphenyl-4-yloxy)-2-methyl-benzoic acid

4′-{2-[4-(2,4-Dichloro-phenyl)-1-ethyl-1H-imidazol-2-yl]-(E)-vinyl}-biphenyl-4-ol (44 mg, 0.1 mmol) was treated as described in general procedure J using methyl 4-bromo-2-methyl-benzoate followed by ester hydrolysis as described in general procedure F to give 4-(4′-{2-[4-(2,4-dichloro-phenyl)-1-ethyl-1H-imidazol-2-yl]-(E)-vinyl}-biphenyl-4-yloxy)-2-methyl-benzoic acid (17 mg, 30% yield).

LCMS: m/z 569 (M+H)⁺; ¹H NMR (DMSO-d₆, 400 MHz): δ 1.37 (t, 3H), 2.39 (s, 3H), 4.31 (q, 2H), 7.06 (dd, 1H), 7.24 (d, 2H), 7.32 (d, 1H), 7.37 (d, 1H), 7.52 (dd, 1H), 7.62 (d, 1H), 7.67 (m, 1H), 7.74 (d, 2H), 7.80-7.87 (m, 5H), 7.99 (s, 1H), 8.14 (d, 1H) ppm.

### Example 142

### 5-(4′-{2-[4-(2,4-Dichloro-phenyl)-1-ethyl-1H-imidazol-2-yl]-(E)-vinyl}-biphenyl-4-yloxy)-furan-2-carboxylic acid methyl ester

4′-{2-[4-(2,4-Dichloro-phenyl)-1-ethyl-1H-imidazol-2-yl]-(E)-vinyl}-biphenyl-4-ol (44 mg, 0.1 mmol) was treated with 5-bromofuroic acid methyl ester as described in general procedure J to give 5-(4′-{2-[4-(2,4-dichloro-phenyl)-1-ethyl-1H-imidazol-2-yl]-(E)-vinyl}-biphenyl-4-yloxy)-furan-2-carboxylic acid methyl ester (21 mg, 38% yield).

LCMS: m/z 559 (M+H)⁺; ¹H NMR (DMSO-d₆, 400 MHz): δ 1.37 (t, 3H), 3.79 (s, 3H), 4.27 (q, 2H), 6.86 (d, 1H), 7.12 (d, 2H), 7.33 (d, 1H), 7.48 (dd, 1H), 7.57 (d, 1H), 7.63 (d, 1H), 7.68 (d, 2H), 7.74 (m, 3H), 7.82 (d, 2H), 7.95 (s, 1H), 8.24 (d, 1H) ppm.

### Example 143

### 5-(4′-{2-[4-(2,4-Dichloro-phenyl)-1-ethyl-1H-imidazol-2-yl]-(E)-vinyl}-biphenyl-4-yloxy)-furan-2-carboxylic acid

5-(4′-{2-[4-(2,4-Dichloro-phenyl)-1-ethyl-1H-imidazol-2-yl]-(E)-vinyl}-biphenyl-4-yloxy)-furan-2-carboxylic acid methyl ester (18 mg, 0.03 mmol) was treated as described in general procedure F to give 5-(4′-{2-[4-(2,4-dichloro-phenyl)-1-ethyl-1H-imidazol-2-yl]-(E)-vinyl}-biphenyl-4-yloxy)-furan-2-carboxylic acid (14 mg, 80% yield).

LCMS: m/z 545 (M+H)⁺; ¹H NMR (DMSO-d₆, 400 MHz): δ 1.35 (t, 3H), 4.26 (q, 2H), 6.85 (d, 1H), 7.12 (d, 2H), 7.32 (d, 1H), 7.48 (dd, 1H), 7.56 (d, 1H), 7.62 (d, 1H), 7.68 (d, 2H), 7.73 (m, 3H), 7.81 (d, 2H), 7.95 (s, 1H), 8.23 (d, 1H) ppm.

### Example 144

### 5-(4′-{2-[4-(2,4-Dichloro-phenyl)-1-ethyl-1H-imidazol-2-yl]-(E)-vinyl}-biphenyl-4-yloxy)-nicotinic acid

4′-{2-[4-(2,4-Dichloro-phenyl)-1-ethyl-1H-imidazol-2-yl]-(E)-vinyl}-biphenyl-4-ol (44 mg, 0.1 mmol) was treated as described in general procedure J using ethyl 5-bromonicotinate followed by ester hydrolysis as described in general procedure F to give 5-(4′-{2-[4-(2,4-dichloro-phenyl)-1-ethyl-1H-imidazol-2-yl]-(E)-vinyl}-biphenyl-4-yloxy)-nicotinic acid (13 mg, 23% yield).

LCMS: m/z 556 (M+H)⁺.

### Example 145

### 5-(4′-{2-[4-(2,4-Dichloro-phenyl)-1-ethyl-1H-imidazol-2-yl]-(E)-vinyl}-biphenyl-4-yloxy)-thiophene-2-carboxylic acid

4′-{2-[4-(2,4-Dichloro-phenyl)-1-ethyl-1H-imidazol-2-yl]-(E)-vinyl}-biphenyl-4-ol (44 mg, 0.1 mmol) was treated with methyl 5-bromothiophene-2-carboxylate as described in general procedure J followed by ester hydrolysis as described in general procedure F to give 5-(4′-{2-[4-(2,4-dichloro-phenyl)-1-ethyl-1H-imidazol-2-yl]-(E)-vinyl}-biphenyl-4-yloxy)-thiophene-2-carboxylic acid (14 mg, 25% yield).

LCMS: m/z 561 (M+H)⁺.

### Example 146

### 2-(4′-{2-[4-(2,4-Dichloro-phenyl)-1-ethyl-1H-imidazol-2-yl]-(E)-vinyl}-biphenyl-4-yloxy)-thiazole-4-carboxylic acid

4′-{2-[4-(2,4-Dichloro-phenyl)-1-ethyl-1H-imidazol-2-yl]-(E)-vinyl}-biphenyl-4-ol (44 mg, 0.1 mmol) was treated with ethyl 2-bromothiazole-4-carboxylate as described in general procedure J followed by ester hydrolysis as described in general procedure F to give 2-(4′-{2-[4-(2,4-dichloro-phenyl)-1-ethyl-1H-imidazol-2-yl]-(E)-vinyl}-biphenyl-4-yloxy)-thiazole-4-carboxylic acid (12 mg, 21% yield).

LCMS: m/z 562 (M+H)⁺; ¹H NMR (DMSO-d₆, 400 MHz): δ 1.42 (t, 3H), 4.10 (q, 2H), 6.86 (d, 2H), 7.46 (d, 1H), 7.58 (d, 2H), 7.66 (dd, 1H), 7.70 (d, 2H), 7.82 (d, 2H), 7.85-7.92 (m, 3H), 8.00 (s, 1H), 8.19 (s, 1H) ppm.

### Example 147

### 6-(4′-{2-[4-(2,4-Dichloro-phenyl)-1-ethyl-1H-imidazol-2-yl]-(E)-vinyl}-biphenyl-4-yloxy)-naphthalene-2-carboxylic acid

4′-{2-[4-(2,4-Dichloro-phenyl)-1-ethyl-1H-imidazol-2-yl]-(E)-vinyl}-biphenyl-4-ol (44 mg, 0.1 mmol) was treated with methyl 6-bromo-2-naphthoate as described in general procedure J followed by ester hydrolysis as described in general procedure F to give 6-(4′-{2-[4-(2,4-dichloro-phenyl)-1-ethyl-1H-imidazol-2-yl]-(E)-vinyl}-biphenyl-4-yloxy)-naphthalene-2-carboxylic acid (21 mg, 35% yield).

LCMS: m/z 605 (M+H)⁺; ¹H NMR (DMSO-d₆, 400 MHz): δ 1.38 (t, 3H), 4.31 (q, 2H), 7.06 (dd, 1H), 7.24 (d, 2H), 7.32 (d, 1H), 7.37 (d, 1H), 7.52 (dd, 1H), 7.62 (d, 1H), 7.67 (m, 1H), 7.74 (d, 2H), 7.73-7.89 (m, 8H), 7.99 (s, 1H), 8.17 (d, 1H) ppm.

### Example 148

### 2-(4′-{2-[4-(2,4-Dichloro-phenyl)-1-ethyl-1H-imidazol-2-yl]-(E)-vinyl}-biphenyl-4-yl)-1H-benzoimidazole-5-carboxylic acid

2-[2-(4-Bromo-phenyl)-(E)-vinyl]-4-(2,4-dichloro-phenyl)-1-ethyl-1H-imidazole (127 mg, 0.3 mmol) was treated with 4-formylphenylboronic acid as described in general procedure B. The resulting intermediate was heated in anhydrous EtOH (0.1-0.5 M) with 1.5 equivalents of methyl 3,4-diaminobenzoate at 100° C. for 5 to 6 hours. At completion, the reaction mixture was worked up with EtOAc and water. The combined organic layer was washed, condensed and purified by silica gel chromatography to afford the ester intermediate which was then hydrolyzed as described in general procedure F to afford 2-(4′-{2-[4-(2,4-dichloro-phenyl)-1-ethyl-1H-imidazol-2-yl]-(E)-vinyl}-biphenyl-4-yl)-1H-benzoimidazole-5-carboxylic acid (40 mg, 23% total yield).

LCMS: m/z 579 (M+H)⁺; ¹H NMR (CD₃OD, 400 MHz): δ 1.40 (t, 3H), 4.36 (q, 2H), 7.04 (d, 2H), 7.32 (d, 1H), 7.42-7.51 (m, 3H), 7.57 (d, 1H), 7.64-7.67 (m, 6H), 7.79 (d, 2H), 7.96 (s, 1H), 8.27 (d, 1H) ppm.

### Example 149

### 2-(4′-{2-[4-(2,4-Dichloro-phenyl)-1-ethyl-1H-imidazol-2-yl]-(E)-vinyl}-biphenyl-4-yl)-3-ethyl-3H-benzoimidazole-5-carboxylic acid

2-(4′-{2-[4-(2,4-Dichloro-phenyl)-1-ethyl-1H-imidazol-2-yl]-(E)-vinyl}-biphenyl-4-yl)-1H-benzoimidazole-5-carboxylic acid (29 mg, 0.05 mmol) was treated with 2 equivalents of ethyl bromide as described in general procedure E followed by ester hydrolysis as described in general procedure F to afford 2-(4′-{2-[4-(2,4-dichloro-phenyl)-1-ethyl-1H-imidazol-2-yl]-(E)-vinyl}-biphenyl-4-yl)-3-ethyl-3H-benzoimidazole-5-carboxylic acid (14 mg, 44% yield).

LCMS: m/z 607 (M+H)⁺; ¹H NMR (CD₃OD, 400 MHz): δ 1.43 (m, 6H), 4.35 (m, 4H), 7.04 (d, 2H), 7.32 (d, 1H), 7.42-7.51 (m, 3H), 7.57 (d, 1H), 7.64-7.67 (m, 6H), 7.79 (d, 2H), 7.96 (s, 1H), 8.25 (d, 1H) ppm.

### Example 150

### 2-(4-{2-[4-(2,4-Dichloro-phenyl)-1-ethyl-1H-imidazol-2-yl]-(E)-vinyl}-phenyl)-1H-benzoimidazole-5-carboxylic acid

Trans-4-formylcinnamic acid (88 mg, 0.5 mmol) was treated with 2,4-dichlorophenacyl bromide as described in general procedure A followed by reaction with ethyl bromide as described in general procedure E. The resulting intermediate was heated in anhydrous EtOH (0.1-0.5 M) with 1.5 equivalents of methyl-3,4-diaminobenzoate at 100° C. for 5 to 6 hours. At completion, the reaction mixture was worked up with EtOAc and water. The combined organic layer was washed, condensed and purified by silica gel chromatography to afford the ester intermediate which was then hydrolyzed as described in general procedure F to afford 2-(4-{2-[4-(2,4-dichloro-phenyl)-1-ethyl-1H-imidazol-2-yl]-(E)-vinyl}-phenyl)-1H-benzoimidazole-5-carboxylic acid (48 mg, 19% total yield).

LCMS: m/z 503 (M+H)⁺; ¹H NMR (CD₃OD, 400 MHz): δ 1.38 (t, 3H), 4.34 (q, 2H), 7.04 (d, 2H), 7.32 (d, 1H), 7.42-7.47 (m, 2H), 7.57 (d, 1H), 7.64-7.68 (m, 3H), 7.79 (d, 2H), 7.96 (s, 1H), 8.27 (d, 1H) ppm.

### Example 151

### 2-Bromo-4-(4′-{2-[4-(2,4-dichloro-phenyl)-1-ethyl-1H-imidazol-2-yl]-(E)-vinyl}-biphenyl-4-yloxy)-benzoic acid methyl ester

4′-{2-[4-(2,4-Dichloro-phenyl)-1-ethyl-1H-imidazol-2-yl]-(E)-vinyl}-biphenyl-4-ol (44 mg, 0.1 mmol) was treated as described in general procedure I using methyl 2-bromo-4-fluorobenzoate to give 2-bromo-4-(4′-{2-[4-(2,4-dichloro-phenyl)-1-ethyl-1H-imidazol-2-yl]-(E)-vinyl}-biphenyl-4-yloxy)-benzoic acid methyl ester (44 mg, 68% yield).

LCMS: m/z 648 (M+H)⁺; ¹H NMR (DMSO-d₆, 400 MHz): δ 1.40 (t, 3H), 3.82 (s, 3H), 4.29 (q, 2H), 7.07 (dd, 1H), 7.25 (d, 2H), 7.33 (d, 1H), 7.38 (d, 1H), 7.52 (dd, 1H), 7.63 (d, 1H), 7.68 (d, 1H), 7.74 (d, 2H), 7.80-7.87 (m, 5H), 8.00 (s, 1H), 8.17 (d, 1H) ppm.

### Example 152

### 2-Bromo-4-(4′-{2-[4-(2,4-dichloro-phenyl)-1-ethyl-1H-imidazol-2-yl]-(E)-vinyl}-biphenyl-4-yloxy)-benzoic acid

2-Bromo-4-(4′-{2-[4-(2,4-dichloro-phenyl)-1-ethyl-1H-imidazol-2-yl]-(E)-vinyl}-biphenyl-4-yloxy)-benzoic acid methyl ester (33 mg, 0.05 mmol) was treated as described in general procedure F to give 2-bromo-4-(4′-{2-[4-(2,4-dichloro-phenyl)-1-ethyl-1H-imidazol-2-yl]-(E)-vinyl}-biphenyl-4-yloxy)-benzoic acid (24 mg, 75% yield).

LCMS: m/z 634 (M+H)⁺; ¹H NMR (DMSO-d₆, 400 MHz): δ 1.37 (t, 3H), 4.30 (q, 2H), 7.06 (dd, 1H), 7.24 (d, 2H), 7.32 (d, 1H), 7.37 (d, 1H), 7.52 (dd, 1H), 7.62 (d, 1H), 7.67 (d, 1H), 7.74 (d, 2H), 7.80-7.86 (m, 5H), 7.99 (s, 1H), 8.17 (d, 1H) ppm.

### Example 153

### 4-(4′-{2-[4-(2,4-Dichloro-phenyl)-1-ethyl-1H-imidazol-2-yl]-(E)-vinyl}-biphenyl-4-yloxy)-2-trifluoromethyl-benzoic acid methyl ester

4′-{2-[4-(2,4-Dichloro-phenyl)-1-ethyl-1H-imidazol-2-yl]-(E)-vinyl}-biphenyl-4-ol (44 mg, 0.1 mmol) was treated as described in general procedure I using methyl 4-fluoro-2-(trifluoromethyl)benzoate to give 4-(4′-{2-[4-(2,4-dichloro-phenyl)-1-ethyl-1H-imidazol-2-yl]-(E)-vinyl}-biphenyl-4-yloxy)-2-trifluoromethyl-benzoic acid methyl ester (46 mg, 73% yield).

LCMS: m/z 637 (M+H)⁺; ¹H NMR (DMSO-d₆, 400 MHz): δ 1.38 (t, 3H), 3.83 (s, 3H), 4.31 (q, 2H), 7.27 (d, 2H), 7.31 (dd, 1H), 7.35 (d, 1H), 7.45 (d, 1H), 7.49 (dd, 1H), 7.58 (d, 1H), 7.63 (d, 1H), 7.73 (d, 2H), 7.81-7.84 (m, 4H), 7.91 (d, 1H), 7.96 (s, 1H), 8.26 (d, 1H) ppm.

### Example 154

### 4-(4′-{2-[4-(2,4-Dichloro-phenyl)-1-ethyl-1H-imidazol-2-yl]-(E)-vinyl}-biphenyl-4-yloxy)-2-trifluoromethyl-benzoic acid

4-(4′-{2-[4-(2,4-Dichloro-phenyl)-1-ethyl-1H-imidazol-2-yl]-(E)-vinyl}-biphenyl-4-yloxy)-2-trifluoromethyl-benzoic acid methyl ester (32 mg, 0.05 mmol) was treated as described in general procedure F to give 4-(4′-{2-[4-(2,4-dichloro-phenyl)-1-ethyl-1H-imidazol-2-yl]-(E)-vinyl}-biphenyl-4-yloxy)-2-trifluoromethyl-benzoic acid (26 mg, 85% yield).

LCMS: m/z 623 (M+H)⁺; ¹H NMR (DMSO-d₆, 400 MHz): δ 1.36 (t, 3H), 4.29 (q, 2H), 7.26 (d, 2H), 7.30 (dd, 1H), 7.34 (d, 1H), 7.45 (d, 1H), 7.49 (dd, 1H), 7.57 (d, 1H), 7.62 (d, 1H), 7.73 (d, 2H), 7.82-7.85 (m, 4H), 7.90 (d, 1H), 7.95 (s, 1H), 8.24 (d, 1H) ppm.

### Example 155

### 4-(4′-{2-[4-(2,4-Dichloro-phenyl)-1-ethyl-1H-imidazol-2-yl]-(E)-vinyl}-biphenyl-4-yloxy)-2-nitro-benzoic acid methyl ester

4′-{2-[4-(2,4-Dichloro-phenyl)-1-ethyl-1H-imidazol-2-yl]-(E)-vinyl}-biphenyl-4-ol (87 mg, 0.2 mmol) was treated as described in general procedure I using methyl 4-fluoro-2-nitrobenzoate to give 4-(4′-{2-[4-(2,4-dichloro-phenyl)-1-ethyl-1H-imidazol-2-yl]-(E)-vinyl}-biphenyl-4-yloxy)-2-nitro-benzoic acid methyl ester (96 mg, 78% yield).

LCMS: m/z 614 (M+H)⁺; ¹H NMR (DMSO-d₆, 400 MHz): δ 1.38 (t, 3H), 3.84 (s, 3H), 4.28 (q, 2H), 7.20 (d, 1H), 7.30 (d, 2H), 7.35 (d, 1H), 7.48 (dd, 1H), 7.58 (d, 1H), 7.63 (d, 1H), 7.73 (d, 2H), 7.82-7.84 (m, 4H), 7.97 (s, 1H), 8.17 (dd, 1H), 8.24 (d, 1H), 8.53 (d, 1H) ppm.

### Example 156

### 4-(4′-{2-[4-(2,4-Dichloro-phenyl)-1-ethyl-1H-imidazol-2-yl]-(E)-vinyl}-biphenyl-4-yloxy)-2-nitro-benzoic acid

4-(4′-{2-[4-(2,4-Dichloro-phenyl)-1-ethyl-1H-imidazol-2-yl]-(E)-vinyl}-biphenyl-4-yloxy)-2-nitro-benzoic acid methyl ester (31 mg, 0.05 mmol) was treated as described in general procedure F to give 4-(4′-{2-[4-(2,4-dichloro-phenyl)-1-ethyl-1H-imidazol-2-yl]-(E)-vinyl}-biphenyl-4-yloxy)-2-nitro-benzoic acid (24 mg, 81% yield).

LCMS: m/z 600 (M+H)⁺; ¹H NMR (DMSO-d₆, 400 MHz): δ 1.37 (t, 3H), 4.27 (q, 2H), 7.19 (d, 1H), 7.30 (d, 2H), 7.34 (d, 1H), 7.48 (dd, 1H), 7.57 (d, 1H), 7.62 (d, 1H), 7.73 (d, 2H), 7.82-7.84 (m, 4H), 7.95 (s, 1H), 8.16 (dd, 1H), 8.24 (d, 1H), 8.51 (d, 1H) ppm.

### Example 157

### 2-Amino-4-(4′-{2-[4-(2,4-dichloro-phenyl)-1-ethyl-1H-imidazol-2-yl]-(E)-vinyl}-biphenyl-4-yloxy)-benzoic acid methyl ester

4-(4′-{2-[4-(2,4-Dichloro-phenyl)-1-ethyl-1H-imidazol-2-yl]-(E)-vinyl}-biphenyl-4-yloxy)-2-nitro-benzoic acid methyl ester (61 mg, 0.1 mmol) was treated as described in general procedure K to afford 2-amino-4-(4′-{2-[4-(2,4-dichloro-phenyl)-1-ethyl-1H-imidazol-2-yl]-(E)-vinyl}-biphenyl-4-yloxy)-benzoic acid methyl ester (44 mg, 76% yield).

LCMS: m/z 584 (M+H)⁺; ¹H NMR (DMSO-d₆, 400 MHz): δ 1.43 (t, 3H), 3.81 (s, 3H), 4.45 (q, 2H), 6.92 (d, 1H), 7.19 (d, 2H), 7.47 (dd, 1H), 7.51 (d, 1H), 7.67 (dd, 1H), 7.77-7.83 (m, 8H), 8.01 (d, 1H), 8.10-8.24 (m, 2H) ppm.

### Example 158

### 2-Amino-4-(4′-{2-[4-(2,4-dichloro-phenyl)-1-ethyl-1H-imidazol-2-yl]-(E)-vinyl}-biphenyl-4-yloxy)-benzoic acid

2-Amino-4-(4′-{2-[4-(2,4-dichloro-phenyl)-1-ethyl-1H-imidazol-2-yl]-(E)-vinyl}-biphenyl-4-yloxy)-benzoic acid methyl ester (12 mg, 0.02 mmol) was treated as described in general procedure F to afford 2-amino-4-(4′-{2-[4-(2,4-dichloro-phenyl)-1-ethyl-1H-imidazol-2-yl]-(E)-vinyl}-biphenyl-4-yloxy)-benzoic acid (8 mg, 72% yield).

LCMS: m/z 570 (M+H)⁺; ¹H NMR (DMSO-d₆, 400 MHz): δ 1.41 (t, 3H), 4.41 (q, 2H), 6.91 (d, 1H), 7.18 (d, 2H), 7.46 (dd, 1H), 7.51 (d, 1H), 7.65 (dd, 1H), 7.76-7.83 (m, 8H), 8.01 (d, 1H), 8.10-8.22 (m, 2H) ppm.

### Example 159

### 4-(4′-{2-[4-(2,4-Dichloro-phenyl)-1-ethyl-1H-imidazol-2-yl]-(E)-vinyl}-biphenyl-4-yloxy)-2-methanesulfonylamino-benzoic acid methyl ester

2-Amino-4-(4′-{2-[4-(2,4-dichloro-phenyl)-1-ethyl-1H-imidazol-2-yl]-(E)-vinyl}-biphenyl-4-yloxy)-benzoic acid methyl ester (29 mg, 0.05 mmol) was treated as described in general procedure L using methanesulfonyl chloride to afford 4-(4′-{2-[4-(2,4-dichloro-phenyl)-1-ethyl-1H-imidazol-2-yl]-(E)-vinyl}-biphenyl-4-yloxy)-2-methanesulfonylamino-benzoic acid methyl ester (22 mg, 67% yield).

LCMS: m/z 662 (M+H)⁺; ¹H NMR (DMSO-d₆, 400 MHz): δ 1.39 (t, 3H), 3.07 (s, 3H), 3.77 (s, 3H), 4.32 (q, 2H), 6.98 (d, 1H), 7.27 (d, 2H), 7.37 (d, 1H), 7.51 (dd, 1H), 7.60 (d, 1H), 7.65 (d, 1H), 7.73 (d, 2H), 7.77 (dd, 1H), 7.80-7.85 (m, 4H), 7.98 (s, 1H), 8.01 (d, 1H), 8.26 (d, 1H) ppm.

### Example 160

### 4-(4′-{2-[4-(2,4-Dichloro-phenyl)-1-ethyl-1H-imidazol-2-yl]-(E)-vinyl}-biphenyl-4-yloxy)-2-methanesulfonylamino-benzoic acid

4-(4′-{2-[4-(2,4-Dichloro-phenyl)-1-ethyl-1H-imidazol-2-yl]-(E)-vinyl}-biphenyl-4-yloxy)-2-methanesulfonylamino-benzoic acid methyl ester (20 mg, 0.03 mmol) was treated as described in general procedure F to give 4-(4′-{2-[4-(2,4-dichloro-phenyl)-1-ethyl-1H-imidazol-2-yl]-(E)-vinyl}-biphenyl-4-yloxy)-2-methanesulfonylamino-benzoic acid (14 mg, 73% yield).

LCMS: m/z 648 (M+H)⁺; ¹H NMR (DMSO-d₆, 400 MHz): δ 1.38 (t, 3H), 3.07 (s, 3H), 4.29 (q, 2H), 6.97 (d, 1H), 7.24 (d, 2H), 7.35 (d, 1H), 7.50 (dd, 1H), 7.59 (d, 1H), 7.64 (d, 1H), 7.73 (d, 2H), 7.77 (dd, 1H), 7.80-7.86 (m, 4H), 7.97 (s, 1H), 8.01 (d, 1H), 8.25 (d, 1H) ppm.

### Example 161

### 3-Amino-4-(4′-{2-[4-(2,4-dichloro-phenyl)-1-ethyl-1H-imidazol-2-yl]-(E)-vinyl}-biphenyl-4-yloxy)-benzoic acid

4′-{2-[4-(2,4-Dichloro-phenyl)-1-ethyl-1H-imidazol-2-yl]-(E)-vinyl}-biphenyl-4-ol (435 mg, 1 mmol) was treated as described in general procedure I using methyl 4-fluoro-3-nitrobenzoate to give the nitro compound intermediate, which was then reduced as described in general procedure K to give the ester (327 mg, 56% yield). The resulted ester (29 mg, 0.05 mmol) was treated as described in general procedure F to afford 3-amino-4-(4′-{2-[4-(2,4-dichloro-phenyl)-1-ethyl-1H-imidazol-2-yl]-(E)-vinyl}-biphenyl-4-yloxy)-benzoic acid (22 mg, 77% yield).

LCMS: m/z 570 (M+H)⁺; ¹H NMR (DMSO-d₆, 400 MHz): δ 1.41 (t, 3H), 4.42 (q, 2H), 6.91 (d, 1H), 7.18 (d, 2H), 7.46 (dd, 1H), 7.51 (d, 1H), 7.65 (dd, 1H), 7.76-7.83 (m, 8H), 8.01 (d, 1H), 8.10-8.22 (m, 2H) ppm.

### Example 162

### 4-(4′-{2-[4-(2,4-Dichloro-phenyl)-1-ethyl-1H-imidazol-2-yl]-(E)-vinyl}-biphenyl-4-yloxy)-3-methanesulfonylamino-benzoic acid

4′-{2-[4-(2,4-Dichloro-phenyl)-1-ethyl-1H-imidazol-2-yl]-(E)-vinyl}-biphenyl-4-ol (435 mg, 1 mmol) was treated as described in general procedure I using methyl 4-fluoro-3-nitrobenzoate to give the nitro compound intermediate, which was then reduced as described in general procedure K to give the ester (327 mg, 56% total yield). The resulted ester (59 mg, 0.1 mmol) was treated as described in general procedure L using methanesulfonyl chloride to give methanesulfonamide, which was then hydrolyzed as described in general procedure F to give 4-(4′-{2-[4-(2,4-dichloro-phenyl)-1-ethyl-1H-imidazol-2-yl]-(E)-vinyl}-biphenyl-4-yloxy)-3-methanesulfonylamino-benzoic acid (26 mg, 41% yield).

LCMS: m/z 648 (M+H)⁺; ¹H NMR (DMSO-d₆, 400 MHz): δ 1.38 (t, 3H), 3.07 (s, 3H), 4.29 (q, 2H), 6.97 (d, 1H), 7.23 (d, 2H), 7.35 (d, 1H), 7.50 (dd, 1H), 7.59 (d, 1H), 7.64 (d, 1H), 7.73 (d, 2H), 7.77 (dd, 1H), 7.79-7.85 (m, 4H), 7.97 (s, 1H), 8.01 (d, 1H), 8.24 (d, 1H) ppm.

### Example 163

### 4-(4′-{2-[4-(2,4-Dichloro-phenyl)-1-ethyl-1H-imidazol-2-yl]-(E)-vinyl}-biphenyl-4-yloxy)-3-trifluoromethanesulfonylamino-benzoic acid

4′-{2-[4-(2,4-Dichloro-phenyl)-1-ethyl-1H-imidazol-2-yl]-(E)-vinyl}-biphenyl-4-ol (435 mg, 1 mmol) was treated as described in general procedure I using methyl 4-fluoro-3-nitrobenzoate to give the nitro compound intermediate, which was then reduced as described in general procedure K to give the ester (327 mg, 56% yield). The resulted ester (59 mg, 0.1 mmol) was treated as described in general procedure L using trifluoromethanesulfonic acid anhydride to give trifluoromethanesulfonamide, which was then hydrolyzed as described in general procedure F to give 4-(4′-{2-[4-(2,4-dichloro-phenyl)-1-ethyl-1H-imidazol-2-yl]-(E)-vinyl}-biphenyl-4-yloxy)-3-trifluoromethanesulfonyl-amino-benzoic acid (26 mg, 37% yield).

LCMS: m/z 702 (M+H)⁺; ¹H NMR (DMSO-d₆, 400 MHz): δ 1.38 (t, 3H), 4.29 (q, 2H), 6.98 (d, 1H), 7.12 (d, 2H), 7.36 (d, 1H), 7.41 (dd, 1H), 7.60 (d, 1H), 7.64 (d, 1H), 7.74 (d, 2H), 7.77 (dd, 1H), 7.79-7.85 (m, 4H), 7.98 (s, 1H), 8.01 (d, 1H), 8.22 (d, 1H) ppm.

### Example 164

### 5-(4′-{2-[4-(2,4-Dichloro-phenyl)-1-ethyl-1H-imidazol-2-yl]-(E)-vinyl}-biphenyl-4-yloxy)-2-methanesulfonylamino-benzoic acid

4′-{2-[4-(2,4-Dichloro-phenyl)-1-ethyl-1H-imidazol-2-yl]-(E)-vinyl}-biphenyl-4-ol (435 mg, 1 mmol) was treated as described in general procedure I using methyl 2-amino-5-bromobenzoate to give the ester (245 mg, 42% yield). The ester (59 mg, 0.1 mmol) was treated as described in general procedure L using methanesulfonyl chloride to give the methanesulfonamide, which was then hydrolyzed as described in general procedure F to give 5-(4′-{2-[4-(2,4-dichloro-phenyl)-1-ethyl-1H-imidazol-2-yl]-(E)-vinyl}-biphenyl-4-yloxy)-2-methanesulfonylamino-benzoic acid (25 mg, 39% yield).

LCMS: m/z 648 (M+H)⁺; ¹H NMR (DMSO-d₆, 400 MHz): δ 1.37 (t, 3H), 3.17 (s, 3H), 4.28 (q, 2H), 7.14 (d, 2H), 7.34 (d, 1H), 7.44 (dd, 1H), 7.50 (dd, 1H), 7.58 (d, 1H), 7.60-7.66 (m, 3H), 7.71 (d, 2H), 7.77 (d, 2H), 7.83 (d, 2H), 7.97 (s, 1H), 8.24 (d, 1H) ppm.

### Example 165

### 5-(4′-{2-[4-(2,4-Dichloro-phenyl)-1-ethyl-1H-imidazol-2-yl]-(E)-vinyl}-biphenyl-4-yloxy)-2-trifluoromethanesulfonylamino-benzoic acid

4′-{2-[4-(2,4-Dichloro-phenyl)-1-ethyl-1H-imidazol-2-yl]-(E)-vinyl}-biphenyl-4-ol (435 mg, 1 mmol) was treated as described in general procedure I using methyl 2-amino-5-bromobenzoate to give the ester (245 mg, 42% yield). The ester (59 mg, 0.1 mmol) was treated as described in general procedure L using trifluoromethanesulfonic anhydride to give trifluoromethanesulfonamide, which was then hydrolyzed as described in general procedure F to give 5-(4′-{2-[4-(2,4-dichloro-phenyl)-1-ethyl-1H-imidazol-2-yl]-(E)-vinyl}-biphenyl-4-yloxy)-2-trifluoromethanesulfonylamino-benzoic acid (31 mg, 44% total yield).

LCMS: m/z 702 (M+H)⁺; ¹H NMR (DMSO-d₆, 400 MHz): δ 1.38 (t, 3H), 4.29 (q, 2H), 7.08 (d, 2H), 7.25 (dd, 1H), 7.36 (d, 1H), 7.51 (m, 2H), 7.60 (d, 1H), 7.62 (d, 1H), 7.66 (d, 1H), 7.71 (d, 2H), 7.74 (d, 2H), 7.83 (d, 2H), 7.98 (s, 1H), 8.22 (d, 1H) ppm.

### Example 165

### 4-(4′-{2-[4-(2,4-Dichloro-phenyl)-1-ethyl-1H-imidazol-2-yl]-(E)-vinyl}-biphenyl-4-yloxy)-butyric acid 2,2-dimethyl-propionyloxymethyl ester

To a solution of 4-(4′-{2-[4-(2,4-dichloro-phenyl)-1-ethyl-1H-imidazol-2-yl]-(E)-vinyl}-biphenyl-4-yloxy)-butyric acid (52 mg, 0.1 mmol) in anhydrous DMF (5 mL) is added chloromethyl pivalate (30 mg, 0.2 mmol) followed by freshly ground K₂CO₃ (56 mg, 0.4 mmol). The reaction mixture is heated at 65° C. under nitrogen for 2 to 4 hours. At completion, the mixture is then diluted with water/EtOAc and the layers separated. The aqueous layer is further extracted with EtOAc, and the organic layers combined and dried over Na₂SO₄. The solvent is removed in vacuo and the residue is purified by silica gel chromatography to afford (56 mg, 88% yield) 4-(4′-{2-[4-(2,4-dichloro-phenyl)-1-ethyl-1H-imidazol-2-yl]-(E)-vinyl}-biphenyl-4-yloxy)-butyric acid 2,2-dimethyl-propionyloxymethyl ester.

LCMS: m/z 635 (M+H)⁺; ¹H NMR (DMSO-d₆, 400 MHz): δ 1.11 (s, 9H), 1.42 (t, 3H), 1.99 (m, 2H), 2.54 (t, 2H), 4.03 (t, 2H), 4.41 (q, 2H), 5.70 (s, 2H), 7.01 (d, 2H), 7.46 (d, 1H), 7.65 (dd, 1H), 7.68 (d, 2H), 7.74 (d, 2H), 7.84 (d, 2H), 7.85 (s, 1H), 8.01 (d, 1H), 8.05 (d, 1H), 8.19 (s, 1H) ppm.

### Example 167

### 4-(4-Chloro-phenyl)-2-[2-(4-ethoxy-phenyl)-(E)-vinyl-1H-imidazole

4-(4-Chloro-phenyl)-2-[2-(4-ethoxy-phenyl)-(E)-vinyl-1H-imidazole (258 mg, 79%) was synthesized using trans-4-ethoxycinnamic acid (192 mg, 1 mmol) and 4-chlorophenacyl bromide (233 mg 1 mmol) according to general procedure A.

LCMS: m/z 325 (M+H)⁺; ¹H NMR (CDCl₃, 400 MHz): δ 1.43 (t, 2H), 1.62 (d, 1H), 4.08 (q, 2H), 6.88 (d, 1H), 6.95 (d, 2H), 7.33 (d, 1H), 7.51 (d, 2H), 7.52 (d, 2H), 7.54 (s, 1H), 7.66 (d, 1H), 7.93 (s, 1H) ppm.

### Example 168

### 4-(2,4-Difluoro-phenyl)-2-[2-(4-ethoxy-phenyl)-(E)-vinyl]-1H-imidazole

4-(2,4-Difluoro-phenyl)-2-[2-(4-ethoxy-phenyl)-(E)-vinyl]-1H-imidazole (249 mg, 76%) was prepared using trans-4-ethoxycinnamic acid (192 mg, 1 mmol) and 4-fluorophenacyl bromide (217 mg 1 mmol) according to general procedure A.

LCMS: m/z 327 (M+H)⁺; ¹H NMR (CDCl₃, 400 MHz): δ 1.43 (t, 2H), 1.62 (d, 1H), 4.08 (q, 2H), 6.88 (d, 1H), 6.95 (d, 2H), 7.33 (d, 1H), 7.51 (d, 2H), 7.52 (d, 1H), 7.54 (s, 1H), 7.66 (d, 1H), 7.93 (s, 1H) ppm.

### Example 169

### 2-[2-(4-Ethoxy-phenyl)-(E)-vinyl]-4-(4-methoxy-phenyl)-1H-imidazole

2-[2-(4-Ethoxy-phenyl)-(E)-vinyl]-4(4-methoxy-phenyl)-1H-imidazole (221 mg, 69%) was prepared according to general procedure A using trans-4-ethoxycinnamic acid (198 mg, 1 mmol) and 4-methoxyphenacyl bromide (229 mg, 1 mmol).

LCMS: m/z 321 (M+H)⁺.

### Example 170

### 2-[2-(4-Ethoxy-phenyl)-(E)-vinyl]-4-(2,3,4-trichloro-phenyl)-1H-imidazole

2-[2-(4-Ethoxy-phenyl)-(E)-vinyl]-4-(2,3,4-trichloro-phenyl)-1H-imidazole (279 mg, 70%) was prepared according to general procedure A using trans-4-ethoxycinnamic acid (198 mg, 1 mmol) and 2,3,4-trichlorophenacyl bromide (302 mg. 1 mmol).

LCMS: m/z 393 (M+H)⁺; ¹H NMR (CDCl₃, 400 MHz): δ 1.43 (t, 2H), 1.62 (d, 1H), 4.08 (q, 2H), 6.38 (d, 1H), 6.81 (d, 1H), 6.90 (d, 1H), 7.28 (d, 2H), 7.38 (d, 1H), 7.48 (d, 2H), 7.74 (d, 1H), 9.1 (d, 1H) ppm.

### Example 171

### 4-[2-(4-Naphthalen-1-yl-1H-imidazole-2-yl)-(E)-vinyl]-phenol

4-[2-(4-Naphthalen-1-yl-1H-imidazole-2-yl)-(E)-vinyl]-phenol (241 mg, 78%) was prepared according to general procedure A using trans-4-hydroxycinnamic acid (164 mg, 1 mmol) and 1-naphthaleneacylbromide (249 mg, 1 mmol).

LCMS: m/z 313 (M+H)⁺; ¹H NMR (CDCl₃, 400 MHz): δ 6.69 (s, 1H), 6.95 (d, 2H), 7.42 (d, 1H), 7.55 (d, 2H), 7.63 (d, 2H), 7.65 (d, 2H), 7.89-7.77 (m, 4H) ppm.

### Example 172

### 4-{2-[4-(4-Chloro-phenyl)-5-phenyl-1H-imidazole-2-yl]-(E)-vinyl}-phenol

4-{2-[4-(4-Chloro-phenyl)-5-phenyl-1H-imidazole-2-yl]-(E)-vinyl}-phenol (285 mg, 76%) was prepared according to general procedure A using trans-4-hydroxycinnamic acid (164 mg, 1 mmol) and 2-bromo-1-(4-chlorophenyl)-2-phenylethan 1-one (309 mg, 1 mmol).

LCMS: m/z 373 (M+H)⁺.

### Example 173

### 4-Biphenyl-4-yl-2-[2-(4-methoxy-phenyl)-(E)-vinyl]-1H-imidazole

4-Biphenyl-4-yl-2-[2-(4-methoxy-phenyl)-(E)-vinyl]-1H-imidazole (281 mg, 80%) was prepared according to general procedure A using trans-4-methoxycinnamic acid (178 mg, 1 mmol) and 2-bromo-4-phenylacetophenone (275 mg, 1 mmol).

LCMS: m/z 353 (M+H)⁺; ¹H NMR (CDCl₃, 400 MHz): δ 3.78 (s, 3H), 6.95-6.93 (m, 2H), 7.36-7.33 (m, 2H), 7.48-7.44 (m, 2H), 7.55-7.53 (m, 2H), 7.71-7.64 (m, 6H), 7.90-7.88 (m, 2H) ppm.

### Example 174

### (4-{2-[2-(4-Methoxy-phenyl)-(E)-vinyl]-1H-imidazole-4-yl}-phenyl-diazene

(4-{2-[2-(4-Methoxy-phenyl)-(E)-vinyl]-1H-imidazole-4-yl}-phenyl-diazene (291 mg, 77%) was prepared according to general procedure A using trans 4-methoxycinnamic acid (178 mg, 1 mmol) and 2-bromo-4-phenylazoacetophenone (303 mg, 1 mmol).

LCMS: m/z 381 (M+H)⁺; ¹H NMR (CDCl₃, 400 MHz): δ 3.77 (s, 3H), 6.80 (d, 2H), 6.85 (d, 2H), 7.27 (s, 1H), 7.36 (d, 1H), 7.53 (m, 4H), 7.83 (d, 2H), 7.91 (d, 2H), 7.93 (d, 2H) ppm.

### Example 175

### {4-Biphenyl-4-yl-2-[2-(4-methoxy-phenyl)-(E)-vinyl]-imidazole-1yl}-acetic acid methyl ester

4-Biphenyl-4-yl-2-[2-(4-methoxy-phenyl)-(E)-vinyl]-1H-imidazole (352 mg, 1 mmol) was treated with methyl bromoacetate (153 mg, 1 mmol) according to general procedure E to give {4-biphenyl-4-yl-2-[2-(4-methoxy-phenyl)-(E)-vinyl]-imidazole-1-yl}-acetic acid methyl ester (375 mg, 88%).

LCMS: m/z 425 (M+H)⁺; ¹H NMR (CDCl₃, 400 MHz): δ 3.78 (s, 3H), 3.96 (s, 3H), 5.17 (s, 2H), 6.95-6.93 (m, 2H), 7.36-7.33 (m, 2H), 7.48-7.44 (m, 2H), 7.55-7.53 (m, 2H), 7.71-7.64 (m, 6H), 7.90-7.88 (m, 2H) ppm.

### Example 176

### {4-Biphenyl-4-yl-2-[2-(4-methoxy-phenyl)-(E)-vinyl]-imidazole-1yl}-acetic acid

{4-Biphenyl-4-yl-2-[2-(4-methoxy-phenyl)-(E)-vinyl]-imidazole-1yl)-acetic acid methyl ester (212 mg, 0.5 mmol) was hydrolyzed according to general procedure F to give {4-biphenyl-4-yl-2-[2-(4-methoxy-phenyl)-(E)-vinyl]-imidazole-1yl}-acetic acid (212 mg, 80%).

LCMS: m/z 411 (M+H)⁺; ¹H NMR (CDCl₃, 400 MHz): δ 3.78 (s, 3H), 5.17 (s, 2H), 6.95-6.93 (m, 2H), 7.36-7.33 (m, 2H), 7.48-7.44 (m, 2H), 7.55-7.53 (m, 2H), 7.71-7.64 (m, 6H), 7.90-7.88 (m, 2H) ppm.

### Example 177

### 4-(4-Chloro-phenyl)-2-[2-(4-methoxy-phenyl)-(E)-vinyl]-5-p-tolyl-1H-imidazole

4-(4-Chloro-phenyl)-2-[2-(4-methoxy-phenyl)-(E)-vinyl]-5-p-tolyl-1H-imidazole (299 mg, 75%) was prepared according to general procedure A using trans-4-methoxycinnamic acid (178 mg, 1 mmol) and 2-bromo-1-(4-chlorophenyl)-2-(4-methyl phenyl)-ethan-1-one (323 mg, 1 mmol).

LCMS: m/z 401 (M+H)⁺; ¹H NMR (CDCl₃, 400 MHz): δ 2.40 (s, 3H), 3.85 (s, 3H), 6.89 (d, 1H), 6.95 (d, 2H), 7.22 (d, 2H), 7.37 (d, 1H), 7.52-7.50 (m, 4H), 7.64-7.53 (m, 4H) ppm.

### Example 178

### 2-{4-Biphenyl-4-yl-2-[2-(4-methoxy-phenyl)-(E)-vinyl]-imidazole-1yl}-N-(1-naphthalen-1-yl-ethyl)-acetamide

{4-Biphenyl-4-yl-2-[2-(4-methoxy-phenyl)-(E)-vinyl]-imidazole-1yl)-acetic acid (410 mg, 1 mmol) was coupled with DL-1-(1-naphthyl)-ethyl amine (171 mg, 1 mmol) following general procedure G to give 2-{4-biphenyl-4-yl-2-[2-(4-methoxy-phenyl)-(E)-vinyl]-imidazole-1yl}-N-(1-naphthalen-1-yl-ethyl)-acetamide (497 mg, 88%).

LCMS: m/z 564 (M+H)⁺; ¹H NMR (CDCl₃, 400 MHz): δ 1.59 (d, 3H), 3.85 (s, 3H), 4.73 (d, 2H), 5.91 (d, 1H), 5.97 (m, 1H), 6.59 (d, 1H), 6.89 (d, 2H), 7.14 (s, 1H), 7.22-7.41 (m, 2H), 7.50-7.42 (m, 7H), 7.60-7.42 (m, 4H), 7.64-7.62 (m, 3H), 7.71 (d, 1H), 7.82 (d, 1H), 8.04 (d, 1H) ppm.

### Example 179

### 4-(4-Bromo-phenyl)-2-[2-(4-methoxy-phenyl)-(E)-vinyl]-1H-imidazole

4-(4-Bromo-phenyl)-2-[2-(4-methoxy-phenyl)-(E)-vinyl]-1H-imidazole (281 mg, 79%) was prepared according to general procedure A using trans-4-methoxycinnamic acid (178 mg, 1 mmol) and 2,4-dibromo acetophenone (278 mg, 1 mmol).

LCMS: m/z 356 (M+H)⁺.

### Example 180

### Diethyl-(4-{2-[2-(4-methoxy-phenyl)-(E)-vinyl]-1H-imidazol-4yl}-phenyl)-amine

Diethyl-(4-{2-[2-(4-methoxy-phenyl)-(E)-vinyl]-1H-imidazol-4yl}-phenyl)-amine (247 mg, 72%) was prepared according to general procedure A using trans-4-methoxycinnamic acid (178 mg, 1 mmol) and 2-bromo-1-(4-diethylamino-phenyl)-ethan-1-one (270 mg, 1 mmol).

LCMS: m/z 348 (M+H)⁺.

### Example 181

### 2-[2-(4-Methoxy-phenyl)-(E)-vinyl]-4-pentafluorophenyl-1H-imidazole

2-[2-(4-Methoxy-phenyl)-(E)-vinyl]-4-pentafluorophenyl-1H-imidazole (271 mg, 74%) was prepared according to general procedure A using trans-4-methoxycinnamic acid (178 mg, 1 mmol) and bromoacetyl pentafluorobenzene (288 mg, 1 mmol).

LCMS: 367 (M+H)⁺. ¹H NMR (CDCl₃, 400 MHz): δ 3.86 (s, 3H), 6.38 (d, 1H), 6.58 (d, 2H), 7.33 (d, 1H), 7.51 (d, 2H), 7.93 (s, 1H) ppm.

### Example 182

### 4-(3′,5′-Dichloro-biphenyl-4-yl)-2-[2-(4-methoxy-phenyl)-(E)-vinyl]-1H-imidazole

4-(3′,5′-Dichloro-biphenyl-4-yl)-2-[2-(4-methoxy-phenyl)-(E)-vinyl]-1H-imidazole (313 mg, 74%) was prepared according to general procedure A using trans-4-methoxycinnamic acid (178 mg, 1 mmol) and 2-bromo-4-(3,5-dichloro-phenyl)acetophenone (344 mg, 1 mmol).

LCMS: 421 (M+H)⁺. ¹H NMR (DMSO-d₆, 400 MHz): δ 3.78 (s, 3H), 6.94-6.96 (m, 2H), 7.31-7.34 (m, 2H), 7.44-7.48 (m, 2H), 7.55 (d, 2H), 7.61-7.71 (m, 4H), 7.90 (s, 1H), 12.40 (s, 1H) ppm.

### Example 183

### 2-[2-(4-Methoxy-phenyl)-(E)-vinyl]-4-(4-pentyl-phenyl)-1H-imidazole

2-[2-(4-Methoxy-phenyl)-(E)-vinyl]-4-(4-pentyl-phenyl)-1H-imidazole (240 mg, 70%) was prepared according to general procedure A using trans-4-methoxycinnamic acid (178 mg, 1 mmol) and 2-bromo-1-(4-pentyl phenyl)-ethan-1-one (269 mg, 1 mmol).

LCMS: m/z 347 (M+H)⁺.

### Example 184

### 4-{2-[2-(4-Methoxy-phenyl)-(E)-vinyl]-1H-imidazol-4-yl}-benzoic acid phenyl ester

4-{2-[2-(4-Methoxy-phenyl)-(E)-vinyl]-1H-imidazol-4-yl}-benzoic acid phenyl ester (259 mg, 65%) was prepared according to general procedure A using trans-4-methoxycinnamic acid (178 mg, 1 mmol) and 2-bromo-(4-phenyl benzoate) acetophenone (319 mg, 1 mmol).

LCMS: m/z 397 (M+H)⁺.

### Example 185

### 4-(3′,5′-Dichloro-biphenyl-4-yl)-1-ethyl-2-[-2-(4-methoxy-phenyl)-(E)-vinyl]-1H-imidazole

4-(3′,5′-Dichloro-biphenyl-4-yl)-2-[2-(4-methoxy-phenyl)-(E)-vinyl]-1H-imidazole (421 mg, 1 mmol) was treated with bromoethane (109 mg, 1 mmol) according to general procedure E to give 4-(3′,5′-dichloro-biphenyl-4-yl)-1-ethyl-2-[-2-(4-methoxy-phenyl)-(E)-vinyl]-1H-imidazole (401 mg, 89%).

LCMS: m/z 449 (M+H)⁺; ¹H NMR (DMSO-d₆, 400 MHz): δ 1.21 (t, 3H), 3.78 (s, 3H), 3.93 (q, 2H), 6.94-6.96 (m, 2H), 7.31-7.34 (m, 2H), 7.44-7.48 (m, 2H), 7.55 (d, 2H), 7.61-7.71 (m, 4H), 7.90 (s, 1H), 12.40 (s, 1H) ppm.

### Example 186

### 4-(4-tert-Butyl-phenyl)-2-[2-(4-methoxy-phenyl)-(E)-vinyl]-1H-imidazole

4-(4-tert-Butyl-phenyl)-2-[2-(4-methoxy-phenyl)-(E)-vinyl]-1H-imidazole (218 mg, 66%) was prepared according to general procedure A using trans-4-methoxycinnamic acid (178 mg, 1 mmol) and 4-(tert-butyl)-phenacyl bromide (255 mg, 1 mmol).

LCMS: m/z 333 (M+H)⁺.

### Example 187

### 2-[2-(4-Methoxy-phenyl)-(E)-vinyl]-4-(3-trifluoromethyl-phenyl)-1H-imidazole

2-[2-(4-Methoxy-phenyl)-(E)-vinyl]-4-(3-trifluoromethyl-phenyl)-1H-imidazole (229 mg, 67%) was prepared according to general procedure A using trans-4-methoxycinnamic acid (178 mg, 1 mmol) and 2-bromo-1-(3-trifluoromethyl)-phenyl-1-ethanone (267 mg, 1 mmol).

LCMS: m/z 345 (M+H)⁺.

### Example 188

### 4-(2,3-Dihydro-benzo[1,4]dioxin-5-yl)-2-[2-(4-methoxy-phenyl)-(E)-vinyl]-1H-imidazole

4-(2,3-Dihydro-benzo[1,4]dioxin-5-yl)-2-[2-(4-methoxy-phenyl)-(E)-vinyl]-1H-imidazole (219 mg, 65%) was prepared according to general procedure A using trans-4-methoxycinnamic acid (178 mg, 1 mmol) and 2-bromo-1-(2-3-dihydro-1-4-benzodioxepin-6-yl)-ethan-1-one (257 mg, 1 mmol).

LCMS: m/z 335 (M+H)⁺.

### Example 189

### 2-[2-(4-Bromo-phenyl)-(E)-vinyl]-1-ethyl-4-(4-methoxy-phenyl)-1H-imidazole

2-[2-(4-Bromo-phenyl)-(E)-vinyl]-1-ethyl-4-(4-methoxy-phenyl)-1H-imidazole (249 mg, 65%) was prepared according to general procedure A using trans-4-bromocinnamic acid (227 mg, 1 mmol) and 2-bromo-4-methoxyacetophenone (229 mg, 1 mmol) and obtained 2-[2-(4-bromo-phenyl)-(E)-vinyl]-4-(4-methoxy-phenyl)-1H-imidazole (355 mg, 1 mmol) was treated with bromoethane (109 mg, 1 mmol) following general procedure E.

LCMS: m/z 384 (M+H)⁺.

### Example 190

### 2-[2-(4-Bromo-phenyl)-(E)-vinyl]-1-ethyl-4-(4-cyano-phenyl)-1H-imidazole

2-[2-(4-Bromo-phenyl)-(E)-vinyl]-1-ethyl-4-(4-cyano-phenyl)-1H-imidazole (319 mg, 84%) was prepared according to general procedure A using trans-4-bromocinnamic acid (227 mg, 1 mmol) and 4-cyanophenacyl bromide (224 mg, 1 mmol) and obtained 2-[2-(4-bromo-phenyl)-(E)-vinyl]-4-(4-cyano-phenyl)-1H-imidazole (350 mg, 1 mmol) was treated with bromoethane (109 mg, 1 mmol) following general procedure E.

LCMS: m/z 379 (M+H)⁺.

### Example 191

### 4-(4′-{2-[1-Ethyl-4-(4-methoxy-phenyl)-1H-imidazol-2-yl]-(E)-vinyl}-biphenyl-4-yloxy)-butyric acid methyl ester

2-[2-(4-Bromo-phenyl)-(E)-vinyl]-1-ethyl-4-(4-methoxy-phenyl)-1H-imidazole (383 mg, 1 mmol) was coupled with 4-hydroxyphenylboronic acid (137 mg, 1 mmol) following general procedure B and obtained 4′-{2-[1-ethyl-4-(4-methoxy-phenyl)-1H-imidazol-2-yl]-(E)-vinyl}-biphenyl-4-ol (396 mg, 1 mmol) was alkylated with methyl 4-bromobutyrate (181 mg, 1 mmol) following general procedure E to give 4-(4′-{2-[1-ethyl-4-(4-methoxy-phenyl)-1H-imidazol-2-yl]-(E)-vinyl}-biphenyl-4-yloxy)-butyric acid methyl ester (351 mg, 70%).

LCMS: m/z 497 (M+H)⁺; ¹H NMR (CDCl₃, 400 MHz): 1.51 (t, 3H), 2.16 (m, 2H), 2.57 (m, 2H), 3.70 (s, 3H), 3.83 (s, 3H), 4.09 (q, 2H), 4.13 (t, 2H), 6.92 (d, 2H), 6.94-6.97 (m, 1H), 7.53-7.61 (m, 8H), 7.75 (d, 2H), 7.77 (d, 2H) ppm.

### Example 192

### 4-(4′-{2-[1-Ethyl-4-(4-methoxy-phenyl)-1H-imidazol-2-yl]-(E)-vinyl}-biphenyl-4-yloxy)-butyric acid

4-(4′-{2-[1-Ethyl-4-(4-methoxy-phenyl)-1H-imidazol-2-yl]-(E)-vinyl}-biphenyl-4-yloxy)-butyric acid methyl ester (248 mg, 0.5 mmol) was hydrolyzed according to general procedure F to give 4-(4′-{2-[1-ethyl-4-(4-methoxy-phenyl)-1H-imidazol-2-yl]-(E)-vinyl}-biphenyl-4-yloxy)-butyric acid (192 mg, 80%).

LCMS: m/z 483 (M+H)⁺; ¹H NMR (DMSO, 400 MHz): 1.15 (t, 3H), 1.36 (m, 2H), 1.97 (m, 2H), 2.42 (t, 2H), 3.77 (s, 3H), 4.0 (q, 2H), 4.2 (t, 2H), 6.93 (d, 2H), 7.01 (d, 2H), 7.28 (d, 1H), 7.47 (d, 1H), 7.62-7.66 (m, 4H), 7.75-7.77 (m, 4H) ppm.

### Example 193

### 2-[2-(4-Bromo-phenyl)-(E)-vinyl]-1-ethyl-4-(3-trifluoromethyl-phenyl)-1H-imidazole

2-[2-(4-Bromo-phenyl)-(E)-vinyl]-1-ethyl-4-(3-trifluoromethyl-phenyl)-1H-imidazole (314 mg, 75%) was prepared according to general procedure A using trans-4-bromocinnamic acid (227 mg, 1 mmol) and 2-bromo-1-(3-trifluoromethyl)-phenyl-1-ethanone (267 mg, 1 mmol) and obtained 2-[2-(4-bromo-phenyl)-(E)-vinyl]-4-(3-trifluoromethyl-phenyl)-1H-imidazole (393 mg, 1 mmol) was treated with bromoethane (109 mg, 1 mmol) following general procedure E.

LCMS: m/z 422 (M+H)⁺; ¹H NMR (CDCl₃, 400 MHz): δ 1.52 (t, 3H), 4.12 (q, 2H), 6.91 (d, 2H), 7.31 (d, 1H), 7.41 (d, 2H), 7.43-7.49 (m, 2H), 7.68 (d, 2H), 7.99 (d, 2H), 8.08 (s, 1H) ppm.

### Example 194

### 4-(4′-[2-[1-Ethyl-4-(3-trifluoromethyl-phenyl)-1H-imidazol-2-yl]-(E)-vinyl}-biphenyl-4-yloxy)-butyric acid methyl ester

2-[2-(4-Bromo-phenyl)-(E)-vinyl]-1-ethyl-4-(3-trifluoromethyl-phenyl)-1H-imidazole (421 mg, 1 mmol) was coupled with 4-hydroxy phenyl boronic acid (137 mg, 1 mmol) following general procedure B and obtained 4′-{2-[1-ethyl-4-(3-trifluoromethyl-phenyl)-1H-imidazol-2-yl]-(E)-vinyl}-biphenyl-4-ol (434 mg, 1 mmol) was alkylated with methyl 4-bromobutyrate (181 mg, 1 mmol) following general procedure E to give 4-(4′-[2-[1-ethyl-4-(3-trifluoromethyl-phenyl)-1H-imidazol-2-yl]-(E)-vinyl}-biphenyl-4-yloxy)-butyric acid methyl ester (432 mg, 80%).

LCMS: m/z 535 (M+H)⁺; ¹H NMR (CDCl₃, 400 MHz): 1.55 (t, 3H), 2.16 (m, 2H), 2.58 (m, 2H), 3.70 (s, 3H), 4.07 (q, 2H), 4.16 (t, 2H), 6.91 (s, 1H), 6.98 (d, 2H), 7.30 (s, 1H), 7.48 (d, 2H), 7.54-7.56 (m, 4H), 7.61 (d, 1H), 7.78 (s, 1H), 8.01 (d, 2H), 8.09 (s, 1H) ppm.

### Example 195

### 4-(4′-[2-[1-Ethyl-4-(3-trifluoromethyl-phenyl)-1H-imidazol-2-yl]-(E)-vinyl}-biphenyl-4-yloxy)-butyric acid

4-(4′-[2-[1-Ethyl-4-(3-trifluoromethyl-phenyl)-1H-imidazol-2-yl]-(E)-vinyl}-biphenyl-4-yloxy)-butyric acid methyl ester (267 mg, 0.5 mmol) was hydrolyzed according to general procedure F to give 4-(4′-[2-[1-ethyl-4-(3-trifluoromethyl-phenyl)-1H-imidazol-2-yl]-(E)-vinyl}-biphenyl-4-yloxy)-butyric acid (216 mg, 83%).

LCMS: m/z 521 (M+H)⁺; ¹H NMR (DMSO-d₆, 400 MHz): δ 1.15 (t, 3H), 1.36 (m, 2H), 1.97 (m, 2H), 2.42 (t, 2H), 4.0 (q, 2H), 4.2 (t, 2H), 6.93 (d, 2H), 7.01 (d, 2H), 7.28 (d, 1H), 7.47 (d, 1H), 7.62-7.66 (m, 4H), 7.75-7.77 (m, 4H) ppm.

### Example 196

### 2-[2-(4-Bromo-phenyl)-(E)-vinyl]-4-(4-tert-butyl-phenyl)-1-ethyl-1H-imidazole

2-[2-(4-Bromo-phenyl)-(E)-vinyl]-4-(4-tert-butyl-phenyl)-1-ethyl-1H-imidazole (316 mg, 77%) was prepared according to general procedure A using trans-4-bromocinnamic acid (227 mg, 1 mmol) and 4-(tert-butyl)-phenacyl bromide (255 mg, 1 mmol) and obtained 2-[2-(4-bromo-phenyl)-(E)-vinyl]-4-(4-tert-butyl-phenyl)-1H-imidazole (381 mg, 1 mmol) was treated with bromoethane (109 mg, 1 mmol) following general procedure E.

LCMS: m/z 410 (M+H)⁺; ¹H NMR (CDCl₃, 400 MHz): δ 1.41 (s, 9H), 1.57 (t, 3H), 4.16 (q, 2H), 6.98 (d, 2H), 7.33 (s, 1H), 7.47-7.50 (m, 4H), 7.55 (d, 1H), 7.57 (d, 1H), 7.73 (d, 1H), 7.82 (d, 1H) ppm.

### Example 197

### 4-(4′-{2-[4-tert-Butyl-phenyl)-1-ethyl-1H-imidazol-2-yl]-(E)-vinyl}-biphenyl-4-yloxy)-butyric acid

Step 1: 2-[2-(4-Bromo-phenyl)-(E)-vinyl]-4-(4-tert-butyl-phenyl)-1-ethyl-1H-imidazole (409 mg, 1 mmol) was coupled with 4-hydroxyphenylboronic acid (137 mg, 1 mmol) following general procedure B and obtained 4′-{2-[4-(4-tert-Butyl-phenyl)-1-ethyl-1H-imidazol-2-yl]-(E)-vinyl}-biphenyl-4-ol (422 mg, 1 mmol) was alkylated with methyl 4-bromobutyrate (181 mg, 1 mmol) following general procedure E to give 4-(4′-{2-[4-tert-butyl-phenyl)-1-ethyl-1H-imidazol-2-yl]-(E)-vinyl}-biphenyl-4-yloxy)-butyric acid methyl ester (411 mg, 78%).

LCMS: m/z 523 (M+H)⁺; ¹H NMR (CDCl₃, 400 MHz): δ 1.41 (s, 9H), 1.57 (t, 3H), 2.23 (m, 2H), 2.65 (t, 2H), 3.78 (s, 3H), 4.14 (q, 2H), 4.18 (t, 2H), 6.99 (s, 1H), 7.05 (d, 2H) 7.33 (s, 1H), 7.48 (d, 2H), 7.61-7.67 (m, 4H), 7.69 (d, 2H), 7.78 (s, 1H), 7.83 (d, 2H) ppm.

Step 2: 4-(4′-{2-[4-tert-Butyl-phenyl)-1-ethyl-1H-imidazol-2-yl]-(E)-vinyl}-biphenyl-4-yloxy)-butyric acid methyl ester (261 mg, 0.5 mmol) was hydrolyzed according to general procedure F to give 4-(4′-{2-[4-tert-butyl-phenyl)-1-ethyl-1H-imidazol-2-yl]-(E)-vinyl}-biphenyl-4-yloxy)-butyric acid (218 mg, 85%).

LCMS: m/z 509 (M+H)⁺; ¹H NMR (DMSO-d₆, 400 MHz): δ 0.89 (s, 9H), 1.30 (t, 3H), 1.50 (m, 2H), 2.17 (t, 2H), 4.06 (q, 2H), 4.10 (t, 2H), 6.82 (d, 2H), 6.93 (d, 2H) 7.14 (s, 1H), 7.39-7.41 (m, 4H), 7.43 (d, 1H), 7.54 (d, 2H), 7.71 (d, 2H), 7.75 (s, 1H) ppm.

### Example 198

### 2-[2-(4-Bromo-phenyl)-(E)-vinyl]-1-ethyl-4-(4-trifluoromethyl-phenyl)-1H-imidazole

2-[2-(4-Bromo-phenyl)-(E)-vinyl]-1-ethyl-4-(4-trifluoromethyl-phenyl)-1H-imidazole (372 mg, 88%) was prepared according to general procedure A using trans-4-bromocinnamic acid (227 mg, 1 mmol) and 2-bromo-1-(4-trifluoromethyl)-phenyl-1-ethanone (267 mg, 1 mmol) and obtained 2-[2-(4-bromo-phenyl)-(E)-vinyl]l-4-(4-trifluoromethyl-phenyl)-1H-imidazole (393 mg, 1 mmol) was treated with bromoethane (109 mg, 1 mmol) following general procedure E.

LCMS: 422 (M+H)⁺; ¹H NMR (CDCl₃, 400 MHz): δ 1.52 (t, 3H), 4.11 (q, 2H), 6.91 (d, 1H), 7.31 (d, 1H), 7.41 (d, 2H), 7.43 (d, 2H), 7.51 (d, 1H), 7.61-7.68 (m, 2H), 7.68 (s, 1H), 7.93 (d, 1H) ppm.

### Example 199

### 4-(−4′-{2-[1-Ethyl-4-(4-trifluoromethyl-phenyl)-1H-imidazol-2-yl]-(E)-vinyl}-biphenyl-4-yloxy)-butyric acid

Step 1: 2-[2-(4-Bromo-phenyl)-(E)-vinyl]-1-ethyl-4-(4-trifluoromethyl-phenyl)-1H-imidazole (421 mg, 1 mmol) was coupled with 4-hydroxyphenylboronic acid (137 mg, 1 mmol) following general procedure B and obtained 4′-{2-[1-ethyl-4-(4-trifluoromethyl-phenyl)-1H-imidazol-2-yl]-(E)-vinyl}-biphenyl-4-ol (434 mg, 1 mmol) was alkylated with methyl 4-bromobutyrate (181 mg, 1 mmol) following general procedure E to give 4-(−4′-{2-[1-ethyl-4-(4-trifluoromethyl-phenyl)-1H-imidazol-2-yl]-(E)-vinyl}-biphenyl-4-yloxy)-butyric acid methyl ester (409 mg, 77%).

LCMS: m/z 535 (M+H)⁺; ¹H NMR (CDCl₃, 400 MHz): 1.51 (t, 3H), 2.17 (m, 2H), 2.59 (m, 2H), 3.71 (s, 3H), 4.06 (q, 2H), 4.15 (t, 2H), 6.92 (s, 1H), 6.99 (d, 2H), 7.32 (s, 1H), 7.54-7.59 (m, 4H), 7.61-7.64 (m, 2H), 7.74 (d, 1H), 7.78 (s, 2H), 7.95 (d, 2H) ppm.

Step 2: 4-(−4′-{2-[1-Ethyl-4-(4-trifluoromethyl-phenyl)-1H-imidazol-2-yl]-(E)-vinyl}-biphenyl-4-yloxy)-butyric acid methyl ester (267 mg, 0.5 mmol) was hydrolyzed according to general procedure F to give 4-(−4′-{2-[1-ethyl-4-(4-trifluoromethyl-phenyl)-1H-imidazol-2-yl]-(E)-vinyl}-biphenyl-4-yloxy)-butyric acid (209 mg, 80%).

LCMS: m/z 521 (M+H)⁺; ¹H NMR (DMSO-d₆, 400 MHz): δ 1.37 (t, 3H), 1.98 (m, 2H), 2.40 (t, 2H), 4.02 (q, 2H), 4.25 (t, 2H), 7.02 (d, 2H), 7.04 (s, 1H), 7.34 (d, 1H), 7.59 (d, 1H), 7.65-7.72 (m, 4H), 7.74-7.80 (m, 4H), 7.97 (s, 1H), 8.03 (d, 1H) ppm.

### Example 200

### 4-(−4′-{2-[1-Ethyl-4-(4-cyano-phenyl)-1H-imidazol-2-yl]-(E)-vinyl}-biphenyl-4-yloxy)-butyric acid

Step 1: 2-[2-(4-Bromo-phenyl)-(E)-vinyl]-1-ethyl-4-(4-cyano-phenyl)-1H-imidazole (378 mg, 1 mmol) was coupled with 4-hydroxyphenylboronic acid (137 mg, 1 mmol) following general procedure B and obtained 4′-{2-[1-ethyl-4-(4-cyanophenyl)-1H-imidazol-2-yl]-(E)-vinyl}-biphenyl-4-ol (391 mg, 1 mmol) was alkylated with methyl 4-bromobutyrate (181 mg, 1 mmol) following general procedure E to give 4-(−4′-{2-[1-ethyl-4-(4-cyanophenyl)-1H-imidazol-2-yl]-(E)-vinyl}-biphenyl-4-yloxy)-butyric acid methyl ester (352 mg, 71%).

LCMS: m/z 492 (M+H)⁺; ¹H NMR (CDCl₃, 400 MHz): 1.51 (t, 3H), 2.16 (m, 2H), 2.57 (m, 2H), 3.83 (s, 3H), 4.09 (q, 2H), 4.13 (t, 2H), 6.92 (d, 2H), 6.94-6.97 (m, 1H), 7.53-7.61 (m, 8H), 7.75 (d, 2H), 7.77 (d, 2H) ppm

Step 2: 4-(−4′-{2-[1-Ethyl-4-(4-cyano-phenyl)-1H-imidazol-2-yl]-(E)-vinyl}-biphenyl-4-yloxy)-butyric acid methyl ester (246 mg, 0.5 mmol) was hydrolyzed according to general procedure F to give 4-(−4′-{2-[1-ethyl-4-(4-cyano-phenyl)-1H-imidazol-2-yl]-(E)-vinyl}-biphenyl-4-yloxy)-butyric acid (197 mg, 82%).

LCMS: m/z 478 (M+H)⁺; ¹H NMR (DMSO-d₆, 400 MHz): 1.15 (t, 3H), 1.36 (m, 2H), 1.97 (m, 2H), 2.42 (t, 2H), 4.0 (q, 2H), 4.2 (t, 2H), 6.93 (d, 2H), 7.01 (d, 2H), 7.28 (d, 1H), 7.47 (d, 1H), 7.62-7.66 (m, 4H), 7.75-7.77 (m, 4H) ppm.

### Example 201

### 2-[2-(4-Bromo-phenyl)-(E)-vinyl]-1-ethyl-4-(4-chloro-phenyl)-1H-imidazole

2-[2-(4-Bromo-phenyl)-(E)-vinyl]-1-ethyl-4-(4-chloro-phenyl)-1H-imidazole (292 mg, 75%) was prepared according to general procedure A using trans-4-bromocinnamic acid (227 mg, 1 mmol) and 4-chlorophenacyl bromide (233 mg, 1 mmol) and obtained 2-[2-(4-bromo-phenyl)-(E)-vinyl]-4-(4-chloro-phenyl)-1H-imidazole (359 mg, 1 mmol) was treated with bromoethane (109 mg, 1 mmol) following general procedure E.

LCMS: m/z 388 (M+H)⁺; ¹H NMR (CDCl₃, 400 MHz): δ 1.47 (t, 3H), 4.12 (q, 2H), 6.90 (d, 2H), 7.33 (s, 1H), 7.35-7.40 (m, 2H), 7.41-7.42 (m, 2H), 7.48 (d, 1H), 7.50 (d, 1H), 7.76 (d, 2H) ppm

### Example 202

### 4-(−4′-{2-[1-Ethyl-4-(4-chloro-phenyl)-1H-imidazol-2-yl]-(E)-vinyl}-biphenyl-4-yloxy)-butyric acid

Step 1: 2-[2-(4-Bromo-phenyl)-(E)-vinyl]-1-ethyl-4-(4-chloro-phenyl)-1H-imidazole (387 mg, 1 mmol) was coupled with 4-hydroxyphenylboronic acid (137 mg, 1 mmol) following general procedure B and obtained 4′-{2-[1-ethyl-4-(4-chloro-phenyl)-1H-imidazol-2-yl]-(E)-vinyl}-biphenyl-4-ol (401 mg, 1 mmol) was alkylated with methyl 4-bromobutyrate (181 mg, 1 mmol) following general procedure E to give 4-(−4′-{2-[1-ethyl-4-(4-chloro-phenyl)-1H-imidazol-2-yl]-(E)-vinyl}-biphenyl-4-yloxy)-butyric acid methyl ester (381 mg, 76%).

LCMS: m/z 501 (M+H)⁺; ¹H NMR (CDCl₃, 400 MHz): 1.51 (t, 3H), 2.16 (m, 2H), 2.58 (m, 2H), 3.70 (s, 3H), 4.06 (q, 2H), 4.16 (t, 2H), 6.96-6.98 (m, 2H), 7.17-7.19 (m, 2H), 7.33-7.39 (m, 2H), 7.40-7.42 (m, 2H), 7.54-7.60 (m, 4H), 7.68 (s, 1H), (d, 2H) ppm.

Step 2: 4-(−4′-{2-[1-Ethyl-4-(4-chloro-phenyl)-1H-imidazol-2-yl]-(E)-vinyl}-biphenyl-4-yloxy)-butyric acid methyl ester (251 mg, 0.5 mmol) was hydrolyzed according to general procedure F to give 4-(−4′-{2-[1-ethyl-4-(4-chloro-phenyl)-1H-imidazol-2-yl]-(E)-vinyl}-biphenyl-4-yloxy)-butyric acid (196 mg, 80%).

LCMS: m/z 487 (M+H)⁺; ¹H NMR (DMSO-d₆, 400 MHz): 1.15 (t, 3H), 1.39 (m, 2H), 1.98 (m, 2H), 2.42 (t, 2H), 4.05 (q, 2H), 4.30 (t, 2H), 7.02 (d, 2H), 7.18 (s, 1H), 7.42 (d, 1H), 7.46 (d, 1H), 7.57-7.70 (m, 4H), 7.79-7.97 (m, 4H) ppm.

### Example 203

### 4-{2-[2-(4-Bromo-phenyl)-(E)-vinyl]-1-ethyl-1H-imidazol-4-yl}-benzoic acid methyl ester

4-{2-[2-(4-Bromo-phenyl)-(E)-vinyl]-1-ethyl-1H-imidazol-4-yl}-benzoic acid methyl ester (306 mg, 75%) was prepared according to general procedure A using trans-4-bromocinnamic acid (227 mg, 1 mmol) and 4-(−2-bromoacetyl)benzoic acid methyl ester (257 mg, 1 mmol) and obtained 4-{2-[2-(4-bromo-phenyl)-(E)-vinyl]-1H-imidazol-4-yl}-benzoic acid methyl ester (383 mg, 1 mmol) was treated with bromoethane (109 mg, 1 mmol) following general procedure E.

LCMS: m/z 412 (M+H)⁺; ¹H NMR (CDCl₃, 400 MHz): δ 1.52 (t, 3H), 3.92 (s, 3H), 4.12 (q, 2H), 6.87 (s, 1H), 6.91 (s, 1H), 7.34 (d, 2H), 7.41-7.43 (m, 2H), 7.49-7.51 (m, 2H), 7.64 (d, 1H), 7.88 (d, 1H), 8.06 (d, 1H) ppm.

### Example 204

### 4-(1-Ethyl-2-{2-[4′-(3-Methoxycarbonyl-propoxy)-biphenyl-4-yl}-1H-imidazol-4-yl)-benzoic acid

Step 1: 4-{2-[2-(4-Bromo-phenyl)-(E)-vinyl]-1-ethyl-1H-imidazol-4-yl}-benzoic acid methyl ester (411 mg, 1 mmol) was coupled with 4-hydroxyphenylboronic acid (137 mg, 1 mmol) following general procedure B and obtained 4-{1-ethyl-2-[2-(4-hydroxy-biphenyl-4-yl)-(E)-vinyl]-1H-imidazol-4-yl}-benzoic acid methyl ester (424 mg, 1 mmol) was alkylated with methyl 4-bromobutyrate (181 mg, 1 mmol) following general procedure E to give 4-(1-ethyl-2-{2-[4′-(3-methoxycarbonyl-propoxy)-biphenyl-4-yl}-1H-imidazol-4-yl)-benzoic acid methyl ester (404 mg, 77%).

LCMS: m/z 525 (M+H)⁺; ¹H NMR (CDCl₃, 400 MHz): 1.50 (t, 3H), 2.16 (m, 2H), 2.58 (m, 2H), 3.70 (s, 3H), 3.92 (s, 3H), 4.06 (q, 2H), 4.15 (t, 2H), 6.92 (s, 1H), 6.96-6.98 (m, 2H), 7.34 (s, 1H), 7.35-7.61 (m, 4H), 7.63 (s, 1H), 7.74 (s, 1H), 7.78 (s, 1H), 7.92 (d, 2H), 8.07 (d, 2H) ppm.

Step 2: 4-(1-Ethyl-2-{2-[4′-(3-Methoxycarbonyl-propoxy)-biphenyl-4-yl}-1H-imidazol-4-yl)-benzoic acid methyl ester (262 mg, 0.5 mmol) was hydrolyzed according to general procedure F to give 4-(1-ethyl-2-{2-[4′-(3-methoxycarbonyl-propoxy)-biphenyl-4-yl}-1H-imidazol-4-yl)-benzoic acid (189 mg, 64%).

LCMS: m/z 497 (M+H)⁺; ¹H NMR (DMSO-d₆, 400 MHz): 1.36 (t, 3H), 1.96 (m, 2H), 2.37 (m, 2H), 4.03 (q, 2H), 4.23 (t, 2H), 7.02 (d, 2H), 7.27 (s, 1H), 7.31 (s, 1H), 7.52 (d, 1H), 7.56 (d, 1H), 7.63 (d, 2H), 7.78 (d, 2H), 7.90 7.95 (m, 4H) ppm.

### Example 205

### 4-(4′-{2-[1-Ethyl-4-(4-methylcarbamoyl-phenyl)-1H-imidazol-2yl]-(E)-vinyl}-biphenyl-4-yloxy)-butyric acid

Step 1: 4-{2-[2-(4-Bromo-phenyl)-(E)-vinyl]-1-ethyl-1H-imidazol-4-yl}-benzoic acid (397 mg, 1 mmol) was coupled with methylamine according to general procedure G to give 4-{2-[2-(4-bromo-phenyl)-(E)-vinyl]-1-ethyl-1H-imidazol-4-yl}-N-methyl-benzamide.

4-{2-[2-(4-Bromo-phenyl)-(E)-vinyl]-1-ethyl-1H-imidazol-4-yl}-N-methyl-benzamide (410 mg, 1 mmol) was coupled with 4-hydroxyphenylboronic acid (137 mg, 1 mmol) following general procedure B and obtained 4-{1-ethyl-2-[2-(4′-hydroxy-biphenyl-4-yl)-(E)-vinyl]-1H-imidazol-4-yl}-N-methyl-benzamide (423 mg, 1 mmol) was alkylated with methyl 4-bromobutyrate (181 mg, 1 mmol) following general procedure E to give 4-(4′-{2-[1-ethyl-4-(4-methylcarbamoyl-phenyl)-1H-imidazol-2-yl]-(E)-vinyl}-biphenyl-4-yloxy)-butyric acid methyl ester (406 mg, 78%).

LCMS: m/z 524 (M+H)⁺; ¹H NMR (DMSO-d₆, 400 MHz): 1.40 (t, 3H), 2.01 (m, 2H), 2.79 (d, 2H), 3.33 (s, 3H), 3.61 (s, 3H), 4.05 (q, 2H), 4.25 (t, 2H), 7.03 (d, 2H), 7.32 (d, 1H), 7.57 (d, 1H), 7.67 (d, 2H), 7.77 (d, 2H), 7.80-7.89 (m, 6H), 8.41 (d, 2H) ppm.

Step 2: 4-(4′-{2-[1-Ethyl-4-(4-methylcarbamoyl-phenyl)-1H-imidazol-2-yl]-(E)-vinyl}-biphenyl-4-yloxy)-butyric acid methyl ester (262 mg, 0.5 mmol) was hydrolyzed according to general procedure F to give 4-(4′-{2-[1-ethyl-4-(4-methylcarbamoyl-phenyl)-1H-imidazol-2-yl]-(E)-vinyl}-biphenyl-4-yloxy)-butyric acid (199 mg, 78%).

LCMS: m/z 510 (M+H)⁺; ¹H NMR (DMSO-d₆, 400 MHz): 1.40 (t, 3H), 1.96 (m, 2H), 2.35 (m, 2H), 2.79 (s, 3H), 4.05 (q, 2H), 4.23 (t, 2H), 7.04 (d, 2H), 7.28 (s, 1H), 7.32 (s, 1H), 7.53 (s, 1H), 7.56 (s, 1H), 7.64 (d, 2H), 7.77 (d, 2H), 7.83-7.89 (m, 4H), 8.41 (d, 2H) ppm.

### Example 206

### 4-{4′-[2-(4-Biphenyl-4-yl-1-ethyl-1H-imidazol-2-yl)-(E)-vinyl]-biphenyl-4-yloxy}-butyric acid

Step 1: 4-Biphenyl-4-yl-2-[2-(4-bromo-phenyl)-(E)-vinyl]-1-ethyl-1H-imidazole (429 mg, 1 mmol) was coupled with 4-hydroxyphenylboronic acid (137 mg, 1 mmol) following general procedure B and obtained 4′-[2-(4-biphenyl-4-yl-1-ethyl-1H-imidazol-2-yl)-(E)-vinyl]-biphenyl-4-ol (442 mg, 1 mmol) was alkylated with methyl 4-bromobutyrate (181 mg, 1 mmol) following general procedure E to give 4-{4′-[2-(4-biphenyl-4-yl-1-ethyl-1H-imidazol-2-yl)-(E)-vinyl]-biphenyl-4-yloxy}-butyric acid methyl ester (399 mg, 74%).

LCMS: m/z 543 (M+H)⁺; ¹H NMR (CDCl₃, 400 MHz): 1.54 (t, 3H), 2.17 (m, 2H), 2.59 (m, 2H), 3.71 (s, 3H), 4.05 (q, 2H), 4.15 (t, 2H), 6.94 (s, 1H), 6.96-6.99 (m, 2H), 7.29 (s, 1H), 7.34-7.43 (m, 2H), 7.45-7.47 (m, 2H), 7.55-7.58 (m, 4H), 7.62-7.67 (m, 5H), 7.79 (s, 1H), 7.93 (d, 2H) ppm.

Step 2: 4-{4′-[2-(4-Biphenyl-4-yl-1-ethyl-1H-imidazol-2-yl)-(E)-vinyl]-biphenyl-4-yloxy}-butyric acid methyl ester (271 mg, 0.5 mmol) was hydrolyzed according to general procedure F to give 4-{4′-[2-(4-biphenyl-4-yl-1-ethyl-1H-imidazol-2-yl)-(E)-vinyl]-biphenyl-4-yloxy}-butyric acid (201 mg, 76%).

LCMS: m/z 529 (M+H)⁺; ¹H NMR (DMSO-d₆, 400 MHz): 1.41 (t, 3H), 1.97 (m, 2H), 2.42 (t, 2H), 4.04 (q, 2H), 4.23 (t, 2H), 7.03 (d, 2H), 7.28 (s, 1H), 7.32-7.37 (m, 2H), 7.37-7.44 (m, 2H), 7.46-7.48 (m, 4H), 7.53 (s, 1H), 7.57 (s, 1H), 7.78-7.82 (m, 5H), 7.92 (d, 2H) ppm.

### Example 207

### 4-Biphenyl-3-yl-2-[2-(4-bromo-phenyl)-(E)-vinyl]-1-ethyl-1H-imidazole

4-Biphenyl-3-yl-2-[2-(4-bromo-phenyl)-(E)-vinyl]-1-ethyl-1H-imidazole (314 mg, 73%) was prepared according to general procedure A using trans-4-bromocinnamic acid (227 mg, 1 mmol) and α-bromo-3-phenyl-acetophenone (275 mg, 1 mmol) and obtained 4-biphenyl-3-yl-2-[2-(4-bromo-phenyl)-(E)-vinyl]-1H-imidazole (401 mg, 1 mmol) was treated with bromoethane (109 mg, 1 mmol) following general procedure E.

LCMS: m/z 430 (M+H)⁺; ¹H NMR (CDCl₃, 400 MHz): δ 1.54 (t, 3H), 4.17 (q, 2H), 6.90 (s, 1H), 7.34 (d, 2H), 7.43 (d, 2H), 7.44-7.51 (m, 4H), 7.61-7.65 (m, 4H), 7.91 (d, 2H), 8.01 (s, 1H) ppm.

### Example 208

### 4-{-4′-[2-(4-Biphenyl-3-yl-1-ethyl-1H-imidazol-2-yl)-(E)-vinyl]-biphenyl-4-yloxy}-butyric acid

Step 1: 4-Biphenyl-3-yl-2-[2-(4-bromo-phenyl)-(E)-vinyl]-1-ethyl-1H-imidazole (429 mg, 1 mmol) was coupled with 4-hydroxyphenylboronic acid (137 mg, 1 mmol) following general procedure B and obtained 4′-[2-(4-biphenyl-3-yl-1-ethyl-1H-imidazol-2-yl)-(E)-vinyl]-biphenyl-4-ol (442 mg, 1 mmol) was alkylated with 4-bromomethyl butyrate (181 mg, 1 mmol) following general procedure E to give 4-{-4′-[2-(4-biphenyl-3-yl-1-ethyl-1H-imidazol-2-yl)-(E)-vinyl]-biphenyl-4-yloxy}-butyric acid methyl ester (418 mg, 77%).

LCMS: m/z 543 (M+H)⁺; ¹H NMR (CDCl₃, 400 MHz): δ 1.51 (t, 3H), 2.14 (m, 2H), 2.56 (m, 2H), 3.70 (s, 3H), 4.07 (q, 2H), 4.13 (t, 2H), 6.93 (s, 1H), 6.95-6.97 (m, 2H), 7.29 (s, 1H), 7.35-7.37 (m, 2H), 7.44-7.46 (m, 2H), 7.47-7.57 (m, 4H), 7.61-7.70 (m, 5H), 7.74-7.8 (m, 2H), 8.07 (s, 1H) ppm

Step 2: 4-{-4′-[2-(4-Biphenyl-3-yl-1-ethyl-1H-imidazol-2-yl)-(E)-vinyl]-biphenyl-4-yloxy}-butyric acid methyl ester (271 mg, 0.5 mmol) was hydrolyzed according to general procedure F to give 4-{-4′-[2-(4-biphenyl-3-yl-1-ethyl-1H-imidazol-2-yl)-(E)-vinyl]-biphenyl-4-yloxy}-butyric acid (201 mg, 76%).

LCMS: m/z 529 (M+H)⁺; ¹H NMR (DMSO-d₆, 400 MHz): δ 1.41 (t, 3H), 1.97 (m, 2H), 2.42 (t, 2H), 4.04 (q, 2H), 4.23 (t, 2H), 7.03 (d, 2H), 7.28 (s, 1H), 7.32-7.37 (m, 2H), 7.37-7.44 (m, 2H), 7.46-7.48 (m, 4H), 7.53 (s, 1H), 7.78-7.82 (m, 5H), 7.92 (d, 2H), 8.02 (s, 1H) ppm.

### Example 209

### 4-(4′-{2-[4-(2-Chloro-phenyl)-1-ethyl-1H-imidazole-2-yl]-(E)-vinyl}-biphenyl-4-yloxy)-butyric acid methyl ester

Trans-4-bromocinnamic acid (227 mg, 1 mmol) was reacted with 2-chloro phenacylbromide (233 mg, 1 mmol) according to general procedure A and obtained 2-[2-(4-bromo-phenyl)-(E)-vinyl]-4-(2-chloro-phenyl)-1H-imidazole (359 mg, 1 mmol) was treated with bromoethane (109 mg, 1 mmol) following general procedure. The resulted 2-[2-(4-bromo-phenyl)-(E)-vinyl]-4-(2-chloro-phenyl)-1-ethyl-1H-imidazole (387 mg, 1 mmol) was coupled with 4-hydroxyphenylboronic acid (137 mg, 1 mmol) following general procedure B and obtained 4′-{2-[4-(2-chloro-phenyl)-1-ethyl-1H-imidazol-2-yl]-(E)-vinyl}-biphenyl-4-ol (401 mg, 1 mmol) was alkylated with methyl 4-bromobutyrate (181 mg, 1 mmol) following general procedure E to give 4-(4′-{2-[4-(2-chloro-phenyl)-1-ethyl-1H-imidazole-2-yl]-(E)-vinyl}-biphenyl-4-yloxy)-butyric acid methyl ester (399 mg, 79%).

LCMS: m/z 501 (M+H)⁺; ¹H NMR (CDCl₃, 400 MHz): δ 1.51 (t, 3H), 2.16 (m, 2H), 2.58 (m, 2H), 3.70 (s, 3H), 4.06 (q, 2H), 4.14 (t, 2H), 6.92 (s, 1H), 6.96-6.98 (m, 2H), 7.17-7.19 (m, 2H), 7.33-7.40 (m, 2H), 7.42 (d, 2H), 7.54-7.59 (m, 2H), 7.60-7.67 (m, 2H), 7.72 (s, 1H), 7.76 (s, 1H) ppm

### Example 210

### 4-(4′-{2-[4-(2-Chloro-phenyl)-1-ethyl-1H-imidazole-2-yl]-(E)-vinyl}-biphenyl-4-yloxy)-butyric acid

4-(4′-{2-[4-(2-Chloro-phenyl)-1-ethyl-1H-imidazole-2-yl]-(E)-vinyl}-biphenyl-4-yloxy)-butyric acid methyl ester (250 mg, 0.5 mmol) was hydrolyzed according to general procedure F to give 4-(4′-{2-[4-(2-chloro-phenyl)-1-ethyl-1H-imidazole-2-yl]-(E)-vinyl}-biphenyl-4-yloxy)-butyric acid (196 mg, 80%).

LCMS: m/z 487 (M+H)⁺; ¹H NMR (DMSO-d₆, 400 MHz): δ 1.39 (t, 3H), 1.98 (m, 2H), 2.42 (t, 2H), 4.05 (q, 2H), 4.30 (t, 2H), 7.04 (d, 2H), 7.23-7.29 (m, 2H), 7.33 (s, 1H), 7.38-7.40 (m, 2H), 7.42 (d, 1H), 7.47 (s, 1H), 7.49 (s, 1H), 7.54-7.67 (m, 2H), 7.80 (d, 1H), 7.91 (s, 1H), 8.21 (d, 1H) ppm.

### Example 211

### 4-(4′-{2-[4-(2-Methoxy-phenyl)-1-ethyl-1H-imidazole-2-yl]-(E)-vinyl}-biphenyl-4-yloxy)-butyric acid methyl ester

Trans-4-bromocinnamic acid (227 mg, 1 mmol) was reacted with 2-methoxy phenacylbromide (229 mg, 1 mmol) according to general procedure A and obtained 2-[2-(4-bromo-phenyl)-(E)-vinyl]-4-(2-methoxy-phenyl)-1H-imidazole (355 mg, 1 mmol) was treated with bromoethane (109 mg, 1 mmol) following general procedure E. The resulted 2-[2-(4-bromo-phenyl)-(E)-vinyl]-4-(2-methoxy-phenyl)-1-ethyl-1H-imidazole (383 mg, 1 mmol) was coupled with 4-hydroxyphenylboronic acid (137 mg, 1 mmol) following general procedure B and obtained 4′-{2-[4-(2-methoxy-phenyl)-1-ethyl-1H-imidazol-2-yl]-(E)-vinyl}-biphenyl-4-ol (396 mg, 1 mmol) was alkylated with methyl 4-bromobutyrate (181 mg, 1 mmol) following general procedure E to give 4-(4′-{2-[4-(2-methoxy-phenyl)-1-ethyl-1H-imidazole-2-yl]-(E)-vinyl}-biphenyl-4-yloxy)-butyric acid methyl ester (375 mg, 75%).

LCMS: m/z 497 (M+H)⁺; ¹H NMR (CDCl₃, 400 MHz): δ 1.52 (t, 3H), 2.16 (m, 2H), 2.58 (m, 2H), 3.70 (s, 3H), 3.96 (s, 3H), 4.07 (q, 2H), 4.13 (t, 2H), 6.93 (s, 1H), 6.95-6.96 (m, 2H), 6.97-7.07 (m, 2H), 7.23-7.25 (m, 2H), 7.53-7.55 (m, 2H), 7.57-7.60 (m 2H), 7.72 (s, 1H), 7.76 (s, 1H), 8.35 (d, 2H) ppm.

### Example 212

### 4-(4′-{2-[4-(2-Methoxy-phenyl)-1-ethyl-1H-imidazole-2-yl]-(E)-vinyl}-biphenyl-4-yloxy)-butyric acid

4-(4′-{2-[4-(2-Methoxy-phenyl)-1-ethyl-1H-imidazole-2-yl]-(E)-vinyl}-biphenyl-4-yloxy)-butyric acid methyl ester (248 mg, 0.5 mmol) was hydrolyzed according to general procedure F to give 4-(4′-{2-[4-(2-methoxy-phenyl)-1-ethyl-1H-imidazole-2-yl]-(E)-vinyl}-biphenyl-4-yloxy)-butyric acid (189 mg, 78%).

LCMS: m/z 483 (M+H)⁺; ¹H NMR (CDCl₃, 400 MHz): δ 1.52 (t, 3H), 2.16 (m, 2H), 2.58 (m, 2H), 3.95 (s, 3H), 4.03 (q, 2H), 4.13 (t, 2H), 6.84 (d, 2H), 6.91 (s, 1H), 6.95 (d, 1H), 6.97-7.09 (m, 2H), 7.23-7.25 (m, 2H), 7.44-7.46 (m, 2H), 7.52-7.57 (m 2H), 7.74 (s, 1H), 7.78 (s, 1H), 8.24 (d, 1H) ppm.

### Example 213

### 4-(4′-{2-[4-(2,4-dichloro-phenyl)-1-ethyl-1H-imidazol-2-yl]-(E)-vinyl}-3′-fluoro-biphenyl-4-yloxy)-butyric acid

Step 1: 2-[2-(4-Bromo-2-fluoro-phenyl)-(E)-vinyl]-4-(2,4-dichloro-phenyl)-1-ethyl-1H-imidazole (321 mg, 73%) was prepared according to general procedure A using trans-4-bromo-2-fluorocinnamic acid (245 mg, 1 mmol) and α-bromo-2,4-dichloroacetophenone (267 mg, 1 mmol) and obtained 2-[2-(4-bromo-2-fluoro-phenyl)-(E)-vinyl]-4-(2,4-dichloro-phenyl)-1H-imidazole (412 mg, 1 mmol) which was then treated with bromoethane (109 mg, 1 mmol) following general procedure E.

LCMS: m/z 440 (M+H)⁺; ¹H NMR (CDCl₃, 400 MHz): δ 1.52 (t, 3H), 4.08 (q, 2H), 4.14 (t, 2H), 7.07 (d, 1H), 7.25-7.28 (m, 2H), 7.29-7.39 (m, 2H), 7.42 (s, 1H), 8.24 (d, 1H) ppm.

Step 2: 2-[2-(4-Bromo-2-fluoro-phenyl)-(E)-vinyl]-4-(2,4-dichloro-phenyl)-1-ethyl-1H-imidazole (440 mg, 1 mmol) was coupled with 4-hydroxy phenyl boronic acid (137 mg, 1 mmol) following general procedure B and obtained 4′-{2-[4-(2,4-dichloro-phenyl)-1-ethyl-1H-imidazole-2-yl]-(E)-vinyl}-3′-fluoro-biphenyl-4-ol (453 mg, 1 mmol) was alkylated with 4-bromomethyl butyrate (181 mg, 1 mmol) following general procedure E to give 4-(4′-{2-[4-(2,4-dichloro-phenyl)-1-ethyl-1H-imidazol-2-yl]-(E)-vinyl}-3′-fluoro-biphenyl-4-yloxy)-butyric acid methyl ester (453 mg, 81%).

LCMS: m/z 553 (M+H)⁺; ¹H NMR (CDCl₃, 400 MHz): δ 1.52 (t, 3H), 2.17 (m, 2H), 2.58 (m, 2H), 3.71 (s, 3H), 4.07 (q, 2H), 4.15 (t, 2H), 6.96 (d, 2H), 7.08 (s, 1H), 7.12 (s, 1H), 7.28-7.37 (m, 2H), 7.43 (s, 1H), 7.53-7.61 (m, 4H), 7.69 (s, 1H), 8.29 (d, 1H) ppm.

Step 3: 4-(4′-{2-[4-(2,4-dichloro-phenyl)-1-ethyl-1H-imidazol-2-yl]-(E)-vinyl}-3′-fluoro-biphenyl-4-yloxy)-butyric acid methyl ester (276 mg, 0.5 mmol) was hydrolyzed according to general procedure F to give 4-(4′-{2-[4-(2,4-dichloro-phenyl)-1-ethyl-1H-imidazol-2-yl]-(E)-vinyl}-3′-fluoro-biphenyl-4-yloxy)-butyric acid (212 mg, 79%).

LCMS: m/z 539 (M+H)⁺; ¹H NMR (DMSO-d₆, 400 MHz): δ 1.40 (t, 3H), 1.97 (m, 2H), 2.42 (t, 2H), 4.04 (q, 2H), 4.30 (t, 2H), 7.05 (d, 2H), 7.38 (s, 1H), 7.42 (s, 1H), 7.50 (d, 1H), 7.53 (s, 1H), 7.58 (d, 2H), 7.67-7.73 (m, 2H), 8.01-8.05 (m, 2H), 8.21 (d, 1H) ppm.

### Example 214

### 4-(4′-{2-[4-(2,4-dichloro-phenyl)-1-ethyl-1H-imidazol-2-yl]-(E)-vinyl}-3′-fluoro-biphenyl-3-yloxy)-butyric acid methyl ester

2-[2-(4-Bromo-2-fluoro-phenyl)-(E)-vinyl]-4-(2,4-dichloro-phenyl)-1-ethyl-1H-imidazole (440 mg, 1 mmol) was coupled with 3-hydroxyphenylboronic acid (137 mg, 1 mmol) following general procedure B and obtained 4′-{2-[4-(2,4-dichloro-phenyl)-1-ethyl-1H-imidazole-2-yl]-(E)-vinyl}-3′-fluoro-biphenyl-3-ol (453 mg, 1 mmol) was alkylated with 4-bromomethyl butyrate (181 mg, 1 mmol) following general procedure E to give 4-(4′-{2-[4-(2,4-dichloro-phenyl)-1-ethyl-1H-imidazol-2-yl]-(E)-vinyl}-3′-fluoro-biphenyl-3-yloxy)-butyric acid methyl ester (409 mg, 74%).

LCMS: m/z 553 (M+H)⁺; ¹H NMR (CDCl₃, 400 MHz): δ 1.53 (t, 3H), 2.16 (m, 2H), 2.56 (m, 2H), 3.69 (s, 3H), 4.05 (q, 2H), 4.15 (t, 2H), 6.88 (d, 2H), 6.90-7.08 (m, 2H), 7.11 (d, 1H), 7.12 (s, 1H), 7.17-7.32 (m, 2H), 7.57-7.68 (m, 2H), 7.79 (s, 1H), 8.27 (d, 1H), 8.27 (d, 1H) ppm.

### Example 215

### 4-(4′-{2-[4-(2,4-dichloro-phenyl)-1-ethyl-1H-imidazol-2-yl]-(E)-vinyl}-3′-fluoro-biphenyl-3-yloxy)-butyric acid

4-(4′-{2-[4-(2,4-dichloro-phenyl)-1-ethyl-1H-imidazol-2-yl]-(E)-vinyl}-3′-fluoro-biphenyl-3-yloxy)-butyric acid methyl ester (276 mg, 0.5 mmol) was hydrolyzed according to general procedure F to give-4-(4′-{2-[4-(2,4-dichloro-phenyl)-1-ethyl-1H-imidazol-2-yl]-(E)-vinyl}-3′-fluoro-biphenyl-3-yloxy)-butyric acid (210 mg, 78%).

LCMS: m/z 539 (M+H)⁺; ¹H NMR (DMSO-d₆, 400 MHz): δ 1.37 (t, 3H), 1.97 (m, 2H), 2.41 (t, 2H), 4.06 (q, 2H), 4.29 (t, 2H), 6.98 (d, 2H), 7.29-7.37 (m, 2H), 7.39-7.48 (m, 2H), 7.50-7.64 (m, 2H), 7.70 (s, 1H), 7.99 (s, 1H), 8.06-8.08 (m, 2H), 8.25 (d, 1H) ppm.

### Example 216

### 4-(3′-{2-[4-(2,4-dichloro-phenyl)-1-ethyl-1H-imidazol-2-yl]-(E)-vinyl}-biphenyl-3-yloxy)-butyric acid methyl ester

Step 1: 2-[2-(3-Bromo-phenyl)-(E)-vinyl]-4-(2,4-dichloro-phenyl)-1-ethyl-1H-imidazole (312 mg, 74%) was prepared according to general procedure A using trans 3-bromo cinnamic acid (227 mg, 1 mmol) and 2-bromo-2,4-dichloro acetophenone (267 mg, 1 mmol) and obtained 2-[2-(3-Bromo-phenyl)-(E)-vinyl]-4-(2,4-dichloro-phenyl)-1H-imidazole (394 mg, 1 mmol) was treated with bromo ethane (109 mg, 1 mmol) following general procedure E.

LCMS: m/z 422 (M+H)⁺.

Step 2: 2-[2-(3-Bromo-phenyl)-(E)-vinyl]-4-(2,4-dichloro-phenyl)-1-ethyl-1H-imidazole (422 mg, 1 mmol) was coupled with 3-hydroxy phenyl boronic acid (137 mg, 1 mmol) following general procedure B and obtained 3′-{2-[4-(2,4-dichloro-phenyl)-1-ethyl-1H-imidazol-2-yl]-(E)-vinyl}-biphenyl-3-ol (435 mg, 1 mmol) was alkylated with 4-bromomethyl butyrate (181 mg, 1 mmol) following general procedure E to give 4-(3′-{2-[4-(2,4-dichloro-phenyl)-1-ethyl-1H-imidazol-2-yl]-(E)-vinyl}-biphenyl-3-yloxy)-butyric acid methyl ester (429 mg, 80%).

LCMS: m/z 535 (M+H)⁺; ¹H NMR (CDCl₃, 400 MHz): δ 1.53 (t, 3H), 2.15 (m, 2H), 2.58 (m, 2H), 3.69 (s, 3H), 4.07 (q, 2H), 4.15 (t, 2H), 6.88 (d, 2H), 6.95 (s, 1H), 6.98 (s, 1H), 7.14 (d, 1H), 7.21 (d, 1H), 7.30-7.33 (m, 2H), 7.35-7.46 (m, 2H), 7.50-7.53 (m, 2H), 7.74 (d, 1H), 8.26 (d, 1H) ppm.

### Example 217

### 4-(3′-{2-[4-(2,4-dichloro-phenyl)-1-ethyl-1H-imidazol-2-yl]-(E)-vinyl}-4-methoxy-biphenyl-4-yloxy)-butyric acid methyl ester

Step 1: 2-[2-(5-Bromo-2-methoxy-phenyl)-(E)-vinyl]-4-(2,4-dichloro-phenyl)-1-ethyl-1H-imidazole (318 mg, 70%) was prepared according to general procedure A using trans-5-bromo-2-methoxycinnamic acid (257 mg, 1 mmol) and 2-bromo-2,4-dichloro-acetophenone (267 mg, 1 mmol) and obtained 2-[2-(5-bromo-2-methoxy-phenyl)-(E)-vinyl]-4-(2,4-dichloro-phenyl)-1H-imidazole (424 mg, 1 mmol) was treated with bromo ethane (109 mg, 1 mmol) following general procedure E.

LCMS: m/z 452 (M+H)⁺; ¹H NMR (CDCl₃, 400 MHz): δ 1.52 (t, 3H), 3.88 (s, 3H), 4.14 (q, 2H), 4.14 (t, 2H), 6.80 (d, 1H), 7.29-7.32 (m, 2H), 7.41 (s, 1H), 7.66 (d, 1H), 7.90 (d, 1H), 8.27 (d, 1H) ppm.

Step 2: 2-[2-(5-Bromo-2-methoxy-phenyl)-(E)-vinyl]-4-(2,4-dichloro-phenyl)-1-ethyl-1H-imidazole (452 mg, 1 mmol) was coupled with 4-hydroxyphenylboronic acid (137 mg, 1 mmol) following general procedure B and obtained 3′-{2-[4-(2,4-dichloro-phenyl)-1-ethyl-1H-imidazol-2-yl]-(E)-vinyl}-4-methoxy-biphenyl-4-ol (465 mg, 1 mmol) was alkylated with 4-bromomethyl butyrate (181 mg, 1 mmol) following general procedure E to give 4-(3′-{2-[4-(2,4-dichloro-phenyl)-1-ethyl-1H-imidazol-2-yl]-(E)-vinyl}-4-methoxy-biphenyl-4-yloxy)-butyric acid methyl ester (417 mg, 74%).

LCMS: m/z 565 (M+H)⁺; ¹H NMR (CDCl₃, 400 MHz): δ 1.51 (t, 3H), 2.15 (m, 2H), 2.57 (m, 2H), 3.71 (s, 3H), 3.95 (s, 3H), 4.05 (q, 2H), 4.14 (t, 2H), 6.96-6.99 (m, 2H), 7.12 (d, 2H), 7.31 (d, 2H), 7.32-7.42 (m, 2H), 7.44-7.52 (m, 2H), 7.67 (s, 1H), 7.90 (d, 1H), 8.3 (d, 1H) ppm.

### Example 218

### 4-(3′-{2-[4-(2,4-dichloro-phenyl)-1-ethyl-1H-imidazol-2-yl]-(E)-vinyl}-4-methoxy-biphenyl-4-yloxy)-butyric acid

4-(3′-{2-[4-(2,4-dichloro-phenyl)-1-ethyl-1H-imidazol-2-yl]-(E)-vinyl}-4-methoxy-biphenyl-4-yloxy)-butyric acid methyl ester (283 mg, 0.5 mmol) was hydrolyzed according to general procedure F to give 4-(3′-{2-[4-(2,4-dichloro-phenyl)-1-ethyl-1H-imidazol-2-yl]-(E)-vinyl}-4-methoxy-biphenyl-4-yloxy)-butyric acid title compound (219 mg, 79%).

LCMS: m/z 551 (M+H)⁺; ¹H NMR (DMSO-d₆, 400 MHz): δ 1.35 (t, 3H), 1.97 (m, 2H), 2.41 (t, 2H), 3.91 (s, 3H), 4.03 (q, 2H), 4.27 (t, 2H), 7.01 (d, 2H), 7.11 (d, 2H), 7.33 (s, 1H), 7.37 (s, 1H), 7.48 (d, 1H), 7.50 (d, 1H), 7.64 (d, 1H), 7.85 (d, 1H), 7.94 (s, 1H), 8.02 (d, 1H), 8.24 (d, 1H) ppm.

### Example 219

### 4-(3′-{2-[4-(2,4-Dichloro-phenyl)-1-ethyl-1H-imidazol-2-yl]-(E)-vinyl}-4′-methoxy-biphenyl-3-yloxy)-butyric acid methyl ester

2-[2-(5-Bromo-2-methoxy-phenyl)-(E)-vinyl]-4-(2,4-dichloro-phenyl)-1-ethyl-1H-imidazole (452 mg, 1 mmol) was coupled with 3-hydroxyphenylboronic acid (137 mg, 1 mmol) following general procedure B and obtained 3′-{2-[4-(2,4-dichloro-phenyl)-1-ethyl-1H-imidazol-2-yl]-(E)-vinyl}-4-methoxy-biphenyl-3-ol (465 mg, 1 mmol) was alkylated with 4-bromomethyl butyrate (181 mg, 1 mmol) following general procedure E to give 4-(3′-{2-[4-(2,4-Dichloro-phenyl)-1-ethyl-1H-imidazol-2-yl]-(E)-vinyl}-4′-methoxy-biphenyl-3-yloxy)-butyric acid methyl ester (413 mg, 73%).

LCMS: 565 (M+H)⁺; ¹H NMR (CDCl₃, 400 MHz): δ 1.51 (t, 3H), 2.15 (m, 2H), 2.59 (m, 2H), 3.69 (s, 3H), 3.96 (s, 3H), 4.08 (q, 2H), 4.15 (t, 2H), 6.86 (d, 2H), 7.00 (d, 1H), 7.09 (s, 1H), 7.11-7.17 (m, 2H), 7.19 (d, 1H), 7.31-7.42 (m, 2H), 7.48 (d, 1H), 7.76 (s, 1H), 8.00 (d, 1H), 8.31 (d, 1H) ppm.

### Example 220

### 4-(3′-{2-[4-(2,4-Dichloro-phenyl)-1-ethyl-1H-imidazol-2-yl]-(E)-vinyl}-4′-methoxy-biphenyl-3-yloxy)-butyric acid

4-(3′-{2-[4-(2,4-Dichloro-phenyl)-1-ethyl-1H-imidazol-2-yl]-(E)-vinyl}-4′-methoxy-biphenyl-3-yloxy)-butyric acid methyl ester (283 mg, 0.5 mmol) was hydrolyzed according to general procedure F to give 4-(3′-{2-[4-(2,4-dichloro-phenyl)-1-ethyl-1H-imidazol-2-yl]-(E)-vinyl}-4′-methoxy-biphenyl-3-yloxy)-butyric acid (212 mg, 77%).

LCMS: m/z 551 (M+H)⁺; ¹H NMR (DMSO-d₆, 400 MHz): δ 1.36 (t, 3H), 1.98 (m, 2H), 2.41 (t, 2H), 3.92 (s, 3H), 4.06 (q, 2H), 4.27 (t, 2H), 6.92 (d, 2H), 7.12 (d, 2H), 7.23 (s, 1H), 7.27 (s, 1H), 7.29 (d, 1H), 7.47 (d, 1H), 7.49-7.63 (m, 2H), 7.84 (s, 1H), 8.06 (d, 1H), 8.24 (d, 1H) ppm.

### Example 221

### 4-(3′-{2-[4-(2,4-Dichloro-phenyl)-1-ethyl-1H-imidazol-2-yl]-(E)-vinyl}-4′-fluoro-biphenyl-4-yloxy)-butyric acid methyl ester

2-[2-(5-Bromo-2-fluoro-phenyl)-(E)-vinyl]-4-(2,4-dichloro-phenyl)-1-ethyl-1H-imidazole (369 mg, 84%) was prepared according to general procedure A using trans-5-bromo-2-fluorocinnamic acid (245 mg, 1 mmol) and 2-bromo-2,4-dichloroacetophenone (267 mg, 1 mmol) and obtained 2-[2-(5-bromo-2-fluoro-phenyl)-(E)-vinyl]-4-(2,4-dichloro-phenyl)-1H-imidazole (412 mg, 1 mmol) was treated with bromo ethane (109 mg, 1 mmol) following general procedure E.

LCMS: m/z 440 (M+H)⁺.

2-[2-(5-Bromo-2-fluoro-phenyl)-(E)-vinyl]-4-(2,4-dichloro-phenyl)-1-ethyl-1H-imidazole (440 mg, 1 mmol) was coupled with 4-hydroxyphenylboronic acid (137 mg, 1 mmol) following general procedure B and obtained 3′-{2-[4-(2,4-dichloro-phenyl)-1-ethyl-1H-imidazole-2-yl]-(E)-vinyl}-4′-fluoro-biphenyl-4-ol (453 mg, 1 mmol) was alkylated with 4-bromomethyl butyrate (181 mg, 1 mmol) following general procedure E to give 4-(3′-{2-[4-(2,4-Dichloro-phenyl)-1-ethyl-1H-imidazol-2-yl]-(E)-vinyl}-4′-fluoro-biphenyl-4-yloxy)-butyric acid methyl ester (415 mg, 75%).

LCMS: m/z 553 (M+H)⁺; ¹H NMR (CDCl₃, 400 MHz): δ 1.52 (t, 3H), 2.17 (m, 2H), 2.58 (m, 2H), 3.71 (s, 3H), 4.07 (q, 2H), 4.15 (t, 2H), 6.96 (d, 2H), 7.08-7.12 (m, 2H), 7.16 (s, 1H), 7.18 (d, 1H), 7.21 (d, 2H), 7.36 (d, 2H), 7.53 (d, 1H), 7.89 (s, 1H), 8.29 (d, 1H) ppm.

### Example 222

### 4-(3′-{2-[4-(2,4-Dichloro-phenyl)-1-ethyl-1H-imidazol-2-yl]-(E)-vinyl}-4′-fluoro-biphenyl-4-yloxy)-butyric acid

4-(3′-{2-[4-(2,4-Dichloro-phenyl)-1-ethyl-1H-imidazol-2-yl]-(E)-vinyl}-4′-fluoro-biphenyl-4-yloxy)-butyric acid methyl ester (276 mg, 0.5 mmol) was hydrolyzed according to general procedure F to give 4-(3′-{2-[4-(2,4-Dichloro-phenyl)-1-ethyl-1H-imidazol-2-yl]-(E)-vinyl}-4′-fluoro-biphenyl-4-yloxy)-butyric acid (214 mg, 805).

LCMS: m/z 539 (M+H)⁺; ¹H NMR (DMSO-d₆, 400 MHz): δ 1.37 (t, 3H), 1.98 (m, 2H), 2.42 (t, 2H), 4.04 (q, 2H), 4.28 (t, 2H), 7.05 (d, 2H), 7.31-7.46 (m, 2H), 7.47 (d, 2H), 7.50 (s, 1H), 7.64-7.69 (m 2H), 7.73 (d, 1H), 7.98 (s, 1H), 8.18 (d, 1H), 8.25 (d, 1H) ppm.

### Example 223

### 4-(4′-{2-[4-(2,4-Dichloro-phenyl)-1-ethyl-1H-imidazol-2-yl]-(E)-vinyl-}-3′-fluoro biphenyl-4-yloxymethyl)-benzoic acid methyl ester

2-[2-(4-Bromo-2-fluoro-phenyl)-(E)-vinyl]-4-(2,4-dichloro-phenyl)-1-ethyl-1H-imidazole (440 mg, 1 mmol) was coupled with 4-hydroxy phenyl boronic acid (137 mg, 1 mmol) following general procedure B and obtained 4′-{2-[4-(2,4-dichloro-phenyl)-1-ethyl-1H-imidazol-2-yl]-(E)-vinyl}-3′-fluoro-biphenyl-4-ol (453 mg, 1 mmol) was alkylated with methyl 4-(bromomethyl)benzoate (229 mg, 1 mmol) following general procedure E to give 4-(4′-{2-[4-(2,4-dichloro-phenyl)-1-ethyl-1H-imidazol-2-yl]-(E)-vinyl-}-3′-fluoro biphenyl-4-yloxymethyl)-benzoic acid methyl ester (423 mg, 70%).

LCMS: 601 (M+H)⁺. ¹H NMR (CDCl₃, 400 MHz): δ 1.53 (t, 3H), 3.92 (s, 3H), 4.15 (q, 2H), 5.18 (d, 2H), 7.03-7.07 (m, 2H), 7.11 (s, 1H), 7.27 (d, 2H), 7.30-7.36 (m, 2H), 7.42 (d, 2H), 7.51-7.60 (m, 4H), 7.68 (s, 1H), 7.78 (d, 1H), 8.08 (d, 1H), 8.28 (d, 1H) ppm.

### Example 224

### 4-(4′-{2-[4-(2,4-Dichloro-phenyl)-1-ethyl-1H-imidazol-2-yl]-(E)-vinyl-}-3′-fluoro-biphenyl-4-yloxymethyl)-benzoic acid

4-(4′-{2-[4-(2,4-Dichloro-phenyl)-1-ethyl-1H-imidazol-2-yl]-(E)-vinyl-}-3′-fluoro-biphenyl-4-yloxymethyl)-benzoic acid methyl ester (301 mg, 0.5 mmol) was hydrolyzed according to general procedure F to give 4-(4′-{2-[4-(2,4-Dichloro-phenyl)-1-ethyl-1H-imidazol-2-yl]-(E)-vinyl-}-3′-fluoro biphenyl-4-yloxymethyl)-benzoic acid (227 mg, 78%).

LCMS: m/z 587 (M+H)⁺; ¹H NMR (DMSO-d₆, 400 MHz): δ 1.39 (t, 3H), 4.29 (q, 2H), 5.28 (d, 2H), 7.11 (d, 2H), 7.37 (s, 1H), 7.49 (d, 2H), 7.51-7.58 (m, 2H), 7.60 (d, 1H), 7.65-7.74 (m, 4H), 7.96-8.0 (m 4H), 8.22 (d, 1H) ppm.

### Example 225

### 4-(4′-{2-[4-(2,4-Dichloro-phenyl)-1-ethyl-1H-imidazol-2-yl]-(E)-vinyl-}-3′-fluorobiphenyl-3-yloxymethyl)-benzoic acid methyl ester

2-[2-(4-Bromo-2-fluoro-phenyl)-(E)-vinyl]-4-(2,4-dichloro-phenyl)-1-ethyl-1H-imidazole (440 mg, 1 mmol) was coupled with 3-hydroxy phenyl boronic acid (137 mg, 1 mmol) following general procedure B and obtained 4′-{2-[4-(2,4-dichloro-phenyl)-1-ethyl-1H-imidazol-2-yl]-(E)-vinyl}-3′-fluoro-biphenyl-3-ol (453 mg, 1 mmol) was alkylated with methyl 4-(bromomethyl)benzoate (229 mg, 1 mmol) following general procedure E to give 4-(4′-{2-[4-(2,4-Dichloro-phenyl)-1-ethyl-1H-imidazol-2-yl]-(E)-vinyl-}-3′-fluoro biphenyl-3-yloxymethyl)-benzoic acid methyl ester (449 mg, 75%).

LCMS: m/z 601 (M+H)⁺; ¹H NMR (CDCl₃, 400 MHz): δ 1.53 (t, 3H), 3.92 (s, 3H), 4.14 (q, 2H), 5.19 (d, 2H), 7.03-7.07 (m, 2H), 7.11 (s, 1H), 7.20 (d, 2H), 7.30-7.49 (m, 4H), 7.52-7.63 (m, 4H), 7.68 (s, 1H), 7.80 (d, 1H), 8.08 (d, 1H), 8.27 (d, 1H) ppm.

### Example 226

### 4-(4′-{2-[4-(2,4-Dichloro-phenyl)-1-ethyl-1H-imidazol-2-yl]-(E)-vinyl-}-3′-fluorobiphenyl-3-yloxymethyl)-benzoic acid

4-(4′-{2-[4-(2,4-Dichloro-phenyl)-1-ethyl-1H-imidazol-2-yl]-(E)-vinyl-}-3′-fluoro-biphenyl-3-yloxymethyl)-benzoic acid methyl ester (301 mg, 0.5 mmol) was hydrolyzed according to general procedure F to give 4-(4′-{2-[4-(2,4-Dichloro-phenyl)-1-ethyl-1H-imidazol-2-yl]-(E)-vinyl-}-3′-fluoro biphenyl-3-yloxymethyl)-benzoic acid (226 mg, 77%).

LCMS: m/z 587 (M+H)⁺; ¹H NMR (DMSO-d₆, 400 MHz): δ 1.37 (t, 3H), 4.28 (q, 2H), 5.29 (d, 2H), 7.05 (d, 2H), 7.35 (d, 2H), 7.37-7.46 (m 4H), 7.48 (d, 1H), 7.58-7.68 (m, 4H), 7.95 (d, 2H), 8.21 (d, 1H), 8.23 (d, 1H) ppm.

### Example 227

### 4-(3′-{2-[4-(2,4-Dichloro-phenyl)-1-ethyl-1H-imidazol-2-yl]-(E)-vinyl-}-4′-fluorobiphenyl-4-yloxymethyl)-benzoic acid methyl ester

2-[2-(5-Bromo-2-fluoro-phenyl)-(E)-vinyl]-4-(2,4-dichloro-phenyl)-1-ethyl-1H-imidazole (440 mg, 1 mmol) was coupled with 4-hydroxyphenylboronic acid (137 mg, 1 mmol) following general procedure B and obtained 3′-{2-[4-(2,4-dichloro-phenyl)-1-ethyl-1H-imidazol-2-yl]-(E)-vinyl}-4′-fluoro-biphenyl-4-ol (453 mg, 1 mmol) was alkylated with methyl 4-(bromomethyl)benzoate (229 mg, 1 mmol) following general procedure E to give 4-(3′-{2-[4-(2,4-dichloro-phenyl)-1-ethyl-1H-imidazol-2-yl]-(E)-vinyl-}-4′-fluoro biphenyl-4-yloxymethyl)-benzoic acid methyl ester (429 mg, 72%).

LCMS: m/z 601 (M+H)⁺; ¹H NMR (CDCl₃, 400 MHz): δ 1.51 (t, 3H), 3.91 (s, 3H), 4.13 (q, 2H), 5.18 (d, 2H), 7.06 (d, 2H), 7.10-7.16 (m, 2H), 7.31 (d, 1H), 7.42 (d, 2H), 7.44-7.54 (m, 4H), 7.68 (s, 1H), 8.02-8.08 (m, 4H), 8.28 (d, 1H) ppm.

### Example 228

### 4-(3′-{2-[4-(2,4-Dichloro-phenyl)-1-ethyl-1H-imidazol-2-yl]-(E)-vinyl-}-4′-fluorobiphenyl-4-yloxymethyl)-benzoic acid

4-(3′-{2-[4-(2,4-Dichloro-phenyl)-1-ethyl-1H-imidazol-2-yl]-(E)-vinyl-}-4′-fluorobiphenyl-4-yloxymethyl)-benzoic acid methyl ester (301 mg, 0.5 mmol) was hydrolyzed according to general procedure F to give 4-(3′-{2-[4-(2,4-dichloro-phenyl)-1-ethyl-1H-imidazol-2-yl]-(E)-vinyl-}-4′-fluorobiphenyl-4-yloxymethyl)-benzoic acid (226 mg, 77%).

LCMS: m/z 587 (M+H)⁺; ¹H NMR (DMSO-d₆, 400 MHz): δ 1.37 (t, 3H), 4.29 (q, 2H), 5.28 (d, 2H), 7.15 (d, 2H), 7.31-7.41 (m, 2H), 7.31-7.46 (m, 4H), 7.58 (d, 1H), 7.63-7.72 (m, 4H), 7.90 (d, 1H), 7.98 (d, 1H), 8.18 (d, 1H), 8.24 (d, 1H) ppm.

### Example 229

### 4-(3′-{2-[4-(2,4-Dichloro-phenyl)-1-ethyl-1H-imidazol-2-yl]-(E)-vinyl-}-4′-methoxy-biphenyl-4-yloxymethyl)-benzoic acid methyl ester

2-[2-(5-Bromo-2-methoxy-phenyl)-(E)-vinyl]-4-(2,4-dichloro-phenyl)-1-ethyl-1H-imidazole (452 mg, 1 mmol) was coupled with 4-hydroxyphenylboronic acid (137 mg, 1 mmol) following general procedure B and obtained 3′-{2-[4-(2,4-dichloro-phenyl)-1-ethyl-1H-imidazol-2-yl]-(E)-vinyl}-4′-methoxy-biphenyl-4-ol (465 mg, 1 mmol) was alkylated with methyl 4-(bromomethyl)benzoate (229 mg, 1 mmol) following general procedure E to give 4-(3′-{2-[4-(2,4-dichloro-phenyl)-1-ethyl-1H-imidazol-2-yl]-(E)-vinyl-}-4′-methoxy-biphenyl-4-yloxymethyl)-benzoic acid methyl ester (467 mg, 76%).

LCMS: m/z 613 (M+H)⁺; ¹H NMR (CDCl₃, 400 MHz): δ 1.51 (t, 3H), 3.85 (s, 3H), 3.89 (s, 3H), 4.10 (q, 2H), 5.18 (d, 2H), 6.89 (d, 2H), 6.92-6.96 (m, 2H), 6.98-7.05 (m, 2H), 7.34 (d, 1H), 7.35-7.45 (m, 4H), 7.48 (d, 1H), 7.89-8.01 (m, 4H), 8.23 (d, 1H) ppm.

### Example 230

### 4-(3′-{2-[4-(2,4-Dichloro-phenyl)-1-ethyl-1H-imidazol-2-yl]-(E)-vinyl-}-4′-methoxy-biphenyl-4-yloxymethyl)-benzoic acid

4-(3′-{2-[4-(2,4-Dichloro-phenyl)-1-ethyl-1H-imidazol-2-yl]-(E)-vinyl-}-4′-methoxy-biphenyl-4-yloxymethyl)-benzoic acid methyl ester (301 mg, 0.5 mmol) was hydrolyzed according to general procedure F to give 4-(3′-{2-[4-(2,4-dichloro-phenyl)-1-ethyl-1H-imidazol-2-yl]-(E)-vinyl-}-4′-methoxy-biphenyl-4-yloxymethyl)-benzoic acid (229 mg, 78%).

LCMS: m/z 599 (M+H)⁺; ¹H NMR (DMSO-d₆, 400 MHz): δ 1.37 (t, 3H), 3.92 (s, 3H), 4.26 (q, 2H), 5.26 (d, 2H), 7.10 (d, 2H), 7.21-7.31 (m, 2H), 7.32-7.36 (m, 2H), 7.38 (d, 1H), 7.42-7.57 (m, 4H), 7.69 (d, 1H), 7.78-8.26 (m, 4H), 8.18 (d, 1H) ppm.

### Example 231

### 4-(3′-{2-[4-(2,4-Dichloro-phenyl)-1-ethyl-1H-imidazol-2-yl]-(E)-vinyl-}-4′-methoxy-biphenyl-3-yloxymethyl)-benzoic acid methyl ester

2-[2-(5-Bromo-2-methoxy-phenyl)-(E)-vinyl]-4-(2,4-dichloro-phenyl)-1-ethyl-1H-imidazole (452 mg, 1 mmol) was coupled with 3-hydroxyphenylboronic acid (137 mg, 1 mmol) following general procedure B and obtained 3′-{2-[4-(2,4-dichloro-phenyl)-1-ethyl-1H-imidazol-2-yl]-(E)-vinyl}-4′-methoxy-biphenyl-3-ol (465 mg, 1 mmol) was alkylated with methyl 4-(bromomethyl)benzoate (229 mg, 1 mmol) following general procedure E to give 4-(3′-{2-[4-(2,4-dichloro-phenyl)-1-ethyl-1H-imidazol-2-yl]-(E)-vinyl-}-4′-methoxy-biphenyl-3-yloxymethyl)-benzoic acid methyl ester (479 mg, 78%).

LCMS: m/z 613 (M+H)⁺; ¹H NMR (CDCl₃, 400 MHz): δ 1.52 (t, 3H), 3.90 (s, 3H), 3.95 (s, 3H), 4.13 (q, 2H), 5.20 (d, 2H), 6.92 (d, 2H), 6.94 (d, 1H), 6.97 (d, 1H), 7.01-7.11 (m, 2H), 7.20-7.21 (m, 2H), 7.30-7.38 (m, 2H), 7.41 (d, 1H), 7.46 (d, 1H), 7.47-7.49 (m, 2H), 7.74 (d, 1H), 8.06 (d, 1H), 8.29 (d, 1H) ppm.

### Example 232

### 4-(3′-{2-[4-(2,4-Dichloro-phenyl)-1-ethyl-1H-imidazol-2-yl]-(E)-vinyl-}-4′-methoxy-biphenyl-3-yloxymethyl)-benzoic acid

4-(3′-{2-[4-(2,4-Dichloro-phenyl)-1-ethyl-1H-imidazol-2-yl]-(E)-vinyl-}-4′-methoxy-biphenyl-3-yloxymethyl)-benzoic acid methyl ester (301 mg, 0.5 mmol) was hydrolyzed according to general procedure F to give 4-(3′-{2-[4-(2,4-dichloro-phenyl)-1-ethyl-1H-imidazol-2-yl]-(E)-vinyl-}-4′-methoxy-biphenyl-3-yloxymethyl)-benzoic acid (227 mg, 77%).

LCMS: m/z 599 (M+H)⁺; ¹H NMR (DMSO-d₆, 400 MHz): δ 1.39 (t, 3H), 3.90 (s, 3H), 4.24 (q, 2H), 5.28 (d, 2H), 7.09 (d, 2H), 7.11-7.21 (m, 2H), 7.28-7.36 (m, 2H), 7.38 (d, 1H), 7.41-7.56 (m, 4H), 7.71 (d, 1H), 7.76-8.02 (m. 4H), 8.16 (d, 1H) ppm.

### Example 233

### 4-(3′-{2-[4-(2,4-Dichloro-phenyl)-1-ethyl-1H-imidazol-2-yl]-(E)-vinyl-}-biphenyl-4-yloxymethyl)-benzoic acid methyl ester

2-[2-(3-Bromo-phenyl)-(E)-vinyl]-4-(2,4-dichloro-phenyl)-1-ethyl-1H-imidazole (422 mg, 1 mmol) was coupled with 4-hydroxyphenylboronic acid (137 mg, 1 mmol) following general procedure B and obtained 3′-{2-[4-(2,4-dichloro-phenyl)-1-ethyl-1H-imidazol-2-yl]-(E)-vinyl}-biphenyl-4-ol (435 mg, 1 mmol) was alkylated with methyl 4-(bromomethyl)benzoate (229 mg, 1 mmol) following general procedure E to give 4-(3′-{2-[4-(2,4-dichloro-phenyl)-1-ethyl-1H-imidazol-2-yl]-(E)-vinyl-}-biphenyl-4-yloxymethyl)-benzoic acid methyl ester (419 mg, 72%).

LCMS: m/z 583 (M+H)⁺; ¹H NMR (CDCl₃, 400 MHz): δ 1.53 (t, 3H), 3.92 (s, 3H), 4.14 (q, 2H), 5.19 (d, 2H), 6.97 (d, 2H), 7.07 (d, 1H), 7.30 (d, 1H), 7.41-7.54 (m, 8H), 7.56-7.67 (m, 4H), 8.08 (d, 1H), 8.26 (d, 1H) ppm.

### Example 234

### 4-(3′-{2-[4-(2,4-Dichloro-phenyl)-1-ethyl-1H-imidazol-2-yl]-(E)-vinyl-}-biphenyl-4-yloxymethyl)-benzoic acid

4-(3′-{2-[4-(2,4-Dichloro-phenyl)-1-ethyl-1H-imidazol-2-yl]-(E)-vinyl-}-biphenyl-4-yloxymethyl)-benzoic acid methyl ester (292 mg, 0.5 mmol) was hydrolyzed according to general procedure F to give 4-(3′-{2-[4-(2,4-Dichloro-phenyl)-1-ethyl-1H-imidazol-2-yl]-(E)-vinyl-}-biphenyl-4-yloxymethyl)-benzoic acid (219 mg, 77%).

LCMS: m/z 569 (M+H)⁺; ¹H NMR (DMSO-d₆, 400 MHz): δ 1.39 (t, 3H), 4.29 (q, 2H), 5.28 (d, 2H), 7.12 (d, 2H), 7.41-7.57 (m, 4H), 7.59-7.72 (m, 8H), 7.89 (d, 1H), 7.91 (d, 1H), 7.99 (d, 1H), 8.2 (d, 1H) ppm.

### Example 235

### 4-(3′-{2-[4-(2,4-Dichloro-phenyl)-1-ethyl-1H-imidazol-2-yl]-(E)-vinyl-}-biphenyl-3-yloxymethyl)-benzoic acid methyl ester

2-[2-(3-Bromo-phenyl)-(E)-vinyl]-4-(2,4-dichloro-phenyl)-1-ethyl-1H-imidazole (422 mg, 1 mmol) was coupled with 3-hydroxyphenylboronic acid (137 mg, 1 mmol) following general procedure B and obtained 3′-{2-[4-(2,4-dichloro-phenyl)-1-ethyl-1H-imidazol-2-yl]-(E)-vinyl}-biphenyl-3-ol (435 mg, 1 mmol) was alkylated with methyl 4-(bromomethyl)benzoate (229 mg, 1 mmol) following general procedure E to give 4-(3′-{2-[4-(2,4-dichloro-phenyl)-1-ethyl-1H-imidazol-2-yl]-(E)-vinyl-}-biphenyl-3-yloxymethyl)-benzoic acid methyl ester (449 mg, 77%).

LCMS: m/z 583 (M+H)⁺; ¹H NMR (CDCl₃, 400 MHz): δ 1.50 (t, 3H), 3.92 (s, 3H), 4.13 (q, 2H), 5.21 (d, 2H), 6.97 (d, 2H), 6.99 (d, 1H), 7.23 (d, 1H), 7.31-7.51 (m, 8H), 7.54-7.67 (m, 4H), 8.04 (d, 1H), 8.27 (d, 1H) ppm.

### Example 236

### 4-(3′-{2-[4-(2,4-Dichloro-phenyl)-1-ethyl-1H-imidazol-2-yl]-(E)-vinyl-}-biphenyl-3-yloxymethyl)-benzoic acid

4-(3′-{2-[4-(2,4-Dichloro-phenyl)-1-ethyl-1H-imidazol-2-yl]-(E)-vinyl-}-biphenyl-3-yloxymethyl)-benzoic acid methyl ester (292 mg, 0.5 mmol) was hydrolyzed according to general procedure F to give 4-(3′-{2-[4-(2,4-dichloro-phenyl)-1-ethyl-1H-imidazol-2-yl]-(E)-vinyl-}-biphenyl-3-yloxymethyl)-benzoic acid (225 mg, 79%).

LCMS: m/z 569 (M+H)⁺; ¹H NMR (DMSO-d₆, 400 MHz): δ 1.37 (t, 3H), 4.29 (q, 2H), 5.31 (d, 2H), 7.06 (d, 2H), 7.34-7.42 (m, 4H), 7.44-7.60 (m, 6H), 7.62-7.74 (m, 2H), 7.76 (d, 1H), 7.96-7.99 (m 2H), 8.23 (d, 1H) ppm.

### Example 237

### 4-(4-(2,4-Dichloro-phenyl)-2-{2-[4′-(3-methoxycarbonyl-propoxy)-biphenyl-3yl]-(E)-vinyl}-imidazol-1yl)-butyric acid methyl ester

4-(4-(2,4-Dichloro-phenyl)-2-{2-[4′-(3-methoxycarbonyl-propoxy)-biphenyl-3-yl]-(E)-vinyl}-imidazol-1-yl)-butyric acid methyl ester (421 mg, 69%) was prepared according to general procedure A using trans-3-bromocinnamic acid (227 mg, 1 mmol) and 2-bromo-2,4-dichloroacetophenone (267 mg, 1 mmol) and obtained 2-[2-(3-bromo-phenyl)-(E)-vinyl]-4-(2,4-dichloro-phenyl)-1H-imidazole (394 mg, 1 mmol) was coupled with 4-hydroxyphenylboronic acid (137 mg, 1 mmol) following general procedure B and resulting 3′-{2-[4-(2,4-dichloro-phenyl)-1H-imidazol-2-yl]-(E)-vinyl}-biphenyl-4-ol (407 mg, 1 mmol) was di-alkylated with methyl 4-bromobutyrate (362 mg, 2 mmol) following general procedure E.

LCMS: m/z 607 (M+H)⁺; ¹H NMR (CDCl₃, 400 MHz): δ 2.18 (m, 2H), 2.42 (t, 3H), 2.56 (t, 3H), 3.66 (s, 3H), 3.70 (s, 3H), 4.06 (q, 2H), 4.20 (q, 2H), 6.96 (d, 2H), 7.07 (d, 2H), 7.31 (d, 1H), 7.33-7.42 (m, 2H), 7.44-7.52 (m, 2H), 7.56 (d, 2H), 7.64 (s, 1H), 7.77 (d, 1H), 8.27 (d, 1H) ppm.

### Example 238

### 4-[2-{2-[4′-(3-Carboxy-propoxy)-biphenyl-3-yl]-(E)-vinyl}-4-(2,4-dichloro-phenyl)-imidazol-1-yl]-butyric acid

4-(4-(2,4-Dichloro-phenyl)-2-{2-[4′-(3-methoxycarbonyl-propoxy)-biphenyl-3-yl]-(E)-vinyl}-imidazol-1-yl)-butyric acid methyl ester (304 mg, 0.5 mmol) was hydrolyzed according to general procedure F to give 4-[2-{2-[4′-(3-carboxy-propoxy)-biphenyl-3-yl]-(E)-vinyl}-4-(2,4-dichloro-phenyl)-imidazol-1-yl]-butyric acid (212 mg, 73%).

LCMS: m/z 579 (M+H)⁺; ¹H NMR (DMSO-d₆, 400 MHz): δ 1.96 (m, 2H), 2.28 (t, 3H), 2.42 (t, 3H), 4.03 (q, 2H), 4.25 (q, 2H), 7.03 (d, 2H), 7.40-7.55 (m 4H), 7.61-7.65 (m, 4H), 7.67-7.69 (m, 2H), 7.94 (d, 1H), 8.26 (d, 1H) ppm.

### Example 239

### 4-(3′-{2-[4-(2,4-Dichloro-phenyl)-1-methoxycarbonylmethyl-1H-imidazol-2-yl]-(E)-vinyl}-biphenyl-4-yloxy)-butyric acid methyl ester

4-(3′-{2-[4-(2,4-Dichloro-phenyl)-1-methoxycarbonylmethyl-1H-imidazol-2-yl]-(E)-vinyl}-biphenyl-4-yloxy)-butyric acid methyl ester (379 mg, 65%) was prepared according to general procedure A using trans 3-bromo cinnamic acid (227 mg, 1 mmol) and 2-bromo-2,4-dichloro acetophenone (267 mg, 1 mmol) and obtained 2-[2-(3-Bromo-phenyl)-(E)-vinyl]-4-(2,4-dichloro-phenyl)-1H-imidazole (394 mg, 1 mmol) was alkylated with methyl bromo acetate (153 mg, 1 mmol) following general procedure E. The obtained 2-[2-(3-Bromo-phenyl)-(E)-vinyl]-4-(2,4-dichloro-phenyl)-1H-imidazol-1-yl]-acetic acid methyl ester (466 mg, 1 mmol) was coupled with 4-hydroxy phenyl boronic acid (137 mg, 1 mmol) following general procedure B and resulting 4{-(2,4-dichloro-phenyl)-2-[2-(4′-hydroxy-biphenyl-3-yl]-imidazol-1-yl}acetic acid methyl ester (479 mg, 1 mmol) was alkylated with 4-bromomethyl butyrate (181 mg, 1 mmol) following general procedure E.

LCMS: m/z 579 (M+H)⁺.

### Example 240

### 4-(3′-{2-[4-(2,4-Dichloro-phenyl)-1-methoxycarbonylmethyl-1H-imidazol-2-yl]-(E)-vinyl}-biphenyl-4-yloxy)-butyric acid

4-(3′-{2-[4-(2,4-Dichloro-phenyl)-1-methoxycarbonylmethyl-1H-imidazol-2-yl]-(E)-vinyl}-biphenyl-4-yloxy)-butyric acid methyl ester (290 mg, 0.5 mmol) was hydrolyzed according to general procedure F to give 4-(3′-{2-[4-(2,4-dichloro-phenyl)-1-methoxycarbonylmethyl-1H-imidazol-2-yl]-(E)-vinyl}-biphenyl-4-yloxy)-butyric acid (382 mg, 69%).

LCMS: m/z 551 (M+H)⁺; ¹H NMR (DMSO-d₆, 400 MHz): δ 1.98 (m, 2H), 2.42 (t, 2H), 4.03 (t, 2H), 5.17 (d, 2H), 7.03 (d, 1H), 7.30 (s, 1H), 7.34 (s, 1H), 7.38-7.49 (m, 2H), 7.50-7.54 (m, 2H), 7.55-7.71 (m, 4H), 7.94 (d, 1H), 7.97 (d, 1H), 8.30 (d, 1H) ppm.

### Example 241

### 4-(6-{2-[4-(2,4-Dichloro-phenyl)-1-ethyl-1H-imidazol-2-yl]-(E)-vinyl}-naphthalen-2-yloxy)-butyric acid

Trans-3-(6-methoxynaphthalene-2-yl)acrylic acid (228 mg, 1 mmol) was reacted with 2-bromo-2,4-dichloroacetophenone (267 mg, 1 mmol) according to general procedure A and obtained 4-(2,4-dichloro-phenyl)-2[2-(6-methoxy-naphthalen-2-yl)-(E)-vinyl]-1H-imidazol (198 mg, 0.5 mmol) was treated with bromo ethane (55 mg, 1 mmol) following general procedure E. The resulted 4-(2,4-dichloro-phenyl)-1-ethyl-2[2-(6-methoxy-naphthalen-2-yl)-(E)-vinyl]-1H-imidazole (211 mg, 0.5 mmol) was de-alkylated as described in general procedure C and obtained 6-{2-[4-(2,4-dichloro-phenyl)-1-ethyl-1H-imidazole-2-yl]-(E)-vinyl}-naphthalen-2-ol (205 mg, 0.5 mmol) was alkylated with methyl 4-bromobutyrate (91 mg, 0.5 mmol) following general procedure E. The resulted 4-(6-{2-[4-(2,4-dichloro-phenyl)-1-ethyl-1H-imidazol-2-yl]-(E)-vinyl}-naphthalen-2-yloxy)-butyric acid methyl ester (255 mg, 0.5 mmol) was hydrolyzed according to general procedure F to give 4-(6-{2-[4-(2,4-Dichloro-phenyl)-1-ethyl-1H-imidazol-2-yl]-(E)-vinyl}-naphthalen-2-yloxy)-butyric acid (327 mg, 66%).

LCMS: m/z 495 (M+H)⁺.

### Example 242

### 2-[2-(6-Benzyloxy-naphthalen-2-yl)-(E)-vinyl]-4-(2,4-dichloro-phenyl)-1-ethyl-1H-imidazole

2-[2-(6-Benzyloxy-naphthalen-2-yl)-(E)-vinyl]-4-(2,4-dichloro-phenyl)-1-ethyl-1H-imidazole (141 mg, 57%) was prepared according to general procedure A using trans-3-(6-methoxy naphthalene-2-yl)acrylic acid (Rwerechem-BKHW-0217) (228 mg, 1 mmol) and 2-bromo-2,4-dichloro acetophenone (267 mg, 1 mmol) and obtained 4-(2,4-dichloro-phenyl)-2[2-(6-methoxy-naphthalen-2-yl)-(E)-vinyl]-1H-imidazol (197 mg, 0.5 mmol) was treated with bromo ethane (99 mg, 0.5 mmol) following general procedure E. The resulted 4-(2,4-dichloro-phenyl)-1-ethyl-2-[2-(6-methoxy-naphthalen-2-yl)-(E)-vinyl]-1H-imidazole (212 mg, 0.5 mmol) was de-alkylated as described in general procedure C and obtained 6-{2-[4-(2,4-dichloro-phenyl)-1-ethyl-1H-imidazol-2-yl]-(E)-vinyl}-naphthalen-2-ol (204 mg, 0.5 mmol) was alkylated with benzyl bromide (86 mg, 0.5 mmol) following general procedure E.

LCMS: m/z 499 (M+H)⁺; ¹H NMR (DMSO-d₆, 400 MHz): δ 1.40 (t, 3H), 4.29 (q, 2H), 5.23 (s, 2H), 7.33 (d, 1H), 7.37-7.45 (m, 5H), 7.51-7.53 (m, 2H), 7.63 (d, 1H), 7.65 (d, 1H), 7.83-7.96 (m, 4H), 7.97 (d, 1H), 8.06 (s, 1H), 8.27 (d, 1H) ppm.

### Example 243

### 2-[2-(6-Benzyloxy-naphthalen-2-yl)-(E)-vinyl]-4-(2,4-dichlorophenyl)-imidazol-1-yl]-acetic acid methyl ester

2-[2-(6-Benzyloxy-naphthalen-2-yl)-(E)-vinyl]-4-(2,4-dichloro-phenyl)-imidazol-1-yl]-acetic acid methyl ester (139 mg, 51%) was prepared according to general procedure A using trans-3-(6-methoxy naphthalene-2-yl)acrylic acid (Rwerechem-BKHW-0217) (228 mg, 1 mmol) and 2-bromo-2,4-dichloro acetophenone (267 mg, 1 mmol) and obtained 4-(2,4-dichloro-phenyl)-2[2-(6-methoxy-naphthalen-2-yl)-(E)-vinyl]-1H-imidazol (197 mg, 0.5 mmol) was alkylated with methyl bromo acetate (77 mg, 0.5 mmol) following general procedure E. The resulted 4-(2,4-dichloro-phenyl)-2-[2-(6-methoxy-naphthalen-2-yl)-(E)-vinyl]-imidazol-1-yl}-acetic acid methyl ester (233 mg, 0.5 mmol) was de-alkylated as described in general procedure C and obtained 4-(2,4-dichloro-phenyl)-2-[2-(6-hydroxy-naphthalen-2-yl)-(E)-vinyl]-imidazol-1-yl}-acetic acid methyl ester (227 mg, 0.5 mmol) was alkylated with benzyl bromide (171 mg, 1 mmol) following general procedure E.

LCMS: m/z 543 (M+H)⁺.

### Example 244

### 2-[2-(6-Benzyloxy-naphthalen-2-yl)-(E)-vinyl]-4-(2,4-dichlorophenyl)-imidazol-1-yl]-acetic acid

2-[2-(6-Benzyloxy-naphthalen-2-yl)-(E)-vinyl]-4-(2,4-dichloro-phenyl)-imidazol-1-yl]-acetic acid methyl ester (135 mg, 0.25 mmol) was hydrolyzed according to general procedure F to give 2-[2-(6-Benzyloxy-naphthalen-2-yl)-(E)-vinyl]-4-(2,4-dichloro-phenyl)-imidazol-1-yl]-acetic acid methyl ester (75 mg, 57%).

LCMS: m/z 529 (M+H)⁺; ¹H NMR (DMSO-d₆, 400 MHz): δ 5.17 (s, 2H), 5.23 (s, 2H), 7.15 (d, 1H), 7.19-7.28 (m, 2H), 7.32-7.37 (m, 2H), 7.40-7.48 (m, 2H), 7.51-7.55 (m, 2H), 7.68 (d, 1H), 7.80-7.95 (m, 3H), 7.98 (s, 1H), 8.04 (s, 1H), 8.20 (d, 1H), 8.31 (d, 1H) ppm

### Example 245

### 2-[2-(6-Benzyloxy-naphthalen-21)-(E)-vinyl]-4-(2,4-dichloro-phenyl)-1H-imidazole

Trans-3-(6-methoxy naphthalene-2-yl)acrylic acid methyl ester (242 mg, 1 mmol) was de-alkylated as described in general procedure C and obtained 3-(6-hydroxy-naphthalen-2-yl)-acrylic acid methyl ester (228 mg, 1 mmol) was alkylated with benzyl bromide (171 mg, 1 mmol) following general procedure E. The resulted 3-(6-benzyloxy-naphthalen-2-yl)-acrylic acid methyl ester (159 mg, 0.5 mmol) was hydrolyzed according to general procedure F and obtained 3-(6-benzyloxy-naphthalen-2-yl)-acrylic acid (152 mg, 0.5 mmol) was treated with 2-bromo-2,4-dichloroacetophenone (134 mg, 0.5 mmol) following general procedure A to give 2-[2-(6-benzyloxy-naphthalen-2-yl)-(E)-vinyl]-4-(2,4-dichloro-phenyl)-1H-imidazole (119 mg, 50%).

LCMS: m/z 471 (M+H)⁺; ¹H NMR (DMSO-d₆, 400 MHz): δ 5.23 (s, 2H), 7.15 (d, 1H), 7.16 (d, 1H), 7.19-7.27 (m, 2H), 7.35-7.37 (m, 2H), 7.40-7.49 (m, 2H), 7.50-7.56 (m, 2H), 7.64 (d, 1H), 7.80 (d, 2H), 7.83 (d, 1H), 8.22 (d, 1H), 11.99 (s, 1H), 12.6 (s, 1H) ppm.

### Example 246

### 2-[2-(6-Butoxy-naphthalen-2-yl)-(E)-vinyl]-4-(2,4-dichloro-phenyl)-1H-imidazole

Trans-3-(6-methoxynaphthalene-2-yl)acrylic acid methyl ester (242 mg, 1 mmol) was de-alkylated as described in general procedure C and obtained 3-(6-hydroxy-naphthalen-2-yl)-acrylic acid methyl ester (228 mg, 1 mmol) was alkylated with bromo butane (137 mg, 1 mmol) following general procedure E. The resulted 3-(6-butoxy-naphthalen-2-yl)-acrylic acid methyl ester (142 mg, 0.5 mmol) was hydrolyzed according to general procedure F and obtained 3-(6-butoxy-naphthalen-2-yl)-acrylic acid (135 mg, 0.5 mmol) was treated with 2-bromo-2,4-dichloroacetophenone (134 mg, 0.5 mmol) following general procedure A to give 2-[2-(6-butoxy-naphthalen-2-yl)-(E)-vinyl]-4-(2,4-dichloro-phenyl)-1H-imidazole (109 mg, 50%).

LCMS: m/z 437 (M+H)⁺.

### Example 247

### 4-(3-{2-[-4-(2,4-Dichloro-phenyl)-1H-imidazol-2-yl]-(E)-vinyl}-biphenyl-4-yloxy)-butyric acid

Trans-3-bromocinnamic acid (227 mg, 1 mmol) was reacted with 2-bromo-2,4-dichloroacetophenone (267 mg, 1 mmol) according to general procedure A and obtained 2-[2-(3-bromo-phenyl)-(E)-vinyl]-4-(2,4-dichloro-phenyl)-1H-imidazole (394 mg, 1 mmol) was coupled with 4-hydroxy phenyl boronic acid (137 mg, 1 mmol) following general procedure B and resulted 3′-(2-[4-(2,4-dichloro-phenyl)-1H-imidazol-2-yl]-(E)-vinyl}-biphenyl-4-ol (407 mg, 1 mmol) was protected with di-tert-butyl-dicarbonate according to general procedure N. The obtained 4-(2,4-dichloro-phenyl)-2-[2-(4′-hydroxy-biphenyl-3-yl)-(E)-vinyl]-imidazole-1-carboxylic acid tert-butyl ester (507 mg, 1 mmol) was alkylated with 4-bromomethyl butyrate (181 mg, 1 mmol) following general procedure E and resulted 4-(2,4-dichloro-phenyl)-2-[2-(4′-(3-methoxy-carbonyl-propoxy)-biphenyl-3-yl)-(E)-vinyl]-imidazole-1-carboxylic acid tert-butyl ester ester (303 mg, 0.5 mmol) was hydrolyzed & de-protected according to general procedure F & O to give 4-(3-{2-[-4-(2,4-Dichloro-phenyl)-1H-imidazol-2-yl]-(E)-vinyl}-biphenyl-4-yloxy)-butyric acid (121 mg, 50%).

LCMS: m/z 493 (M+H)⁺; ¹H NMR (DMSO-d₆, 400 MHz): δ 1.49 (m, 2H), 1.98 (m, 2H), 2.21 (t, 2H), 4.22 (t, 2H), 6.88 (d, 2H), 7.38-7.40 (m, 2H), 7.46-7.48 (m, 2H), 7.49-7.57 (m, 2H), 7.61 (d, 1H), 7.87 (d, 2H), 8.24 (d, 1H) ppm.

### Example 248

### 4-(3′-{2-[-4-(2,4-Dichloro-phenyl)-1H-imidazol-2-yl]-(E)-vinyl}-biphenyl-4-yloxymethyl)-benzoic acid

Trans-bromocinnamic acid (227 mg, 1 mmol) was reacted with 2-bromo-2,4-dichloro acetophenone (267 mg, 1 mmol) according to general procedure A and obtained 2-[2-(3-bromo-phenyl)-(E)-vinyl]-4-(2,4-dichloro-phenyl)-1H-imidazole (394 mg, 1 mmol) was coupled with 4-hydroxy phenyl boronic acid (137 mg, 1 mmol) following general procedure B and resulted 3′-(2-[4-(2,4-dichloro-phenyl)-1H-imidazol-2-yl]-(E)-vinyl}-biphenyl-4-ol (407 mg, 1 mmol) was protected with di-tert-butyl-dicarbonate according to general procedure N. The obtained 4-(2,4-dichloro-phenyl)-2-[2-(4′-hydroxy-biphenyl-3-yl)-(E)-vinyl]-imidazole-1-carboxylic acid tert-butyl ester (507 mg, 1 mmol) was alkylated with methyl omethyl)benzoate (229 mg, 1 mmol) following general procedure E and resulted 4-(2,4-dichloro-phenyl)-2-[2-(4′-(4-methoxy-carbonyl-benzyloxy)-biphenyl-3-yl)-(E)-vinyl]-imidazole-1-carboxylic acid tert-butyl ester ester (327 mg, 0.5 mmol) was hydrolyzed & de-protected according to general procedure F & O to give 4-(3-{2-[-4-(2,4-dichloro-phenyl)-1H-imidazol-2-yl]-(E)-vinyl}-biphenyl-4-yloxymethyl)-benzoic acid (129 mg, 48%).

LCMS: m/z 541 (M+H)⁺.

### Example 249

### 4-(4-{2-[4-(2,4-Dichloro-phenyl)-1-ethyl-1H-imidazol-2-yl]-(E)-vinyl}-phenoxy)-benzoic acid

4-{2-[4-(2,4-Dichloro-phenyl)-1-ethyl-1H-imidazol-2-yl]-(E)-vinyl}-phenol (300 mg, 0.84 mmol) was treated with ethyl 4-iodobenzoate using general procedure J, followed by ester hydrolysis according to general procedure F to give 4-(4-{2-[4-(2,4-dichloro-phenyl)-1-ethyl-1H-imidazol-2-yl]-(E)-vinyl}-phenoxy)-benzoic acid (5.7 mg, 1.4% yield).

LCMS: m/z 479 (M+H)⁺; ¹H NMR (DMSO-d₆, 400 MHz): δ 1.34 (t, 3H), 4.24 (q, 2H), 7.06 (d, 2H), 7.13 (d, 2H), 7.25 (d, 1H), 7.47 (dd, 1H), 7.54 (d, 1H), 7.62 (d, 1H), 7.81 (d, 2H), 7.94 (m, 3H), 8.22 (d, 1H) ppm.

### Example 250

### 7-(4′-{2-[4-(2,4-Dichloro-phenyl)-1-ethyl-1H-imidazol-2-yl]-(E)-vinyl}-biphenyl-4-yloxy)-heptanoic acid

4′-{2-[4-(2,4-Dichloro-phenyl)-1-ethyl-1H-imidazol-2-yl]-(E)-vinyl}-biphenyl-4-ol (100 mg, 0.23 mmol) was treated with ethyl 7-bromoheptanoate using general procedure E, followed by ester hydrolysis according to general procedure F to give 7-(4′-{2-[4-(2,4-dichloro-phenyl)-1-ethyl-1H-imidazol-2-yl]-(E)-vinyl}-biphenyl-4-yloxy)-heptanoic acid (2 mg, 1.5% yield).

LCMS: m/z 563 (M+H)⁺; ¹H NMR (DMSO-d₆, 400 MHz): δ 1.35 (t, 3H), 1.42-1.56 (m, 4H), 1.70 (m, 4H), 2.20 (t, 2H), 4.00 (t, 2H) 4.25 (q, 2H), 7.01 (d, 2H), 7.30 (d, 1H), 7.48 (dd, 1H), 7.55 (d, 1H), 7.62-7.67 (m, 5H), 7.77 (d, 2H), 7.94 (s, 1H), 8.24 (d, 1H) ppm.

### Example 251

### 4-(4′-{2-[4-(2,4-Dichloro-phenyl)-1-(3-methyl-butyl)-1H-imidazol-2-yl]-(E)-vinyl}-biphenyl-4-yloxy)-butyric acid

4-(2,4-Dichloro-phenyl)-2-[2-(4′-methoxy-biphenyl-4-yl)-(E)-vinyl]-1H-imidazole (350 mg, 0.83 mmol) was treated with 1-bromo-3-methyl-butane using general procedure E, followed by ether cleavage according to general procedure C. Treatment with methyl 4-bromobutyrate, followed by ester hydrolysis according to general procedures E and F respectively gave 4-(4′-{2-[4-(2,4-dichloro-phenyl)-1-(3-methyl-butyl)-1H-imidazol-2-yl]-(E)-vinyl}-biphenyl-4-yloxy)-butyric acid (2 mg, 0.4% yield).

LCMS: m/z 563 (M+H)⁺.

### Example 252

### 5-(4′-{2-[4-(2,4-Dichloro-phenyl)-1-ethyl-1H-imidazol-2-yl]-(E)-vinyl}-biphenyl-4-yloxy)-pentanoic acid

4′-{2-[4-(2,4-Dichloro-phenyl)-1-ethyl-1H-imidazol-2-yl]-(E)-vinyl}-biphenyl-4-ol (100 mg, 0.23 mmol) was treated with methyl 5-bromopentanoate using general procedure E, followed by ester hydrolysis according to general procedure F to give 5-(4′-{2-[4-(2,4-dichloro-phenyl)-1-ethyl-1H-imidazol-2-yl]-(E)-vinyl}-biphenyl-4-yloxy)-pentanoic acid (5 mg, 4% yield).

LCMS: m/z 535 (M+H)⁺.

### Example 253

### 6-(4′-{2-[4-(2,4-Dichloro-phenyl)-1-ethyl-1H-imidazol-2-yl]-(E)-vinyl}-biphenyl-4-yloxy)-hexanoic acid

4′-{2-[4-(2,4-Dichloro-phenyl)-1-ethyl-1H-imidazol-2-yl]-(E)-vinyl}-biphenyl-4-ol (100 mg, 0.23 mmol) was treated with ethyl 6-bromohexanoate using general procedure E, followed by ester hydrolysis according to general procedure F to give 6-(4′-{2-[4-(2,4-dichloro-phenyl)-1-ethyl-1H-imidazol-2-yl]-(E)-vinyl}-biphenyl-4-yloxy)-hexanoic acid (2 mg, 1.6% yield).

LCMS: m/z 549 (M+H)⁺.

### Example 254

### 3-(4′-{2-[4-(2,4-Dichloro-phenyl)-1-ethyl-1H-imidazol-2-yl]-(E)-vinyl}-biphenyl-4-yloxy)-propionic acid

4′-{2-[4-(2,4-Dichloro-phenyl)-1-ethyl-1H-imidazol-2-yl]-(E)-vinyl}-biphenyl-4-ol (57 mg, 0.13 mmol) was treated with 3-bromopropionic acid using general procedure P to give 3-(4′-{2-[4-(2,4-dichloro-phenyl)-1-ethyl-1H-imidazol-2-yl]-(E)-vinyl}-biphenyl-4-yloxy)-propionic acid (8.2 mg, 12% yield).

LCMS: m/z 507 (M+H)⁺; ¹H NMR (CD₃OD, 400 MHz): δ 1.55 (t, 3H), 2.76 (t, 2H), 4.22 (q, 2H), 4.30 (t, 3H), 6.98-7.09 (m, 3H), 7.35 (m, 1H), 7.47 (d, 1H), 7.54-7.69 (m, 8H), 8.00 (d, 1H) ppm.

### Example 255

### 4-(4′-{2-[4-(2,4-Dichloro-phenyl)-1-ethyl-1H-imidazol-2-yl]-(E)-propenyl}-biphenyl-4-yloxy)-butyric acid

4′-{2-[4-(2,4-Dichloro-phenyl)-1-ethyl-1H-imidazol-2-yl]-(E)-propenyl}-biphenyl-4-ol (100 mg, 0.22 mmol) was treated with methyl 4-bromobutyrate using general procedure E, followed by ester hydrolysis according to general procedure F to give 4-(4′-{2-[4-(2,4-dichloro-phenyl)-1-ethyl-1H-imidazol-2-yl]-(E)-propenyl}-biphenyl-4-yloxy)-butyric acid (14 mg, 12% yield).

LCMS: m/z 535 (M+H)⁺; ¹H NMR (CD₃OD, 400 MHz): δ 1.53 (t, 3H), 2.14 (m, 2H), 2.42 (s, 3H), 2.55 (t, 2H), 4.09 (t, 2H), 4.18 (q, 2H), 6.79 (br s, 1H), 7.01 (m, 2H), 7.33 (dd, 1H), 7.45 (d, 1H) 7.50 (d, 2H), 7.58 (d, 2H), 7.63 (d, 2H), 7.66 (s, 1H), 7.97 (d, 1H) ppm.

### Example 256

### 4-(4′-{2-[4-(2,4-Dichloro-phenyl)-1-ethyl-1H-imidazol-2-yl]-(Z)-2-fluoro-vinyl}-biphenyl-4-yloxy)-butyric acid

4′-{2-[4-(2,4-Dichloro-phenyl)-1-ethyl-1H-imidazol-2-yl]-(Z)-2-fluoro-vinyl}-biphenyl-4-ol (20 mg, 0.044 mmol) was treated with methyl 4-bromobutyrate using general procedure E, followed by ester hydrolysis according to general procedure F to give 4-(4′-{2-[4-(2,4-dichloro-phenyl)-1-ethyl-1H-imidazol-2-yl]-(Z)-2-fluoro-vinyl}-biphenyl-4-yloxy)-butyric acid (6 mg, 25% yield).

LCMS: m/z 539 (M+H)⁺; ¹H NMR (CDCl₃, 400 MHz): δ 1.53 (t, 3H), 2.16 (m, 2H), 2.62 (t, 2H), 4.06 (t, 2H), 4.26 (q, 2H), 6.81 (d, 1H), 6.95 (d, 2H), 7.32 (dd, 1H), 7.44 (d, 1H), 7.51-7.59 (m, 4H), 7.68 (m, 3H), 8.14 (d, 1H) ppm.

### Example 257

### 4-(4′-{2-[4-(2,4-Dichloro-phenyl)-1-ethyl-1H-imidazol-2-yl]-(E)-2-fluoro-vinyl}-biphenyl-4-yloxy)-butyric acid

4′-{2-[4-(2,4-Dichloro-phenyl)-1-ethyl-1H-imidazol-2-yl]-(E)-2-fluoro-vinyl}-biphenyl-4-ol (43 mg, 0.095 mmol) was treated with methyl 4-bromobutyrate using general procedure E, followed by ester hydrolysis according to general procedure F to give 4-(4′-{2-[4-(2,4-dichloro-phenyl)-1-ethyl-1H-imidazol-2-yl]-(E)-2-fluoro-vinyl}-biphenyl-4-yloxy)-butyric acid (15 mg, 29% yield).

LCMS: m/z 539 (M+H)⁺; ¹H NMR (CDCl₃, 400 MHz): δ 1.34 (t, 3H), 2.13 (m, 2H), 2.60 (t, 2H), 3.89 (q, 2H), 4.04 (t, 2H), 6.81 (d, 1H), 6.92 (d, 2H), 7.15 (d, 2H), 7.29 (dd, 1H), 7.40-7.49 (m, 5H), 7.75 (s, 1H), 8.14 (d, 1H) ppm.

### Example 258

### 4-(4′-{2-[4-(2,4-Dichloro-phenyl)-1-ethyl-1H-imidazol-2-yl]-(E)-vinyl}-biphenyl-4-yloxy)-2-methyl-butyric acid

4′-{2-[4-(2,4-Dichloro-phenyl)-1-ethyl-1H-imidazol-2-yl]-(E)-vinyl}-biphenyl-4-ol (90 mg, 0.21 mmol) was treated with 4-bromo-2-methylbutyric acid methyl ester using general procedure E, followed by ester hydrolysis according to general procedure F to give 4-(4′-{2-[4-(2,4-dichloro-phenyl)-1-ethyl-1H-imidazol-2-yl]-(E)-vinyl}-biphenyl-4-yloxy)-2-methyl-butyric acid (25 mg, 22% yield).

LCMS: m/z 535 (M+H)⁺; ¹H NMR (CD₃OD, 400 MHz): δ 1.23 (d, 3H), 1.48 (t, 3H), 1.87 (m, 1H), 2.17 (m, 1H), 2.70 (m, 1H), 4.04 (t, 2H), 4.15 (q, 2H), 6.92-6.98 (m, 3H), 7.30 (dd, 1H), 7.41 (d, 1H), 7.50-7.63 (m, 8H), 7.98 (d, 1H) ppm.

### Example 259

### 4-(4′-{2-[4-(2,4-Dichloro-phenyl)-1-ethyl-1H-imidazol-2-yl]-(E)-vinyl}-biphenyl-4-yloxy)-pentanoic acid

4′-{2-[4-(2,4-Dichloro-phenyl)-1-ethyl-1H-imidazol-2-yl]-(E)-vinyl}-biphenyl-4-ol (90 mg, 0.21 mmol) was treated with 4-bromopentanoic acid methyl ester using general procedure E, followed by ester hydrolysis according to general procedure F to give 4-(4′-{2-[4-(2,4-dichloro-phenyl)-1-ethyl-1H-imidazol-2-yl]-(E)-vinyl}-biphenyl-4-yloxy)-pentanoic acid (22 mg, 20% yield).

LCMS: m/z 535 (M+H)⁺; ¹H NMR (CDCl₃, 400 MHz): δ 1.35 d, 3H), 1.52 (t, 3H), 1.96-2.09 (m, 2H), 2.55 (t, 2H), 4.13 (q, 2H), 4.51 (m, 1H), 6.90-6.97 (m, 3H), 7.32 (dd, 1H), 7.43 (d, 1H), 7.48-7.60 (m, 6H), 7.64 (s, 1H), 7.73 (d, 1H), 8.20 (d, 1H) ppm.

### Example 260

### 4-({2-[4-(2,4-Dichloro-phenyl)-1-ethyl-1H-imidazol-2-yl]-3H-benzoimidazole-5-carbonyl}-amino)-butyric acid

4-(2,4-Dichloro-phenyl)-1-ethyl-1H-imidazole-2-carbaldehyde (20 mg, 0.074 mmol) was treated with methyl 3,4-diaminobenzoate using general procedure Q followed by ester hydrolysis according to general procedure F. The resulting acid was coupled with methyl 4-aminobutyrate using general procedure G, then ester hydrolysis according to general procedure F gave 4-({2-[4-(2,4-dichloro-phenyl)-1-ethyl-1H-imidazol-2-yl]-3H-benzoimidazole-5-carbonyl}-amino)-butyric acid (1.6 mg, 4.5% yield).

LCMS: m/z 486 (M+H)⁺; ¹H NMR (CD₃OD, 400 MHz): δ 1.55 (t, 3H), 1.95 (m, 2H), 2.40 (t, 2H), 4.27 (m, 2H), 4.82 (q, 2H), 7.42 (dd, 1H), 7.54 (d, 1H), 7.60-7.65 (m, 2H), 7.72 (m, 1H), 8.04 (s, 1H), 8.27 (d, 1H) ppm.

### Example 261

### 6-{6-[4-(2,4-Dichloro-phenyl)-1-ethyl-1H-imidazol-2-yl]-naphthalen-2-yloxy}-hexanoic acid

6-[4-(2,4-Dichloro-phenyl)-1-ethyl-1H-imidazol-2-yl]-naphthalen-2-ol (40 mg, 0.1 mmol) was treated with 6-bromohexanoic acid ethyl ester using general procedure E, followed by ester hydrolysis according to general procedure F to give 6-{6-[4-(2,4-dichloro-phenyl)-1-ethyl-1H-imidazol-2-yl]-naphthalen-2-yloxy}-hexanoic acid (10 mg, 20% yield).

LCMS: m/z 497 (M+H)⁺; ¹H NMR (CDCl₃, 400 MHz): δ 1.47 (m, 5H), 1.68 (m, 2H), 1.81 (m, 2H), 2.35 (t, 2H), 3.97 (t, 2H), 4.15 (q, 2H), 7.12 (d, 1H), 7.19 (dd, 1H), 7.31 (dd, 1H), 7.44 (d, 1H), 7.69 (dd, 1H), 7.76-7.84 (m, 3H), 8.04 (s, 1H), 8.21 (d, 1H) ppm.

### Example 262

### 6-{2-[4-(2,4-Dichloro-phenyl)-1-ethyl-1H-imidazol-2-yl]-3-ethyl-3H-benzoimidazol-5-yloxy}-hexanoic acid

4-(2,4-Dichloro-phenyl)-1-ethyl-1H-imidazole-2-carbaldehyde (50 mg, 0.186 mmol) was treated with methyl 3,4-diaminoanwasole using general procedure Q followed by benzimidazole alkylation with iodoethane according to general procedure E. The resulting compound was demethylated using general procedure C. The phenol was then treated with 6-bromohexanoic acid ethyl ester using general procedure E, followed by ester hydrolysis according to general procedure F to give 6-{2-[4-(2,4-dichloro-phenyl)-1-ethyl-1H-imidazol-2-yl]-3-ethyl-3H-benzoimidazol-5-yloxy}-hexanoic acid (4 mg, 4.3% yield).

LCMS: m/z 515 (M+H)⁺; ¹H NMR (CD₃OD, 400 MHz): δ 1.47-1.57 (m, 6H), 1.62 (m, 2H), 1.77 (m, 2H), 1.87 (m, 2H), 2.43 (t, 2H), 4.07 (t, 2H), 4.74 (m, 4H), 6.87-6.96 (m, 2H), 7.32 (dd, 1H), 7.46 (d, 1H), 7.68 (d, 1H), 7.86 (s, 1H), 8.21 (d, 1H) ppm.

### Example 263

### 6-{2-[4-(2,4-Dichloro-phenyl)-1-ethyl-1H-imidazol-2-yl]-3H-benzoimidazol-5-yloxy}-hexanoic acid

3,4-dinitrophenol and ethyl 6-bromohexanoate were reacted using general procedure E, followed by nitro reduction using general procedure R. The resulting diamine and 4-(2,4-dichloro-phenyl)-1-ethyl-1H-imidazole-2-carbaldehyde (25 mg, 0.093 mmol) reacted using general procedure Q, followed by ester hydrolysis according to general procedure F to give 6-{2-[4-(2,4-dichloro-phenyl)-1-ethyl-1H-imidazol-2-yl]-3H-benzoimidazol-5-yloxy}-hexanoic acid (3 mg, 6.5% yield).

LCMS: m/z 487 (M+H)⁺; ¹H NMR (CD₃OD, 400 MHz): δ 1.55-1.63 (m, 5H), 1.75 (m, 2H), 1.87 (m, 2H), 2.37 (t, 2H), 4.07 (t, 2H), 4.77 (m, 2H), 6.95 (br s, 1H), 7.06 (br s, 1H), 7.38 (dd, 1H), 7.50 (d, 1H), 7.66 (br s, 1H), 7.86 (s, 1H), 8.12 (d, 1H) ppm.

### Example 264

### (3-{2-[4-(2,4-Dichloro-phenyl)-1-ethyl-1H-imidazol-2-yl]-3H-benzoimidazol-5-ylethynyl}-phenoxy)-acetic acid

6-Bromo-2-[4-(2,4-dichloro-phenyl)-1-ethyl-1H-imidazol-2-yl]-1-(2-trimethylsilanyl-ethoxymethyl)-1H-benzoimidazole (28.3 mg, 0.05 mmol) was treated with (3-ethynyl-phenoxy)-acetic acid methyl ester using general procedure H, followed by silyl group deprotection (with concurrent ester hydrolysis) according to general procedure S to give (3-{2-[4-(2,4-dichloro-phenyl)-1-ethyl-1H-imidazol-2-yl]-3H-benzoimidazol-5-ylethynyl}-phenoxy)-acetic acid (1 mg, 4% yield).

LCMS: m/z 531 (M+H)⁺; ¹H NMR (CD₃OD, 400 MHz): δ 1.48 (t, 3H), 4.39 (s, 2H), 4.77 (q, 2H), 6.88 (m, 1H), 7.01-7.06 (m, 2H), 7.19 (t, 1H), 7.32-7.39 (m, 2H), 7.46 (d, 1H), 7.96 (s, 1H), 8.19 (d, 1H) ppm.

### Example 265

### 4-(3-{2-[4-(2,4-Dichloro-phenyl)-1-ethyl-1H-imidazol-2-yl]-3H-benzoimidazol-5-ylethynyl}-phenoxy)-butyric acid

6-Bromo-2-[4-(2,4-dichloro-phenyl)-1-ethyl-1H-imidazol-2-yl]-1-(2-trimethylsilanyl-ethoxymethyl)-1H-benzoimidazole (28.3 mg, 0.05 mmol) was treated with (3-ethynyl-phenoxy)-butyric acid methyl ester using general procedure H, followed by silyl group deprotection (with concurrent ester hydrolysis) according to general procedure S to give 4-(3-{2-[4-(2,4-dichloro-phenyl)-1-ethyl-1H-imidazol-2-yl]-3H-benzoimidazol-5-ylethynyl}-phenoxy)-butyric acid (2 mg, 8% yield).

LCMS: m/z 559 (M+H)⁺; ¹H NMR (CDCl₃, 400 MHz): δ 1.60 (t, 3H), 2.18 (m, 2H), 2.60 (t, 2H), 4.09 (t, 2H), 4.90 (q, 2H), 6.87 (d, 1H), 7.13 (d, 2H), 7.35 (d, 1H), 7.43-7.50 (m, 2H), 7.66 (s, 1H), 7.70-7.77 (m, 2H), 7.86 (d, 1H) 7.96 (s, 1H) ppm.

### Example 266

### {3-[2-[4-(2,4-Dichloro-phenyl)-1-ethyl-1H-imidazol-2-yl]-3-(2-trimethylsilanyl-ethoxymethyl)-3 H-benzoimidazol-5-ylethynyl]-phenoxy}-acetic acid

6-Bromo-2-[4-(2,4-dichloro-phenyl)-1-ethyl-1H-imidazol-2-yl]-1-(2-trimethylsilanyl-ethoxymethyl)-1H-benzoimidazole (36 mg, 0.06 mmol) was treated with (3-ethynyl-phenoxy)-acetic acid methyl ester using general procedure H, followed by ester hydrolysis according to general procedure F to give {3-[2-[4-(2,4-dichloro-phenyl)-1-ethyl-1H-imidazol-2-yl]-3-(2-trimethylsilanyl-ethoxymethyl)-3H-benzoimidazol-5-ylethynyl]-phenoxy}-acetic acid (2 mg, 5% yield).

LCMS: m/z 661 (M+H)⁺; ¹H NMR (CDCl₃, 400 MHz): δ 0.13 (s, 9H), 1.10 (m, 2H), 1.68 (t, 3H), 3.73 (m, 2H), 4.81-4.95 (m, 4H), 6.51 (d, 2H), 7.10 (m, 1H), 7.26 (s, 1H), 7.38 (d, 1H), 7.42-7.49 (m, 2H), 7.61 (d, 1H), 7.63-7.72 (m, 2H), 7.90 (d, 1H), 8.07 (s, 1H), 8.31 (d, 1H) ppm.

### Example 267

### 3-[2-[4-(2,4-Dichloro-phenyl)-1-ethyl-1H-imidazol-2-yl]-3-(2-trimethylsilanyl-ethoxymethyl)-3H-benzoimidazol-5-ylethynyl]-benzoic acid

6-Bromo-2-[4-(2,4-dichloro-phenyl)-1-ethyl-1H-imidazol-2-yl]-1-(2-trimethylsilanyl-ethoxymethyl)-1H-benzoimidazole (59 mg, 0.1 mmol) was treated with trimethylsilylacetylene using general procedure H, followed by selective TMS group removal using general procedure T. The resulting acetylene was treated with ethyl 3-iodobenzoate using general procedure H, followed by ester hydrolysis according to general procedure F to give 3-[2-[4-(2,4-dichloro-phenyl)-1-ethyl-1H-imidazol-2-yl]-3-(2-trimethylsilanyl-ethoxymethyl)-3H-benzoimidazol-5-ylethynyl]-benzoic acid (0.3 mg, 0.5% yield).

LCMS: m/z 631 (M+H)⁺.

### Example 268

### 4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid methyl ester

4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid methyl ester (179 mg, 55%) was prepared according to General Procedure A using trans 4-bromo cinnamic acid (227 mg, 1 mmol) and 2-bromo-2,4-dichloro acetophenone (267 mg, 1 mmol) and obtained 2-[2-(4-Bromo-phenyl)-(E)-vinyl]-4-(2,4-dichloro-phenyl)-1H-imidazole (394 mg, 1 mmol) was alkylated with methyl bromo acetate (153 mg, 1 mmol) following general procedure E. The obtained 2-[2-(4-Bromo-phenyl)-(E)-vinyl]-4-(2,4-dichloro-phenyl)-1H-imidazol-1-yl]-acetic acid methyl ester (466 mg, 1 mmol) was coupled with 4-ethoxy phenyl boronic acid (165 mg, 1 mmol) following General Procedure B and resulting 4{-(2,4-dichloro-phenyl)-2-[2-(4′-ethoxy-biphenyl-3-yl]-imidazol-1-yl}acetic acid methyl ester (479 mg, 1 mmol) was hydrolyzed according to General Procedure F and resulted {4-(2,4-Dichloro-phenyl)-2-[2-(4′-ethoxy-biphenyl-4-yl)-(E)-vinyl]-imidazol-1-yl}-acetic acid (247 mg, 0.5 mmol) was coupled with 4-(aminomethyl)-benzoic acid-methyl ester (83 mg, 0.5 mmol) following general procedure G.

LCMS: 640 (M+H)⁺

### Example 269

### 4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid

4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid methyl ester (160 mg, 0.25 mmol) was hydrolyzed according to General Procedure F to give 4-[(2-{4-(2,4-Dichloro-phenyl)-2-[2-(4′-ethoxy-biphenyl-4-yl)-(E)-vinyl]-imidazol-1-yl}-acetylamino)-methyl]-benzoic acid (99 mg, 63%).

LCMS: 626 (M+H)⁺

### Example 270

### 4-[4′-(2-{4-(2,4-Dichloro-phenyl)-1-[(4-fluoro-benzylcarbamoyl)-methyl]-1H-imidazol-2-yl}-(E)-vinyl)-biphenyl-4-yloxy]1-butyric acid methyl ester

4-[4′-(2-{4-(2,4-Dichloro-phenyl)-1-[(4-fluoro-benzylcarbamoyl)-methyl]-1H-imidazol-2-yl}-(E)-vinyl)-biphenyl-4-yloxy]-butyric acid methyl ester (189 mg, 56%) was prepared according to General Procedure A using trans 4-bromo cinnamic acid (227 mg, 1 mmol) and 2-bromo-2,4-dichloro acetophenone (267 mg, 1 mmol) and obtained 2-[2-(4-Bromo-phenyl)-(E)-vinyl]-4-(2,4-dichloro-phenyl)-1H-imidazole (394 mg, 1 mmol) was alkylated with methyl bromo acetate (153 mg, 1 mmol) following general procedure E. The obtained 2-[2-(4-Bromo-phenyl)-(E)-vinyl]-4-(2,4-dichloro-phenyl)-1H-imidazol-1yl]-acetic acid methyl ester (466 mg, 1 mmol) was coupled with 4-hydroxy phenyl boronic acid (138 mg, 1 mmol) following General Procedure B and resulting {4-(2,4-Dichloro-phenyl)-2-[2-(4′-hydroxy-biphenyl-4-yl)-(E)-vinyl]-imidazol-1-yl}-acetic acid methyl ester (240 mg, 0.5 mmol) was hydrolyzed according to General Procedure F. The resulted {4-(2,4-Dichloro-phenyl)-2-[2-(4′-hydroxy-biphenyl-4-yl)-(E)-vinyl]-imidazol-1-yl}-acetic acid (233 mg, 0.5 mmol) was coupled with 4-fluoro benzylamine (63 mg, 0.5 mmol) following general procedure G and obtained 2-{4-(2,4-Dichloro-phenyl)-2-[2-(4′-hydroxy-biphenyl-4-yl)-(E)-vinyl]-imidazol-1-yl}-N-(4-fluoro-benzyl)-acetamide (286 mg, 0.5 mmol) was alkylated with 4-bromobutyric acid methyl ester (91 mg, 0.5 mmol) according to general procedure E.

LCMS: 672 (M+H)⁺

### Example 271

### 4-[4′-(2-{4-(2,4-Dichloro-phenyl)-1-[(4-fluoro-benzylcarbamoyl)-methyl]-1H-imidazol-2-yl}-(E)-vinyl)-biphenyl-4-yloxy]1-butyric acid

4-[4′-(2-{4-(2,4-Dichloro-phenyl)-1-[(4-fluoro-benzylcarbamoyl)-methyl]-1H-imidazol-2-yl}-(E)-vinyl)-biphenyl-4-yloxy]-butyric acid methyl ester (168 mg, 0.25 mmol) was hydrolyzed according to General Procedure F to give 4-[4′-(2-{4-(2,4-Dichloro-phenyl)-1-[(4-fluoro-benzylcarbamoyl)-methyl]-1H-imidazol-2-yl}-(E)-vinyl)-biphenyl-4-yloxy]-butyric acid (101 mg, 62%).

LCMS: 658 (M+H)⁺

### Example 272

### 4-[4′-(2-{4-(2,4-Dichloro-phenyl)-1-[(4-methoxy-benzylcarbamoyl)-methyl]-1H-imidazol-2-yl}-(E)-vinyl)-biphenyl-4-yloxy]1-butyric acid methyl ester

4-[4′-(2-{4-(2,4-Dichloro-phenyl)-1-[(4-methoxy-benzylcarbamoyl)-methyl]-1H-imidazol-2-yl}-(E)-vinyl)-biphenyl-4-yloxy]-butyric acid methyl ester (191 mg, 55%) was prepared according to General Procedure A using trans 4-bromo cinnamic acid (227 mg, 1 mmol) and 2-bromo-2,4-dichloro acetophenone (267 mg, 1 mmol) and obtained 2-[2-(4-Bromo-phenyl)-(E)-vinyl]-4-(2,4-dichloro-phenyl)-1H-imidazole (394 mg, 1 mmol) was alkylated with methyl bromoacetate (153 mg, 1 mmol) following general procedure E. Thus obtained 2-[2-(4-Bromo-phenyl)-(E)-vinyl]-4-(2,4-dichloro-phenyl)-1H-imidazol-1yl]-acetic acid methyl ester (466 mg, 1 mmol) was coupled with 4-hydroxy phenyl boronic acid (138 mg, 1 mmol) following General Procedure B and resulting {4-(2,4-Dichloro-phenyl)-2-[2-(4′-hydroxy-biphenyl-4-yl)-(E)-vinyl]-imidazol-1-yl}-acetic acid methyl ester (240 mg, 0.5 mmol) was hydrolyzed according to General Procedure F. The resulted {4-(2,4-Dichloro-phenyl)-2-[2-(4′-hydroxy-biphenyl-4-yl)-(E)-vinyl]-imidazol-1-yl}-acetic acid (233 mg, 0.5 mmol) was coupled with 4-methoxy benzylamine (69 mg, 0.5 mmol) following general procedure G and obtained 2-{4-(2,4-Dichloro-phenyl)-2-[2-(4′-hydroxy-biphenyl-4-yl)-(E)-vinyl]-imidazol-1-yl}-N-(4-methoxy-benzyl)-acetamide (292 mg, 0.5 mmol) was alkylated with 4-bromobutyric acid methyl ester (91 mg, 0.5 mmol) according to general procedure E.

LCMS: 684 (M+H)⁺

### Example 273

### 4-[4′-(2-{4-(2,4-Dichloro-phenyl)-1-[(4-methoxy-benzylcarbamoyl)-methyl]-1H-imidazol-2-yl}-(E)-vinyl)-biphenyl-4-yloxy]-butyric acid

4-[4′-(2-{4-(2,4-Dichloro-phenyl)-1-[(4-methoxy-benzylcarbamoyl)-methyl]-1H-imidazol-2-yl}-(E)-vinyl)-biphenyl-4-yloxy]-butyric acid methyl ester (171 mg, 0.25 mmol) was hydrolyzed according to General Procedure F to give 4-[4′-(2-{4-(2,4-Dichloro-phenyl)-1-[(4-methoxy-benzylcarbamoyl)-methyl]-1H-imidazol-2-yl}-(E)-vinyl)-biphenyl-4-yloxy]-butyric acid (112 mg, 67%).

LCMS: 670 (M+H)⁺

### Example 274

### 4-[4′-(2-{4-(2,4-Dichloro-phenyl)-1-[(4-trifluoromethoxy-benzylcarbamoyl)-methyl]-1H-imidazol-2-yl}-(E)-vinyl)-biphenyl-4-yloxy]1-butyric acid methyl ester

4-[4′-(2-{4-(2,4-Dichloro-phenyl)-1-[(4-trifluoromethoxy-benzylcarbamoyl)-methyl]-1H-imidazol-2-yl}-(E)-vinyl)-biphenyl-4-yloxy]-butyric acid methyl ester (201 mg, 54%) was prepared according to General Procedure A using trans 4-bromo cinnamic acid (227 mg, 1 mmol) and 2-bromo-2,4-dichloro acetophenone (267 mg, 1 mmol) and obtained 2-[2-(4-Bromo-phenyl)-(E)-vinyl]-4-(2,4-dichloro-phenyl)-1H-imidazole (394 mg, 1 mmol) was alkylated with methyl bromo acetate (153 mg, 1 mmol) following general procedure E. The obtained 2-[2-(4-Bromo-phenyl)-(E)-vinyl]-4-(2,4-dichloro-phenyl)-1H-imidazol-1-yl]-acetic acid methyl ester (466 mg, 1 mmol) was coupled with 4-hydroxy phenyl boronic acid (138 mg, 1 mmol) following General Procedure B and resulting {4-(2,4-Dichloro-phenyl)-2-[2-(4′-hydroxy-biphenyl-4-yl)-(E)-vinyl]-imidazol-1-yl}-acetic acid methyl ester (240 mg, 0.5 mmol) was hydrolyzed according to General Procedure F. The resulted {4-(2,4-Dichloro-phenyl)-2-[2-(4′-hydroxy-biphenyl-4-yl)-(E)-vinyl]-imidazol-1-yl}-acetic acid (233 mg, 0.5 mmol) was coupled with 4-trifluoromethoxy benzylamine (96 mg, 0.5 mmol) following general procedure G and obtained 2-{4-(2,4-Dichloro-phenyl)-2-[2-(4′-hydroxy-biphenyl-4-yl)-(E)-vinyl]-imidazol-1-yl}-N-(4-trifluoromethoxy-benzyl)-acetamide (319 mg, 0.5 mmol) was alkylated with 4-bromobutyric acid methyl ester (91 mg, 0.5 mmol) according to general procedure E.

LCMS: m/z 738 (M+H)⁺

### Example 275

### 4-[4′-(2-{4-(2,4-Dichloro-phenyl)-1-[(4-trifluoromethoxy-benzylcarbamoyl)-methyl]-1H-imidazol-2-yl}-(E)-vinyl)-biphenyl-4-yloxy]1-butyric acid

4-[4′-(2-{4-(2,4-Dichloro-phenyl)-1-[(4-trifluoromethoxy-benzylcarbamoyl)-methyl]-1H-imidazol-2-yl}-(E)-vinyl)-biphenyl-4-yloxy]-butyric acid methyl ester (185 mg, 0.25 mmol) was hydrolyzed according to General Procedure F to give 4-[4′-(2-{4-(2,4-Dichloro-phenyl)-1-[(4-trifluoromethoxy-benzylcarbamoyl)-methyl]-1H-imidazol-2-yl}-(E)-vinyl)-biphenyl-4-yloxy]-butyric acid (121 mg, 67%).

LCMS: 724 (M+H)⁺; ¹H NMR (DMSO, 400 MHz): δ 1.60 (m, 2H), 1.95 (m, 2H), 2.19 (m, 2H), 2.36 (m, 2H), 4.36 (m, 2H), 5.05 (s, 2H), 7.02 (d, 1H), 7.15-7.19 (m, 4H), 7.38 (d, 1H), 7.50 (d, 1H), 7.55-7.69 (m, 6H), 7.71 (d, 1H), 7.96 (s, 1H), 8.29 (d, 1H), 8.88 (s, 1H) ppm.

### Example 276

### 4-{4-(2,4-Dichloro-phenyl)-2-[2-(6′-fluoro-2′-methoxy-biphenyl-4-yl)-(E)-vinyl]-imidazol-1-ylmethyl}-benzoic acid

4-[2-[2-(4-Bromo-phenyl)-(E)-vinyl]-4-(2,4-dichloro-phenyl)-imidazol-1-ylmethyl]-benzoic acid methyl ester (300 mg, 0.55 mmol) was treated with 6-fluoro-2-methoxyphenylboronic acid using general procedure B, followed by ester hydrolysis according to general procedure F to give 4-{4-(2,4-dichloro-phenyl)-2-[2-(6′-fluoro-2′-methoxy-biphenyl-4-yl)-(E)-vinyl]-imidazol-1-ylmethyl}-benzoic acid (197 mg, 62% yield).

LCMS: m/z 573 (M+H)⁺; ¹H NMR (DMSO-d₆, 400 MHz): δ 3.74 (s, 3H), 5.62 (s, 2H), 7.08-7.20 (m, 3H), 7.30-7.37 (m, 3H), 7.48-7.53 (m, 3H), 7.56 (d, 1H), 7.63 (d, 1H), 7.69 (d, 2H), 7.93 (d, 2H), 8.10 (s, 1H), 8.27 (d, 1H) ppm.

### Example 277

### 4-[2-[2-(3′-Cyano-biphenyl-4-yl)-(E)-vinyl]-4-(2,4-dichloro-phenyl)-imidazol-1-ylmethyl]-benzoic acid

4-[2-[2-(4-Bromo-phenyl)-(E)-vinyl]-4-(2,4-dichloro-phenyl)-imidazol-1-ylmethyl]-benzoic acid methyl ester (300 mg, 0.55 mmol) was treated with 3-cyanophenyl boronic acid using general procedure B, followed by ester hydrolysis according to general procedure F to give 4-[2-[2-(3′-cyano-biphenyl-4-yl)-(E)-vinyl]-4-(2,4-dichloro-phenyl)-imidazol-1-ylmethyl]-benzoic acid (53 mg, 17% yield).

LCMS: m/z 550 (M+H)⁺; ¹H NMR (DMSO-d₆, 400 MHz): δ 5.64 (s, 2H), 7.33-7.41 (m, 3H), 7.50 (dd, 1H), 7.58 (d, 1H), 7.64 (d, 1H), 7.67 (d, 1H), 7.75-7.79 (m, 4H), 7.82 (d, 1H), 7.93 (d, 2H), 8.06 (d, 1H), 8.10 (s, 1H), 8.20 (s, 1H), 8.27 (d, 1H) ppm.

### Example 278

### 4-[4-(2,4-Dichloro-phenyl)-2-(4′-trifluoromethyl-biphenyl-4-ylmethyl)-imidazol-1-ylmethyl]-benzoic acid methyl ester

Step 1: 4-Bromophenylacetic acid (2.15 g, 10 mmol) is treated according to general procedure A using 2,4-dichlorophenacyl bromide to give the intermediate 2-(4-bromo-benzyl)-4-(2,4-dichloro-phenyl)-1H-imidazole, which is then treated as described in general procedure E using methyl 4-(bromomethyl)benzoate to give 4-[2-(4-bromobenzyl)-4-(2,4-dichloro-phenyl)-imidazol-1-ylmethyl]-benzoic acid methyl ester (1.96 g, 37% total yield).

LCMS: m/z 531 (M+H)⁺; ¹H NMR (DMSO-d₆, 400 MHz): δ 3.79 (s, 3H), 4.11 (s, 2H), 5.36 (s, 2H), 7.46-7.50 (m, 4H), 7.61 (d, 2H), 7.65 (d, 2H), 7.69 (d, 2H), 7.74 (s, 1H), 7.81 (d, 1H) ppm.

Step 2: 4-[4-(2,4-Dichloro-phenyl)-2-(4′-trifluoromethyl-biphenyl-4-ylmethyl)-imidazol-1-ylmethyl]-benzoic acid methyl ester (41 mg, 34% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 4-(trifluoromethyl)benzeneboronic acid (46 mg, 0.24 mmol).

LCMS: m/z 595 (M+H)⁺.

### Example 279

### 4-[4-(2,4-Dichloro-phenyl)-2-(4′-trifluoromethyl-biphenyl-4-ylmethyl)-imidazol-1-ylmethyl]-benzoic acid

4-[4-(2,4-Dichloro-phenyl)-2-(4′-trifluoromethyl-biphenyl-4-ylmethyl)-imidazol-1-ylmethyl]-benzoic acid (32 mg, 91% yield) is prepared according to general procedure F using 4-[4-(2,4-dichloro-phenyl)-2-(4′-trifluoromethyl-biphenyl-4-ylmethyl)-imidazol-1-ylmethyl]-benzoic acid methyl ester (36 mg, 0.06 mmol).

LCMS: m/z 581 (M+H)⁺; ¹H NMR (DMSO-d₆, 400 MHz): δ 4.10 (s, 2H), 5.34 (s, 2H), 7.13 (d, 2H), 7.23 (d, 2H), 7.40 (d, 2H), 7.44 (dd, 1H), 7.48 (d, 2H), 7.60 (d, 1H), 7.68 (d, 2H), 7.81 (d, 2H), 7.94 (s, 1H), 8.18 (d, 1H) ppm.

### Example 280

### 4-[4-(2,4-Dichloro-phenyl)-2-(3′-trifluoromethyl-biphenyl-4-ylmethyl)-imidazol-1-ylmethyl]-benzoic acid methyl ester

4-[4-(2,4-Dichloro-phenyl)-2-(3′-trifluoromethyl-biphenyl-4-ylmethyl)-imidazol-1-ylmethyl]-benzoic acid methyl ester (37 mg, 31% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 3-(trifluoromethyl)benzeneboronic acid (46 mg, 0.24 mmol).

LCMS: m/z 595 (M+H)⁺.

### Example 281

### 4-[4-(2,4-Dichloro-phenyl)-2-(3′-trifluoromethyl-biphenyl-4-ylmethyl)-imidazol-1-ylmethyl]-benzoic acid

4-[4-(2,4-Dichloro-phenyl)-2-(3′-trifluoromethyl-biphenyl-4-ylmethyl)-imidazol-1-ylmethyl]-benzoic acid (26 mg, 89% yield) is prepared according to general procedure F using 4-[4-(2,4-dichloro-phenyl)-2-(3′-trifluoromethyl-biphenyl-4-ylmethyl)-imidazol-1-ylmethyl]-benzoic acid methyl ester (30 mg, 0.05 mmol).

LCMS: m/z 581 (M+H)⁺; ¹H NMR (DMSO-d₆, 400 MHz): δ 4.12 (s, 2H), 5.35 (s, 2H), 7.14 (d, 2H), 7.26 (d, 2H), 7.44 (dd, 1H), 7.57 (d, 2H), 7.60 (d, 1H), 7.65-7.69 (m, 4H), 7.82 (d, 2H), 7.95 (s, 1H), 8.17 (d, 1H) ppm.

### Example 282

### 4-[4-(2,4-Dichloro-phenyl)-2-(4′-trifluoromethoxy-biphenyl-4-ylmethyl)-imidazol-1-ylmethyl]-benzoic acid methyl ester

4-[4-(2,4-Dichloro-phenyl)-2-(4′-trifluoromethoxy-biphenyl-4-ylmethyl)-imidazol-1-ylmethyl]-benzoic acid methyl ester (93 mg, 78% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 4-(trifluoromethoxy)benzeneboronic acid (50 mg, 0.24 mmol).

LCMS: m/z 611 (M+H)⁺.

### Example 283

### 4-[4-(2,4-Dichloro-phenyl)-2-(4′-trifluoromethoxy-biphenyl-4-ylmethyl)-imidazol-1-ylmethyl]-benzoic acid

4-[4-(2,4-Dichloro-phenyl)-2-(4′-trifluoromethoxy-biphenyl-4-ylmethyl)-imidazol-1-ylmethyl]-benzoic acid (54 mg, 90% yield) is prepared according to general procedure F using 4-[4-(2,4-dichloro-phenyl)-2-(4′-trifluoromethoxy-biphenyl-4-ylmethyl)-imidazol-1-ylmethyl]-benzoic acid methyl ester (61 mg, 0.1 mmol).

LCMS: m/z 597 (M+H)⁺; ¹H NMR (DMSO-d₆, 400 MHz): δ 4.11 (s, 2H), 5.34 (s, 2H), 7.13 (d, 2H), 7.23 (d, 2H), 7.39 (d, 2H), 7.43 (dd, 1H), 7.48 (d, 2H), 7.60 (d, 1H), 7.68 (d, 2H), 7.81 (d, 2H), 7.94 (s, 1H), 8.17 (d, 1H) ppm.

### Example 284

### 4-[4-(2,4-Dichloro-phenyl)-2-(3′-trifluoromethoxy-biphenyl-4-ylmethyl)-imidazol-1-ylmethyl]-benzoic acid methyl ester

4-[4-(2,4-Dichloro-phenyl)-2-(3′-trifluoromethoxy-biphenyl-4-ylmethyl)-imidazol-1-ylmethyl]-benzoic acid methyl ester (88 mg, 72% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and 3-(trifluoromethoxy)benzeneboronic acid (50 mg, 0.24 mmol).

LCMS: m/z 611 (M+H)⁺.

### Example 285

### 4-[4-(2,4-Dichloro-phenyl)-2-(3′-trifluoromethoxy-biphenyl-4-ylmethyl)-imidazol-1-ylmethyl]-benzoic acid

4-[4-(2,4-Dichloro-phenyl)-2-(3′-trifluoromethoxy-biphenyl-4-ylmethyl)-imidazol-1-ylmethyl]-benzoic acid (50 mg, 83% yield) is prepared according to general procedure F using 4-[4-(2,4-dichloro-phenyl)-2-(3′-trifluoromethoxy-biphenyl-4-ylmethyl)-imidazol-1-ylmethyl]-benzoic acid methyl ester (61 mg, 0.1 mmol).

LCMS: m/z 597 (M+H)⁺; ¹H NMR (DMSO-d₆, 400 MHz): δ 4.14 (s, 2H), 5.37 (s, 2H), 7.13 (d, 2H), 7.24 (d, 2H), 7.44 (dd, 1H), 7.57 (d, 2H), 7.60 (d, 1H), 7.65-7.69 (m, 4H), 7.81 (d, 2H), 7.94 (s, 1H), 8.17 (d, 1H) ppm.

### Example 286

### 4-[4-(2,4-Dichloro-phenyl)-2-(3′-methanesulfonyl-biphenyl-4-ylmethyl)-imidazol-1-ylmethyl]-benzoic acid methyl ester

4-[4-(2,4-Dichloro-phenyl)-2-(3′-methanesulfonyl-biphenyl-4-ylmethyl)-imidazol-1-ylmethyl]-benzoic acid methyl ester (68 mg, 56% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and (3-methylsulfonylphenyl)boronic acid (48 mg, 0.24 mmol).

LCMS: m/z 605 (M+H)⁺.

### Example 287

### 4-[4-(2,4-Dichloro-phenyl)-2-(3′-methanesulfonyl-biphenyl-4-ylmethyl)-imidazol-1-ylmethyl]-benzoic acid

4-[4-(2,4-Dichloro-phenyl)-2-(3′-methanesulfonyl-biphenyl-4-ylmethyl)-imidazol-1-ylmethyl]-benzoic acid (51 mg, 86% yield) is prepared according to general procedure F using 4-[4-(2,4-dichloro-phenyl)-2-(3′-methanesulfonyl-biphenyl-4-ylmethyl)-imidazol-1-ylmethyl]-benzoic acid methyl ester (61 mg, 0.1 mmol).

LCMS: m/z 591 (M+H)⁺; ¹H NMR (DMSO-d₆, 400 MHz): δ 3.28 (s, 3H), 4.14 (s, 2H), 5.37 (s, 2H), 7.13 (d, 2H), 7.24 (d, 2H), 7.44 (dd, 1H), 7.57 (d, 2H), 7.60 (d, 1H), 7.65-7.69 (m, 4H), 7.81 (d, 2H), 7.94 (s, 1H), 8.17 (d, 1H) ppm.

### Example 288

### 4-[4-(2,4-Dichloro-phenyl)-2-(4′-methanesulfonyl-biphenyl-4-ylmethyl)-imidazol-1-ylmethyl]-benzoic acid methyl ester

4-[4-(2,4-Dichloro-phenyl)-2-(4′-methanesulfonyl-biphenyl-4-ylmethyl)-imidazol-1-ylmethyl]-benzoic acid methyl ester (74 mg, 61% yield) is prepared according to general procedure B using 4-[2-(4-bromo-benzyl)-4-(2,4-dichloro-phenyl)-imidazol-1-ylmethyl]-benzoic acid methyl ester (106 mg, 0.2 mmol) and (4-methylsulfonylphenyl)boronic acid (48 mg, 0.24 mmol).

LCMS: m/z 605 (M+H)⁺.

### Example 289

### 4-[4-(2,4-Dichloro-phenyl)-2-(4′-methanesulfonyl-biphenyl-4-ylmethyl)-imidazol-1-ylmethyl]-benzoic acid

4-[4-(2,4-Dichloro-phenyl)-2-(4′-methanesulfonyl-biphenyl-4-ylmethyl)-imidazol-1-ylmethyl]-benzoic acid (53 mg, 89% yield) is prepared according to general procedure F using 4-[4-(2,4-dichloro-phenyl)-2-(4′-methanesulfonyl-biphenyl-4-ylmethyl)-imidazol-1-ylmethyl]-benzoic acid methyl ester (61 mg, 0.1 mmol).

LCMS: m/z 591 (M+H)⁺; ¹H NMR (DMSO-d₆, 400 MHz): δ 3.26 (s, 3H), 4.13 (s, 2H), 5.36 (s, 2H), 7.13 (d, 2H), 7.24 (d, 2H), 7.44 (dd, 1H), 7.57 (d, 2H), 7.60 (d, 1H), 7.65 (d, 2H), 7.72 (d, 2H), 7.81 (d, 2H), 7.94 (s, 1H), 8.17 (d, 1H) ppm.

### Example 290

### 4-[4-(2,4-Dichloro-phenyl)-2-(4-{[2-(4-methanesulfonyl-phenyl)-acetylamino]-methyl}-phenyl)-imidazol-1-ylmethyl]-benzoic acid methyl ester

4-(tert-Butoxycarbonylamino-methyl)-benzoic acid (502 mg, 2 mmol) is treated according to general procedure A using 2,4-dichlorophenacyl bromide to give {4-[4-(2,4-dichloro-phenyl)-1H-imidazol-2-yl]-benzyl}-carbamic acid tert-butyl ester, which is then treated as described in general procedure E using methyl 4-(bromomethyl)benzoate to give 4-[2-[4-(tert-butoxycarbonylamino-methyl)-phenyl]-4-(2,4-dichloro-phenyl)-imidazol-1-ylmethyl]-benzoic acid methyl ester, which is then treated with hydrogen chloride in ethyl ether and then coupled with 4-methylsulphonylphenylacetic acid according to general procedure G to afford the title compound 4-[4-(2,4-dichloro-phenyl)-2-(4-{[2-(4-methanesulfonyl-phenyl)-acetylamino]-methyl}-phenyl)-imidazol-1-ylmethyl]-benzoic acid methyl ester (239 mg, 18% total yield).

LCMS: m/z 662 (M+H)⁺.

### Example 291

### 4-[4-(2,4-Dichloro-phenyl)-2-(4-{[2-(4-methanesulfonyl-phenyl)-acetylamino]-methyl}-phenyl)-imidazol-1-ylmethyl]-benzoic acid

4-[4-(2,4-Dichloro-phenyl)-2-(4-{[2-(4-methanesulfonyl-phenyl)-acetylamino]-methyl}-phenyl)-imidazol-1-ylmethyl]-benzoic acid (92 mg, 71% yield) is prepared according to general procedure F using 4-[4-(2,4-dichloro-phenyl)-2-(4-{[2-(4-methanesulfonyl-phenyl)-acetylamino]-methyl}-phenyl)-imidazol-1-ylmethyl]-benzoic acid methyl ester (133 mg, 0.2 mmol).

LCMS: m/z 648 (M+H)⁺; ¹H NMR (DMSO-d₆, 400 MHz): δ 3.16 (s, 3H), 3.51 (s, 2H), 4.25 (d, 2H), 5.38 (s, 2H), 7.13 (d, 2H), 7.24 (d, 2H), 7.46-7.58 (m, 3H), 7.60 (d, 1H), 7.65 (d, 2H), 7.72 (d, 2H), 7.81 (d, 2H), 7.94 (s, 1H), 8.15 (d, 1H) ppm.

### Example 292

### 4-{4-(2,4-Difluoro-phenyl)-2-[2-(4′-ethoxy-biphenyl-4-yl)-(E)-vinyl]-imidazol-1-ylmethyl}-benzoic acid

Step 1: Trans-4-bromocinnamic acid (2.27 g, 10 mmol) is treated according to general procedure A using 2,4-difluorophenacyl bromide to give the intermediate 2-[2-(4-bromo-phenyl)-(E)-vinyl]-4-(2,4-difluoro-phenyl)-1H-imidazole, which is then treated as described in general procedure E using methyl 4-(bromomethyl)benzoate to give 4-[2-[2-(4-bromo-phenyl)-(E)-vinyl]-4-(2,4-difluoro-phenyl)-imidazol-1-ylmethyl]-benzoic acid methyl ester (1.68 g, 33% total yield).

LCMS: m/z 510 (M+H)⁺; ¹H NMR (DMSO-d₆, 400 MHz): δ 3.80 (s, 3H), 5.60 (s, 2H), 7.13 (d, 1H), 7.46-7.50 (m, 5H), 7.61 (d, 2H), 7.65 (d, 2H), 7.69 (d, 2H), 7.74 (s, 1H), 7.81 (d, 1H) ppm.

Step 2: 4-{4-(2,4-Difluoro-phenyl)-2-[2-(4′-ethoxy-biphenyl-4-yl)-(E)-vinyl]-imidazol-1-ylmethyl}-benzoic acid (150 mg, 56% total yield) is prepared according to general procedure B using 4-[2-[2-(4-bromo-phenyl)-(E)-vinyl]-4-(2,4-difluoro-phenyl)-imidazol-1-ylmethyl]-benzoic acid methyl ester (255 mg, 0.5 mmol) and 4-ethoxyphenylboronic acid (100 mg, 0.6 mmol), followed by ester-hydrolysis according to general procedure F.

LCMS: m/z 537 (M+H)⁺; ¹H NMR (DMSO-d₆, 400 MHz): δ 1.34 (t, 3H), 4.06 (q, 2H), 5.63 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.17 (d, 1H) ppm.

### Example 293

### 4-{4-(2,4-Difluoro-phenyl)-2-[2-(4′-ethoxy-biphenyl-4-yl)-ethyl]-imidazol-1-ylmethyl}-benzoic acid

4-{4-(2,4-Difluoro-phenyl)-2-[2-(4′-ethoxy-biphenyl-4-yl)-ethyl]-imidazol-1-ylmethyl}-benzoic acid (18 mg, 67% yield) is prepared according to general procedure V using 4-{4-(2,4-difluoro-phenyl)-2-[2-(4′-ethoxy-biphenyl-4-yl)-(E)-vinyl]-imidazol-1-ylmethyl}-benzoic acid (27 mg, 0.05 mmol).

LCMS: m/z 539 (M+H)⁺; ¹H NMR (DMSO-d₆, 400 MHz): δ 1.32 (t, 3H), 2.86 (m, 2H), 2.96 (m, 2H), 4.03 (q, 2H), 5.32 (s, 2H), 7.13 (d, 2H), 7.24 (d, 2H), 7.39 (d, 1H), 7.47 (d, 2H), 7.62 (d, 1H), 7.65-7.69 (m, 4H), 7.81 (d, 2H), 7.94 (s, 1H), 8.17 (d, 1H) ppm.

### Example 294

### 4-{4-(2,4-Difluoro-phenyl)-2-[2-(4′-hydroxy-biphenyl-4-yl)-(E)-vinyl]-imidazol-1-ylmethyl}-benzoic acid

4-{4-(2,4-Difluoro-phenyl)-2-[2-(4′-hydroxy-biphenyl-4-yl)-(E)-vinyl]-imidazol-1-ylmethyl}-benzoic acid (72 mg, 71% total yield) is prepared according to general procedure C using 4-{4-(2,4-difluoro-phenyl)-2-[2-(4′-ethoxy-biphenyl-4-yl)-(E)-vinyl]-imidazol-1-ylmethyl}-benzoic acid (107 mg, 0.2 mmol).

LCMS: m/z 509 (M+H)⁺; ¹H NMR (DMSO-d₆, 400 MHz): δ 5.62 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.16 (d, 1H) ppm.

### Example 295

### 4-[2-[2-(4′-Butoxy-biphenyl-4-yl)-(E)-vinyl]-4-(2,4-difluoro-phenyl)-imidazol-1-ylmethyl]-benzoic acid

4-[2-[2-(4′-Butoxy-biphenyl-4-yl)-(E)-vinyl]-4-(2,4-difluoro-phenyl)-imidazol-1-ylmethyl]-benzoic acid (28 mg, 49% total yield) is prepared according to general procedure E using 4-{4-(2,4-difluoro-phenyl)-2-[2-(4′-hydroxy-biphenyl-4-yl)-(E)-vinyl]-imidazol-1-ylmethyl}-benzoic acid (51 mg, 0.1 mmol) and 1-bromobutane, followed by ester-hydrolysis according to general procedure F.

LCMS: m/z 565 (M+H)⁺; ¹H NMR (DMSO-d₆, 400 MHz): δ 1.04 (t, 3H), 1.46 (m, 2H), 1.90 (m, 2H), 4.18 (t, 2H), 5.61 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.17 (d, 1H) ppm.

### Example 296

### 4-{4-(2,4-Difluoro-phenyl)-2-[2-(3′-trifluoromethyl-biphenyl-4-yl)-(E)-vinyl]-imidazol-1-ylmethyl}-benzoic acid

4-{4-(2,4-Difluoro-phenyl)-2-[2-(3′-trifluoromethyl-biphenyl-4-yl)-(E)-vinyl]-imidazol-1-ylmethyl}-benzoic acid (87 mg, 31% total yield) is prepared according to general procedure B using 4-[2-[2-(4-bromo-phenyl)-(E)-vinyl]-4-(2,4-difluoro-phenyl)-imidazol-1-ylmethyl]-benzoic acid methyl ester (255 mg, 0.5 mmol) and 3-(trifluoromethyl)benzeneboronic acid (114 mg, 0.6 mmol), followed by ester-hydrolysis according to general procedure F.

LCMS: m/z 561 (M+H)⁺; ¹H NMR (DMSO-d₆, 400 MHz): δ 5.60 (s, 2H), 7.13 (d, 2H), 7.24 (d, 2H), 7.33 (d, 1H), 7.39 (d, 1H), 7.47 (d, 2H), 7.58 (d, 1H), 7.62 (d, 1H), 7.65-7.69 (m, 4H), 7.81 (d, 2H), 7.94 (s, 1H), 8.18 (d, 1H) ppm.

### Example 297

### 4-{4-(2,4-Difluoro-phenyl)-2-[2-(3′-trifluoromethyl-biphenyl-4-yl)-ethyl]-imidazol-1-ylmethyl}-benzoic acid

4-{4-(2,4-Difluoro-phenyl)-2-[2-(3′-trifluoromethyl-biphenyl-4-yl)-ethyl]-imidazol-1-ylmethyl}-benzoic acid (21 mg, 74% yield) is prepared according to general procedure V using 4-{4-(2,4-difluoro-phenyl)-2-[2-(3′-trifluoromethyl-biphenyl-4-yl)-(E)-vinyl]-imidazol-1-ylmethyl}-benzoic acid (28 mg, 0.05 mmol).

LCMS: m/z 563 (M+H)⁺; ¹H NMR (DMSO-d₆, 400 MHz): δ 2.88 (m, 2H), 2.97 (m, 2H), 5.32 (s, 2H), 7.13 (d, 2H), 7.24 (d, 2H), 7.39 (d, 1H), 7.47 (d, 2H), 7.62 (d, 1H), 7.65-7.69 (m, 4H), 7.81 (d, 2H), 7.94 (s, 1H), 8.17 (d, 1H) ppm.

### Example 298

### 4-{4-(2,4-Dichloro-phenyl)-2-[2-(4-nitro-phenyl)-(E)-vinyl]-imidazol-1-ylmethyl}-benzoic acid

4-(2,4-Dichloro-phenyl)-2-[2-(4-nitro-phenyl)-(E)-vinyl]-1H-imidazole (1.98 g, 5.5 mmol) was treated with methyl 4-bromomethyl benzoate using general procedure E to provide 4-{4-(2,4-dichloro-phenyl)-2-[2-(4-nitro-phenyl)-(E)-vinyl]-imidazol-1-ylmethyl}-benzoic acid methyl ester (753 mg, 27% yield). 30 mg (0.059 mmol) of the ester was hydrolyzed according to general procedure F to provide 4-{4-(2,4-dichloro-phenyl)-2-[2-(4-nitro-phenyl)-(E)-vinyl]-imidazol-1-ylmethyl}-benzoic acid (24 mg, 82% yield).

LCMS: m/z 494 (M+H)⁺; ¹H NMR (CD₃OD, 400 MHz): δ 5.53 (s, 2H), 7.18 (d, 1H), 7.31 (d, 2H), 7.38 (dd, 1H), 7.49 (d, 1H), 7.65-7.72 (m, 3H), 7.79 (s, 1H), 8.06 (m, 3H), 8.23 (d, 2H) ppm.

### Example 299

### 4-[2-[2-(4-Amino-phenyl)-(E)-vinyl]-4-(2,4-dichloro-phenyl)-imidazol-1-ylmethyl]-benzoic acid methyl ester

4-{4-(2,4-Dichloro-phenyl)-2-[2-(4-nitro-phenyl)-(E)-vinyl]-imidazol-1-ylmethyl}-benzoic acid methyl ester (453 mg, 0.89 mmol) was reduced according to general procedure K to provide 4-[2-[2-(4-amino-phenyl)-(E)-vinyl]-4-(2,4-dichloro-phenyl)-imidazol-1-ylmethyl]-benzoic acid methyl ester (350 mg, 82% yield).

LCMS: m/z 478 (M+H)⁺.

### Example 300

### 4-[2-[2-(4-Amino-phenyl)-(E)-vinyl]-4-(2,4-dichloro-phenyl)-imidazol-1-ylmethyl]-benzoic acid

4-[2-[2-(4-Amino-phenyl)-(E)-vinyl]-4-(2,4-dichloro-phenyl)-imidazol-1-ylmethyl]-benzoic acid methyl ester (17 mg, 0.036 mmol) was hydrolyzed according to general procedure F to provide 4-[2-[2-(4-amino-phenyl)-(E)-vinyl]-4-(2,4-dichloro-phenyl)-imidazol-1-ylmethyl]-benzoic acid (5.4 mg, 33% yield).

LCMS: m/z 464 (M+H)⁺; ¹H NMR (DMSO, 400 MHz): δ 5.52 (s, 2H), 6.54 (d, 2H), 6.90 (d, 1H), 7.25-7.34 (m, 4H), 7.38 (d, 1H), 7.49 (dd, 1H), 7.63 (d, 1H), 7.90 (d, 2H), 8.05 (s, 1H), 8.27 (d, 1H) ppm.

### Example 301

### 4-[2-{2-[4-(Butane-1-sulfonylamino)-phenyl]-(E)-vinyl}-4-(2,4-dichloro-phenyl)-imidazol-1-ylmethyl]-benzoic acid methyl ester

4-[2-[2-(4-Amino-phenyl)-(E)-vinyl]-4-(2,4-dichloro-phenyl)-imidazol-1-ylmethyl]-benzoic acid methyl ester (69 mg, 0.14 mmol) was treated with n-butanesulfonyl chloride according to general procedure L to provide 4-[2-{2-[4-(butane-1-sulfonylamino)-phenyl]-(E)-vinyl}-4-(2,4-dichloro-phenyl)-imidazol-1-ylmethyl]-benzoic acid methyl ester (48 mg, 57% yield).

LCMS: m/z 598 (M+H)⁺; ¹H NMR (CDCl₃, 400 MHz): δ 0.90 (t, 3H), 1.42 (m, 2H), 1.80 (m, 2H), 3.10 (m, 2H), 3.93 (s, 3H), 5.34 (s, 2H), 6.66 (s, 1H), 6.73 (d, 1H), 7.17 (d, 2H), 7.23 (d, 2H), 7.34 (dd, 1H), 7.41 (d, 2H), 7.43 (d, 1H), 7.64 (d, 1H), 7.71 (s, 1H), 8.05 (d, 2H), 8.26 (d, 1H) ppm.

### Example 302

### 4-[2-{2-[4-(Butane-1-sulfonylamino)-phenyl]-(E)-vinyl}-4-(2,4-dichloro-phenyl)-imidazol-1-ylmethyl]-benzoic acid

4-[2-{2-[4-(Butane-1-sulfonylamino)-phenyl]-(E)-vinyl}-4-(2,4-dichloro-phenyl)-imidazol-1-ylmethyl]-benzoic acid methyl ester (45 mg, 0.075 mmol) was hydrolyzed according to general procedure F to provide 4-[2-{2-[4-(butane-1-sulfonylamino)-phenyl]-(E)-vinyl}-4-(2,4-dichloro-phenyl)-imidazol-1-ylmethyl]-benzoic acid (30 mg, 68% yield).

LCMS: m/z 584 (M+H)⁺; ¹H NMR (DMSO, 400 MHz): δ 0.83 (t, 3H), 1.35 (m, 2H), 1.64 (m, 2H), 3.12 (m, 2H), 5.60 (s, 2H), 6.66 (s, 1H), 7.17-7.23 (m, 3H), 7.34 (d, 2H), 7.46-7.53 (m, 2H), 7.62 (d, 2H), 7.65 (d, 1H), 7.93 (d, 2H), 8.09 (s, 1H), 8.28 (d, 1H), 9.93 (br s, 1H), 12.82 (br s, 1H) ppm.

### Example 303

### 4-[2-{2-[4-(4-Butyl-benzenesulfonylamino)-phenyl]-(E)-vinyl}-4-(2,4-dichloro-phenyl)-imidazol-1-ylmethyl]-benzoic acid methyl ester

4-[2-[2-(4-Amino-phenyl)-(E)-vinyl]-4-(2,4-dichloro-phenyl)-imidazol-1-ylmethyl]-benzoic acid methyl ester (71 mg, 0.15 mmol) was treated with 4-n-butylbenzenesulfonyl chloride according to general procedure L to provide 4-[2-{2-[4-(4-butyl-benzenesulfonylamino)-phenyl]-(E)-vinyl}-4-(2,4-dichloro-phenyl)-imidazol-1-ylmethyl]-benzoic acid methyl ester (95 mg, 93% yield).

LCMS: m/z 674 (M+H)⁺; ¹H NMR (CDCl₃, 400 MHz): δ 0.90 (t, 3H), 1.30 (m, 2H), 1.57 (m, 2H), 2.62 (t, 2H), 3.92 (s, 3H), 5.31 (s, 2H), 6.69 (d, 1H), 6.98-7.05 (m, 3H), 7.21 (m, 4H), 7.28-7.33 (m, 3H), 7.42 (d, 1H), 7.58 (d, 1H), 7.68 (m, 3H), 8.03 (d, 2H), 8.24 (d, 1H) ppm.

### Example 304

### 4-[2-{2-[4-(4-Butyl-benzenesulfonylamino)-phenyl]-(E)-vinyl}-4-(2,4-dichloro-phenyl)-imidazol-1-ylmethyl]-benzoic acid

4-[2-{2-[4-(4-Butyl-benzenesulfonylamino)-phenyl]-(E)-vinyl}-4-(2,4-dichloro-phenyl)-imidazol-1-ylmethyl]-benzoic acid methyl ester (92 mg, 0.14 mmol) was hydrolyzed according to general procedure F to provide 4-[2-{2-[4-(4-butyl-benzenesulfonylamino)-phenyl]-(E)-vinyl}-4-(2,4-dichloro-phenyl)-imidazol-1-ylmethyl]-benzoic acid (82 mg, 91% yield).

LCMS: m/z 660 (M+H)⁺; ¹H NMR (DMSO, 400 MHz): δ 0.85 (t, 3H), 1.26 (m, 2H), 1.51 (m, 2H), 2.60 (t, 2H), 5.57 (s, 2H), 7.09 (d, 2H), 7.15 (d, 1H), 7.33 (d, 2H), 7.37 (d, 2H), 7.42 (d, 1H), 7.48-7.54 (m, 3H), 7.64 (d, 1H), 7.69 (d, 2H) 7.92 (d, 2H), 8.07 (s, 1H), 8.25 (d, 1H), 10.40 (S, 1H), 12.94 (br s, 1H) ppm.

### Example 305

### 4-[2-{2-[4-(4-Butyl-benzylamino)-phenyl]-(E)-vinyl}-4-(2,4-dichloro-phenyl)-imidazol-1-ylmethyl]-benzoic acid methyl ester

4-[2-[2-(4-Amino-phenyl)-(E)-vinyl]-4-(2,4-dichloro-phenyl)-imidazol-1-ylmethyl]-benzoic acid methyl ester (70 mg, 0.15 mmol) was treated with 4-n-butylbenzaldehyde according to general procedure U to provide 4-[2-{2-[4-(4-butyl-benzylamino)-phenyl]-(E)-vinyl}-4-(2,4-dichloro-phenyl)-imidazol-1-ylmethyl]-benzoic acid methyl ester (59 mg, 63% yield).

LCMS: m/z 624 (M+H)⁺; ¹H NMR (CDCl₃, 400 MHz): δ 0.92 (t, 3H), 1.35 (m, 2H), 1.58 (m, 2H), 2.60 (t, 2H), 3.90 (s, 3H), 4.29 (s, 2H), 5.28 (s, 2H), 6.54-6.60 (m, 3H), 7.15 (d, 2H), 7.20-7.30 (m, 6H), 7.32 (dd, 1H), 7.41 (d, 1H), 7.59 (d, 1H), 7.65 (s, 1H), 8.03 (d, 2H), 8.29 (d, 1H) ppm.

### Example 306

### 4-[2-{2-[4-(4-Butyl-benzylamino)-phenyl]-(E)-vinyl}-4-(2,4-dichloro-phenyl)-imidazol-1-ylmethyl]-benzoic acid

4-[2-{2-[4-(4-Butyl-benzylamino)-phenyl]-(E)-vinyl}-4-(2,4-dichloro-phenyl)-imidazol-1-ylmethyl]-benzoic acid methyl ester (55 mg, 0.09 mmol) was hydrolyzed according to general procedure F to provide 4-[2-{2-[4-(4-butyl-benzylamino)-phenyl]-(E)-vinyl}-4-(2,4-dichloro-phenyl)-imidazol-1-ylmethyl]-benzoic acid (39 mg, 72% yield).

LCMS: m/z 610 (M+H)⁺; ¹H NMR (DMSO, 400 MHz): δ 0.90 (t, 3H), 1.29 (m, 2H), 1.53 (m, 2H), 2.55 (t, 2H), 4.24 (d, 2H), 5.55 (s, 2H), 6.56 (d, 2H), 6.89 (d, 1H), 7.13 (d, 2H), 7.25 (d, 2H), 7.31-7.40 (m, 5H), 7.49 (dd, 1H), 7.63 (d, 1H), 7.92 (d, 2H), 8.02 (s, 1H), 8.27 (d, 1H), 12.95 (br s, 1H) ppm.

### Example 307

### 4-[2-{2-[4-(4-Butyl-benzenesulfonylamino)-phenyl]-ethyl}-4-(2,4-dichloro-phenyl)-imidazol-1-ylmethyl]-benzoic acid

4-[2-{2-[4-(4-Butyl-benzenesulfonylamino)-phenyl]-(E)-vinyl}-4-(2,4-dichloro-phenyl)-imidazol-1-ylmethyl]-benzoic acid (16 mg, 0.024 mmol) was reduced according to general procedure V to provide 4-[2-{2-[4-(4-butyl-benzenesulfonylamino)-phenyl]-ethyl}-4-(2,4-dichloro-phenyl)-imidazol-1-ylmethyl]-benzoic acid (8 mg, 50% yield).

LCMS: m/z 662 (M+H)⁺; ¹H NMR (CD₃OD, 400 MHz): δ 0.89 (t, 3H), 1.28 (m, 2H), 1.50 (m, 2H), 2.55 (t, 2H), 2.86 (m, 4H), 4.96 (s, 2H), 6.92 (d, 2H), 6.97 (d, 2H), 7.09 (d, 2H), 7.22 (d, 2H), 7.38 (dd, 1H), 7.51 (d, 1H), 7.58 (s, 1H), 7.63 (d, 2H) 7.88 (d, 1H), 7.97 (d, 2H) ppm.

### Example 308

### 4-(4-(2,4-Dichloro-phenyl)-2-{2-[4-(3-trifluoromethyl-benzenesulfonylamino)-phenyl]-(E)-vinyl}-imidazol-1-ylmethyl)-benzoic acid methyl ester

4-[2-[2-(4-Amino-phenyl)-(E)-vinyl]-4-(2,4-dichloro-phenyl)-imidazol-1-ylmethyl]-benzoic acid methyl ester (66 mg, 0.14 mmol) was treated with 3-trifluoromethylbenzenesulfonyl chloride according to general procedure L to provide 4-(4-(2,4-dichloro-phenyl)-2-{2-[4-(3-trifluoromethyl-benzenesulfonylamino)-phenyl]-(E)-vinyl}-imidazol-1-ylmethyl)-benzoic acid methyl ester (87 mg, 92% yield).

LCMS: m/z 686 (M+H)⁺; ¹H NMR (CDCl₃, 400 MHz): δ 3.92 (s, 3H), 5.34 (s, 2H), 6.67 (br s, 1H), 6.71 (d, 1H), 7.03 (d, 2H), 7.22 (d, 2H), 7.31-7.36 (m, 3H), 7.43 (d, 1H), 7.56-7.62 (m, 2H), 7.70 (s, 1H), 7.80 (d, 1H), 7.91 (d, 1H), 8.01-8.06 (m, 3H), 8.24 (d, 1H) ppm.

### Example 309

### 4-(4-(2,4-Dichloro-phenyl)-2-{2-[4-(4-trifluoromethyl-benzenesulfonylamino)-phenyl]-(E)-vinyl}-imidazol-1-ylmethyl)-benzoic acid methyl ester

4-[2-[2-(4-Amino-phenyl)-(E)-vinyl]-4-(2,4-dichloro-phenyl)-imidazol-1-ylmethyl]-benzoic acid methyl ester (66 mg, 0.14 mmol) was treated with 4-trifluoromethylbenzenesulfonyl chloride according to general procedure L to provide 4-(4-(2,4-dichloro-phenyl)-2-{2-[4-(4-trifluoromethyl-benzenesulfonylamino)-phenyl]-(E)-vinyl}-imidazol-1-ylmethyl)-benzoic acid methyl ester (87 mg, 92% yield).

LCMS: m/z 686 (M+H)⁺; ¹H NMR (CDCl₃, 400 MHz): δ 3.92 (s, 3H), 5.33 (s, 2H), 6.69-6.73 (m, 2H), 7.04 (d, 2H), 7.22 (d, 2H), 7.31-7.36 (m, 3H), 7.43 (d, 1H), 7.60 (d, 1H), 7.71 (m, 3H), 7.88 (d, 2H), 8.04 (d, 2H), 8.24 (d, 1H) ppm.

### Example 310

### 4-(4-(2,4-Dichloro-phenyl)-2-{2-[4-(3-trifluoromethyl-benzenesulfonylamino)-phenyl]-(E)-vinyl}-imidazol-1-ylmethyl)-benzoic acid

4-(4-(2,4-Dichloro-phenyl)-2-{2-[4-(3-trifluoromethyl-benzenesulfonylamino)-phenyl]-(E)-vinyl}-imidazol-1-ylmethyl)-benzoic acid methyl ester (79 mg, 0.12 mmol) was hydrolyzed according to general procedure F to provide 4-(4-(2,4-dichloro-phenyl)-2-{2-[4-(3-trifluoromethyl-benzenesulfonylamino)-phenyl]-(E)-vinyl}-imidazol-1-ylmethyl)-benzoic acid (46 mg, 59% yield).

LCMS: m/z 672 (M+H)⁺; ¹H NMR (DMSO, 400 MHz): δ 5.58 (s, 2H), 7.09 (d, 2H), 7.18 (d, 1H), 7.33 (d, 2H), 7.43 (d, 1H), 7.50 (dd, 1H), 7.56 (d, 2H), 7.64 (d, 1H), 7.82 (t, 1H) 7.93 (d, 2H), 8.01-8.06 (m, 3H), 8.08 (s, 1H), 8.25 (d, 1H), 10.59 (s, 1H), 12.96 (br s, 1H) ppm.

### Example 311

### 4-(4-(2,4-Dichloro-phenyl)-2-{2-[4-(4-trifluoromethyl-benzenesulfonylamino)-phenyl]-(E)-vinyl}-imidazol-1-ylmethyl)-benzoic acid

4-(4-(2,4-Dichloro-phenyl)-2-{2-[4-(4-trifluoromethyl-benzenesulfonylamino)-phenyl]-(E)-vinyl}-imidazol-1-ylmethyl)-benzoic acid methyl ester (79 mg, 0.12 mmol) was hydrolyzed according to general procedure F to provide 4-(4-(2,4-dichloro-phenyl)-2-{2-[4-(4-trifluoromethyl-benzenesulfonylamino)-phenyl]-(E)-vinyl}-imidazol-1-ylmethyl)-benzoic acid (54 mg, 70% yield).

LCMS: m/z 672 (M+H)⁺; ¹H NMR (DMSO, 400 MHz): δ 5.59 (s, 2H), 7.10 (d, 2H), 7.17 (d, 1H), 7.33 (d, 2H), 7.43 (d, 1H), 7.49 (dd, 1H), 7.55 (d, 2H), 7.64 (d, 1H), 7.92 (d, 2H) 7.97 (s, 4H), 8.08 (s, 1H), 8.25 (d, 1H), 10.68 (br s, 1H), 12.96 (br s, 1H) ppm.

### Example 312

### 4-(4-(2,4-Dichloro-phenyl)-2-{2-[4-(toluene-4-sulfonylamino)-phenyl]-(E)-vinyl}-imidazol-1-ylmethyl)-benzoic acid methyl ester

4-[2-[2-(4-Amino-phenyl)-(E)-vinyl]-4-(2,4-dichloro-phenyl)-imidazol-1-ylmethyl]-benzoic acid methyl ester (35 mg, 0.073 mmol) was treated with p-toluenesulfonyl chloride according to general procedure L to provide 4-(4-(2,4-dichloro-phenyl)-2-{2-[4-(toluene-4-sulfonylamino)-phenyl]-(E)-vinyl}-imidazol-1-ylmethyl)-benzoic acid methyl ester (39 mg, 84% yield).

LCMS: m/z 632 (M+H)⁺; ¹H NMR (CDCl₃, 400 MHz): δ 2.36 (s, 3H), 3.90 (s, 3H), 5.30 (s, 2H), 6.68 (d, 1H), 7.03 (d, 2H), 7.20 (d, 4H), 7.26-7.32 (m, 3H), 7.41 (d, 1H), 7.57 (d, 1H), 7.65 (d, 2H), 7.68 (s, 1H), 8.03 (d, 2H), 8.23 (d, 1H) ppm.

### Example 313

### 4-(4-(2,4-Dichloro-phenyl)-2-{2-[4-(toluene-4-sulfonylamino)-phenyl]-(E)-vinyl}-imidazol-1-ylmethyl)-benzoic acid

4-(4-(2,4-Dichloro-phenyl)-2-{2-[4-(toluene-4-sulfonylamino)-phenyl]-(E)-vinyl}-imidazol-1-ylmethyl)-benzoic acid methyl ester (36 mg, 0.057 mmol) was hydrolyzed according to general procedure F to provide 4-(4-(2,4-dichloro-phenyl)-2-{2-[4-(toluene-4-sulfonylamino)-phenyl]-(E)-vinyl}-imidazol-1-ylmethyl)-benzoic acid (26 mg, 74% yield).

LCMS: m/z 618 (M+H)⁺; ¹H NMR (CD₃OD, 400 MHz): δ 2.33 (s, 3H), 5.45 (s, 2H), 6.95 (d, 1H), 7.07 (d, 2H), 7.23 (d, 2H), 7.28 (d, 2H), 7.36 (m, 3H), 7.43 (d, 1H), 7.48 (d, 1H), 7.63 (d, 2H) 7.77 (s, 1H), 7.95-8.00 (m, 3H) ppm.

### Example 314

### 4-[2-(2-{4-[(4-Butyl-benzenesulfonyl)-methyl-amino]-phenyl}-(E)-vinyl)-4-(2,4-dichloro-phenyl)-imidazol-1-ylmethyl]-benzoic acid

4-[2-{2-[4-(4-Butyl-benzenesulfonylamino)-phenyl]-(E)-vinyl}-4-(2,4-dichloro-phenyl)-imidazol-1-ylmethyl]-benzoic acid (24 mg, 0.036 mmol) was treated with sodium hydride and methyl iodide according to general procedure P, then the methyl ester which formed was hydrolyzed according to general procedure F to provide 4-[2-(2-{4-[(4-butyl-benzenesulfonyl)-methyl-amino]-phenyl}-(E)-vinyl)-4-(2,4-dichloro-phenyl)-imidazol-1-ylmethyl]-benzoic acid (11 mg, 45% yield).

LCMS: m/z 674 (M+H)⁺; ¹H NMR (CD₃OD, 400 MHz): δ 0.95 (t, 3H), 1.38 (m, 2H), 1.64 (M, 2H), 2.70 (t, 2H), 3.18 (s, 3H), 5.48 (s, 2H), 6.95 (d, 1H), 7.09 (d, 2H), 7.28-7.33 (m, 4H), 7.37 (dd, 1H), 7.43-7.49 (m, 5H), 7.58 (d, 1H) 7.74 (s, 1H), 8.03-8.09 (m, 3H) ppm.

### Example 315

### 4-{4-(2,4-Dichloro-phenyl)-2[2-(4′-trifluoromethyl-biphenyl-4-yl)-(E)-vinyl]-imidazol-1-ylmethyl}-benzoic acid methyl ester

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(trifluoromethyl)-phenyl boronic acid (189 mg, 1 mmol) following General Procedure B to give 4-{4-(2,4-dichloro-phenyl)-2-[2-(4′-trifluoromethyl-biphenyl-4-yl)-(E)-vinyl]-imidazol-1yl-methyl}benzoic acid methyl ester (313 mg, 51%).

LCMS: 607 (M+H)⁺.

### Example 316

### 4-{4-(2,4-Dichloro-phenyl)-2[2-(4′-trifluoromethyl-biphenyl-4-yl)-(E)-vinyl]-imidazol-1-ylmethyl}-benzoic acid

4-{4-(2,4-dichloro-phenyl)-2-[2-(4′-trifluoromethyl-biphenyl-4-yl)-(E)-vinyl]-imidazol-1yl-methyl}benzoic acid methyl ester (303 mg, 0.5 mmol) was hydrolyzed according to General Procedure F to give 4-{4-(2,4-Dichloro-phenyl)-2[2-(4′-trifluoromethyl-biphenyl-4-yl)-(E)-vinyl]-imidazol-1-ylmethyl}-benzoic acid (197 mg, 67%).

LCMS: 593 (M+H)^{+ 1}H NMR (DMSO, 400 MHz): δ 5.82 (s, 2H), 7.48-7.50 (m, 2H), 7.56 (s, 1H), 7.60-7.64 (m, 3H), 7.81-7.88 (m, 4H), 7.91-7.99 (m, 4H), 8.14-8.19 (m, 3H), 8.32 (s, 1H) ppm.

### Example 317

### 4-{4-(2,4-Dichloro-phenyl)-2[2-(4′-trifluoromethoxy-biphenyl-4-yl)-(E)-vinyl]-imidazol-1-ylmethyl}-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(trifluoromethoxy)-phenyl boronic acid (205 mg, 1 mmol) following General Procedure B to give 4-{4-(2,4-dichloro-phenyl)-2-[2-(4′-trifluoromethoxy-biphenyl-4-yl)-(E)-vinyl]-imidazol-1-yl-methyl}benzoic acid methyl ester (324 mg, 52%).

LCMS: 623 (M+H)⁺

### Example 318

### 4-{4-(2,4-Dichloro-phenyl)-2[2-(4′-trifluoromethoxy-biphenyl-4-yl)-(E)-vinyl]-imidazol-1-ylmethyl}-benzoic acid

4-{4-(2,4-dichloro-phenyl)-2-[2-(4′-trifluoromethoxy-biphenyl-4-yl)-(E)-vinyl]-imidazol-1yl-methyl}benzoic acid methyl ester (311 mg, 0.5 mmol) was hydrolyzed according to General Procedure F to give 4-{4-(2,4-Dichloro-phenyl)-2[2-(4′-trifluoromethoxy-biphenyl-4-yl)-(E)-vinyl]-imidazol-1-ylmethyl}-benzoic acid (198 mg, 65%).

LCMS: 609 (M+H)^{+ 1}H NMR (DMSO, 400 MHz): δ 5.66 (s, 2H), 7.36-7.40 (m, 2H), 7.44-7.46 (m, 2H), 7.51 (d, 1H), 7.52 (d, 1H), 7.53 (d, 1H), 7.59 (s, 1H), 7.63-7.66 (m, 2H), 7.70-7.72 (m, 2H), 7.76-7.84 (m, 2H), 7.93-7.95 (m. 2H), 8.13 (s, 1H), 8.27 (d, 1H) ppm.

### Example 319

### 4-[2-[2-(4′-Butoxy-biphenyl-4-yl)-(E)-vinyl]-4-(2,4-dichloro-phenyl)-imidazol-1-ylmethyl]-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-butoxy-phenyl boronic acid (195 mg, 1 mmol) following General Procedure B to give 4-2-[2-(4″-butoxy-biphenyl-4-yl)-(E)-vinyl]-4-{4-(2,4-dichloro-phenyl)-imidazol-1yl-methyl}benzoic acid methyl ester (315 mg, 51%).

LCMS: 611 (M+H)⁺.

### Example 320

### 4-[2-[2-(4′-Butoxy-biphenyl-4-yl)-(E)-vinyl]-4-(2,4-dichloro-phenyl)-imidazol-1-ylmethyl]-benzoic acid

4-2-[2-(4′-butoxy-biphenyl-4-yl)-(E)-vinyl]-4-{4-(2,4-dichloro-phenyl)-imidazol-1yl-methyl}benzoic acid methyl ester (305 mg, 0.5 mmol) was hydrolyzed according to General Procedure F to give 4-[2-[2-(4′-Butoxy-biphenyl-4-yl)-(E)-vinyl]-4-(2,4-dichloro-phenyl)-imidazol-1-ylmethyl]-benzoic acid (198 mg, 66%)

LCMS: 597 (M+H)^{+ 1}H NMR (DMSO, 400 MHz): δ 0.96 (t, 3H), 1.43-1.45 (m, 2H), 1.69-1.73 (m, 2H), 4.02 (q, 2H), 5.64 (s, 2H), 7.02 (d, 1H), 7.29 (s, 1H), 7.33-7.37 (m, 4H), 7.52-7.54 (m, 4H), 7.58-7.64 (m, 4H), 7.65 (d, 1H), 7.92 (d, 1H), 8.10 (s, 1H), 8.27 (d, 1H) ppm.

### Example 321

### 4-{4-(2,4-Dichloro-phenyl)-2[2-(3′-trifluoromethyl-biphenyl-4-yl)-(E)-vinyl]-imidazol-1-ylmethyl}-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(trifluoromethyl)-phenyl boronic acid (189 mg, 1 mmol) following General Procedure B to give 4-{4-(2,4-dichloro-phenyl)-2-[2-(3′-trifluoromethyl-biphenyl-4-yl)-(E)-vinyl]-imidazol-1yl-methyl}benzoic acid methyl ester (312 mg, 52%).

LCMS: 607 (M+H)^{+ 1}H NMR (CDCl₃, 400 MHz): δ 3.91 (s, 3H), 5.37 (s, 2H) 6.87 (d, 1H), 7.33-7.7.36 (m, 4H), 7.43 (d, 1H), 7.53 (s, 1H), 7.55-7.61 (m, 4H), 7.72-7.75 (m, 4H), 7.83 (s, 1H), 8.05 (s, 1H), 8.30 (d, 1H) ppm.

### Example 322

### 4-{4-(2,4-Dichloro-phenyl)-2[2-(3′-trifluoromethyl-biphenyl-4-yl)-(E)-vinyl]-imidazol-1-ylmethyl}-benzoic acid

4-{4-(2,4-Dichloro-phenyl)-2-[2-(3′-trifluoromethyl-biphenyl-4-yl)-(E)-vinyl]-imidazol-1yl-methyl}benzoic acid methyl ester (303 mg, 0.5 mmol) was hydrolyzed according to General Procedure F to give 4-{4-(2,4-Dichloro-phenyl)-2[2-(3′-trifluoromethyl-biphenyl-4-yl)-(E)-vinyl]-imidazol-1-ylmethyl}-benzoic acid (197 mg, 67%).

LCMS: 593 (M+H)^{+ 1}H NMR (DMSO, 400 MHz): δ 5.70 (s, 2H), 7.40-7.42 (m, 4H), 7.47 (s, 1H), 7.55 (d, 2H), 7.71 (d, 2H), 7.81 (s, 1H), 7.94 (d, 2H), 8.01-8.04 (m, 2H), 8.18-8.22 (m, 4H) ppm.

### Example 323

### 4-{4-(2,4-Dichloro-phenyl)-2[2-(3′-trifluoromethoxy-biphenyl-4-yl)-(E)-vinyl]-imidazol-1-ylmethyl}-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(trifluoromethoxy)-phenyl boronic acid (205 mg, 1 mmol) following General Procedure B to give 4-{4-(2,4-dichloro-phenyl)-2-[2-(3′-trifluoromethoxy-biphenyl-4-yl)-(E)-vinyl]-imidazol-1yl-methyl}benzoic acid methyl ester (321 mg, 51%).

LCMS: 623 (M+H)⁺.

### Example 324

### 4-{4-(2,4-Dichloro-phenyl)-2[2-(3′-trifluoromethoxy-biphenyl-4-yl)-(E)-vinyl]-imidazol-1-ylmethyl}-benzoic acid

4-{4-(2,4-dichloro-phenyl)-2-[2-(3′-trifluoromethoxy-biphenyl-4-yl)-(E)-vinyl]-imidazol-1yl-methyl}benzoic acid methyl ester (311 mg, 0.5 mmol) was hydrolyzed according to General Procedure F to give 4-{4-(2,4-Dichloro-phenyl)-2[2-(4′-trifluoromethoxy-biphenyl-4-yl)-(E)-vinyl]-imidazol-1-ylmethyl}-benzoic acid (198 mg, 65%).

LCMS: 609 (M+H)^{+ 1}H NMR (DMSO, 400 MHz): δ 4.81 (s, 2H), 6.51-6.55 (m, 2H), 6.66 (d, 2H), 6.72-6.75 (m, 4H), 6.76 (s, 1H), 6.77 (s, 1H), 6.81-6.93 (m, 4H), 7.10 (d, 2H), 7.27 (s, 1H), 7.45 (d, 1H) ppm.

### Example 325

### 4-{4-(2,4-Dichlorophenyl)-2-[2-(3′-trifluoromethanesulfonylamino-biphenyl-4-yl)-(E)-vinyl]-imidazol-1-ylmethyl}-benzoic acid methyl ester

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-amino-phenyl boronic acid (137 mg, 1 mmol) following General Procedure B and obtained 4-{4-(2,4-dichloro-phenyl)-2-[2-(3′-amino-biphenyl-4-yl)-(E)-vinyl]-imidazol-1yl-methyl}benzoic acid methyl ester (277 mg, 0.5 mmol) was alkylated according to General Procedure P to give 4-{4-(2,4-Dichloro-phenyl)-2[2-(3-trifluoromethanesulfonylamino-biphenyl-4-yl)-(E)-vinyl]-imidazol-1-ylmethyl}-benzoic acid (228 mg, 66%).

LCMS: 686 (M+H)⁺.

### Example 326

### 4-{4-(2,4-Dichloro-phenyl)-2-[2-(3′-trifluoromethanesulfonylamino-biphenyl-4-yl)-(E)-vinyl]-imidazol-1-ylmethyl}-benzoic acid

4-{4-(2,4-Dichloro-phenyl)-2-[2-(3′-trifluoromethanesulfonylamino-biphenyl-4-yl)-(E)-vinyl]-imidazol-1-ylmethyl}-benzoic acid methyl ester (343 mg, 0.5 mmol) was hydrolyzed according to General Procedure F to give 4-{4-(2,4-Dichloro-phenyl)-2-[2-(3′-trifluoromethanesulfonylamino-biphenyl-4-yl)-(E)-vinyl]-imidazol-1-ylmethyl}-benzoic acid (238 mg, 70%).

LCMS: 672 (M+H)^{+ 1}H NMR (DMSO, 400 MHz): δ 5.61 (s, 2H), 6.93 (d, 1H), 7.05 (d, 1H), 7.12-7.14 (m, 2H), 7.24 (s, 1H), 7.30-7.34 (m, 4H), 7.50-7.57 (m, 4H), 7.64 (s, 1H), 7.70 (d, 1H), 7.92 (d, 2H), 8.10 (s, 1H), 8.30 (d, 1H) ppm.

### Example 327

### (4-{4-(2,4-Dichloro-phenyl)-2-[2-(3′-methanesulfonyl-biphenyl-4-yl)-(E)-vinyl]-imidazol-1-ylmethyl}-phenyl)-acetic acid methyl ester

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with (4-Bromomethyl-phenyl)-acetic acid methyl ester (243 mg, 1 mmol) following general procedure E. The resulted {4-[2-[2-(4-Bromo-phenyl)-(E)-vinyl]-4-(2,4-dichloro-phenyl)-imidazol-1-ylmethyl]-phenyl}-acetic acid methyl ester (556 mg, 1 mmol) was coupled with 3-methanesulfonyl-phenyl boronic acid (200 mg, 1 mmol) following General Procedure B to give (4-{4-(2,4-Dichloro-phenyl)-2-[2-(3′-methanesulfonyl-biphenyl-4-yl)-(E)-vinyl]-imidazol-1-ylmethyl}-phenyl)-acetic acid methyl ester (321 mg, 50%).

LCMS: 631 (M+H)⁺

### Example 328

### (4-{4-(2,4-Dichloro-phenyl)-2-[2-(3′-methanesulfonyl-biphenyl-4-yl)-(E)-vinyl]-imidazol-1-ylmethyl}-phenyl)-acetic acid

(4-{4-(2,4-Dichloro-phenyl)-2-[2-(3′-methanesulfonyl-biphenyl-4-yl)-(E)-vinyl]-imidazol-1-ylmethyl}-phenyl)-acetic acid methyl ester (315 mg, 0.5 mmol) was hydrolyzed according to General Procedure F to give (4-{4-(2,4-Dichloro-phenyl)-2-[2-(3′-methanesulfonyl-biphenyl-4-yl)-(E)-vinyl]-imidazol-1-ylmethyl}-phenyl)-acetic acid (198 mg, 64%).

LCMS: 617 (M+H)^{+ 1}H NMR (DMSO, 400 MHz): δ 3.31 (s, 3H), 3.46 (s, 2H), 5.51 (s, 2H), 7.23 (s, 1H), 7.45-7.49 (m, 2H), 7.51-7.57 (m, 2H), 7.61-7.64 (m, 2H), 7.75-7.76 (m, 2H), 7.79-7.82 (m, 2H), 7.84-8.07 (m, 4H), 8.10 (d, 1H), 8.19 (s, 1H), 8.25 (d, 1H) ppm.

### Example 329

### 4-[2-[2-(4′-Ethoxy-biphenyl-4-yl)-(E)-vinyl]-4-(2,4-dichloro-phenyl)-imidazol-1-ylmethyl]-benzoic acid methyl ester

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-ethoxy-phenyl boronic acid (165 mg, 1 mmol) following General Procedure B to give 4-2-[2-(4′-ethoxy-biphenyl-4-yl)-(E)-vinyl]-4-{4-(2,4-dichloro-phenyl)-imidazol-1yl-methyl}benzoic acid methyl ester (305 mg, 52%).

LCMS: 583 (M+H)⁺.

### Example 330

### 4-[2-[2-(4′-Ethoxy-biphenyl-4-yl)-(E)-vinyl]-4-(2,4-dichloro-phenyl)-imidazol-1-ylmethyl]-benzoic acid

4-2-[2-(4′-ethoxy-biphenyl-4-yl)-(E)-vinyl]-4-{4-(2,4-dichloro-phenyl)-imidazo-1-yl-methyl}benzoic acid methyl ester (292 mg, 0.5 mmol) was hydrolyzed according to General Procedure F to give 4-[2-[2-(4′-ethoxy-biphenyl-4-yl)-(E)-vinyl]-4-(2,4-dichloro-phenyl)-imidazol-1-ylmethyl]-benzoic acid (198 mg, 69%)

LCMS: 569 (M+H)^{+ 1}H NMR (DMSO, 400 MHz): δ 0.96 (t, 3H), 4.02 (q, 2H), 5.64 (s, 2H), 7.02 (d, 1H), 7.29 (s, 1H), 7.33-7.37 (m, 4H), 7.52-7.54 (m, 4H), 7.58-7.64 (m, 4H), 7.65 (d, 1H), 7.92 (d, 1H), 8.10 (s, 1H), 8.27 (d, 1H) ppm.

### Example 331

### 4-[2-[2-(4′-hydroxy-biphenyl-4-yl)-(E)-vinyl]-4-(2,4-dichloro-phenyl)-imidazol-1-ylmethyl]-benzoic acid

Step 1: Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-hydroxy-phenyl boronic acid (137 mg, 1 mmol) following General Procedure B to give 4-2-[2-(4′-hydroxy-biphenyl-4-yl)-(E)-vinyl]-4-{4-(2,4-dichloro-phenyl)-imidazol-1yl-methyl}benzoic acid methyl ester (288 mg, 54%)

LCMS: 556 (M+H)⁺

Step 2: 4-2-[2-(4′-hydroxy-biphenyl-4-yl)-(E)-vinyl]-4-{4-(2,4-dichloro-phenyl)-imidazol-1yl-methyl}benzoic acid methyl ester (278 mg, 0.5 mmol) was hydrolyzed according to General Procedure F to give 4-[2-[2-(4′-hydroxy-biphenyl-4-yl)-(E)-vinyl]-4-(2,4-dichloro-phenyl)-imidazol-1-ylmethyl]-benzoic acid (168 mg, 62%)

LCMS: 541 (M+H)^{+ 1}H NMR (DMSO, 400 MHz): δ 5.68 (s, 2H), 7.12 (d, 1H), 7.36 (s, 1H), 7.37-7.40 (m, 4H), 7.52-7.54 (m, 4H), 7.58-7.64 (m, 4H), 7.66 (d, 1H), 7.91 (d, 1H), 8.09 (s, 1H), 8.21 (d, 1H) ppm.

### Example 332

### 4-{4-(2,4-Dichloro-phenyl)-2-[2-(4′-ethoxy-4-methoxy-biphenyl-3-yl)-(E)-vinyl]-imidazol-1-ylmethyl}-benzoic acid methyl ester

Trans 5-bromo 2-methoxy cinnamic acid (257 mg, 1 mmol) was reacted with 2-bromo-2,4-dichloro acetophenone (267 mg, 1 mmol) according to general procedure A and obtained 2-[2-(5-Bromo-2-methoxy-phenyl)-(E)-vinyl]-4-(2,4-dichloro-phenyl)-1H-imidazole (424 mg, 1 mmol) was N-alkylated with methyl-4-(bromomethyl)benzoate (229 mg, 1 mmol) following general procedure E. The resulted 4-[2-[2-(5-Bromo-2-methoxy-phenyl)-(E)-vinyl]-4-(2,4-dichloro-phenyl)-imidazol-1-ylmethyl]-benzoic acid methyl ester (572 mg, 1 mmol) was coupled with 4-ethoxy-phenyl boronic acid (165 mg, 1 mmol) following General Procedure B to give 4-{4-(2,4-Dichloro-phenyl)-2-[2-(4′-ethoxy-4-methoxy-biphenyl-3-yl)-(E)-vinyl]-imidazol-1-ylmethyl}-benzoic acid methyl ester (298 mg, 49%).

LCMS: 613 (M+H)⁺.

### Example 333

### 4-{4-(2,4-Dichloro-phenyl)-2-[2-(4′-ethoxy-4-methoxy-biphenyl-3-yl)-(E)-vinyl]-imidazol-1-ylmethyl}-benzoic acid

4-{4-(2,4-Dichloro-phenyl)-2-[2-(4′-ethoxy-4-methoxy-biphenyl-3-yl)-(E)-vinyl]-imidazol-1-ylmethyl}-benzoic acid methyl ester (154 mg, 0.25 mmol) was hydrolyzed according to General Procedure F to give 4-{4-(2,4-Dichloro-phenyl)-2-[2-(4′-ethoxy-4-methoxy-biphenyl-3-yl)-(E)-vinyl]-imidazol-1-ylmethyl}-benzoic acid (117 mg, 78%).

LCMS: 599 (M+H)⁺. ¹H NMR (DMSO, 400 MHz): δ 1.39 (t, 3H), 3.90 (s, 3H), 4.24 (q, 2H), 5.28 (d, 2H), 7.09 (d, 2H), 7.11-7.21 (m, 2H), 7.28-7.36 (m, 2H), 7.38 (d, 1H), 7.41-7.56 (m, 4H), 7.71 (d, 1H), 7.76-8.02 (m. 4H), 8.16 (d, 1H) ppm

### Example 334

### (4-{4-(2,4-Dichloro-phenyl)-2-[2-(3′-trifluoromethyl-biphenyl-4-yl)-(E)-vinyl]-imidazol-1-ylmethyl}-phenyl)-acetic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with (4-Bromomethyl-phenyl)-acetic acid methyl ester (243 mg, 1 mmol) following general procedure E. The resulted {4-[2-[2-(4-Bromo-phenyl)-(E)-vinyl]-4-(2,4-dichloro-phenyl)-imidazol-1-ylmethyl]-phenyl}-acetic acid methyl ester (556 mg, 1 mmol) was coupled with 3-trifluoromethyl-phenyl boronic acid (189 mg, 1 mmol) following General Procedure B to give (4-{4-(2,4-Dichloro-phenyl)-2-[2-(3′-trifluoromethyl-biphenyl-4-yl)-(E)-vinyl]-imidazol-1-ylmethyl}-phenyl)-acetic acid methyl ester (321 mg, 51%).

LCMS: 621 (M+H)⁺

### Example 335

### (4-{4-(2,4-Dichloro-phenyl)-2-[2-(3′-trifluoromethyl-biphenyl-4-yl)-(E)-vinyl]-imidazol-1-ylmethyl}-phenyl)-acetic acid

(4-{4-(2,4-Dichloro-phenyl)-2-[2-(3′-trifluoromethyl-biphenyl-4-yl)-(E)-vinyl]-imidazol-1-ylmethyl}-phenyl)-acetic acid methyl ester (310 mg, 0.5 mmol) was hydrolyzed according to General Procedure F to give (4-{4-(2,4-Dichloro-phenyl)-2-[2-(3′-trifluoromethyl-biphenyl-4-yl)-(E)-vinyl]-imidazol-1-ylmethyl}-phenyl)-acetic acid (198 mg, 65%).

LCMS: 607 (M+H)^{+ 1}H NMR (DMSO, 400 MHz): δ 3.81 (s, 2H), 5.56 (s, 2H), 7.44-7.48 (m, 2H), 7.50-7.53 (m, 2H), 7.58 (s, 1H), 7.61-7.64 (m, 2H), 7.75-7.76 (m, 2H), 7.79-7.82 (m, 2H), 7.83-8.07 (m, 4H), 8.09 (d, 1H), 8.19 (s, 1H), 8.27 (d, 1H) ppm.

### Example 336

### 4-{4-(2,4-Dichloro-phenyl)-2-[2-(4′-hydroxy-4-methoxy-biphenyl-3-yl)-(E)-vinyl]-imidazol-1-ylmethyl}-benzoic acid methyl ester

Trans 5-bromo 2-methoxy cinnamic acid (257 mg, 1 mmol) was reacted with 2-bromo-2,4-dichloro acetophenone (267 mg, 1 mmol) according to general procedure A and obtained 2-[2-(5-Bromo-2-methoxy-phenyl)-(E)-vinyl]-4-(2,4-dichloro-phenyl)-1H-imidazole (424 mg, 1 mmol) was N-alkylated with methyl-4-(bromomethyl)benzoate (229 mg, 1 mmol) following general procedure E. The resulted 4-[2-[2-(5-Bromo-2-methoxy-phenyl)-(E)-vinyl]-4-(2,4-dichloro-phenyl)-imidazol-1-ylmethyl]-benzoic acid methyl ester (572 mg, 1 mmol) was coupled with 4-hydroxy-phenyl boronic acid (137 mg, 1 mmol) following General Procedure B to give 4-{4-(2,4-Dichloro-phenyl)-2-[2-(4′-hydroxy-4-methoxy-biphenyl-3-yl)-(E)-vinyl]-imidazol-1-ylmethyl}-benzoic acid methyl ester (291 mg, 50%).

LCMS: 585 (M+H)⁺.

### Example 337

### 4-{4-(2,4-Dichloro-phenyl)-2-[2-(4′-hydroxy-4-methoxy-biphenyl-3-yl)-(E)-vinyl]-imidazol-1-ylmethyl}-benzoic acid

4-{4-(2,4-Dichloro-phenyl)-2-[2-(4′-hydroxy-4-methoxy-biphenyl-3-yl)-(E)-vinyl]-imidazol-1-ylmethyl}-benzoic acid methyl ester (146 mg, 0.25 mmol) was hydrolyzed according to General Procedure F to give 4-{4-(2,4-Dichloro-phenyl)-2-[2-(4′-hydroxy-4-methoxy-biphenyl-3-yl)-(E)-vinyl]-imidazol-1-ylmethyl}-benzoic acid (107 mg, 75%).

LCMS: 571 (M+H)⁺. ¹H NMR (DMSO, 400 MHz): 6, 3.87 (s, 3H), 5.26 (d, 2H), 7.13 (d, 2H), 7.16-7.22 (m, 2H), 7.28-7.36 (m, 2H), 7.39 (d, 1H), 7.41-7.56 (m, 4H), 7.70 (d, 1H), 7.76-8.11 (m. 4H), 8.14 (d, 1H) ppm

### Example 338

### 4-[2-[2-(3′-Butoxy-biphenyl-4-yl)-(E)-vinyl]-4-(2,4-dichloro-phenyl)-imidazol-1-ylmethyl]-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-butoxy-phenyl boronic acid (195 mg, 1 mmol) following General Procedure B to give 4-2-[2-(3′-butoxy-biphenyl-4-yl)-(E)-vinyl]-4-{4-(2,4-dichloro-phenyl)-imidazol-1yl-methyl}benzoic acid methyl ester (325 mg, 53%).

LCMS: 611 (M+H)⁺

### Example 339

### 4-[2-[2-(3′-Butoxy-biphenyl-4-yl)-(E)-vinyl]-4-(2,4-dichloro-phenyl)-imidazol-1-ylmethyl]-benzoic acid

4-2-[2-(3′-butoxy-biphenyl-4-yl)-(E)-vinyl]-4-{4-(2,4-dichloro-phenyl)-imidazol-1yl-methyl}benzoic acid methyl ester (305 mg, 0.5 mmol) was hydrolyzed according to General Procedure F to give 4-[2-[2-(3′-Butoxy-biphenyl-4-yl)-(E)-vinyl]-4-(2,4-dichloro-phenyl)-imidazol-1-ylmethyl]-benzoic acid (192 mg, 64%)

LCMS: 597 (M+H)^{+ 1}H NMR (DMSO, 400 MHz): δ 0.94 (t, 3H), 1.41-1.44 (m, 2H), 1.68-1.72 (m, 2H), 4.01 (q, 2H), 5.66 (s, 2H), 7.10 (d, 1H), 7.29 (s, 1H), 7.31-7.36 (m, 4H), 7.51-7.56 (m, 4H), 7.59-7.66 (m, 4H), 7.67 (d, 1H), 7.91 (d, 1H), 8.11 (s, 1H), 8.29 (d, 1H) ppm.

### Example 340

### 3-[2-[2-(4′-Butoxy-biphenyl-4-yl)-(E)-vinyl]-4-(2,4-dichloro-phenyl)-imidazol-1-ylmethyl]-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-3-(bromomethyl)benzoate (229 mg, 1 mmol) following general procedure E. The resulted 3-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-butoxy-phenyl boronic acid (195 mg, 1 mmol) following General Procedure B to give 3-2-[2-(4′-butoxy-biphenyl-4-yl)-(E)-vinyl]-4-{4-(2,4-dichloro-phenyl)-imidazol-1yl-methyl}benzoic acid methyl ester (319 mg, 52%).

LCMS: 611 (M+H)⁺

### Example 341

### 3-[2-[2-(4′-Butoxy-biphenyl-4-yl)-(E)-vinyl]-4-(2,4-dichloro-phenyl)-imidazol-1-ylmethyl]-benzoic acid

3-2-[2-(4′-butoxy-biphenyl-4-yl)-(E)-vinyl]-4-{4-(2,4-dichloro-phenyl)-imidazol-1yl-methyl}benzoic acid methyl ester (305 mg, 0.5 mmol) was hydrolyzed according to General Procedure F to give 3-[2-[2-(4′-Butoxy-biphenyl-4-yl)-(E)-vinyl]-4-(2,4-dichloro-phenyl)-imidazol-1-ylmethyl]-benzoic acid (191 mg, 64%)

LCMS: 597 (M+H)^{+ 1}H NMR (DMSO, 400 MHz): δ 0.97 (t, 3H), 1.42-1.46 (m, 2H), 1.69-1.71 (m, 2H), 4.01 (q, 2H), 5.67 (s, 2H), 7.04 (d, 1H), 7.27 (s, 1H), 7.34-7.38 (m, 4H), 7.51-7.55 (m, 4H), 7.57-7.63 (m, 4H), 7.64 (d, 1H), 7.90 (d, 1H), 8.09 (s, 1H), 8.21 (d, 1H) ppm.

### Example 342

### 4-{4-(2,4-Dichloro-phenyl)-2-[2-(4′-methanesulfonyl-biphenyl-4-yl)-(E)-vinyl]-imidazol-1-ylmethyl}-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(methanesulfonyl)-phenyl boronic acid (200 mg, 1 mmol) following General Procedure B to give 4-2-[2-(4′-methanesulfonyl-biphenyl-4-yl)-(E)-vinyl]-4-{4-(2,4-dichloro-phenyl)-imidazol-1yl-methyl}benzoic acid methyl ester (294 mg, 47%)

LCMS: 617 (M+H)⁺.

### Example 343

### 4-{4-(2,4-Dichloro-phenyl)-2-[2-(4′-methanesulfonyl-biphenyl-4-yl)-(E)-vinyl]-imidazol-1-ylmethyl}-benzoic acid

4-{4-(2,4-Dichloro-phenyl)-2-[2-(4′-methanesulfonyl-biphenyl-4-yl)-(E)-vinyl]-imidazol-1-ylmethyl}-benzoic acid methyl ester (155 mg, 0.25 mmol) was hydrolyzed according to General Procedure F to give 4-{4-(2,4-Dichloro-phenyl)-2-[2-(4′-methanesulfonyl-biphenyl-4-yl)-(E)-vinyl]-imidazol-1-ylmethyl}-benzoic acid (108 mg, 72%)

LCMS: 603 (M+H)^{+ 1}H NMR (DMSO, 400 MHz): δ 3.47 (s, 3H), 5.66 (s, 2H), 7.12 (d, 1H), 7.36 (s, 1H), 7.37-7.40 (m, 4H), 7.52-7.54 (m, 4H), 7.58-7.64 (m, 4H), 7.66 (d, 1H), 7.91 (d, 1H), 8.09 (s, 1H), 8.21 (d, 1H) ppm.

### Example 344

### 4-{4-(2,4-Dichloro-phenyl)-2-[2-(3′-methanesulfonyl-biphenyl-4-yl)-(E)-vinyl]-imidazol-1-ylmethyl}-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(methanesulfonyl)-phenyl boronic acid (200 mg, 1 mmol) following General Procedure B to give 4-2-[2-(3′-methanesulfonyl-biphenyl-4-yl)-(E)-vinyl]-4-{4-(2,4-dichloro-phenyl)-imidazol-1yl-methyl}benzoic acid methyl ester (299 mg, 48%)

LCMS: 617 (M+H)⁺.

### Example 345

### 4-{4-(2,4-Dichloro-phenyl)-2-[2-(3′-methanesulfonyl-biphenyl-4-yl)-(E)-vinyl]-imidazol-1-ylmethyl}-benzoic acid

4-{4-(2,4-Dichloro-phenyl)-2-[2-(3′-methanesulfonyl-biphenyl-4-yl)-(E)-vinyl]-imidazol-1-ylmethyl}-benzoic acid methyl ester (155 mg, 0.25 mmol) was hydrolyzed according to General Procedure F to give 4-{4-(2,4-Dichloro-phenyl)-2-[2-(3′-methanesulfonyl-biphenyl-4-yl)-(E)-vinyl]-imidazol-1-ylmethyl}-benzoic acid (101 mg, 67%)

LCMS: 603 (M+H)^{+ 1}H NMR (DMSO, 400 MHz): δ 3.31 (s, 3H), 5.51 (s, 2H), 7.23 (s, 1H), 7.45-7.49 (m, 2H), 7.51-7.57 (m, 2H), 7.61-7.64 (m, 2H), 7.75-7.76 (m, 2H), 7.79-7.82 (m, 2H), 7.84-8.07 (m, 4H), 8.10 (d, 1H), 8.19 (s, 1H), 8.25 (d, 1H) ppm.

### Example 346

### 2-(4-{2-[4-(2,4-Dichloro-phenyl)-1-(4-methoxycarbonyl-benzyl)-1H-imidazol-2-yl]-(E)-vinyl}-phenyl)-pyrrole-1-carboxylic acid tert-butyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 1-(tert-butoxycarbonyl)-pyrrole-2-boronic acid (211 mg, 1 mmol) following General Procedure B to give 2-(4-{2-[4-(2,4-Dichloro-phenyl)-1-(4-methoxycarbonyl-benzyl)-1H-imidazol-2-yl]-(E)-vinyl}-phenyl)-pyrrole-1-carboxylic acid tert-butyl ester (278 mg, 44%)

LCMS: 628 (M+H)⁺.

### Example 347

### 2-(4-{2-[1-(4-Carboxy-benzyl)-4-(2,4-dichloro-phenyl)-1H-imidazol-2-yl]-(E)-vinyl}-phenyl)-pyrrole-1-carboxylic acid tert-butyl ester

2-(4-{2-[4-(2,4-Dichloro-phenyl)-1-(4-methoxycarbonyl-benzyl)-1H-imidazol-2-yl]-(E)-vinyl}-phenyl)-pyrrole-1-carboxylic acid tert-butyl ester (157 mg, 0.25 mmol) was hydrolyzed according to General Procedure F to give 2-(4-{2-[1-(4-Carboxy-benzyl)-4-(2,4-dichloro-phenyl)-1H-imidazol-2-yl]-(E)-vinyl}-phenyl)-pyrrole-1-carboxylic acid tert-butyl ester (89 mg, 59%)

LCMS: 614 (M+H)⁺.

### Example 348

### 4-(4-(2,4-Dichloro-phenyl)-2-{2-[4-(1H-pyrrol-2-yl)-phenyl]-(E)-vinyl}-imidazol-1-ylmethyl)-benzoic acid

2-(4-{2-[1-(4-Carboxy-benzyl)-4-(2,4-dichloro-phenyl)-1H-imidazol-2-yl]-(E)-vinyl}-phenyl)-pyrrole-1-carboxylic acid tert-butyl ester (62 mg, 0.1 mmol) was de-protected according to General Procedure O to give 4-(4-(2,4-Dichloro-phenyl)-2-{2-[4-(1H-pyrrol-2-yl)-phenyl]-(E)-vinyl}-imidazol-1-ylmethyl)-benzoic acid (29 mg, 55%).

LCMS: 514 (M+H)⁺.

### Example 349

### 4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-hydroxy-phenyl boronic acid (137 mg, 1 mmol) following General Procedure B and obtained 4-2-[2-(4′-hydroxy-biphenyl-4-yl)-(E)-vinyl]-4-{4-(2,4-dichloro-phenyl)-imidazol-1yl-methyl}benzoic acid methyl ester (278 mg, 0.5 mmol) was alkylated with 4-fluoronitro benzene (71 mg, 0.5 mmol) according to general procedure I to give 4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (221 mg, 65%).

LCMS: 676 (M+H)⁺.

### Example 350

### 4-[2-{2-[4′-(4-Nitro-phenoxy)-biphenyl-4-yl]-(E)-vinyl}-4-(2,4-dichloro-phenyl)-imidazol-1-ylmethyl]-benzoic acid

4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (169 mg, 0.25 mmol) was hydrolyzed according to General Procedure F to give 4-[2-{2-[4′-(4-Nitro-phenoxy)-biphenyl-4-yl]-(E)-vinyl}-4-(2,4-dichloro-phenyl)-imidazol-1-ylmethyl]-benzoic acid (125 mg, 75%).

LCMS: 662 (M+H)⁺.

### Example 351

### 4-[2-{2-[4′-(4-Amino-phenoxy)-biphenyl-4-yl]-(E)-vinyl}-4-(2,4-dichloro-phenyl)-imidazol-1-ylmethyl]-benzoic acid methyl ester

4-[2-{2-[4′-(4-Nitro-phenoxy)-biphenyl-4-yl]-(E)-vinyl}-4-(2,4-dichloro-phenyl)-imidazol-1-ylmethyl]-benzoic acid methyl ester (169 mg, 0.25 mmol) was reduced according to general procedure K to give 4-[2-{2-[4′-(4-amino-phenoxy)-biphenyl-4-yl]-(E)-vinyl}-4-(2,4-dichloro-phenyl)-imidazol-1-ylmethyl]-benzoic acid methyl ester (112 mg, 69%).

LCMS: 646 (M+H)⁺.

### Example 352

### 4-(4-(2,4-Dichlorophenyl)-2-{2-[4′-(4-methanesulfonylamino-phenoxy)-biphenyl-4-yl]-(E)-vinyl}-imidazol-1-ylmethyl)-benzoic acid methyl ester

4-[2-{2-[4′-(4-amino-phenoxy)-biphenyl-4-yl]-(E)-vinyl}-4-(2,4-dichloro-phenyl)-imidazol-1-ylmethyl]-benzoic acid methyl ester (65 mg, 0.1 mmol) was coupled with methanesulfonyl chloride (12 mg, 0.1 mmol) following general procedure L to give 4-(4-(2,4-Dichloro-phenyl)-2-{2-[4′-(4-methanesulfonylamino-phenoxy)-biphenyl-4-yl]-(E)-vinyl}-imidazol-1-ylmethyl)-benzoic acid methyl ester (41 mg, 57%).

LCMS: 724 (M+H)⁺.

### Example 353

### 4-(4-(2,4-Dichloro-phenyl)-2-{2-[4′-(4-methanesulfonylamino-phenoxy)-biphenyl-4-yl]-(E)-vinyl}-imidazol-1-ylmethyl)-benzoic acid

4-(4-(2,4-Dichloro-phenyl)-2-{2-[4′-(4-methanesulfonylamino-phenoxy)-biphenyl-4-yl]-(E)-vinyl}-imidazol-1-ylmethyl)-benzoic acid methyl ester (36 mg, 0.05 mmol) was hydrolyzed according to General Procedure F to give 4-(4-(2,4-Dichloro-phenyl)-2-{2-[4′-(4-methanesulfonylamino-phenoxy)-biphenyl-4-yl]-(E)-vinyl}-imidazol-1-ylmethyl)-benzoic acid (20 mg, 64%).

LCMS: 710 (M+H)⁺

### Example 354

### 4-{4-(2,4-Dichloro-phenyl)-2-[2-(3′-methanesulfonylamino-biphenyl-4-yl)-(E)-vinyl]-imidazol-1-ylmethyl}-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(methanesulfonylamino)-phenyl boronic acid (215 mg, 1 mmol) following General Procedure B to give 4-2-[2-(3′-methanesulfonylamino-biphenyl-4-yl)-(E)-vinyl]-4-{4-(2,4-dichloro-phenyl)-imidazol-1yl-methyl}benzoic acid methyl ester (304 mg, 48%)

LCMS: 632 (M+H)⁺.

### Example 355

### 4-{4-(2,4-Dichloro-phenyl)-2-[2-(3′-methanesulfonylamino-biphenyl-4-yl)-(E)-vinyl]-imidazol-1-ylmethyl}-benzoic acid

4-{4-(2,4-Dichloro-phenyl)-2-[2-(3′-methanesulfonylamino-biphenyl-4-yl)-(E)-vinyl]-imidazol-1-ylmethyl}-benzoic acid methyl ester (158 mg, 0.25 mmol) was hydrolyzed according to General Procedure F to give 4-{4-(2,4-Dichloro-phenyl)-2-[2-(3′-methane-sulfonylamino-biphenyl-4-yl)-(E)-vinyl]-imidazol-1-ylmethyl}-benzoic acid (109 mg, 70%)

LCMS: 618 (M+H)⁺; ¹H NMR (DMSO, 400 MHz): δ 3.38 (s, 3H), 5.64 (s, 2H), 7.21 (d, 1H), 7.33-7.42 (m, 4H), 7.43-7.52 (m, 4H), 7.56-7.75 (m, 4H). 7.77 (d, 1H, 7.92 (d, 1H), 8.11 (s, 1H), 8.27 (d, 1H), 9.85 (s. 1H), 13.02 (s, 1H) ppm.

### Example 356

### 4-{4-(2,4-Dichloro-phenyl)-2-[2-(4′-methanesulfonylamino-biphenyl-4-yl)-(E)-vinyl]-imidazol-1-ylmethyl}-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-(methanesulfonylamino)-phenyl boronic acid (215 mg, 1 mmol) following General Procedure B to give 4-2-[2-(4′-methanesulfonylamino-biphenyl-4-yl)-(E)-vinyl]-4-{4-(2,4-dichloro-phenyl)-imidazol-1yl-methyl}benzoic acid methyl ester (308 mg, 48%)

LCMS: 632 (M+H)⁺

### Example 357

### 4-{4-(2,4-Dichloro-phenyl)-2-[2-(4′-methanesulfonylamino-biphenyl-4-yl)-(E)-vinyl]-imidazol-1-ylmethyl}-benzoic acid

4-{4-(2,4-Dichloro-phenyl)-2-[2-(4′-methanesulfonylamino-biphenyl-4-yl)-(E)-vinyl]-imidazol-1-ylmethyl}-benzoic acid methyl ester (158 mg, 0.25 mmol) was hydrolyzed according to General Procedure F to give 4-{4-(2,4-Dichloro-phenyl)-2-[2-(4′-methanesulfonylamino-biphenyl-4-yl)-(E)-vinyl]-imidazol-1-ylmethyl}-benzoic acid (101 mg, 66%)

LCMS: 618 (M+H)^{+ 1}H NMR (DMSO, 400 MHz): δ 3.47 (s, 3H), 5.64 (s, 2H), 6.70 (d, 2H), 7.01 (d, 2H), 7.28-7.30 (m, 2H), 7.35-7.37 (m, 2H), 7.51-7.59 (m, 2H), 7.65-7.72 (m, 2H), 7.74 (d, 1H), 7.93 (s, 1H). 8.11 (s, 1H), 8.27 (d, 1H), 9.18 (s, 1H). 9.37 (s, 1H), 13.01 (s, 1H) ppm.

### Example 358

### 4′-{2-[4-(2,4-Dichloro-phenyl)-1-(4-methoxycarbonyl-benzyl)-1H-imidazol-2-yl]-(E)-vinyl}-biphenyl-3-carboxylic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 3-(methoxycarbonyl)-phenyl boronic acid (179 mg, 1 mmol) following General Procedure B to give 4′-{2-[4-(2,4-Dichloro-phenyl)-1-(4-methoxycarbonyl-benzyl)-1H-imidazol-2-yl]-(E)-vinyl}-biphenyl-3-carboxylic acid methyl ester (289 mg, 48%)

LCMS: 597 (M+H)⁺.

### Example 359

### 4′-{2-[4-(2,4-Dichloro-phenyl)-1-(4-methoxycarbonyl-benzyl)-1H-imidazol-2-yl]-(E)-vinyl}-biphenyl-3-carboxylic acid

4′-{2-[4-(2,4-Dichloro-phenyl)-1-(4-methoxycarbonyl-benzyl)-1H-imidazol-2-yl]-(E)-vinyl}-biphenyl-3-carboxylic acid methyl ester (149 mg, 0.25 mmol) was hydrolyzed according to General Procedure F to give 4′-{2-[4-(2,4-Dichloro-phenyl)-1-(4-methoxycarbonyl-benzyl)-1H-imidazol-2-yl]-(E)-vinyl}-biphenyl-3-carboxylic acid (99 mg, 69%)

LCMS: 569 (M+H)⁺; ¹H NMR (DMSO, 400 MHz): δ 5.70 (s, 2H), 7.39-7.45 (m, 4H), 7.54 (d, 1H), 7.61 (d, 1H), 7.70-7.74 (m, 4H), 7.76 (d, 1H), 7.79-7.96 (m, 4H), 7.98 (s, 1H), 8.17 (d, 1H), 8.22 (d, 1H) ppm.

### Example 360

### 4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 4-hydroxy-phenyl boronic acid (137 mg, 1 mmol) following General Procedure B and obtained 4-2-[2-(4′-hydroxy-biphenyl-4-yl)-(E)-vinyl]-4-{4-(2,4-dichloro-phenyl)-imidazol-1yl-methyl}benzoic acid methyl ester (277 mg, 0.5 mmol) was alkylated with 1-bromo-4,4,4-trifluorobutane following general procedure E to give 4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid methyl ester (214 mg, 64%).

LCMS: 665 (M+H)⁺.

### Example 361

### 4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid

4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid methyl ester (166 mg, 0.25 mmol) was hydrolyzed according to General Procedure F to give 4-(4-(2,4-Dichloro-phenyl)-2-{2-[4′-(4,4,4-trifluoro-butoxy)-biphenyl-4-yl]-(E)-vinyl}-imidazol-1-ylmethyl)-benzoic acid (106 mg, 65%)

LCMS: 651 (M+H)^{+ 1}H NMR (DMSO, 400 MHz): δ 1.41-1.44 (m, 2H), 1.66-1.71 (m, 2H), 2.41-2.47 (m, 2H), 5.66 (s, 2H), 7.12 (d, 1H), 7.19 (s, 1H), 7.33-7.37 (m, 4H), 7.51-7.55 (m, 4H), 7.56-7.62 (m, 4H), 7.65 (d, 1H), 7.91 (d, 1H), 8.11 (s, 1H), 8.29 (d, 1H) ppm.

### Example 362

### 4-(4-(2,4-Dichloro-phenyl)-2-{2-[4-(6-methoxy-pyridin-3-yl)-phenyl]-(E)-vinyl}-imidazol-1-ylmethyl)-benzoic acid methyl ester

Trans-4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with methyl-4-(bromomethyl)benzoate (229 mg, 1 mmol) following general procedure E. The resulted 4-[2-[2-(4-bromo-phenyl)-(E)-vinyl]-4-(2,4-dichloro-phenyl)}-imidazol-1yl-methyl]-benzoic acid methyl ester (542 mg, 1 mmol) was coupled with 2-methoxy-5-pyridine boronic acid (153 mg, 1 mmol) following General Procedure B to give 4-(4-(2,4-Dichloro-phenyl)-2-{2-[4-(6-methoxy-pyridin-3-yl)-phenyl]-(E)-vinyl}-imidazol-1-ylmethyl)-benzoic acid methyl ester (289 mg, 50%)

LCMS: 570 (M+H)⁺

### Example 363

### 4-(4-(2,4-Dichloro-phenyl)-2-{2-[4-(6-methoxy-pyridin-3-yl)-phenyl]-(E)-vinyl}-imidazol-1-ylmethyl)-benzoic acid

4-(4-(2,4-Dichloro-phenyl)-2-{2-[4-(6-methoxy-pyridin-3-yl)-phenyl]-(E)-vinyl}-imidazol-1-ylmethyl)-benzoic acid methyl ester (143 mg, 0.25 mmol) was hydrolyzed according to General Procedure F to give 4-(4-(2,4-Dichloro-phenyl)-2-{2-[4-(6-methoxy-pyridin-3-yl)-phenyl]-(E)-vinyl}-imidazol-1-ylmethyl)-benzoic acid (95 mg, 68%)

LCMS: 556 (M+H)^{+ 1}H NMR (DMSO, 400 MHz): δ 3.79 (s, 3H), 5.68 (s, 2H), 7.01 (d, 1H), 7.26 (s, 1H), 7.36-7.40 (m, 3H), 7.51-7.56 (m, 3H), 7.58-7.64 (m, 4H), 7.67 (d, 1H), 7.92 (d, 1H), 8.11 (s, 1H), 8.27 (d, 1H) ppm.

### Example 364

### 2-[2-(4′-Butoxy-biphenyl-4-yl)-(E)-vinyl]-4-(2,4-dichlorophenyl)-1-(4-trifluoromethoxy-benzyl)-1H-imidazole

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with 4-(trifluoromethoxy)-benzyl bromide (255 mg, 1 mmol) following general procedure E. The resulted 2-[2-(4-Bromo-phenyl)-(E)-vinyl]-4-(2,4-dichloro-phenyl)-1-(4-trifluoromethoxy-benzyl)-1H-imidazole (284 mg, 0.5 mmol) was coupled with 4-butoxy-phenyl boronic acid (98 mg, 0.5 mmol) following General Procedure B to give 2-[2-(4′-Butoxy-biphenyl-4-yl)-(E)-vinyl]-4-(2,4-dichloro-phenyl)-1-(4-trifluoromethoxy-benzyl)-1H-imidazole (155 mg, 48%).

LCMS: 637 (M+H)⁺; ¹H NMR (DMSO, 400 MHz): δ 0.92 (t, 3H), 1.43-1.47 (m, 2H), 1.69-1.72 (m, 2H), 4.02 (q, 1H), 5.59 (s, 2H), 7.02 (d, 2H), 7.34 (s, 1H), 7.39-7.42 (m, 4H), 7.50 (d, 1H), 7.51 (d, 1H), 7.52 (d, 1H), 7.55-7.65 (m, 4H), 7.72 (d, 2H), 8.10 (s, 1H), 8.26 (d, 1H) ppm.

### Example 365

### 4-(4′-{2-[4-(2,4-Dichloro-phenyl)-1-(4-trifluoromethoxy-benzyl)-1H-imidazol-2-yl]-(E)-vinyl}-biphenyl-4-yloxy)-butyric acid methyl ester

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with 4-(trifluoromethoxy)-benzyl bromide (255 mg, 1 mmol) following general procedure E. The resulted 2-[2-(4-Bromo-phenyl)-(E)-vinyl]-4-(2,4-dichloro-phenyl)-1-(4-trifluoromethoxy-benzyl)-1H-imidazole (284 mg, 0.5 mmol) was coupled with 4-hydroxy-phenyl boronic acid (69 mg, 0.5 mmol) following General Procedure B and obtained 2-[2-(4′-hydroxy-biphenyl-4-yl)-(E)-vinyl]-4-(2,4-dichloro-phenyl)-1-(4-trifluoromethoxy-benzyl)-1H-imidazole (145 mg, 0.25 mol) was alkylated with 4-bromobutyric acid methyl ester (45 mg, 0.25 mmol) following general procedure E to give 4-(4′-{2-[4-(2,4-Dichloro-phenyl)-1-(4-trifluoromethoxy-benzyl)-1H-imidazol-2-yl]-(E)-vinyl}-biphenyl-4-yloxy)-butyric acid methyl ester (115 mg, 67%).

LCMS: 681 (M+H)⁺

### Example 366

### 4-(4′-{2-[4-(2,4-Dichloro-phenyl)-1-(4-trifluoromethoxy-benzyl)-1H-imidazol-2-yl]-(E)-vinyl}-biphenyl-4-yloxy)-butyric acid

4-(4′-{2-[4-(2,4-Dichloro-phenyl)-1-(4-trifluoromethoxy-benzyl)-1H-imidazol-2-yl]-(E)-vinyl}-biphenyl-4-yloxy)-butyric acid methyl ester (69 mg, 0.1 mmol) was hydrolyzed according to General Procedure F to give 4-(4′-{2-[4-(2,4-Dichloro-phenyl)-1-(4-trifluoromethoxy-benzyl)-1H-imidazol-2-yl]-(E)-vinyl}-biphenyl-4-yloxy)-butyric acid (46 mg, 68%)

LCMS: 667 (M+H)^{+ 1}H NMR (DMSO, 400 MHz): δ 1.97 (m, 2H), 2.38 (m, 2H), 4.03 (m, 2H), 5.61 (s, 2H), 7.01 (d, 2H), 7.35 (d, 1H), 7.40-7.44 (m, 4H), 7.52 (d, 1H), 7.60-7.67 (m, 6H), 7.74 (d, 2H), 8.14 (s, 1H), 8.23 (d, 1H) ppm.

### Example 367

### 4-(2,4-Dichloro-phenyl)-1-(4-methanesulfonyl-benzyl)-2-[2-(3′-trifluoromethyl-biphenyl-4-yl)-(E)-vinyl]-1H-imidazole

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with 4-(methanesulfonyl)-benzyl bromide (249 mg, 1 mmol) following general procedure E. The resulted 2-[2-(4-Bromo-phenyl)-(E)-vinyl]-4-(2,4-dichloro-phenyl)-1-(4-methanesulfonyl-benzyl)-1H-imidazole (281 mg, 0.5 mmol) was coupled with 3-(trifluoromethyl)-phenyl boronic acid (95 mg, 0.5 mmol) following General Procedure B to give 4-(2,4-Dichloro-phenyl)-1-(4-methanesulfonyl-benzyl)-2-[2-(3′-trifluoromethyl-biphenyl-4-yl)-(E)-vinyl]-1H-imidazole (155 mg, 49%).

LCMS: 627 (M+H)^{+ 1}H NMR (DMSO, 400 MHz): δ 3.35 (s, 3H), 5.71 (s, 2H), 7.41 (s, 1H), 7.45 (s, 1H), 7.51-7.77 (m, 6H), 7.79-7.93 (m, 4H), 7.95-8.12 (m, 4H), 8.28 (d, 1H), 8.39 (s, 1H) ppm.

### Example 368

### 4-(2,4-Dichloro-phenyl)-1-(4-methanesulfonyl-benzyl)-2-[2-(3′-methanesulfonylbiphenyl-4-yl)-(E)-vinyl]-1H-imidazole

Trans 4-bromo cinnamic acid (227 mg, 1 mmol) was reacted with 2-bromo-2,4-dichloro acetophenone (267 mg, 1 mmol) according to general procedure A and obtained 2-[2-(4-bromo-phenyl)-(E)-vinyl]-4-(2,4-dichloro-phenyl)}-1H-imidazole (412 mg, 1 mmol) was N-alkylated with 4-(methanesulfonyl)-benzyl bromide (249 mg, 1 mmol) following general procedure E. The resulted 2-[2-(4-Bromo-phenyl)-(E)-vinyl]-4-(2,4-dichloro-phenyl)-1-(4-methanesulfonyl-benzyl)-1H-imidazole (281 mg, 0.5 mmol) was coupled with 3-(methanesulfonyl)-phenyl boronic acid (100 mg, 0.5 mmol) following General Procedure B to give 4-(2,4-Dichloro-phenyl)-1-(4-methanesulfonyl-benzyl)-2-[2-(3′-methanesulfonyl-biphenyl-4-yl)-(E)-vinyl]-1H-imidazole (165 mg, 52%).

LCMS: 637 (M+H)^{+ 1}H NMR (DMSO, 400 MHz): δ 3.31 (s, 3H), 3.34 (s, 3H), 5.71 (s, 2H), 7.41 (s, 1H), 7.46 (s, 1H), 7.51 (d, 1H), 7.52 (d, 1H), 7.53 (s, 1H), 7.65-7.81 (m, 4H), 7.83-7.85 (m, 4H), 7.93 (d, 1H), 7.95 (s, 1H), 8.15 (d, 1H), 8.19 (d, 1H), 8.28 (d, 1H) ppm.

### Example 369

### 4-[4-(2,4-Dichloro-phenyl)-2-(4′-hydroxy-biphenyl-4-yl)-imidazol-1-ylmethyl]-benzoic acid methyl ester

4-Hydroxy-4-biphenyl carboxylic acid (214 mg, 1 mmol) was reacted with 2-bromo-2,4-dichloro acetophenone (267 mg, 1 mmol) according to general procedure A and obtained 4′-[4-(2,4-Dichloro-phenyl)-1H-imidazol-2-yl]-biphenyl-4-ol (381 mg, 1 mmol) was N-alkylated with methyl-4-(bromomethyl)benzoate (229 mg, 1 mmol) following general procedure E to give 4-[4-(2,4-Dichloro-phenyl)-2-(4′-hydroxy-biphenyl-4-yl)-imidazol-1-ylmethyl]-benzoic acid methyl ester (312 mg, 59%).

LCMS: 529 (M+H)⁺.

### Example 370

### 4-[4-(2,4-Dichloro-phenyl)-2-(4′-hydroxy-biphenyl-4-yl)-imidazol-1-ylmethyl]-benzoic acid

4-[4-(2,4-Dichloro-phenyl)-2-(4′-hydroxy-biphenyl-4-yl)-imidazol-1-ylmethyl]-benzoic acid methyl ester (264 mg, 0.5 mmol) was hydrolyzed according to General Procedure F to give 4-[4-(2,4-Dichloro-phenyl)-2-(4′-hydroxy-biphenyl-4-yl)-imidazol-1-ylmethyl]-benzoic acid (186 mg, 72%).

LCMS: 515 (M+H)^{+ 1}H NMR (DMSO, 400 MHz): δ 5.54 (s, 2H), 6.81-6.86 (m, 5H), 7.23 (d, 1H), 7.41-7.57 (m, 5H), 7.74 (d, 1H), 7.89 (d, 1H), 7.94 (d, 1H), 8.11 (s, 1H), 8.27 (d, 1H) ppm.

### Example 371

### 4-[4-(2,4-Dichloro-phenyl)-2-(4′-ethoxy-biphenyl-4-yl)-imidazol-1-ylmethyl]-benzoic acid methyl ester

4-Hydroxy-4-biphenyl carboxylic acid (214 mg, 1 mmol) was reacted with 2-bromo-2,4-dichloro acetophenone (267 mg, 1 mmol) according to general procedure A and obtained 4′-[4-(2,4-Dichloro-phenyl)-1H-imidazol-2-yl]-biphenyl-4-ol (381 mg, 1 mmol) was N-alkylated with methyl-4-(bromomethyl)benzoate (229 mg, 1 mmol) following general procedure E. The resulted 4-[4-(2,4-Dichloro-phenyl)-2-(4′-hydroxy-biphenyl-4-yl)-imidazol-1-ylmethyl]-benzoic acid methyl ester (265 mg, 0.5 mmol) was alkylated with bromo ethane (55 mg, 0.5 mmol) following general procedure E to give 4-[4-(2,4-Dichloro-phenyl)-2-(4′-ethoxy-biphenyl-4-yl)-imidazol-1-ylmethyl]-benzoic acid methyl ester (191 mg, 68%).

LCMS: 557 (M+H)⁺.

### Example 372

### 4-[4-(2,4-Dichloro-phenyl)-2-(4′-ethoxy-biphenyl-4-yl)-imidazol-1-ylmethyl]-benzoic acid

4-[4-(2,4-Dichloro-phenyl)-2-(4′-ethoxy-biphenyl-4-yl)-imidazol-1-ylmethyl]-benzoic acid methyl ester (278 mg, 0.5 mmol) was hydrolyzed according to General Procedure F to give 4-[4-(2,4-Dichloro-phenyl)-2-(4′-ethoxy-biphenyl-4-yl)-imidazol-1-ylmethyl]-benzoic acid (189 mg, 69%).

LCMS: 543 (M+H)⁺; ¹H NMR (DMSO, 400 MHz): δ 0.94 (t, 3H), 4.07 (q, 2H), 5.56 (s, 2H), 6.83-6.88 (m, 4H), 7.21 (d, 1H), 7.43-7.58 (m, 4H), 7.65-7.69 (m, 2H), 7.71 (d, 1H), 7.90 (d, 1H), 7.94 (d, 1H), 8.12 (s, 1H), 8.28 (d, 1H) ppm.

### Example 373

### 4-[4-(2,4-Dichloro-phenyl)-2-(3′-methanesulfonyl-biphenyl-4-yl)-imidazol-1-ylmethyl]-benzoic acid methyl ester

4-Bromo benzoic acid (201 mg, 1 mmol) was reacted with 2-bromo-2,4-dichloro acetophenone (267 mg, 1 mmol) according to general procedure A and obtained 2-(4-bromo-phenyl)-4-(2,4-dichloro-phenyl)-1H-imidazole (368 mg, 1 mmol) was N-alkylated with methyl-4-(bromomethyl)benzoate (229 mg, 1 mmol) following general procedure E. The resulted 4-[2-(4-Bromo-phenyl)-4-(2,4-dichloro-phenyl)-imidazol-1-ylmethyl]-benzoic acid methylester (516 mg, 1 mmol) was coupled with 3-(methanesulfonyl)-phenyl boronic acid (200 mg, 1 mmol) following General Procedure B to give 4-[4-(2,4-Dichloro-phenyl)-2-(3′-methanesulfonyl-biphenyl-4-yl)-imidazol-1-ylmethyl]-benzoic acid methyl ester (324 mg, 55%).

LCMS: 591 (M+H)⁺

### Example 374

### 4-[4-(2,4-Dichloro-phenyl)-2-(3′-methanesulfonyl-biphenyl-4-yl)-imidazol-11 methyl]-benzoic acid

4-[4-(2,4-Dichloro-phenyl)-2-(3′-methanesulfonyl-biphenyl-4-yl)-imidazol-1-ylmethyl]-benzoic acid methyl ester (295 mg, 0.5 mmol) was hydrolyzed according to General Procedure F to give 4-[4-(2,4-Dichloro-phenyl)-2-(3′-methanesulfonyl-biphenyl-4-yl)-imidazol-1-ylmethyl]-benzoic acid (201 mg, 69%).

LCMS: 577 (M+H)^{+ 1}H NMR (DMSO, 400 MHz): δ 3.31 (s, 3H), 5.64 (s, 2H), 7.25-7.33 (m, 4H), 7.60 (d, 1H), 7.76 (s, 1H), 7.82 (d, 1H), 7.84 (d, 1H), 7.90-7.96 (m, 4H), 8.10 (d, 1H), 8.18 (d, 1H), 8.23 (s, 1H), 8.30 (s, 1H) ppm.

### Example 375

### 4-{4-(2,4-Dichloro-phenyl)-2-[2-(4′-trifluoromethyl-biphenyl-4-yl)-ethyl]-imidazol-1-ylmethyl}-benzoic acid

4-{4-(2,4-dichloro-phenyl)-2-[2-(4′-trifluoromethyl-biphenyl-4-yl)-(E)-vinyl]-imidazol-1yl-methyl}benzoic acid (148 mg, 0.25 mmol) was reduced according to General Procedure V to give 4-{4-(2,4-Dichloro-phenyl)-2-[2-(4′-trifluoromethyl-biphenyl-4-yl)-ethyl]-imidazol-1-ylmethyl}-benzoic acid (79 mg, 53%).

LCMS: 595 (M+H)^{+ 1}H NMR (DMSO, 400 MHz): δ 2.92-2.94 (m, 2H), 2.98-3.0 (m, 2H), 5.64 (d, 2H), 7.20 (d, 1H), 7.31-7.38 (m, 2H), 7.42-7.52 (m, 2H), 7.58-7.65 (m, 2H), 7.75-7.79 (m, 2H), 7.80-7.95 (m, 4H), 8.11 (s, 1H), 8.22 (d, 1H), 8.30 (d, 1H) ppm.

### Biological Assay

The following assay methods are utilized to identify compounds of formula I which are effective in inhibiting the activity of certain phosphatases, an example of which, as used herein, is PTP1B.

### PTP1B Assay

The assay for PTP1B inhibition is based on the detection of the complex between Malachite Green dye and free phosphate, liberated from the phosphopeptide substrate by PTPase action. To each well of a flat-bottom assay plate is added 45 μL assay buffer [-50 mM Imidazole, pH 7.2, 100 mM NaCl, 5 mM DTT, and 1 mM EDTA] and 10 μL of peptide substrate [Tyrosine Phosphopeptide-1, 80 μM FAC, Promega Cat #V256A] to a total volume of 55 μL. Test compound (10 μL in up to 50% DMSO) is then added. The mixture is incubated for 5 min, at 25° C., and 10 μL of PTP-1B [Protein Tyrosine Phosphatase 1B (PTP-1B); FAC 0.8 nM; Upstate Biotechnology, Cat #14-109 lot #19045] is then added. The mixture is incubated for 30 min at 25° C. Subsequently, 25 μL of Malachite Green reagent [10% (w/v) Ammonium Molybdate in water, Sigma Cat #A-7302, 0.2% (w/v) Malachite Green in 4 N HCl, Aldrich Cat #21, 302-0] is then added. After incubation for 15 min at 27° C., the reaction endpoint is measured at 640 nM.

The Malachite Green reagent is prepared by mixing one volume of 10% Ammonium Molybdate with 3 volumes of 0.2% Malachite Green solution, stirring at room temperature for 30 min and then filtering and collecting the filtrate. The Malachite Green reagent is treated with 10 μL of 5% Tween 20 per 990 μL of dye solution before use.

Test compounds are typically examined at six concentrations in the above assay. For this assay, the IC50 (microM) of the enzyme inhibition assay represents the concentration of compound at which 50% signal has been inhibited.

As illustrated by the Examples, embodiments of the present invention demonstrate utility in inhibiting protein tyrosine phosphatase PTP 1B. The compounds of the present invention set forth in the present examples are found to inhibit protein tyrosine phosphatase PTP1B with inhibitory potencies (IC50's) of about 0.01 microM to about 20 microM. In general, embodiments of the present invention useful for pharmaceutical applications will have inhibitory potencies (IC50's) for a protein of interest of below about 100, or in an embodiment below about 50 microM. For particular applications, lower inhibitory potencies are useful, thus compounds that inhibit protein tyrosine phosphatase PTP1B with inhibitory potencies (IC50's) in a range of about 0.01 microM to about 10 microM may be useful. In another embodiment, compounds that inhibit protein tyrosine phosphatase PTP1B with inhibitory potencies (IC50's) of about 0.01 microM to about 3 microM may be useful.

Embodiments of the compounds of the present invention demonstrate utility as inhibitors of protein tyrosine phosphatases (PTPases). Embodiments of the invention described herein are additionally directed to pharmaceutical compositions and methods of inhibiting PTPase activity in a mammal, which methods comprise administering, to a mammal in need of inhibition of PTPase activity, a therapeutically defined amount of a compound of formula (I), defined above, as a single or polymorphic crystalline form or forms, an amorphous form, a single enantiomer, a racemic mixture, a single stereoisomer, a mixture of stereoisomers, a single diastereoisomer, a mixture of diastereoisomers, a solvate, a pharmaceutically acceptable salt, a solvate, a prodrug, a biohydrolyzable ester, or a biohydrolyzable amide thereof.

Thus, the present invention provides a method of inhibiting a PTPase, comprising the step of administering to a mammal in need thereof a pharmacologically effective amount of a compound of the present invention. The invention further provides a pharmaceutical composition comprising a pharmaceutically acceptable carrier and a pharmacologically effective amount of a compound of the present invention sufficient to inhibit a PTPase. A PTPase-inhibiting amount can be an amount that reduces or inhibits a PTPase activity in the subject.

Additionally provided is a pharmaceutical composition comprising a pharmaceutically acceptable carrier and a pharmacologically effective amount of a compound of the present invention sufficient to treat type I diabetes.

Further, the present invention provides a pharmaceutical composition comprising a pharmaceutically acceptable carrier and a pharmacologically effective amount of a compound of the present invention sufficient to treat type II diabetes.

Further, the present invention provides a pharmaceutical composition comprising a pharmaceutically acceptable carrier and a pharmacologically effective amount of a compound of the present invention sufficient to treat immune dysfunction.

Further, the present invention provides a pharmaceutical composition comprising a pharmaceutically acceptable carrier and a pharmacologically effective amount of a compound of the present invention sufficient to treat AIDS.

Further, the present invention provides a pharmaceutical composition comprising a pharmaceutically acceptable carrier and a pharmacologically effective amount of a compound of the present invention sufficient to treat autoimmune diseases

Further, the present invention provides a pharmaceutical composition comprising a pharmaceutically acceptable carrier and a pharmacologically effective amount of a compound of the present invention sufficient to treat glucose intolerance.

Further, the present invention provides a pharmaceutical composition comprising a pharmaceutically acceptable carrier and a pharmacologically effective amount of a compound of the present invention sufficient to treat obesity.

Further, the present invention provides a pharmaceutical composition comprising a pharmaceutically acceptable carrier and a pharmacologically effective amount of a compound of the present invention sufficient to treat cancer.

Further, the present invention provides a pharmaceutical composition comprising a pharmaceutically acceptable carrier and a pharmacologically effective amount of a compound of the present invention sufficient to treat psoriasis.

Further, the present invention provides a pharmaceutical composition comprising a pharmaceutically acceptable carrier and a pharmacologically effective amount of a compound of the present invention sufficient to treat allergic diseases

Further, the present invention provides a pharmaceutical composition comprising a pharmaceutically acceptable carrier and a pharmacologically effective amount of a compound of the present invention sufficient to treat infectious diseases.

Further, the present invention provides a pharmaceutical composition comprising a pharmaceutically acceptable carrier and a pharmacologically effective amount of a compound of the present invention sufficient to treat inflammatory diseases.

Further, the present invention provides a pharmaceutical composition comprising a pharmaceutically acceptable carrier and a pharmacologically effective amount of a compound of the present invention sufficient to treat diseases involving the modulated synthesis of growth hormone.

Further, the present invention provides a pharmaceutical composition comprising a pharmaceutically acceptable carrier and a pharmacologically effective amount of a compound of the present invention sufficient to treat diseases involving the modulated synthesis of growth factors or cytokines which affect the production of growth hormone.

Further, the present invention provides a pharmaceutical composition comprising a pharmaceutically acceptable carrier and a pharmacologically effective amount of a compound of the present invention sufficient to treat Alzheimer's disease.

The compounds of the present invention can be administered to subjects in need of inhibition of PTPase activity. Such subjects can include, for example, horses, cows, sheep, pigs, mice, dogs, cats, primates such as chimpanzees, gorillas, rhesus monkeys, and, most preferably humans.

The pharmaceutical compositions containing a compound of the invention may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatin capsules where the active ingredient is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, poly-vinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Also provided by the present invention are prodrugs of the invention. Pharmaceutically-acceptable salts of the compounds of the present invention, where a basic or acidic group is present in the structure, are also included within the scope of the invention. The term “pharmaceutically acceptable salts” refers to non-toxic salts of the compounds of this invention which are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isethionate, Lactate, Lactobionate, Laurate, Malate, Maleate, Mandelate, Mesylate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts listed in the Journal of Pharmaceutical Science, 66, 2 (1977) p. 1-19.

Other salts which are not pharmaceutically acceptable may be useful in the preparation of compounds of the invention and these form a further aspect of the invention.

In addition, some of the compounds of the present invention may form solvates with water or common organic solvents. Such solvates are also encompassed within the scope of the invention.

Thus, in a further embodiment, there is provided a pharmaceutical composition comprising a compound of the present invention, or a pharmaceutically acceptable salt, solvate, or prodrug thereof, and one or more pharmaceutically acceptable carriers, excipients, or diluents.

The compounds of the present invention selectively act as inhibitors of one PTPase in preference to one or more other PTPases, and therefore may possess advantage in the treatment of one or more PTPase-mediated disease in preference to others.

Thus, in a further aspect, the present invention provides a method for the inhibition of PTPases. In an embodiment of this aspect, the present invention provides a method for treating a disease states including diabetes, cancer, inflammation, Alzheimer's disease, psoriasis, or graft versus host disease, which comprises administering to a subject in need thereof a compound of the present invention. In an embodiment, the amount of compound administered is a pharmacologically effective amount. In another embodiment, the compound administered is a therapeutically effective amount. In another embodiment, at least one compound of Formula (I) is utilized, either alone or in combination with one or more known therapeutic agents. In another embodiment, the present invention provides method of prevention and/or treatment of PTPase-mediated human diseases, treatment comprising alleviation of one or more symptoms resulting from that disorder, to an outright cure for that particular disorder or prevention of the onset of the disorder, the method comprising administration to a human in need thereof a therapeutically effective amount of a compound of the present invention of Formula (I).

In this method, factors which will influence what constitutes an effective amount will depend upon the size and weight of the subject, the biodegradability of the therapeutic agent, the activity of the therapeutic agent, as well as its bioavailability. As used herein, the phrase “a subject in need thereof” includes mammalian subjects, preferably humans, who either suffer from one or more of the aforesaid diseases or disease states or are at risk for such. Accordingly, in the context of the therapeutic method of the invention, this method also is comprised of a method for treating a mammalian subject prophylactically, or prior to the onset of diagnosis such disease(s) or disease state(s).

The following is a non-exhaustive listing of adjuvants and additional therapeutic agents which may be utilized in combination with the PTPase inhibitors of the present invention:

Pharmacologic Classifications of Anticancer Agents:
1. Alkylating agents: Cyclophosphamide, nitrosoureas, carboplatin, cisplatin, procarbazine
2. Antibiotics: Bleomycin, Daunorubicin, Doxorubicin
3. Antimetabolites: Methotrexate, Cytarabine, Fluorouracil
4. Plant alkaloids: Vinblastine, Vincristine, Etoposide, Paclitaxel,
5. Hormones: Tamoxifen, Octreotide acetate, Finasteride, Flutamide
6. Biologic response modifiers: Interferons, Interleukins

Pharmacologic Classifications of Treatment for Rheumatoid Arthritis (Inflammation)
1. Analgesics: Aspirin
2. NSAIDs (Nonsteroidal anti-inflammatory drugs): Ibuprofen, Naproxen, Diclofenac
3. DMARDs (Disease-Modifying Antirheumatic drugs): Methotrexate, gold preparations, hydroxychloroquine, sulfasalazine
4. Biologic Response Modifiers, DMARDs: Etanercept, Infliximab

### Glucocorticoids

Pharmacologic Classifications of Treatment for Diabetes Mellitus
1. Sulfonylureas: Tolbutamide, Tolazamide, Glyburide, Glipizide
2. Biguanides: Metformin
3. Miscellaneous oral agents: Acarbose, PPAR agonists such as Troglitazone, DPP-IV inhibitors, Glucokinase activators
4. Insulin, insulin mimetics, insulin secretagogues, insulin sensitizers
5. GLP-1, GLP-1 mimetics

Pharmacologic Classifications of Treatment for Alzheimer's Disease
1. Cholinesterase Inhibitor: Tacrine, Donepezil
2. Antipsychotics: Haloperidol, Thioridazine
3. Antidepressants: Desipramine, Fluoxetine, Trazodone, Paroxetine
4. Anticonvulsants: Carbamazepine, Valproic acid

Pharmacologic Classifications of Treatment for Hyperlipidemia
1. HMG CoA reductase inhibitors Inhibitor: Mevinolin
2. cholestyramine
3. fibrates
   In another embodiment, the present invention provides a method of treating PTPase mediated diseases, the method comprising administering to a subject in need thereof, a therapeutically effective amount of a compound of Formula (I) in combination with therapeutic agents selected from the group consisting of alkylating agents, antimetabolites, plant alkaloids, antibiotics, hormones, biologic response modifiers, analgesics, NSAIDs, DMARDs, glucocorticoids, sulfonylureas, biguanides, acarbose, PPAR agonists, DPP-IV inhibitors, GK activators, insulin, insulin mimetics, insulin secretagogues, insulin sensitizers, GLP-1, GLP-1 mimetics, cholinesterase inhibitors, antipsychotics, antidepressants, anticonvulsants, HMG CoA reductase inhibitors, cholestyramine, and fibrates. In another embodiment, the present invention provides the pharmaceutical composition of the invention as described above, further comprising one or more therapeutic agents selected from the group consisting of alkylating agents, antimetabolites, plant alkaloids, antibiotics, hormones, biologic response modifiers, analgesics, NSAIDs, DMARDs, glucocorticoids, sulfonylureas, biguanides, acarbose, PPAR agonists, DPP-IV inhibitors, GK activators, insulin, insulin mimetics, insulin secretagogues, insulin sensitizers, GLP-1, GLP-1 mimetics, cholinesterase inhibitors, antipsychotics, antidepressants, anticonvulsants, HMG CoA reductase inhibitors, cholestyramine, and fibrates.

Generally speaking, the compound of the present invention or Formula (I), is administered at a dosage level of from about 0.003 to 500 mg/kg of the body weight of the subject being treated, a dosage range between 0.003 and 200 mg/kg, or a dosage range between 0.1 to 100 mg/kg of body weight per day. The amount of active ingredient that may be combined with the carrier materials to produce a single dosage will vary depending upon the host treated and the particular mode of administration. For example, a formulation intended for oral administration to humans may contain 1 mg to 2 grams of a compound of Formula (I) with an appropriate and convenient amount of carrier material which may vary from about 5 to 95 percent of the total composition. Dosage unit forms will generally contain between from about 5 mg to about 500 mg of active ingredient. Also a dosage form intended for topical administration to the skin may be prepared at 0.1% to 99% compound to topical excipient ratio and a dosage form intended for inhaled administration of 0.01 to 200 mg of compound in a suitable carrier to deliver an inhaled dosage of compound. Dosage unit forms of systemically delivered compound will generally contain between from about 5 mg to about 500 mg of active ingredient. This dosage has to be individualized by the clinician based on the specific clinical condition of the subject being treated. Thus, it will be understood that the specific dosage level for any particular patient will depend upon a variety of factors including the activity of the specific compound employed, the age, body weight, general health, sex, diet, time of administration, route of administration, rate of excretion, drug combination and the severity of the particular disease undergoing therapy.

While the invention has been described and illustrated with reference to certain embodiments thereof, those skilled in the art will appreciate that various changes, modifications and substitutions can be made therein without departing from the spirit and scope of the invention. For example, effective dosages other than the dosages as set forth herein may be applicable as a consequence of variations in the responsiveness of the mammal being treated for PTPase-mediated disease(s). Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

1-63. (canceled)

64. A compound of Formula (I) or a pharmaceutically acceptable salt thereof: 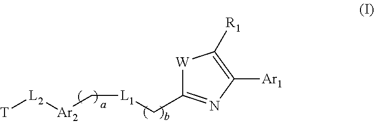
wherein
a and b are, independently, equal to 0 wherein the value of 0 represents a direct bond;
W is —N(R₂)—,
wherein
R₂ is
a) -alkyl;
b) -L₃-D-G;
c) -L₃-D-alkyl:
d) -L₃-D-aryl;
e) -L₃-D-heteroaryl;
f) -L₃-D-cycloalkyl;
g) -L₃-D-heterocyclyl;
h) -L₃-D-arylene-alkyl;
i) -L₃-D-alkylene-arylene-alkyl;
j) -L₃-D-alkylene-aryl;
k) -L₃-D-alkyl-G;
l) -L₃-D-aryl-G;
m) -L₃-D-heteroaryl-G;
n) -L₃-D-cycloalkyl-G;
o) -L₃-D-heterocyclyl-G;
p) -L₃-D-arylene-alkyl-G;
q) -L₃-D-alkylene-arylene-alkyl-G; or
r) -L₃-D-alkylene-aryl-G;
wherein
L₃ is an -alkylene, -alkenylene, or alkynylene;
D is a direct bond, —CH₂—, —O—, —N(R₅)—, —C(O)—, —CON(R₅)—, —N(R₆)C(O)—, —N(R₆)CON(R₅)—, —N(R₅)C(O)O—, —OC(O)N(R₅)—, —N(R₅)SO₂—, —SO₂N(R₅)—, —C(O)—O—, —O—C(O)—, —S—, —S(O)—, —S(O₂)—, or —N(R₅)SO₂N(R₆)—, —N═N—, or —N(R₅)—N(R₆)—;
wherein
R₅ and R₆ are independently selected from the group consisting of: -hydrogen, -alkyl, -aryl, -arylene-alkyl, -alkylene-aryl, and -alkylene-arylene-alkyl; and
G is hydrogen, —CN, —SO₃H, —P(O)(OH)₂, —P(O)(O-alkyl)(OH), —CO₂H, —CO₂-alkyl, an acid isostere, —NR₇R₈, or 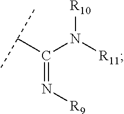
wherein
R₇ and R₈ are independently selected from the group consisting of: hydrogen, -alkyl, -L₄-E-alkyl, -L₄-E-aryl, —C(O)-alkyl, —C(O)-aryl, —SO₂-alkyl, —SO₂-aryl, and 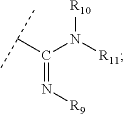
wherein
R₉, R₁₀, and R₁₁ are independently selected from the group consisting of: -hydrogen, -alkyl, -aryl, -arylene-alkyl, -alkylene-aryl, and -alkylene-arylene-alkyl;
L₄ is a direct bond, -alkylene, -alkenylene, or -alkynylene;
E is a direct bond, —CH₂—, —O—, —N(R₁₂)—, —C(O)—, —CON(R₁₂)—, —N(R₁₂)C(O)—, —N(R₁₂)CON(R₁₃)—, —N(R₁₂)C(O)O—, —OC(O)N(R₁₂)—, —N(R₁₂)SO₂—, —SO₂N(R₁₂)—, —C(O)—O—, —O—C(O)—, —S—, —S(O)—, —S(O₂)—, —N(R₁₂)SO₂N(R₁₃)—, —N═N—, or —N(R₁₂)—N(R₁₃)—
wherein
R₁₂ and R₁₃ are independently selected from the group consisting of: -hydrogen, -alkyl, -aryl, -arylene-alkyl, -alkylene-aryl, and -alkylene-arylene-alkyl;
R₁ is
a) -hydrogen;
b) -fluoro;
c) -chloro;
d) -bromo;
e) -iodo;
f) -cyano;
g) -alkyl;
h) -aryl;
i)-alkylene-aryl;
j) -heteroaryl;
k) -alkylene-heteroaryl;
l) -cycloalkyl;
m) -alkylene-cycloalkyl
n) -heterocyclyl; or
o) -alkylene-heterocyclyl;
L₁ is selected from the group consisting of: 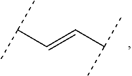
wherein
Ar₁ is a phenyl group optionally substituted 1 to 5 times wherein the substituents are independently selected from the group consisting of
a) -fluoro;
b) -chloro;
c) -bromo;
d) -iodo;
e) -cyano;
f) -nitro;
g) -perfluoroalkyl;
h) -J-R₁₄;
i) -alkyl;
j) -aryl;
k) -heteroaryl;
l) -heterocyclyl;
m) -cycloalkyl;
n) -L₅-aryl;
o) -L₅-arylene-aryl;
p) -L₅-arylene-alkyl;
q) -arylene-alkyl;
r) -arylene-arylene-alkyl;
s)-J-alkyl;
t) -J-aryl;
u) -J-alkylene-aryl;
v) -J-arylene-alkyl;
w) -J-alkylene-arylene-aryl;
x) -J-arylene-arylene-aryl;
y) -J-alkylene-arylene-alkyl;
z) -L₅-J-alkylene-aryl;
aa) -arylene-J-alkyl;
bb) -L₅-J-aryl;
cc) -L₅-J-heteroaryl;
dd) -L₅-J-cycloalkyl;
ee) -L₅-J-heterocyclyl;
ff) -L₅-J-arylene-alkyl;
gg) -L₅-J-alkylene-arylene-alkyl;
hh) -L₅-J-alkyl;
ii) -L₅-J-R₁₄; and
jj) -arylene-J-R₁₄;
wherein
L₅ is a direct bond, -alkylene, -alkenylene, or -alkynylene;
J is a direct bond, —CH₂—, —O—, —N(R₁₅)—, —C(O)—, —CON(R₁₅)—, —N(R₁₅)C(O)—, —N(R₁₅)CON(R₁₆)—, —N(R₁₅)C(O)O—, —OC(O)N(R₁₅)—, —N(R₁₅)SO₂—, —SO₂N(R₁₅)—, —C(O)—O—, —O—C(O)—, —S—, —S(O)—, —S(O₂)—, —N(R₁₅)SO₂N(R₁₆)—, —N═N—, or —N(R₁₅)—N(R₁₆)—,
wherein
R₁₄, R₁₅, and R₁₆ are independently selected from a group consisting of: -hydrogen, -alkyl, -aryl, -arylene-alkyl, -alkylene-aryl, and -alkylene-arylene-alkyl
Ar₂ is a phenyl group optionally substituted 1 to 5 times wherein the substituents are independently selected from the group consisting of
a) -fluoro;
b) -chloro;
c) -bromo;
d) -iodo;
e) -cyano;
f) -nitro;
g) -perfluoroalkyl;
h) -Q-R₁₇;
i) -alkyl;
j) -aryl;
k) -heteroaryl;
l) -heterocyclyl;
m) -cycloalkyl;
n) -L₆-aryl;
o) -L₆-arylene-aryl;
p) -L₆-arylene-alkyl;
q) -arylene-alkyl;
r) -arylene-arylene-alkyl;
s) -Q-alkyl;
t) -Q-aryl;
u) -Q-alkylene-aryl;
v) -Q-arylene-alkyl;
w) -Q-alkylene-arylene-aryl;
x) -Q-arylene-arylene-aryl;
y)-Q-alkylene-arylene-alkyl;
z) -L₆-Q-alkylene-aryl;
aa) -arylene-Q-alkyl;
bb) -L₆-Q-aryl;
cc) -L₆-Q-heteroaryl;
dd) -L₆-Q-cycloalkyl;
ee) -L₆-Q-heterocyclyl;
ff) -L₆-Q-arylene-alkyl;
gg) -L₆-Q-alkylene-arylene-alkyl;
hh) -L₆-Q-alkyl;
ii)-L₆-Q-alkylene-aryl-R₁₇;
jj) -L₆-Q-alkylene-heteroaryl-R₁₇;
kk) -arylene-Q-alkylene-R₁₇;
ll) -heteroarylene-Q-alkylene-R₁₇;
mm) -L₆-Q-aryl-R₁₇;
nn) -L₆-Q-heteroarylene-R₁₇;
oo) -L₆-Q-heteroaryl-R₁₇;
pp) -L₆-Q-cycloalkyl-R₁₇;
qq) -L₆-Q-heterocyclyl-R₁₇;
rr) -L₆-Q-arylene-alkyl-R₁₇;
ss) -L₆-Q-heteroarylene-alkyl-R₁₇;
tt) -L₆-Q-alkylene-arylene-alkyl-R₁₇;
uu) -L₆-Q-alkylene-heteroarylene-alkyl-R₁₇;
vv) -L₆-Q-alkylene-cycloalkylene-alkyl-R₁₇;
ww) -L₆-Q-alkylene-heterocyclylene-alkyl-R₁₇;
xx) -L₆-Q-alkyl-R₁₇;
yy) -L₆-Q-R₁₇;
zz) -arylene-Q-R₁₇;
aaa) -heteroarylene-Q-R₁₇;
bbb) -heterocyclylene-Q-R₁₇;
ccc) -Q-alkylene-R₁₇;
ddd) -Q-arylene-R₁₇;
eee) -Q-heteroarylene-R₁₇;
fff) -Q-alkylene-arylene-R₁₇;
ggg) -Q-alkylene-heteroarylene-R₁₇;
hhh) -Q-heteroarylene-alkylene-R₁₇;
iii) -Q-arylene-alkylene-R₁₇;
jjj) -Q-cycloalkylene-alkylene-R₁₇;
kkk) -Q-heterocyclylene-alkylene-R₁₇
lll)-Q-alkylene-arylene-alkyl-R₁₇;
mmm) -Q-alkylene-heteroarylene-alkyl-R₁₇;
nnn) 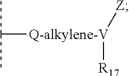
and
ooo) 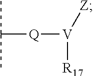
wherein
L₆ is a direct bond, -alkylene, -alkenylene, or -alkynylene;
Q is a direct bond, —CH₂—, —O—, —N(R₁₈)—, —C(O)—, —CON(R₁₈)—, —N(R₁₈)C(O)—, —N(R₁₈)CON(R₁₉)—, —N(R₁₈)C(O)O—, —OC(O)N(R₁₈)—, —N(R₁₈)SO₂—, —SO₂N(R₁₈)—, —C(O)—O—, —O—C(O)—, —S—, —S(O)—, —S(O₂)—, —N(R₁₅)SO₂N(R₁₉)—, —N═N—, or —N(R₁₈)—N(R₁₉)—;
wherein
R₁₈ and R₁₉ are independently selected from the group consisting of: -hydrogen, -alkyl, -aryl, -arylene-alkyl, -alkylene-aryl, and -alkylene-arylene-alkyl;
V is 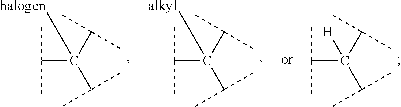
Z is hydrogen, -alkylene-aryl, -alkyl, -aryl, -heteroaryl, -heterocyclyl, -cycloalkyl, -alkylene-heteroaryl, or -alkylene-cycloalkyl;
R₁₇ is —SO₃H, —P(O)(OH)₂, —P(O)(O-alkyl)(OH), —CO₂H, —CO₂-alkyl, an acid isostere, hydrogen, -alkyl, -aryl, -arylene-alkyl, -alkylene-aryl, or -alkylene-arylene-alkyl L₂ is a direct bond,
T is a phenyl group optionally substituted 1 to 5 times wherein the substituents are independently selected from the group consisting of
a) -fluoro;
b) -chloro;
c) -bromo;
d) -iodo;
e) -cyano;
f) -nitro;
g) -perfluoroalkyl;
h) -U-R₂₂;
i) -alkyl;
j) -aryl;
k) -heteroaryl;
l) -heterocyclyl;
m) -cycloalkyl;
n) -L₇-aryl;
o) -L₇-arylene-aryl;
p) -L₇-arylene-alkyl;
q) -arylene-alkyl;
r) -arylene-arylene-alkyl;
s) -U-alkyl;
t) -U-aryl;
u) -U-alkylene-aryl;
v) -U-arylene-alkyl;
w) -U-alkylene-arylene-aryl;
x) -U-arylene-arylene-aryl;
y) -U-alkylene-arylene-alkyl;
z) -L₇-U-alkylene-aryl;
aa) -arylene-U-alkyl;
bb) -L₇-U-aryl;
cc) -L₇-U-heteroaryl;
dd) -L₇-U-cycloalkyl;
ee) -L₇-U-heterocyclyl;
ff) -L₇-U-arylene-alkyl;
gg) -L₇-U-alkylene-arylene-alkyl;
hh) -L₇-U-alkyl;
ii) -L₇-U-alkylene-aryl-R₂₂;
jj) -L₇-U-alkylene-heteroaryl-R₂₂;
kk) -arylene-U-alkylene-R₂₂;
ll) -heteroarylene-U-alkylene-R₂₂;
mm) -L₇-U-aryl-R₂₂;
nn) -L₇-U-heteroarylene-R₂₂;
oo) -L₇-U-heteroaryl-R₂₂;
pp) -L₇-U-cycloalkyl-R₂₂;
qq) -L₇-U-heterocyclyl-R₂₂;
rr) -L₇-U-arylene-alkyl-R₂₂;
ss) -L₇-U-heteroarylene-alkyl-R₂₂;
tt) -L₇-U-alkylene-arylene-alkyl-R₂₂;
uu) -L₇-U-alkylene-heteroarylene-alkyl-R₂₂;
vv) -L₇-Q-alkylene-cycloalkylene-alkyl-R₂₂;
ww) -L₇-Q-alkylene-heterocyclylene-alkyl-R₂₂;
xx) -L₇-U-alkyl-R₂₂;
yy) -L₇-U-R₂₂;
zz)-arylene-U-R₂₂;
aaa) -heteroarylene-U-R₂₂;
bbb) -heterocyclylene-U-R₂₂;
ccc) -U-alkylene-R₂₂;
ddd) -U-arylene-R₂₂;
eee) -U-heteroarylene-R₂₂;
fff) -U-alkylene-arylene-R₂₂;
ggg) -U-alkylene-heteroarylene-R₂₂;
hhh) -U-heteroarylene-alkylene-R₂₂;
iii) -U-arylene-alkylene-R₂₂;
jjj) -U-cycloalkylene-alkylene-R₂₂;
kkk) -U-heterocyclylene-alkylene-R₂₂;
lll) -U-alkylene-arylene-alkyl-R₂₂;
mmm) -U-alkylene-heteroarylene-alkyl-R₂₂;
nnn) 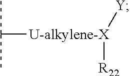
and
ooo) 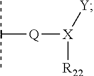
wherein
L₇ is a direct bond, -alkylene, -alkenylene, or -alkynylene;
U is a direct bond, —CH₂—, —O—, —N(R₂₃)—, —C(O)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —C(O)—O—, —O—C(O)—, —S—, —S(O)—, —S(O₂)—, —N(R₂₃)SO₂N(R₂₄)—, —N═N—, or —N(R₂₃)—N(R₂₄)—;
wherein
R₂₃ and R₂₄ are independently selected from the group consisting of: -hydrogen, -alkyl, -aryl, -arylene-alkyl, -alkylene-aryl, and -alkylene-arylene-alkyl;
X is 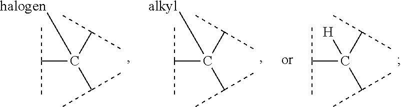
Y is hydrogen, -alkylene-aryl, -alkyl, -aryl, -heteroaryl, -heterocyclyl, -cycloalkyl, -alkylene-heteroaryl, or -alkylene-cycloalkyl; and
R₂₂ is —SO₃H, —P(O)(OH)₂, —P(O)(O-alkyl)(OH), —CO₂H, —CO₂-alkyl, an acid isostere, -hydrogen, -alkyl, -aryl, -arylene-alkyl, -alkylene-aryl, or -alkylene-arylene-alkyl;
or a pharmaceutically acceptable solvate of any of the foregoing.

65. The compound of claim 64, wherein W is —N(R₂)—, wherein R₂ is alkyl, or -L₃-D-alkylene-aryl, wherein L₃ is alkylene, and D is —CO(NR₅)—, wherein R₅ is hydrogen.

66. The compound of claim 64, wherein R₁ is hydrogen or aryl.

67. The compound of claim 64, wherein R₁ is hydrogen.

68. The compound of claim 64, wherein Ar₁ is a phenyl group having 1 to 5 substituents, wherein the substituents are independently selected from the group consisting of:
a) -fluoro;
b) -chloro;
c) -bromo;
d) -iodo;
e) -cyano;
f) -nitro;
g) -perfluoroalkyl;
h) -J-R₁₄;
i)-alkyl;
j) -aryl;
k) -heteroaryl;
l) -heterocyclyl;
m) -cycloalkyl;
n) -L₅-aryl;
o) -L₅-arylene-aryl;
p) -L₅-arylene-alkyl;
q) -arylene-alkyl;
r) -arylene-arylene-alkyl;
s) -J-alkyl;
t) -J-aryl;
u) -J-alkylene-aryl;
v) -J-arylene-alkyl;
w) -J-alkylene-arylene-aryl;
x) -J-arylene-arylene-aryl;
y) -J-alkylene-arylene-alkyl;
z) -L₅-J-alkylene-aryl;
aa) -arylene-J-alkyl;
bb) -L₅-J-aryl;
cc) -L₅-J-heteroaryl;
dd) -L₅-J-cycloalkyl;
ee) -L₅-J-heterocyclyl;
ff) -L₅-J-arylene-alkyl;
gg) -L₅-J-alkylene-arylene-alkyl;
hh) -L₅-J-alkyl;
ii) -L₅-J-R₁₄; and
jj) -arylene-J-R₁₄;
wherein
L₅ is a direct bond, -alkylene, -alkenylene, or -alkynylene;
J is a direct bond, —CH₂—, —O—, —N(R₁₅)—, —C(O)—, —CON(R₁₅)—, —N(R₁₅)C(O)—, —N(R₁₅)CON(R₁₆)—, —N(R₁₅)C(O)O—, —OC(O)N(R₁₅)—, —N(R₁₅)SO₂—, —SO₂N(R₁₅)—, —C(O)—O—, —O—C(O)—, —S—, —S(O)—, —S(O₂)—, —N(R₁₅)SO₂N(R₁₆)—, —N═N—, or —N(R₁₅)—N(R₁₆)—,
wherein
R₁₄, R₁₅, and R₁₆ are independently selected from a group consisting of: -hydrogen, -alkyl, -aryl, -arylene-alkyl, -alkylene-aryl, and -alkylene-arylene-alkyl.

69. The compound of claim 64, wherein Ar₁ is a phenyl group optionally substituted 1 to 5 times, wherein the substituents are independently selected from the group consisting of:
a) -fluoro;
b) -chloro;
c) -bromo;
d) -iodo;
e) -cyano;
f) -nitro; and
g) -aryl.

70. The compound of claim 64 or a pharmaceutically acceptable salt or solvate thereof, wherein Ar₁ is a phenyl group substituted 1 to 5 times, wherein the substituents are selected from the group consisting of: -chloro and -fluoro.

71. The compound of claim 64, wherein Ar₂ is a phenylene group having 1 to 5 substituents, wherein the substituents are independently selected from the group consisting of:
a) -fluoro;
b) -chloro;
c) -bromo;
d) -iodo;
e) -cyano;
f) -nitro;
g) -perfluoroalkyl;
h) -Q-R₁₇;
i)-alkyl;
j) -aryl;
k) -heteroaryl;
l) -heterocyclyl;
m) -cycloalkyl;
n) -L₆-aryl;
o) -L₆-arylene-aryl;
p) -L₆-arylene-alkyl;
q) -arylene-alkyl;
r) -arylene-arylene-alkyl;
s)-Q-alkyl;
t) -Q-aryl;
u) -Q-alkylene-aryl;
v) -Q-arylene-alkyl;
w) -Q-alkylene-arylene-aryl;
x) -Q-arylene-arylene-aryl;
y) -Q-alkylene-arylene-alkyl;
z) -L₆-Q-alkylene-aryl;
aa) -arylene-Q-alkyl;
bb) -L₆-Q-aryl;
cc) -L₆-Q-heteroaryl;
dd) -L₆-Q-cycloalkyl;
ee) -L₆-Q-heterocyclyl;
ff) -L₆-Q-arylene-alkyl;
gg) -L₆-Q-alkylene-arylene-alkyl;
hh) -L₆-Q-alkyl;
ii) -L₆-Q-alkylene-aryl-R₁₇;
jj) -L₆-Q-alkylene-heteroaryl-R₁₇;
kk) -arylene-Q-alkylene-R₁₇;
ll) -heteroarylene-Q-alkylene-R₁₇;
mm) -L₆-Q-aryl-R₁₇;
nn) -L₆-Q-heteroarylene-R₁₇;
oo) -L₆-Q-heteroaryl-R₁₇;
pp) -L₆-Q-cycloalkyl-R₁₇;
qq) -L₆-Q-heterocyclyl-R₁₇;
rr) -L₆-Q-arylene-alkyl-R₁₇;
ss) -L₆-Q-heteroarylene-alkyl-R₁₇;
tt) -L₆-Q-alkylene-arylene-alkyl-R₁₇;
uu) -L₆-Q-alkylene-heteroarylene-alkyl-R₁₇;
vv) -L₆-Q-alkylene-cycloalkylene-alkyl-R₁₇;
ww) -L₆-Q-alkylene-heterocyclylene-alkyl-R₁₇;
xx) -L₆-Q-alkyl-R₁₇;
yy) -L₆-Q-R₁₇;
zz) -arylene-Q-R₁₇;
aaa) -heteroarylene-Q-R₁₇;
bbb) -heterocyclylene-Q-R₁₇;
ccc) -Q-alkylene-R₁₇;
ddd) -Q-arylene-R₁₇;
eee) -Q-heteroarylene-R₁₇;
fff) -Q-alkylene-arylene-R₁₇;
ggg) -Q-alkylene-heteroarylene-R₁₇;
hhh) -Q-heteroarylene-alkylene-R₁₇;
iii) -Q-arylene-alkylene-R₁₇;
jjj) -Q-cycloalkylene-alkylene-R₁₇;
kkk) -Q-heterocyclylene-alkylene-R₁₇
lll) -Q-alkylene-arylene-alkyl-R₁₇;
mmm) -Q-alkylene-heteroarylene-alkyl-R₁₇;
nnn) 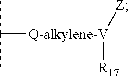
and
ooo) 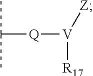
wherein
L₆ is a direct bond, -alkylene, -alkenylene, or -alkynylene;
Q is a direct bond, —CH₂—, —O—, —N(R₁₈)—, —C(O)—, —CON(R₁₈)—, —N(R₁₈)C(O)—, —N(R₁₈)CON(R₁₉)—, —N(R₁₈)C(O)O—, —OC(O)N(R₁₈)—, —N(R₁₈)SO₂—, —SO₂N(R₁₈)—, —C(O)—O—, —O—C(O)—, —S—, —S(O)—, —S(O₂)—, —N(R₁₅)SO₂N(R₁₉)—, —N═N—, or —N(R₁₈)—N(R₁₉)—;
wherein
R₁₈ and R₁₉ are independently selected from the group consisting of: -hydrogen, -alkyl, -aryl, -arylene-alkyl, -alkylene-aryl, and -alkylene-arylene-alkyl;
V is 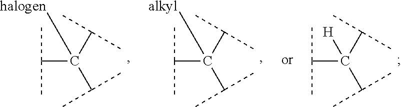
Z is hydrogen, -alkylene-aryl, -alkyl, -aryl, -heteroaryl, -heterocyclyl, -cycloalkyl, -alkylene-heteroaryl, or -alkylene-cycloalkyl;
R₁₇ is —SO₃H, —P(O)(OH)₂, —P(O)(O-alkyl)(OH), —CO₂H, —CO₂-alkyl, an acid isostere, hydrogen, -alkyl, -aryl, -arylene-alkyl, -alkylene-aryl, or -alkylene-arylene-alkyl.

72. The compound of claim 64, wherein Ar₂ is a phenyl group optionally substituted 1 to 5 times, wherein the substituents are independently selected from the group consisting of:
a) -fluoro;
b) -chloro;
c) -bromo;
d) -iodo;
e) -Q-R₁₇;
f) -alkyl;
g) -aryl;
h) -arylene-alkyl;
i) -Q-alkyl; and
j) -arylene-Q-alkyl;
wherein
Q is —CH₂—, —O—, —C(O)—, or —C(O)—O—, and
R₁₇ is: -hydrogen, -alkyl, -aryl, —CO₂H, or an acid isostere.

73. The compound of claim 64, wherein Ar₂ is a phenyl group substituted 1 to 5 times, wherein the substituents are independently selected from the group consisting of:
a) -fluoro;
b) -chloro;
c) -bromo;
d) -iodo;
e) -Q-R₁₇;
f) -alkyl;
g) -phenyl;
h) -phenylene-alkyl;
i) -Q-alkyl; and
j) -phenylene-Q-alkyl;
wherein
Q is: —CH₂—, —O—, —C(O)—, or —C(O)—O—, and
R₁₇ is: -hydrogen, -alkyl, -phenyl, or —CO₂H.

74. The compound of claim 64, wherein T is an aryl group having 1 to 5 substituents, wherein the substituents are independently selected from the group consisting of:
a) -fluoro;
b) -chloro;
c) -bromo;
d) -iodo;
e) -cyano;
f) -nitro;
g) -perfluoroalkyl;
h) -U-R₂₂;
i) -alkyl;
j) -aryl;
k) -heteroaryl;
l) -heterocyclyl;
m) -cycloalkyl;
n) -L₇-aryl;
o) -L₇-arylene-aryl;
p) -L₇-arylene-alkyl;
q) -arylene-alkyl;
r) -arylene-arylene-alkyl;
s) -U-alkyl;
t) -U-aryl;
u) -U-alkylene-aryl;
v) -U-arylene-alkyl;
w) -U-alkylene-arylene-aryl;
x) -U-arylene-arylene-aryl;
y) -U-alkylene-arylene-alkyl;
z) -L₇-U-alkylene-aryl;
aa) -arylene-U-alkyl;
bb) -L₇-U-aryl;
cc) -L₇-U-heteroaryl;
dd) -L₇-U-cycloalkyl;
ee) -L₇-U-heterocyclyl;
ff) -L₇-U-arylene-alkyl;
gg) -L₇-U-alkylene-arylene-alkyl;
hh) -L₇-U-alkyl;
ii) -L₇-U-alkylene-aryl-R₂₂;
jj) -L₇-U-alkylene-heteroaryl-R₂₂;
kk) -arylene-U-alkylene-R₂₂;
ll) -heteroarylene-U-alkylene-R₂₂;
mm) -L₇-U-aryl-R₂₂;
nn) -L₇-U-heteroarylene-R₂₂;
oo) -L₇-U-heteroaryl-R₂₂;
pp) -L₇-U-cycloalkyl-R₂₂;
qq) -L₇-U-heterocyclyl-R₂₂;
rr) -L₇-U-arylene-alkyl-R₂₂;
ss) -L₇-U-heteroarylene-alkyl-R₂₂;
tt) -L₇-U-alkylene-arylene-alkyl-R₂₂;
uu) -L₇-U-alkylene-heteroarylene-alkyl-R₂₂;
vv) -L₇-Q-alkylene-cycloalkylene-alkyl-R₂₂;
ww) -L₇-Q-alkylene-heterocyclylene-alkyl-R₂₂;
xx) -L₇-U-alkyl-R₂₂;
yy) -L₇-U-R₂₂;
zz) -arylene-U-R₂₂;
aaa) -heteroarylene-U-R₂₂;
bbb) -heterocyclylene-U-R₂₂;
ccc) -U-alkylene-R₂₂;
ddd) -U-arylene-R₂₂;
eee) -U-heteroarylene-R₂₂;
fff) -U-alkylene-arylene-R₂₂;
ggg) -U-alkylene-heteroarylene-R₂₂;
hhh) -U-heteroarylene-alkylene-R₂₂;
iii) -U-arylene-alkylene-R₂₂;
jjj) -U-cycloalkylene-alkylene-R₂₂;
kkk) -U-heterocyclylene-alkylene-R₂₂;
lll) -U-alkylene-arylene-alkyl-R₂₂;
mmm) -U-alkylene-heteroarylene-alkyl-R₂₂;
nnn) 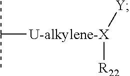
and
ooo) 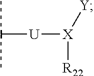
wherein
L₇ is a direct bond, -alkylene, -alkenylene, or -alkynylene;
U is a direct bond, —CH₂—, —O—, —N(R₂₃)—, —C(O)—, —CON(R₂₃)—, —N(R₂₃)C(O)—, —N(R₂₃)CON(R₂₄)—, —N(R₂₃)C(O)O—, —OC(O)N(R₂₃)—, —N(R₂₃)SO₂—, —SO₂N(R₂₃)—, —C(O)—O—, —O—C(O)—, —S—, —S(O)—, —S(O₂)—, —N(R₂₃)SO₂N(R₂₄)—, —N═N—, or —N(R₂₃)—N(R₂₄)—;
wherein
R₂₃ and R₂₄ are independently selected from the group consisting of: -hydrogen, -alkyl, -aryl, -arylene-alkyl, -alkylene-aryl, and -alkylene-arylene-alkyl;
X is 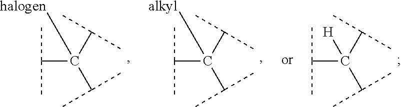
Y is hydrogen, -alkylene-aryl, -alkyl, -aryl, -heteroaryl, -heterocyclyl, -cycloalkyl, -alkylene-heteroaryl, or -alkylene-cycloalkyl;
R₂₂ is —SO₃H, —P(O)(OH)₂, —P(O)(O-alkyl)(OH), —CO₂H, —CO₂-alkyl, an acid isostere, -hydrogen, -alkyl, -aryl, -arylene-alkyl, -alkylene-aryl, or -alkylene-arylene-alkyl.

75. The compound of claim 64, wherein T is an aryl group substituted by -U-alkylene-R₂₂, wherein U is —O— or a direct bond, and R₂₂ is —CO₂H or an acid isostere.

76. The compound of claim 74, wherein a and b are equal to zero;
L₁ is 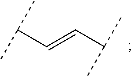
Ar₂ is a phenylene group optionally substituted 1 time with a group consisting of: -Q-alkyl, wherein Q is —O—;
L₂ is a direct bond; and
T is an aryl group substituted with at least one substituent selected from the group consisting of:
a) -U-R₂₂;
b) -U-alkylene-arylene-R₂₂;
c) -U-alkylene-R₂₂;
d) -U-arylene-R₂₂;
e) -U-arylene-R₂₂ wherein the arylene is substituted with at least one of a halogen, methanesulfonylamino, or trifluoromethanesulfonylamino group;
f) -U-arylene wherein the arylene is substituted with at least one trifluoromethanesulfonylamino group;
g) —R₂₂; and
h) -halogen;
wherein R₂₂ is —CO₂H or an acid isotere.

77. The compound of claim 64, wherein a and b are equal to zero;
R₁ is hydrogen;
W is —N(R₂)—, wherein R₂ is alkyl; and
Ar₁ is phenyl substituted 2 times wherein the substituent groups are -chloro.

78. The compound of claim 64, wherein W is —N(R₂)—, wherein
R₂ is -alkylene-arylene-G,
wherein
G is —CN, —SO₃H, —P(O)(OH)₂, —P(O)(O-alkyl)(OH), —CO₂H, —CO₂-alkyl, or an acid isostere.

79. The compound of claim 64, wherein a and b are equal to 0, and T, L₂, Ar₂, and L₁ together form a group selected from a group consisting of:
(E)-2-(1,1′-biphenyl-4-yl)vinyl,
(E)-2-(4′-methoxy-1,1′-biphenyl-4-yl)vinyl,
(E)-2-(3′-methoxy-1,1′-biphenyl-4-yl)vinyl,
(E)-2-(4′-carboxymethyloxy-1,1′-biphenyl-4-yl)vinyl,
(E)-2-(4′-(3-methoxycarbonyl-1-propyloxy)-1,1′-biphenyl-4-yl)vinyl,
(E)-2-(4′-(3-carboxy-1-propyloxy)-1,1′-biphenyl-4-yl)vinyl,
(E)-2-(4′-phenoxy-1,1′-biphenyl-4-yl)vinyl, and
(E)-2-(4′-benzyloxy-1,1′-biphenyl-4-yl)vinyl.

80. The compound of claim 64, wherein Ar₁ is: 2,4-dichlorophenyl.

81. The compound of claim 64, where the compound is a compound selected from the group consisting of:
4-(4′-{2-[4-(2,4-dichloro-phenyl)-1-ethyl-1H-imidazol-2-yl]-(E)-vinyl}-3-fluoro-biphenyl-4-yloxymethyl)-benzoic acid;
4-[4′-(2-{4-(2,4-dichloro-phenyl)-1-[(1-naphthalen-1-yl-ethylcarbamoyl)-methyl]1H-imidazol-2-yl}-(E)-vinyl)-biphenyl-4-yloxy]-butyric acid;
4-(4′-{2-[4-(2,4-dichloro-phenyl)-1-ethyl-1H-imidazol-2-yl]-(E)-vinyl}-biphenyl-4-yloxy)-butyric acid;
5-(4′-{2-[4-(2,4-dichloro-phenyl)-1-methyl-1H-imidazol-2-yl]-(E)-vinyl}-biphenyl-4-yloxy)-pentanoic acid
2-bromo-4-(4′-{2-[4-(2,4-dichloro-phenyl)-1-methyl-1H-imidazol-2-yl]-(E)-vinyl}-biphenyl-4-yloxy)-benzoic acid;
4-(4′-{2-[4-(2,4-dichloro-phenyl)-1-ethyl-1H-imidazol-2-yl]-(E)-vinyl}-biphenyl-4-yloxymethyl)-benzoic acid;
4-(4′-{2-[4-(2,4-dichloro-phenyl)-1-ethyl-1H-imidazol-2-yl]-(E)-vinyl}-biphenyl-4-yloxy)-benzoic acid;
2-bromo-4-(4′-{2-[4-(2,4-dichloro-phenyl)-1-ethyl-1H-imidazol-2-yl]-(E)-vinyl}-biphenyl-4-yloxy)-benzoic acid;
4-(4′-{2-[4-(2,4-dichloro-phenyl)-1-ethyl-1H-imidazol-2-yl]-(E)-vinyl}-biphenyl-4-yloxy)-3-methanesulfonylamino-benzoic acid;
4-(4′-{2-[4-(2,4-dichloro-phenyl)-1-ethyl-1H-imidazol-2-yl]-(E)-vinyl}-biphenyl-4-yloxy)-3-trifluoromethanesulfonyl-amino-benzoic acid;
5-(4′-{2-[4-(2,4-dichloro-phenyl)-1-ethyl-1H-imidazol-2-yl]-(E)-vinyl}-biphenyl-4-yloxy)-2-methanesulfonylamino-benzoic acid;
5-(4′-{2-[4-(2,4-dichloro-phenyl)-1-ethyl-1H-imidazol-2-yl]-(E)-vinyl}-biphenyl-4-yloxy)-2-trifluoromethane-sulfonylamino-benzoic acid; and
4-(4′-{2-[4-(2,4-Dichloro-phenyl)-1-ethyl-1H-imidazol-2-yl]-(E)-vinyl}-biphenyl-4-yloxy)-butyric acid 2,2-dimethyl-propionyloxymethyl ester,
or a pharmaceutically acceptable salt thereof, or a pharmaceutically acceptable solvate of any of the foregoing.

82. A pharmaceutical composition comprising a compound of claim 64.

83. The pharmaceutical composition of claim 82, wherein said pharmaceutical composition is a topical formulation.
